(19) United States
(12) Patent Application Publication
(10) Pub. No.: US 2026/0365606 A1
(43) Pub. Date: Nov. 19, 2026

(54) TROPOLONE DERIVATIVES AND TAUTOMERS THEREOF FOR IRON REGULATION IN ANIMALS

(71) Applicant: Kinesid Therapeutics, Inc., Champaign, IL (US)

(72) Inventors: Jianhua Chao, Palo Alto, CA (US); Stanley J. Hollenbach, Raleigh, NC (US); Michael Christopher Holmes, Oakland, CA (US); Douglas M. Matje, San Francisco, CA (US); Nicholas M. Snead, San Francisco, CA (US); Gustave Bergnes, Pacifica, CA (US); Kevin T. Mellem, Redwood City, CA (US); David John Morgans, JR., Los Altos, CA (US)

(21) Appl. No.: 19/727,889

(22) Filed: Jul. 1, 2026

Related U.S. Application Data
(62) Division of application No. 17/769,518, filed on Oct. 16, 2020.
(60) Provisional application No. 62/916,018, filed on Oct. 16, 2019.

(51) Int. Cl. C07C 49/717; A61P 7/06; C07C 49/753; C07C 323/22; C07D 211/32; C07D 305/06; C07D 305/08; C07D 307/12; C07D 307/20; C07D 307/93; C07D 309/06; C07D 311/94; C07D 333/22; C07D 493/08

(52) U.S. Cl. CPC C07C 49/717; A61P 7/06; C07C 49/753; C07C 323/22; C07D 211/32; C07D 305/06; C07D 305/08; C07D 307/12; C07D 307/20; C07D 307/93; C07D 309/06; C07D 311/94; C07D 333/22; C07D 493/08; C07C 2601/02; C07C 2601/04; C07C 2601/08; C07C 2601/18; C07C 2602/18; C07C 2602/50

(57) ABSTRACT

Disclosed are a series of compounds or their tautomers having a general structure represented by Formula (la), (lb), (Ila), (lIb), or (lIc) and pharmaceutically acceptable salts thereof. The present disclosure also relates to pharmaceutical compositions comprising said compounds or tautomers. The present disclosure further relates to a method of treating a disease or condition associated with iron dysregulation or dysfunctional iron homeostasis comprising administering to a subject in need thereof a therapeutically effective amount of Formula (la), (lb), (Ila), (lIb), or (lIc) compounds or tautomers.

### RELATED APPLICATION

This application claims the benefit of priority to U.S. Provisional Patent Application No. 62/916,018, filed Oct. 16, 2019.

### FIELD

Provided are compounds, pharmaceutical compositions comprising the compounds, and methods useful for treating a disease or condition associated with iron dysregulation or dysfunctional iron homeostasis.

### BACKGROUND

Iron is an essential element in all living systems. Together with oxygen, it forms the basis of life's energy production engine. Iron also needs to be tightly regulated via the endogenous iron homeostasis and metabolism network in order to maintain iron sufficiency; either iron overload or deficiency can cause damages to the cellular systems. Iron overload can lead to many diseases, including primary hemochromatosis (genetically based) and secondary hemochromatosis (resulting from thalassemia, chronic hepatitis C infection or alcoholic liver disease). Iron deficiency, on the other hand, leads to reduced erythropoiesis which subsequently contributes to anemia. One cause of iron deficiency is malabsorption of iron. Another cause is associated with anemia of inflammation, which reduces the systemic functional iron level. Anemia of inflammation has become a key factor of many systemic chronic disease etiologies and contributes to the disease progression in a few classic chronic systemic inflammatory disorders such as chronic kidney disease, inflammatory bowel disease, chronic heart failure, chronic obstructive pulmonary disease, and even cystic fibrosis (Ganz, T. (2019) N. Eng. J. Med. 381(12):1148-57; Andrews, N.C. (1999) N. Eng. J. Med. 341(26):1986-95).

Regulation of systemic iron homeostasis and metabolism is accomplished by a complex network of sensors, transport proteins, storage proteins, carrier protein, and hormones. Two transport proteins that play a critical role in the maintenance and regulation of iron level are divalent metal transport 1 (DMT1) and ferroportin (Fpn1) (Ganz, T (2019) N. Eng. J. Med. 381(12):1148-57; Nemeth, E. et al. (2014) Hematol. Oncol. Clin. North. Am. 28(4):671-81; Johnson, E. E. et al. (2007) Nutr. Rev. 65(7):341-5)

Dietary iron is absorbed into enterocytes via divalent metal transport 1 (DMT1), which transfers iron (Ferrous) across the apical membrane into the cells. DMT1 transport deficiency has been implicated in the malabsorption of iron, resulting in iron deficiency anemia; such a deficiency can also reduce the effectiveness of oral iron treatment for anemia.

At the center of iron homeostasis are the transporter protein ferroportin (FPN1) and the iron regulatory hormone hepcidin. Ferroportin is the only known cellular iron exporter. It facilitates the export of iron (Ferrous) from storage cells and absorptive cells to the blood including hepatocytes, macrophages in the liver and spleen, and enterocytes. Hepcidin is produced in the liver and its main function is to inhibit ferroportin, reducing its iron transport function. Many inflammatory disorders induce an over production of hepcidin, which leads to abnormal suppression of FPN1 function. As a result, high levels of iron are sequestered in the storage cells, lowering functional iron in blood circulation and contributing to anemia.

Iron transport protein DMT1 deficiency contributes to poor absorption of iron and iron deficiency anemia, while iron transport protein FPN1 deficiency leads to the sequestration of iron in storage cells and reduction of functional iron in circulation, compounding anemia of inflammation. Because iron deficiency anemia and anemia of inflammation commonly coexist, and are the most common anemias worldwide, therapeutics targeting to relieve the deficiency in DMT1 and FPN1 function and achieve normalcy in iron homeostasis can be beneficial to people who are living with said anemias.

Deficiency of frataxin in the central nerve system (“CNS”) will lead to mitochondrial iron overload and the resulting excess iron creates extra ROS, which causes cellular damage and neurodegeneration. Typical disease associated with deficiency of frataxin in CNS is Friedreich's Ataxia.

Lastly, iron dyshomeostasis is a contributor to iron overload in iron-sensitive brain regions, such as basal ganglia, and induces neuronal damage. High iron levels and iron related pathogenic triggers, though not well understood, have been implicated in neurodegenerative disorders including Parkinson's disease (PD) and Alzheimer's disease (AD). Currently available iron chelators have thus far proven ineffective in removing iron from the brains of neurodegenerative disease (e.g. PD) patients. New small molecule therapies that can relieve the brain iron overload condition and restore iron homeostasis are much needed (Ndaylsaba. A. et al. (2019) Front. Neurosci. 13:180; Crichton. R. R (2019) Pharmaceuticals 12:138)

It is unexpectedly found that the Formula Ia and Ib compounds, tautomers thereof, and pharmaceutically acceptable salts of either (for example tropolone derivatives, their tautomers, and pharmaceutically acceptable salts of either) can effectively regulate Fe(III) efflux across liposomes and increase Fe absorption in DMT1-deficient Caco-2 cells. It was also found that Formula Ia and Ib compounds and tautomers thereof, and pharmaceutically acceptable salts of either, demonstrate desirable ADME and DMPK characteristics.

### SUMMARY

In certain embodiments, the present disclosure provides a compound or a tautomer thereof, or a pharmaceutically acceptable salt of either, represented by Formula Ia:

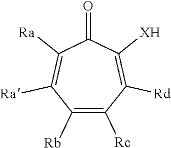

wherein:
   X represents oxygen or sulfur;
   Rₐ represents hydrogen, halo, alkyl, substituted alkyl, heteroalkyl, alkoxy, substituted alkoxy, alkoxyalkyl, substituted alkoxyalkyl, cycloalkyl, substituted cycloalkyl, heterocycloalkyl, substituted heterocycloalkyl, alkenyl, substituted alkenyl, heteroalkenyl, cycloalkenyl, substituted cycloalkenyl, heterocycloalkenyl, substituted heterocycloalkenyl, alkynyl, substituted alkynyl, or heteroalkynyl;
   Rₐ′ represents hydrogen, halo, alkyl, or substituted alkyl; and R_{b}, R_{c}, and R_{d} are independently selected from the group consisting of hydrogen, halo, alkyl, substituted alkyl, heteroalkyl, alkylcycloalkyl, alkylheterocycloalkyl, alkoxy, substituted alkoxy, alkoxyalkyl, substituted alkoxyalkyl, cycloalkyloxy, substituted cycloalkyloxy, heterocycloalkyloxy, substituted heterocycloalkyloxy, cycloalkyl, substituted cycloalkyl, heterocycloalkyl, substituted heterocycloalkyl, alkenyl, substituted alkenyl, heteroalkenyl, cycloalkenyl, substituted cycloalkenyl, heterocycloalkenyl, substituted heterocycloalkenyl, alkynyl, substituted alkynyl, and heteroalkynyl; provided that Rₐ, R_{b}, R_{c}, and R_{d} are not all hydrogen.

In certain embodiments, the present disclosure provides a compound or a tautomer thereof, or a pharmaceutically acceptable salt of either, represented by Formula Ib:

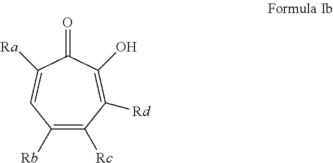

wherein:
   Rₐ represents hydrogen, halo, alkyl, substituted alkyl, heteroalkyl, alkoxy, substituted alkoxy, alkoxyalkyl, substituted alkoxyalkyl, cycloalkyl, substituted cycloalkyl, heterocycloalkyl, substituted heterocycloalkyl, alkenyl, substituted alkenyl, heteroalkenyl, cycloalkenyl, substituted cycloalkenyl, heterocycloalkenyl, substituted heterocycloalkenyl, alkynyl, substituted alkynyl, or heteroalkynyl; and
   R_{b}, R_{c}, and R_{d} are independently selected from the group consisting of hydrogen, halo, alkyl, substituted alkyl, heteroalkyl, alkoxy, substituted alkoxy, alkoxyalkyl, substituted alkoxyalkyl, cycloalkyl, substituted cycloalkyl, heterocycloalkyl, substituted heterocycloalkyl, alkenyl, substituted alkenyl, heteroalkenyl, cycloalkenyl, substituted cycloalkenyl, heterocycloalkenyl, substituted heterocycloalkenyl, alkynyl, substituted alkynyl, and heteroalkynyl;
provided that Rₐ, R_{b}, R_{c}, and R_{d} are not all hydrogen.

In certain embodiments, the present disclosure provides a compound or a tautomer thereof, or a pharmaceutically acceptable salt of either of them, represented by Formula IIa, Formula IIb, or Formula IIc:

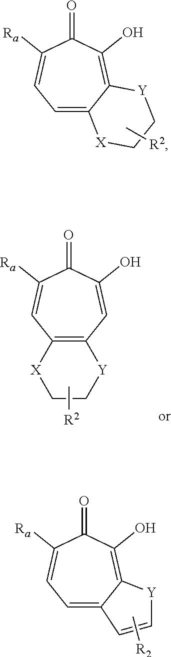

wherein:
   Rₐ represents hydrogen, halo, alkyl, substituted alkyl, heteroalkyl, hydroxy, alkoxy, substituted alkoxy, alkoxyalkyl, substituted alkoxyalkyl, cycloalkyl, substituted cycloalkyl, heterocycloalkyl, substituted heterocycloalkyl, alkenyl, substituted alkenyl, heteroalkenyl, cycloalkenyl, substituted cycloalkenyl, heterocycloalkenyl, substituted heterocycloalkenyl, alkynyl, substituted alkynyl, or heteroalkynyl;
   X and Y independently represent O, S, NH, or CR₅R₆;
   R₂ represents —F, alkyl, haloalkyl, or alkoxy; and
   R₅ and R₆ represent independently for each occurrence H, (C1-C15) alkyl, or substituted (C1-C15)alkyl;
   provided the compound is not

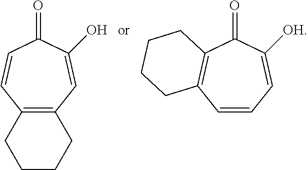 [SMILES: O=c1ccc2c(cc1O)CCCC2 ; "or" ; O=c1c(O)cccc2c1CCCC2]

In certain embodiments, the present disclosure provides a pharmaceutical composition, comprising a compound disclosed herein or its tautomer in a pharmaceutically acceptable carrier.

In certain embodiments, the present disclosure provides a method of treating a disease or condition associated with iron dysregulation or dysfunctional iron homeostasis, comprising administering to a subject in need thereof a therapeutically effective amount of a compound or tautomer disclosed herein.

### DETAILED DESCRIPTION

One aspect of the present disclosure relates to a compound or a tautomer thereof, or a pharmaceutically acceptable salt of either, represented by Formula Ia:

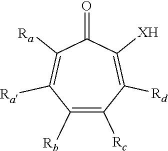

wherein:
   X represents oxygen or sulfur;
   Rₐ represents hydrogen, halo, alkyl, substituted alkyl, heteroalkyl, alkoxy, substituted alkoxy, alkoxyalkyl, substituted alkoxyalkyl, cycloalkyl, substituted cycloalkyl, heterocycloalkyl, substituted heterocycloalkyl, alkenyl, substituted alkenyl, heteroalkenyl, cycloalkenyl, substituted cycloalkenyl, heterocycloalkenyl, substituted heterocycloalkenyl, alkynyl, substituted alkynyl, or heteroalkynyl;
   Rₐ′ represents hydrogen, halo, alkyl, or substituted alkyl; and R_{b}, R_{c}, and R_{d} are independently selected from the group consisting of hydrogen, halo, alkyl, substituted alkyl, heteroalkyl, alkylcycloalkyl, alkylheterocycloalkyl, alkoxy, substituted alkoxy, alkoxyalkyl, substituted alkoxyalkyl, cycloalkyloxy, substituted cycloalkyloxy, heterocycloalkyloxy, substituted heterocycloalkyloxy, cycloalkyl, substituted cycloalkyl, heterocycloalkyl, substituted heterocycloalkyl, alkenyl, substituted alkenyl, heteroalkenyl, cycloalkenyl, substituted cycloalkenyl, heterocycloalkenyl, substituted heterocycloalkenyl, alkynyl, substituted alkynyl, and heteroalkynyl; provided that Rₐ, R_{b}, R_{c}, and R_{d} are not all hydrogen.

One aspect of the present disclosure relates to a compound or a tautomer thereof, or a pharmaceutically acceptable salt of either, represented by Formula Ib:

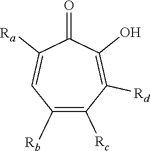

wherein:
   Rₐ represents hydrogen, halo, alkyl, substituted alkyl, heteroalkyl, alkoxy, substituted alkoxy, alkoxyalkyl, substituted alkoxyalkyl, cycloalkyl, substituted cycloalkyl, heterocycloalkyl, substituted heterocycloalkyl, alkenyl, substituted alkenyl, heteroalkenyl, cycloalkenyl, substituted cycloalkenyl, heterocycloalkenyl, substituted heterocycloalkenyl, alkynyl, substituted alkynyl, or heteroalkynyl; and
   R_{b}, R_{c}, and R_{d} are independently selected from the group consisting of hydrogen, halo, alkyl, substituted alkyl, heteroalkyl, alkoxy, substituted alkoxy, alkoxyalkyl, substituted alkoxyalkyl, cycloalkyl, substituted cycloalkyl, heterocycloalkyl, substituted heterocycloalkyl, alkenyl, substituted alkenyl, heteroalkenyl, cycloalkenyl, substituted cycloalkenyl, heterocycloalkenyl, substituted heterocycloalkenyl, alkynyl, substituted alkynyl, and heteroalkynyl;
provided that Rₐ, R_{b}, R_{c}, and R_{d} are not all hydrogen.

Another aspect of the present disclosure relates to a compound or a tautomer thereof, or a pharmaceutically acceptable salt of either of them, represented by Formula IIa, Formula IIb, or Formula IIc:

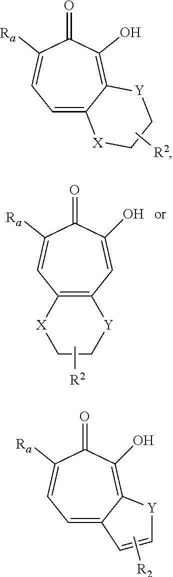

wherein:
   Rₐ represents hydrogen, halo, alkyl, substituted alkyl, heteroalkyl, alkoxy, substituted alkoxy, alkoxyalkyl, substituted alkoxyalkyl, cycloalkyl, substituted cycloalkyl, heterocycloalkyl, substituted heterocycloalkyl, alkenyl, substituted alkenyl, heteroalkenyl, cycloalkenyl, substituted cycloalkenyl, heterocycloalkenyl, substituted heterocycloalkenyl, alkynyl, substituted alkynyl, or heteroalkynyl;
   X and Y independently represent O, S, NH, or CR₅R₆;
   R₂ represents —F, alkyl, haloalkyl, or alkoxy; and
   R₅ and R₆ represent independently for each occurrence H, (C1-C15) alkyl, or substituted (C1-C15)alkyl;
provided the compound is not

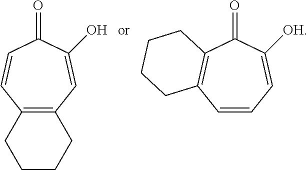 [SMILES: O=c1ccc2c(cc1O)CCCC2 ; "or" ; O=c1c(O)cccc2c1CCCC2]

One aspect of the present disclosure relates to a pharmaceutical composition, comprising a compound of the invention or its tautomer in a pharmaceutically acceptable carrier.

One aspect of the present disclosure relates to a method of treating a disease or condition associated with iron dysregulation or dysfunctional iron homeostasis, comprising administering to a subject in need thereof a therapeutically effective amount of a compound or tautomer of the present disclosure. In one embodiment, disease or condition associated with iron dysregulation or dysfunctional iron homeostasis comprises anemia, iron deficiency anemia, anemia of inflammation, anemia of chronic inflammatory disorders, anemia of chronic kidney disease, anemia in inflammatory bowel disease, chemotherapy-induced anemia, cancer associated anemia, primary hemochromatosis, secondary hemochromatosis, liver failure, Parkinson's disease, or Alzheimer's disease. In one embodiment, the disease or condition associated with iron dysregulation or dysfunctional iron homeostasis is liver failure; and the liver failure is chronic or acute. In one embodiment, the disease or condition associated with iron dysregulation or dysfunctional iron homeostasis is selected from the group consisting of anemia of chronic inflammation, inflammatory bowel disease, chronic heart failure, chronic obstructive pulmonary disease, anemia of chronic kidney disease, rheumatoid arthritis, primary hemochromatosis, secondary hemochromatosis, and lupus. In one embodiment, the disease or condition associated with iron dysregulation or dysfunctional iron homeostasis is a CNS disease, such as Friedreich's Ataxia.

In one aspect, the compounds described herein can be used in the treatment of anemia of inflammation, for example the anemia of inflammation in chronic kidney disease (CKD), either as monotherapy or in combination with standard of cares.

In one aspect, the compounds described herein can be used in the treatment of chemotherapy-induced anemia where a functional iron-deficiency develops in the setting of inflammation that leads to the iron sequestration in macrophages and enterocytes and reduces iron availability for bone marrow in the erythrocyte production.

In one aspect, the compounds described herein can be also applied in the prevention of acute chronic liver failure (ACLF) in patients of cirrhosis where anemia is one of contributing factors.

In another aspect, the compounds described herein may also be applied, in combination with other therapies, in the treatment of neurodegenerative diseases such as Parkinson's (PD) and Alzheimer's disease (AD), where iron dysregulation contributes to the disease progression.

In another aspect, the compounds described herein can be applied to mobilize the extra iron out of the CNS of a subject and, therefore, can be applied to treat Friedreich's Ataxia.

### DEFINITIONS

The term “alkyl” as used herein is a term of art and refers to saturated aliphatic groups, including straight-chain alkyl groups, branched-chain alkyl groups, cycloalkyl (alicyclic) groups, alkyl substituted cycloalkyl groups, and cycloalkyl substituted alkyl groups. In certain embodiments, a straight-chain or branched-chain alkyl has about 30 or fewer carbon atoms in its backbone (e.g., C1-C30 for straight chain, C3-C30 for branched chain), and alternatively, about 20 or fewer. In one embodiment, the term “alkyl” refers to a C1-C10 straight-chain alkyl group. In one embodiment, the term “alkyl” refers to a C1-C6 straight-chain alkyl group. In one embodiment, the term “alkyl” refers to a C3-C12 branched-chain alkyl group. In one embodiment, the term “alkyl” refers to a C3-C8, branched-chain alkyl group. Cycloalkyls have from about 3 to about 10 carbon atoms in their ring structure, and alternatively about 5, 6, or 7 carbons in the ring structure.

The term “alkenyl” or “alkenyl group” means a group formed by removing a hydrogen from a carbon of an alkene, where an alkene is an acyclic or cyclic compound consisting entirely of hydrogen atoms and carbon atoms, and including at least one carbon-carbon double bond. An alkenyl group may include one or more substituent groups.

The term “alkynyl group” means a group formed by removing a hydrogen from a carbon of an alkyne, where an alkyne is an acyclic or cyclic compound consisting entirely of hydrogen atoms and carbon atoms, and including at least one carbon-carbon triple bond. An alkynyl group may include one or more substituent groups.

The term “substituent” or “substituent group” means a group that replaces one or more hydrogen atoms in a molecular entity. Except as may be specified otherwise, substituent groups can include, without limitation, alkyl, alkenyl, alkynyl, halo, haloalkyl, fluoroalkyl, hydroxy, alkoxy, alkyenyloxy, alkynyloxy, carbocyclyloxy, heterocyclyloxy, haloalkoxy, fluoroalkyloxy, sulfhydryl, alkylthio, haloalkylthio, fluoroalkylthio, alkyenylthio, alkynylthio, sulfonic acid, alkylsulfonyl, haloalkylsulfonyl, fluoroalkylsulfonyl, alkenylsulfonyl, alkynylsulfonyl, alkoxysulfonyl, haloalkoxysulfonyl, fluoroalkoxysulfonyl, alkenyloxysulfonyl, alkynyloxysulfony, aminosulfonyl, sulfinic acid, alkylsulfinyl, haloalkylsulfinyl, fluoroalkylsulfinyl, alkenylsulfinyl, alkynylsulfinyl, alkoxysulfinyl, haloalkoxysulfinyl, fluoroalkoxysulfinyl, alkenyloxysulfinyl, alkynyloxysulfiny, aminosulfinyl, formyl, alkylcarbonyl, haloalkylcarbonyl, fluoroalkylcarbonyl, alkenylcarbonyl, alkynylcarbonyl, carboxyl, alkoxycarbonyl, haloalkoxycarbonyl, fluoroalkoxycarbonyl, alkenyloxycarbonyl, alkynyloxycarbonyl, alkylcarbonyloxy, haloalkylcarbonyloxy, fluoroalkylcarbonyloxy, alkenylcarbonyloxy, alkynylcarbonyloxy, alkylsulfonyloxy, haloalkylsulfonyloxy, fluoroalkylsulfonyloxy, alkenylsulfonyloxy, alkynylsulfonyloxy, haloalkoxysulfonyloxy, fluoroalkoxysulfonyloxy, alkenyloxysulfonyloxy, alkynyloxysulfonyloxy, alkylsulfinyloxy, haloalkylsulfinyloxy, fluoroalkylsulfinyloxy, alkenylsulfinyloxy, alkynylsulfinyloxy, alkoxysulfinyloxy, haloalkoxysulfinyloxy, fluoroalkoxysulfinyloxy, alkenyloxysulfinyloxy, alkynyloxysulfinyloxy, aminosulfinyloxy, amino, amido, aminosulfonyl, aminosulfinyl, cyano, nitro, azido, phosphinyl, phosphoryl, silyl, and silyloxy.

The term “heteroalkyl group” means a group formed by removing a hydrogen from a carbon of a heteroalkane, where a heteroalkane is an acyclic or cyclic compound consisting entirely of hydrogen atoms, saturated carbon atoms, and one or more heteroatoms. A heteroalkyl group may include one or more substituent groups.

The term “heterocyclyl” as used herein refers to a radical of a non-aromatic ring system, including, but not limited to, monocyclic, bicyclic, and tricyclic rings, which can be completely saturated or which can contain one or more units of unsaturation, for the avoidance of doubt, the degree of unsaturation does not result in an aromatic ring system, and having 3 to 12 atoms including at least one heteroatom, such as nitrogen, oxygen, or sulfur. For purposes of exemplification, which should not be construed as limiting the scope of this disclosure, the following are examples of heterocyclic rings: aziridinyl, azirinyl, oxiranyl, thiiranyl, thiirenyl, dioxiranyl, diazirinyl, azetyl, oxetanyl, oxetyl, thietanyl, thietyl, diazetidinyl, dioxetanyl, dioxetenyl, dithietanyl, dithietyl, furyl, dioxalanyl, pyrrolyl, oxazolyl, thiazolyl, imidazolyl, oxadiazolyl, thiadiazolyl, triazolyl, triazinyl, isothiazolyl, isoxazolyl, thiophenyl, pyrazolyl, tetrazolyl, pyridyl, pyridazinyl, pyrimidinyl, pyrazinyl, triazinyl, tetrazinyl, quinolinyl, isoquinolinyl, quinoxalinyl, quinazolinyl, pyridopyrazinyl, benzoxazolyl, benzothiophenyl, benzimidazolyl, benzothiazolyl, benzoxadiazolyl, benzthiadiazolyl, indolyl, benztriazolyl, naphthyridinyl, azepines, azetidinyl, morpholinyl, oxopiperidinyl, oxopyrrolidinyl, piperazinyl, piperidinyl, pyrrolidinyl, quinicludinyl, thiomorpholinyl, tetrahydropyranyl and tetrahydrofuranyl.

The term “alkoxy” or “alkoxy group” as used herein means an alkyl group, as defined herein, appended to the parent molecular moiety through an oxygen atom. Representative examples of alkoxy include, but are not limited to, methoxy, ethoxy, propoxy, 2-propoxy, butoxy, tert-butoxy, pentyloxy, and hexyloxy. The terms “alkyenyloxy”, “alkynyloxy”, “carbocyclyloxy”, and “heterocyclyloxy” are likewise defined.

The term “heteroatom” is art-recognized, and includes an atom of any element other than carbon or hydrogen. Illustrative heteroatoms include boron, nitrogen, oxygen, phosphorus, sulfur and selenium, and alternatively oxygen, nitrogen or sulfur.

The term “cycloalkylalkyl” as used herein refers to an alkyl group substituted with one or more cycloalkyl groups.

The term “heteroalkyl group” means a group formed by removing a hydrogen from a carbon of a heteroalkane, where a heteroalkane is an acyclic or cyclic compound consisting entirely of hydrogen atoms, saturated carbon atoms, and one or more heteroatoms. A heteroalkyl group may include one or more substituent groups.

The term “heterocycloalkylalkyl” as used herein refers to an alkyl group substituted with one or more heterocycloalkyl (i.e., heterocyclyl) groups.

The term “alkenyl” as used herein means a straight or branched chain hydrocarbon radical containing from 2 to 10 carbons and containing at least one carbon-carbon double bond formed by the removal of two hydrogens. Representative examples of alkenyl include, but are not limited to, ethenyl, 2-propenyl, 2-methyl-2-propenyl, 3-butenyl, 4-pentenyl, 5-hexenyl, 2-heptenyl, 2-methyl-1-heptenyl, and 3-decenyl.

The term “alkynyl” as used herein means a straight or branched chain hydrocarbon radical containing from 2 to 10 carbon atoms and containing at least one carbon-carbon triple bond. Representative examples of alkynyl include, but are not limited, to acetylenyl, 1-propynyl, 2-propynyl, 3-butynyl, 2-pentynyl, and 1-butynyl.

The term “alkylene” is art-recognized, and as used herein pertains to a diradical obtained by removing two hydrogen atoms of an alkyl group, as defined above. In one embodiment an alkylene refers to a disubstituted alkane, i.e., an alkane substituted at two positions with substituents such as halogen, azide, alkyl, aralkyl, alkenyl, alkynyl, cycloalkyl, hydroxyl, alkoxyl, amino, nitro, sulfhydryl, imino, amido, phosphonate, phosphinate, carbonyl, carboxyl, silyl, ether, alkylthio, sulfonyl, sulfonamido, ketone, aldehyde, ester, heterocyclyl, aromatic or heteroaromatic moieties, fluoroalkyl (such as trifluromethyl), cyano, or the like. That is, in one embodiment, a “substituted alkyl” is an “alkylene”.

The term “amino” is a term of art and as used herein refers to both unsubstituted and substituted amines, e.g., a moiety that may be represented by the general formulas:

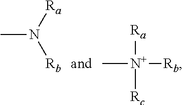

wherein Rₐ, R_{b}, and R_{c} each independently represent a hydrogen, an alkyl, an alkenyl, —(CH₂)ₓ—R_{d}, or Rₐ and R_{b}, taken together with the N atom to which they are attached complete a heterocycle having from 4 to 8 atoms in the ring structure; R_{d} represents an aryl, a cycloalkyl, a cycloalkenyl, a heterocyclyl or a polycyclyl; and x is zero or an integer in the range of 1 to 8. In certain embodiments, only one of Rₐ or R_{b} may be a carbonyl, e.g., Rₐ, R_{b}, and the nitrogen together do not form an imide. In other embodiments, Rₐ and R_{b} ach independently represent a hydrogen, an alkyl, an alkenyl, or —(CH₂)ₓ—R_{d}. In one embodiment, the term “amino” refers to —NH₂.

The term “acyl” is a term of an and as used herein refers to any group or radical of the form RCO— where R is any organic group, e.g., alkyl, aryl, heteroaryl, aralkyl, and heteroaralkyl. Representative acyl groups include acetyl, benzoyl, and malonyl.

The term “aminoalkyl” as used herein refers to an alkyl group substituted with one or more one amino groups. In one embodiment, the term “aminoalkyl” refers to an aminomethyl group.

The term “aminoacyl” is a term of an and as used herein refers to an acyl group substituted with one or more amino groups.

The term “aminothionyl” as used herein refers to an analog of an aminoacyl in which the O of RC(O)— has been replaced by sulfur, hence is of the form RC(S)—.

The term “carbonyl” as used herein refers to —C(O)—.

The term “thiocarbonyl” as used herein refers to —C(S)—.

The term “alkylthio” as used herein refers to alkyl-S—.

The term “aryl” is a term of art and as used herein refers to includes monocyclic, bicyclic and polycyclic aromatic hydrocarbon groups, for example, benzene, naphthalene, anthracene, and pyrene. The aromatic ring may be substituted at one or more ring positions with one or more substituents, such as halogen, azide, alkyl, aralkyl, alkenyl, alkynyl, cycloalkyl, hydroxyl, alkoxyl, amino, nitro, sulfhydryl, imino, amido, phosphonate, phosphinate, carbonyl, carboxyl, silyl, ether, alkylthio, sulfonyl, sulfonamido, ketone, aldehyde, ester, heterocyclyl, aromatic or heteroaromatic moieties, fluoroalkyl (such as trifluromethyl), cyano, or the like. The term “aryl” also includes polycyclic ring systems having two or more cyclic rings in which two or more carbons are common to two adjoining rings (the rings are “fused rings”) wherein at least one of the rings is an aromatic hydrocarbon, e.g., the other cyclic rings may be cycloalkyls, cycloalkenyls, cycloalkynyls, aryls, heteroaryls, and/or heterocyclyls. In one embodiment, the term “aryl” refers to a phenyl group.

The term “heteroaryl” is a term of art and as used herein refers to a monocyclic, bicyclic, and polycyclic aromatic group having one or more heteroatoms in the ring structure, for example, pyrrole, furan, thiophene, imidazole, oxazole, thiazole, triazole, pyrazole, pyridine, pyrazine, pyridazine and pyrimidine, and the like. The “heteroaryl” may be substituted at one or more ring positions with one or more substituents such as halogen, azide, alkyl, aralkyl, alkenyl, alkynyl, cycloalkyl, hydroxyl, alkoxyl, amino, nitro, sulfhydryl, imino, amido, phosphonate, phosphinate, carbonyl, carboxyl, silyl, ether, alkylthio, sulfonyl, sulfonamido, ketone, aldehyde, ester, heterocyclyl, aromatic or heteroaromatic moieties, fluoroalkyl (such as trifluromethyl), cyano, or the like. The term “heteroaryl” also includes polycyclic ring systems having two or more cyclic rings in which two or more carbons are common to two adjoining rings (the rings are “fused rings”) wherein at least one of the rings is an aromatic group having one or more heteroatoms in the ring structure, e.g., the other cyclic rings may be cycloalkyls, cycloalkenyls, cycloalkynyls, aryls, heteroaryls, and/or heterocyclyls.

The term “aralkyl” or “arylalkyl” is a term of art and as used herein refers to an alkyl group substituted with an aryl group.

The term “heteroaralkyl” or “heteroarylalkyl” is a term of art and as used herein refers to an alkyl group substituted with a heteroaryl group.

The term “alkoxy” as used herein means an alkyl group, as defined herein, appended to the parent molecular moiety through an oxygen atom. Representative Examples of alkoxy include, but are not limited to, methoxy, ethoxy, propoxy. 2-propoxy, butoxy, tert-butoxy, pentyloxy, and hexyloxy.

The term “aryloxy” as used herein means an aryl group, as defined herein, appended to the parent molecular moiety through an oxygen atom.

The term “heteroaryloxy” as used herein means a heteroaryl group, as defined herein, appended to the parent molecular moiety through an oxygen atom.

The term “carbocyclyl” as used herein means a monocyclic or multicyclic (e.g., bicyclic, tricyclic, etc.) hydrocarbon radical containing from 3 to 12 carbon atoms that is completely saturated or has one or more unsaturated bonds, and for the avoidance of doubt, the degree of unsaturation does not result in an aromatic ring system (e.g., phenyl). Examples of carbocyclyl groups include 1-cyclopropyl, 1-cyclobutyl, 2-cyclopentyl, 1-cyclopentenyl, 3-cyclohexyl, 1-cyclohexenyl and 2-cyclopentenylmethyl.

The term “cyano” is a term of art and as used herein refers to —CN.

The term “fluoroalkyl” as used herein refers to an alkyl group, as defined herein, wherein some or all of the hydrogens are replaced with fluorines.

The term “halo” is a term of art and as used herein refers to —F, —Cl, —Br, or —I.

The term “hydroxy” is a term of art and as used herein refers to —OH.

Certain compounds contained in compositions of the present disclosure may exist in particular geometric or stereoisomeric forms. In addition, compounds of the present disclosure may also be optically active. The present disclosure contemplates all such compounds, including cis- and trans-isomers, (R)- and (S)-enantiomers, diastereoisomers, (D)-isomers, (L)-isomers, the racemic mixtures thereof, and other mixtures thereof, as falling within the scope of the present disclosure. Additional asymmetric carbon atoms may be present in a substituent such as an alkyl group. All such isomers, as well as mixtures thereof, are intended to be included in this disclosure.

If, for instance, a particular enantiomer of compound of the present disclosure is desired, it may be prepared by asymmetric synthesis, or by derivation with a chiral auxiliary, where the resulting diastereomeric mixture is separated and the auxiliary group cleaved to provide the pure desired enantiomers. Alternatively, where the molecule contains a basic functional group, such as amino, or an acidic functional group, such as carboxyl, diastereomeric salts are formed with an appropriate optically-active acid or base, followed by resolution of the diastereomers thus formed by fractional crystallization or chromatographic means well known in the art, and subsequent recovery of the pure enantiomers.

It will be understood that “substitution” or “substituted with” includes the implicit proviso that such substitution is in accordance with permitted valence of the substituted atom and the substituent, and that the substitution results in a stable compound, e.g., which does not spontaneously undergo transformation such as by rearrangement, fragmentation, decomposition, cyclization, elimination, or other reaction.

The term “substituted” is also contemplated to include all permissible substituents of organic compounds. In a broad aspect, the permissible substituents include acyclic and cyclic, branched and unbranched, carbocyclic and heterocyclic, aromatic and nonaromatic substituents of organic compounds. Illustrative substituents include, for example, those described herein above. The permissible substituents may be one or more and the same or different for appropriate organic compounds. For purposes of this disclosure, the heteroatoms such as nitrogen may have hydrogen substituents and/or any permissible substituents of organic compounds described herein which satisfy the valences of the heteroatoms. This disclosure is not intended to be limited in any manner by the permissible substituents of organic compounds.

For purposes of the present disclosure, the chemical elements are identified in accordance with the Periodic Table of the Elements, CAS version, Handbook of Chemistry and Physics, 67th Ed., 1986-87, inside cover.

Other chemistry terms herein are used according to conventional usage in the art, as exemplified by The McGraw-Hill Dictionary of Chemical Terms (ed. Parker, S., 1985), McGraw-Hill, San Francisco, incorporated herein by reference). Unless otherwise defined, all technical and scientific terms used herein have the same meaning as commonly understood by one of ordinary skill in the art to which this disclosure pertains.

The term “pharmaceutically acceptable salt” as used herein includes salts derived from inorganic or organic acids including, for example, hydrochloric, hydrobromic, sulfuric, nitric, perchloric, phosphoric, formic, acetic, lactic, maleic, fumaric, succinic, tartaric, glycolic, salicylic, citric, methanesulfonic, benzenesulfonic, benzoic, malonic, trifluoroacetic, trichloroacetic, naphthalene-2-sulfonic, and other acids. Pharmaceutically acceptable salt forms can include forms wherein the ratio of molecules comprising the salt is not 1:1. For example, the salt may comprise more than one inorganic or organic acid molecule per molecule of base, such as two hydrochloric acid molecules per molecule of compound of Formula Ia or Ib. As another example, the salt may comprise less than one inorganic or organic acid molecule per molecule of base, such as two molecules of compound of Formula Ia or Ib per molecule of tartaric acid.

The terms “carrier” and “pharmaceutically acceptable carrier” as used herein refer to a diluent, adjuvant, excipient, or vehicle with which a compound is administered or formulated for administration. Non-limiting examples of such pharmaceutically acceptable carriers include liquids, such as water, saline, and oils; and solids, such as gum acacia, gelatin, starch paste, talc, keratin, colloidal silica, urea, and the like. In addition, auxiliary, stabilizing, thickening, lubricating, flavoring, and coloring agents may be used. Other examples of suitable pharmaceutical carriers are described in Remington's Pharmaceutical Sciences by E. W. Martin, herein incorporated by reference in its entirety.

The term “treat” as used herein means prevent, halt or slow the progression of, or eliminate a disease or condition in a subject. In one embodiment “treat” means halt or slow the progression of, or eliminate a disease or condition in a subject. In one embodiment, “treat” means reduce at least one objective manifestation of a disease or condition in a subject.

The term “effective amount” as used herein refers to an amount that is sufficient to bring about a desired biological effect.

The term “therapeutically effective amount” as used herein refers to an amount that is sufficient to bring about a desired therapeutic effect.

The term “inhibit” as used herein means decrease by an objectively measurable amount or extent. In various embodiments “inhibit” means decrease by at least 5, 10, 20, 30, 40, 50, 60, 70, 80, 90, or 95 percent compared to relevant control. In one embodiment “inhibit” means decrease 100 percent, i.e., halt or eliminate.

The term “subject” as used herein refers to a mammal. In various embodiments, a subject is a mouse, rat, rabbit, cat, dog, pig, sheep, horse, cow, or non-human primate. In one embodiment, a subject is a human.

### Compounds

In some aspects, the present disclosure provides a compound or a tautomer thereof, or a pharmaceutically acceptable salt of either, represented by Formula Ia:

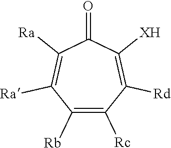

wherein:
   X represents oxygen or sulfur;
   Rₐ represents hydrogen, halo, alkyl, substituted alkyl, heteroalkyl, alkoxy, substituted alkoxy, alkoxyalkyl, substituted alkoxyalkyl, cycloalkyl, substituted cycloalkyl, heterocycloalkyl, substituted heterocycloalkyl, alkenyl, substituted alkenyl, heteroalkenyl, cycloalkenyl, substituted cycloalkenyl, heterocycloalkenyl, substituted heterocycloalkenyl, alkynyl, substituted alkynyl, or heteroalkynyl;
   Rₐ′ represents hydrogen, halo, alkyl, or substituted alkyl; and R_{b}, R_{c}, and R_{d} are independently selected from the group consisting of hydrogen, halo, alkyl, substituted alkyl, heteroalkyl, alkylcycloalkyl, alkylheterocycloalkyl, alkoxy, substituted alkoxy, alkoxyalkyl, substituted alkoxyalkyl, cycloalkyloxy, substituted cycloalkyloxy, heterocycloalkyloxy, substituted heterocycloalkyloxy, cycloalkyl, substituted cycloalkyl, heterocycloalkyl, substituted heterocycloalkyl, alkenyl, substituted alkenyl, heteroalkenyl, cycloalkenyl, substituted cycloalkenyl, heterocycloalkenyl, substituted heterocycloalkenyl, alkynyl, substituted alkynyl, and heteroalkynyl; provided that Rₐ, R_{b}, R_{c}, and R_{d} are not all hydrogen.

In certain embodiments, in Formula Ia, Rₐ′ is hydrogen.

In certain embodiments, in Formula Ia, Rₐ′ is halo.

In certain embodiments, in Formula Ia, Rₐ′ is alkyl, or substituted alkyl.

In certain embodiments, in Formula Ia, at least one of Rₐ, R_{b}, R_{c}, and R_{d} is selected from the group consisting of halo, alkyl, substituted alkyl, heteroalkyl, alkylcycloalkyl, alkylheterocycloalkyl, alkoxy, substituted alkoxy, alkoxyalkyl, substituted alkoxyalkyl, cycloalkyloxy, substituted cycloalkyloxy, heterocycloalkyloxy, substituted heterocycloalkyloxy, cycloalkyl, substituted cycloalkyl, heterocycloalkyl, and substituted heterocycloalkyl.

In some aspects, the present disclosure provides a compound or a tautomer thereof, or a pharmaceutically acceptable salt of either, represented by Formula Ib:

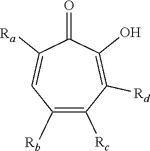

wherein:
   Rₐ represents hydrogen, halo, alkyl, substituted alkyl, heteroalkyl, alkoxy, substituted alkoxy, alkoxyalkyl, substituted alkoxyalkyl, cycloalkyl, substituted cycloalkyl, heterocycloalkyl, substituted heterocycloalkyl, alkenyl, substituted alkenyl, heteroalkenyl, cycloalkenyl, substituted cycloalkenyl, heterocycloalkenyl, substituted heterocycloalkenyl, alkynyl, substituted alkynyl, or heteroalkynyl; and
   R_{b}, R_{c}, and R_{d} are independently selected from the group consisting of hydrogen, halo, alkyl, substituted alkyl, heteroalkyl, alkoxy, substituted alkoxy, alkoxyalkyl, substituted alkoxyalkyl, cycloalkyl, substituted cycloalkyl, heterocycloalkyl, substituted heterocycloalkyl, alkenyl, substituted alkenyl, heteroalkenyl, cycloalkenyl, substituted cycloalkenyl, heterocycloalkenyl, substituted heterocycloalkenyl, alkynyl, substituted alkynyl, and heteroalkynyl;
provided that Rₐ, R_{b}, R_{c}, and R_{d} are not all hydrogen.

In certain embodiments, in Formula Ia or Ib,
each occurrence of heterocycloalkyl is independently selected from the group consisting of:

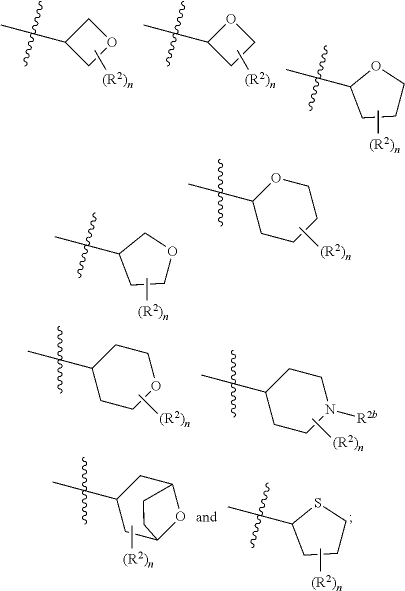

n is independently for each occurrence an integer selected from 0-5 inclusive;
R^{2b} is independently for each occurrence hydrogen, alkyl, substituted alkyl, heteroalkyl, hydroxy, alkoxy, substituted alkoxy, alkoxyalkyl, substituted alkoxyalkyl, cycloalkyl, substituted cycloalkyl, alkenyl, substituted alkenyl, cycloalkenyl, substituted cycloalkenyl, alkynyl, and substituted alkynyl; and
each instance of R² independently represents —F, alkyl, haloalkyl, or alkoxy; or two geminal instances of R² represent carbonyl.

In certain embodiments, in Formula Ia or Ib, Rₐ represents —F, —CF₃, (C2-C15)alkyl, or substituted (C1-C15)alkyl.

In certain embodiments, in Formula Ia or Ib,
Rₐ represents —F, —CF₃, (C2-C15)alkyl, or substituted (C1-C15)alkyl;
R_{b}, R_{c}, and R_{d} independently represent hydrogen, (C1-C15)alkyl, 3-12 membered cycloalkyl, 3-12 membered heterocycloalkyl, (C1-C15)alkylene-R₁, 3-12 membered cycloalkyl-R₁, or 3-12 membered heterocycloalkyl-R₁; and
R₁ represents independently for each occurrence halo, alkyl, alkoxy, or hydroxyl.

In certain embodiments, the compound of Formula Ia or Ib is selected from the group consisting of:

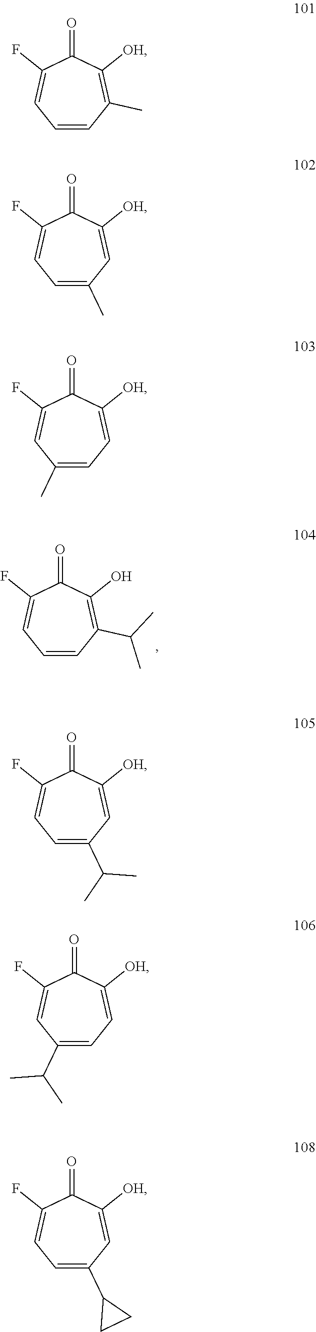

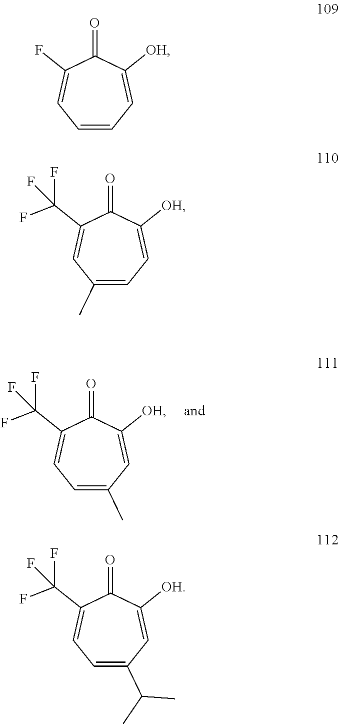

In certain embodiments, the compound of Formula Ia or Ib is selected from the group consisting of:

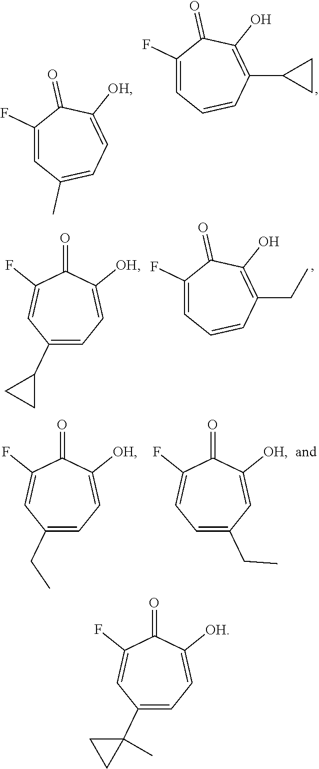

In certain embodiments, in Formula Ia or Ib, Rₐ represents —Cl, —F, —CF₃, (C2-C15)alkyl, or substituted (C1-C15)alkyl.

In certain embodiments, in Formula Ia or Ib,
R_{b}, R_{c}, and R_{d} independently represent hydrogen, halo (C1-C15)alkyl, 3-12 membered cycloalkyl, 3-12 membered heterocycloalkyl, (C1-C15)alkylene-R₁, 3-12 membered cycloalkyl-R₁, or 3-12 membered heterocycloalkyl-R₁; and
R₁ represents independently for each occurrence halo, alkyl, alkoxy, or hydroxyl.

In certain embodiments, the compound of Formula Ia or Ib is selected from the group consisting

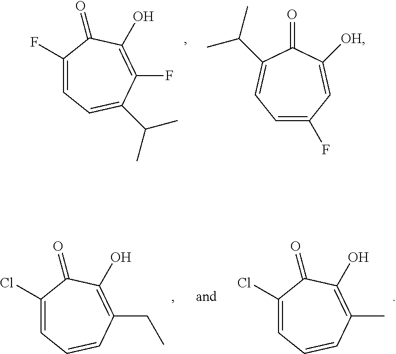

In certain embodiments, in Formula Ia or Ib,
R_{b}, R_{c}, and R_{d} independently represent hydrogen or 3-12 membered heterocycloalkyl optionally substituted with one or two instances of R₂;
   wherein said heterocycloalkyl comprises one or two oxygen atoms, one or two nitrogen atoms, one or two sulfur atoms, or any combination of two atoms selected from the group consisting of oxygen, nitrogen, and sulfur atoms; and
each instance of R₂ independently represents —F, alkyl, haloalkyl, carbonyl, or alkoxy.

In certain embodiments, the compound of Formula Ia or Ib is selected from the group consisting of:

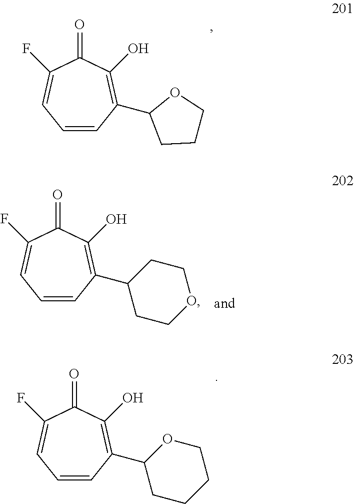 [SMILES: O=c1c(F)cccc(C2CCCO2)c1O ; O=c1c(F)cccc(C2CCOCC2)c1O ; O=c1c(F)cccc(C2CCCCO2)c1O]

and

In certain embodiments, in Formula Ia or Ib,
R_{b}, R_{c}, and R_{d} independently represent hydrogen or 3-12 membered heterocycloalkyl optionally substituted with one or two instances of R₂;
   wherein said heterocycloalkyl comprises one or two oxygen atoms, one or two nitrogen atoms, one or two sulfur atoms, or any combination of two atoms selected from the group consisting of oxygen, nitrogen, and sulfur atoms;
each instance of R₂ independently represents —F, alkyl, haloalkyl, carbonyl, or alkoxy.
said heterocycloalkyl comprises one or two nitrogen atoms; and at least one of said nitrogen atoms is represented by N(R_{2b});
R_{2b} independently represents hydrogen, —C(O)R₅, or —C(O)OR₅; and
R₅ independently represents hydrogen, alkyl, or substituted alkyl.

In certain embodiments, in Formula Ia or Ib,
R_{b}, R_{c}, and R_{d} independently represent hydrogen or —OR₃; and
R₃ independently represents (C1-C15)alkyl, 3-12 membered cycloalkyl or bicycloalkyl, 3-12 membered heterocycloalkyl or heterobicylcloalkyl comprises one or two oxygen atoms, one or two nitrogen atoms, one or two sulfur atoms, or any combination of two atoms selected from the group consisting of oxygen, nitrogen, and sulfur atoms.

In certain embodiments, the compound of Formula Ia or Ib is:

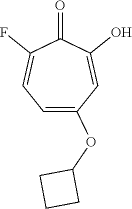 [SMILES: O=c1c(O)cc(OC2CCC2)ccc1F]

In certain embodiments, the compound of Formula Ia or Ib is selected from the group consisting of:

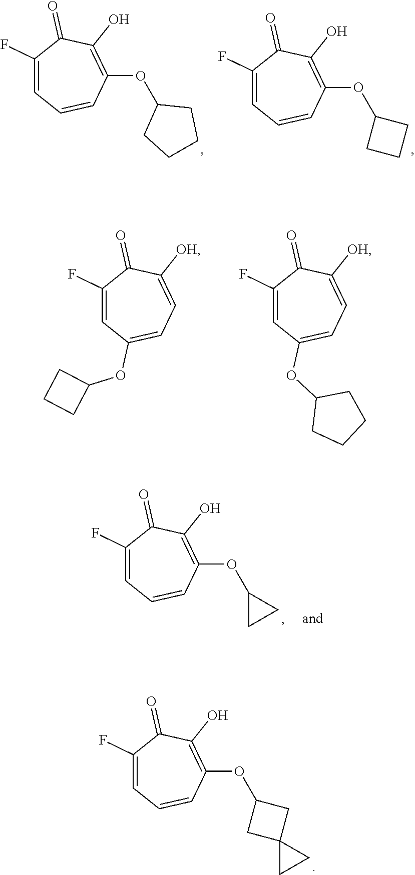 [SMILES: O=c1c(F)cccc(OC2CCCC2)c1O ; O=c1c(F)cccc(OC2CCC2)c1O ; O=c1c(O)ccc(OC2CCC2)cc1F ; O=c1c(O)ccc(OC2CCCC2)cc1F ; O=c1c(F)cccc(OC2CC2)c1O ; O=c1c(F)cccc(OC2CC3(CC3)C2)c1O]

In certain embodiments, in Formula Ia or Ib,
R_{b}, R_{c}, and R_{d} independently represent hydrogen or —OR₃;
R₃ independently represents (C1-C15)alkyl, 3-12 membered cycloalkyl or bicycloalkyl, 3-12 membered heterocycloalkyl or heterobicylcloalkyl comprises one or two oxygen atoms, one or two nitrogen atoms, one or two sulfur atoms, or any combination of two atoms selected from the group consisting of oxygen, nitrogen, and sulfur atoms;
each instance of R₃ is optionally substituted with R₄; and R₄ independently represents alkyl, halogen-substituted alkyl, alkoxy, or hydroxy.

In certain embodiments, in Formula Ia or Ib, Rₐ represents —CH₃.

In certain embodiments, in Formula Ia or Ib,
Rₐ represents —CH₃;
R_{b}, R_{c}, and R_{d} independently represent hydrogen, (C1-C15)alkyl, 3-12 membered cycloalkyl, 3-12 membered heterocycloalkyl, (C1-C15)alkylene-R₁, 3-12 membered cycloalkyl-R₁, or 3-12 membered heterocycloalkyl-R₁; and
R₁ represents independently for each occurrence halo, alkyl, alkoxy, or hydroxyl.

In certain embodiments, the compound of Formula Ia or Ib is selected from the group consisting of:

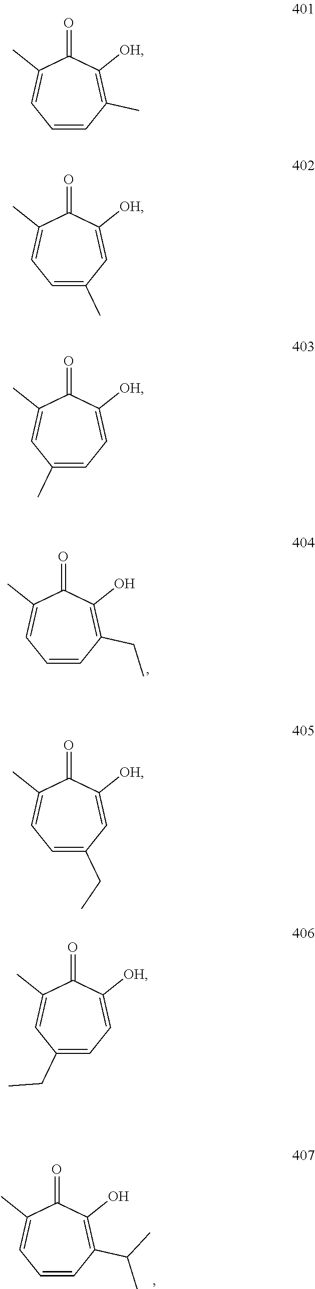

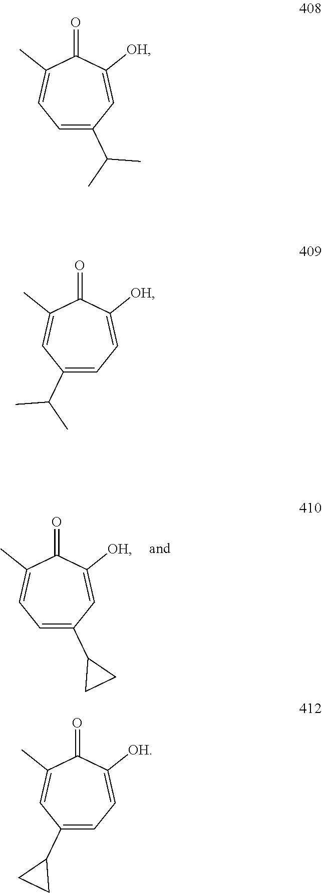

In certain embodiments, in Formula Ia or Ib,
Rₐ represents —CH₃;
R_{b}, R_{c}, and R_{d} independently represent hydrogen or 3-12 membered heterocycloalkyl optionally substituted with one or two instances of R₂;
   wherein said heterocycloalkyl comprises one or two oxygen atoms, one or two nitrogen atoms, one or two sulfur atoms, or any combination of two atoms selected from the group consisting of oxygen, nitrogen, and sulfur atoms; and
each instance of R₂ independently represents —F, alkyl, haloalkyl, carbonyl, or alkoxy.

In certain embodiments, the compound of Formula Ia or Ib is selected from the group consisting of:

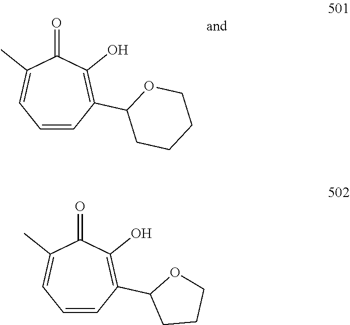 [SMILES: Cc1cccc(C2CCCCO2)c(O)c1=O ; Cc1cccc(C2CCCO2)c(O)c1=O]

In certain embodiments, in Formula Ia or Ib,
said heterocycloalkyl comprises one or two nitrogen atoms; and at least one of said nitrogen atoms is represented by N(R_{2b});
R_{2b} independently represents hydrogen, —C(O)R₅, or —C(O)OR₅; and
R₅ independently represents hydrogen, alkyl, or substituted alkyl.

In certain embodiments, in Formula Ia or Ib,
R_{b}, R_{c}, and R_{d} independently represent hydrogen or —OR₃; and
R₃ independently represents (C1-C15)alkyl, 3-12 membered cycloalkyl or bicycloalkyl, 3-12 membered heterocycloalkyl or heterobicylcloalkyl comprising one or two oxygen atoms, one or two nitrogen atoms, one or two sulfur atoms, or any combination of two atoms selected from the group consisting of oxygen, nitrogen, and sulfur atoms.

In certain embodiments, in Formula Ia or Ib, each instance of R₃ is optionally substituted with R₄; and R₄ independently represents alkyl, halogen-substituted alkyl, alkoxy, or hydroxy.

In certain embodiments, in Formula Ia or Ib, Rₐ represents hydrogen.

In certain embodiments, in Formula Ia or Ib, R_{b}, R_{c}, and R_{d} independently represent hydrogen, (C1-C15)alkyl, —F or —CF₃.

In certain embodiments, in Formula Ia or Ib, the compound is selected from the group consisting of:

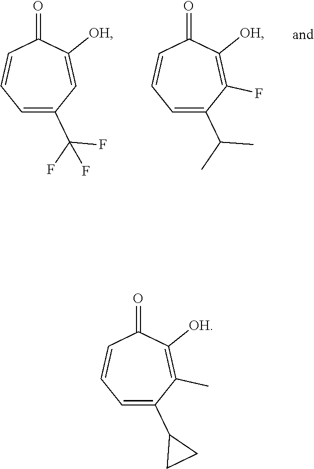

In certain embodiments, in Formula Ia or Ib, the compound is selected from the group

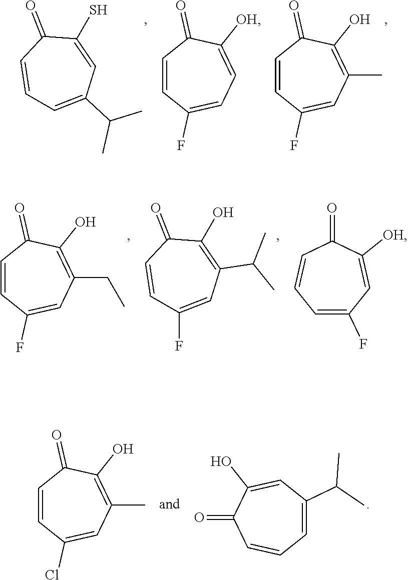

In certain embodiments, in Formula Ia or Ib, R_{b}, R_{c}, and R_{d} independently represent hydrogen, (C1-C15)alkyl, —Cl, —F or —CF₃.

In certain embodiments, in Formula Ia or Ib, the compound is

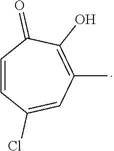

In certain embodiments, in Formula Ia or Ib,
Rₐ represents —CH₃;
R_{b}, R_{c}, and R_{d} independently represent hydrogen or 3-12 membered heterocycloalkyl optionally substituted with one or two instances of R₂;
   wherein said heterocycloalkyl comprises one or two oxygen atoms, one or two nitrogen atoms, one or two sulfur atoms, or any combination of two atoms selected from the group consisting of oxygen, nitrogen, and sulfur atoms; and
each instance of R₂ independently represents —F, alkyl, haloalkyl, carbonyl, or alkoxy.
said heterocycloalkyl comprises one or two nitrogen atoms; at least one of said nitrogen atoms is represented by N(R_{2b});
R_{2b} independently represents hydrogen, —C(O)R₅, or —C(O)OR₅; and
R₅ independently represents hydrogen, alkyl, or substituted alkyl.

In certain embodiments, in Formula Ia or Ib,
Rₐ represents —CH₃;
R_{b}, R_{c}, and R_{d} independently represent hydrogen or —OR₃;
R₃ independently represents (C1-C15)alkyl, 3-12 membered cycloalkyl or bicycloalkyl, 3-12 membered heterocycloalkyl or heterobicylcloalkyl comprises one or two oxygen atoms, one or two nitrogen atoms, one or two sulfur atoms, or any combination of two atoms selected from the group consisting of oxygen, nitrogen, and sulfur atoms.

In certain embodiments, in Formula Ia or Ib,
Rₐ represents —CH₃;
R_{b}, R_{c}, and R_{d} independently represent hydrogen or —OR₃;
R₃ independently represents (C1-C15)alkyl, 3-12 membered cycloalkyl or bicycloalkyl, 3-12 membered heterocycloalkyl or heterobicylcloalkyl comprises one or two oxygen atoms, one or two nitrogen atoms, one or two sulfur atoms, or any combination of two atoms selected from the group consisting of oxygen, nitrogen, and sulfur atoms.
each instance of R₁ is optionally substituted with R₄; and R₄ independently represents alkyl, halogen-substituted alkyl, alkoxy, or hydroxy.

In certain embodiments, in Formula Ia or Ib, Rₐ represents hydrogen.

In certain embodiments, in Formula Ia or Ib,
Rₐ represents hydrogen; and
R_{b}, R_{c}, and R_{d} independently represent hydrogen, (C1-C15)alkyl, —F or —CF₃.

In certain embodiments, Formula Ia or Ib represents

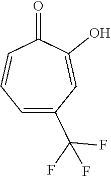 [SMILES: O=c1cccc(C(F)(F)F)cc1O]

In certain embodiments, in Formula Ia or Ib,
Rₐ represents hydrogen;
R_{b}, R_{c}, and R_{d} independently represent hydrogen or 3-12 membered heterocycloalkyl optionally substituted with one or two instances of R₂;
   wherein said heterocycloalkyl comprises one or two oxygen atoms, one or two nitrogen atoms, one or two sulfur atoms, or any combination of two atoms selected from the group consisting of oxygen, nitrogen, and sulfur atoms; and
each instance of R₂ independently represents —F, alkyl, haloalkyl, carbonyl, or alkoxy.

In certain embodiments, in Formula Ia or Ib,
R_{b}, R_{c}, and R_{d} independently represent hydrogen, 3-12 membered cycloalkyl or or 3-12 membered heterocycloalkyl each optionally substituted with one or two instances of R₂;
   wherein said heterocycloalkyl comprises one or two oxygen atoms, one or two nitrogen atoms, one or two sulfur atoms, or any combination of two atoms selected from the group consisting of oxygen, nitrogen, and sulfur atoms; and
each instance of R₂ independently represents —F, alkyl, haloalkyl, carbonyl, or alkoxy.

In certain embodiments, the compound of Formula Ia or Ib is selected from the group consisting of:

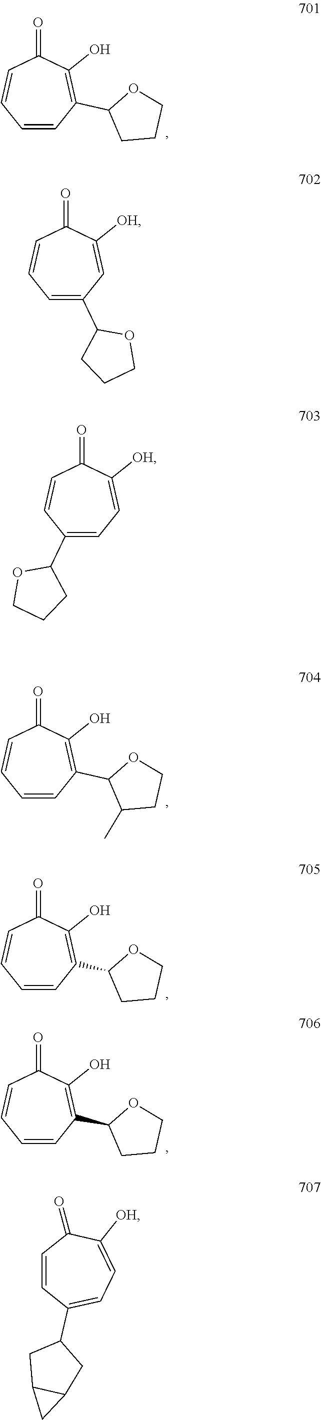

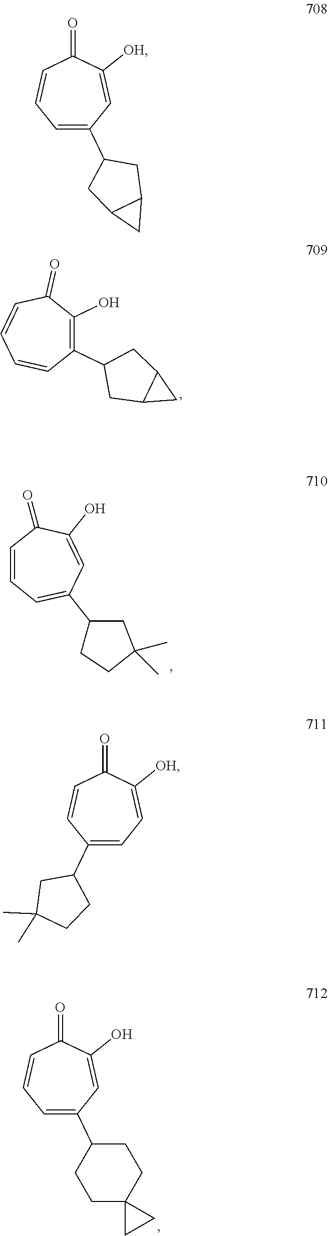

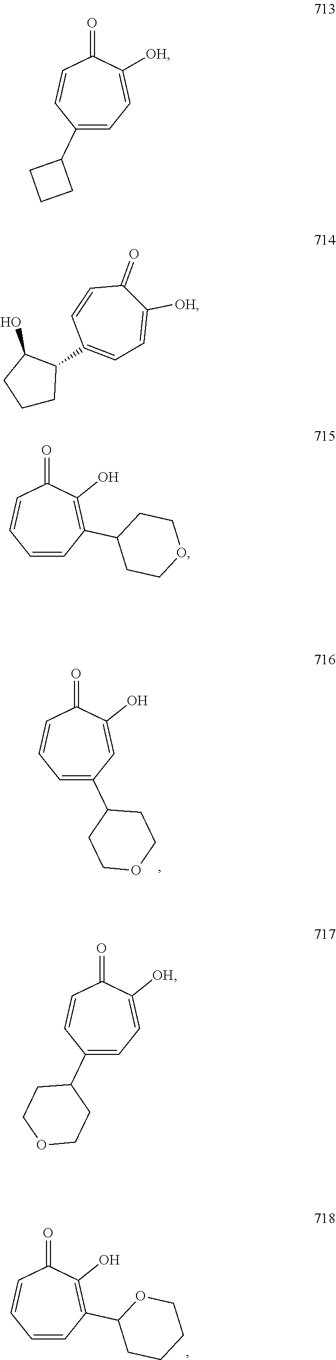 [SMILES: O=c1ccc(C2CCC2)ccc1O ; O=c1ccc([C@@H]2CCC[C@H]2O)ccc1O ; O=c1ccccc(C2CCOCC2)c1O ; O=c1cccc(C2CCOCC2)cc1O ; O=c1ccc(C2CCOCC2)ccc1O ; O=c1ccccc(C2CCCCO2)c1O]

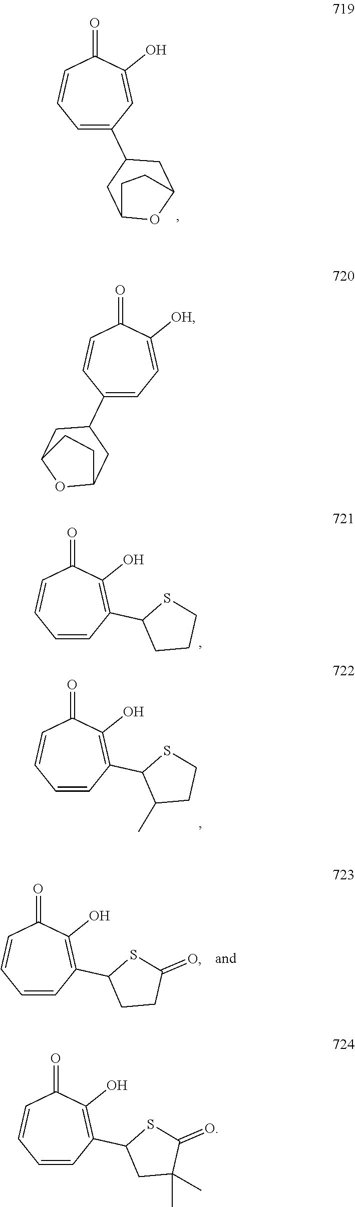 [SMILES: O=c1cccc(C2CC3CCC(C2)O3)cc1O ; O=c1ccc(C2CC3CCC(C2)O3)ccc1O ; O=c1ccccc(C2CCCS2)c1O ; CC1CCSC1c1ccccc(=O)c1O ; O=C1CCC(c2ccccc(=O)c2O)S1 ; CC1(C)CC(c2ccccc(=O)c2O)SC1=O]

In certain embodiments, in Formula Ia or Ib, the compound is selected from the group consisting of:

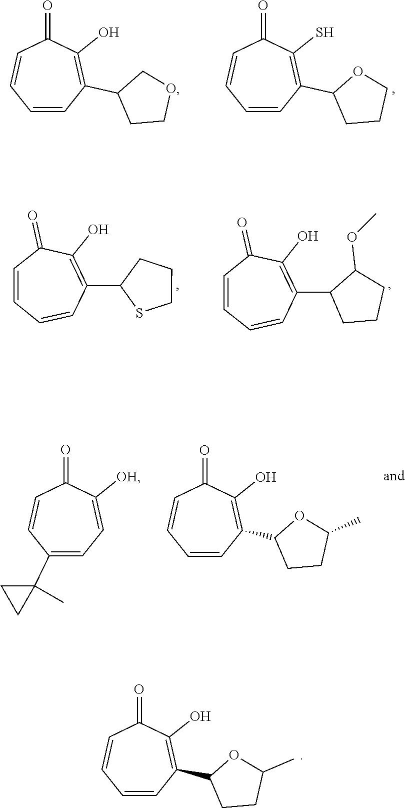

In certain embodiments, in Formula Ia or Ib,
R_{b} is halo;
R_{c} and R_{d} independently represent hydrogen, 3-12 membered cycloalkyl, 3-12 membered heterocycloalkyl, cycloalkyloxy, or heterocycloalkyloxy,
each cycloalkyl heterocycloalkyl, cycloalkyloxy, or heterocycloalkyloxy is optionally substituted with one or two instances of R₂;
   wherein said heterocycloalkyl comprises one or two oxygen atoms, one or two nitrogen atoms, one or two sulfur atoms, or any combination of two atoms selected from the group consisting of oxygen, nitrogen, and sulfur atoms; and
each instance of R₂ independently represents —F, alkyl, haloalkyl, carbonyl, or alkoxy.

In certain embodiments, in Formula Ia or Ib, the compound is selected from the group consisting of:

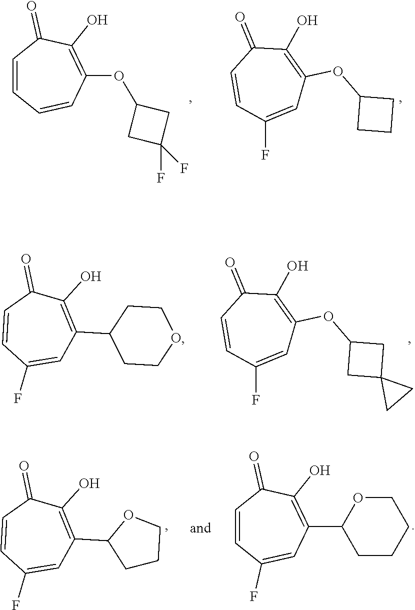 [SMILES: O=c1ccccc(OC2CC(F)(F)C2)c1O ; O=c1ccc(F)cc(OC2CCC2)c1O ; O=c1ccc(F)cc(C2CCOCC2)c1O ; O=c1ccc(F)cc(OC2CC3(CC3)C2)c1O ; O=c1ccc(F)cc(C2CCCO2)c1O ; O=c1ccc(F)cc(C2CCCCO2)c1O]

In certain embodiments, in Formula Ia or Ib, the compound is selected from the group consisting of:
said heterocycloalkyl comprises one or two nitrogen atoms; and at least one of said nitrogen atoms is represented by N(R_{2b});
R_{2b} independently represents hydrogen, —C(O)R₅, or —C(O)OR₅; and
R₅ independently represents hydrogen, alkyl, or substituted alkyl.

In certain embodiments, in Formula Ia or Ib,
Rₐ represents hydrogen;
R_{b}, R_{c}, and R_{d} independently represent hydrogen or 3-12 membered heterocycloalkyl optionally substituted with one or two instances of R₂;
   wherein said heterocycloalkyl comprises one or two oxygen atoms, one or two nitrogen atoms, one or two sulfur atoms, or any combination of two atoms selected from the group consisting of oxygen, nitrogen, and sulfur atoms;
each instance of R₂ independently represents —F, alkyl, haloalkyl, carbonyl, or alkoxy.
said heterocycloalkyl comprises one or two nitrogen atoms; at least one of said nitrogen atoms is represented by N(R_{2b});
R_{2b} independently represents hydrogen, —C(O)R₅, or —C(O)OR₅; and
R₅ independently represents hydrogen, alkyl, or substituted alkyl.

In certain embodiments, the compound of Formula Ia or Ib is selected from the group consisting of:

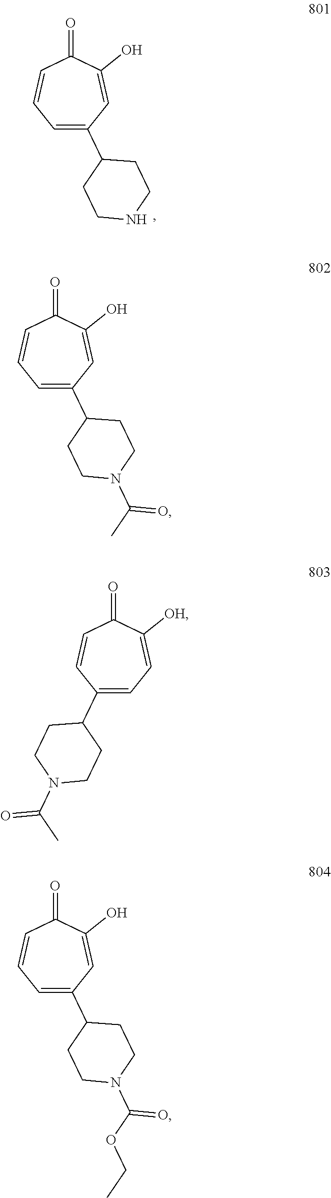

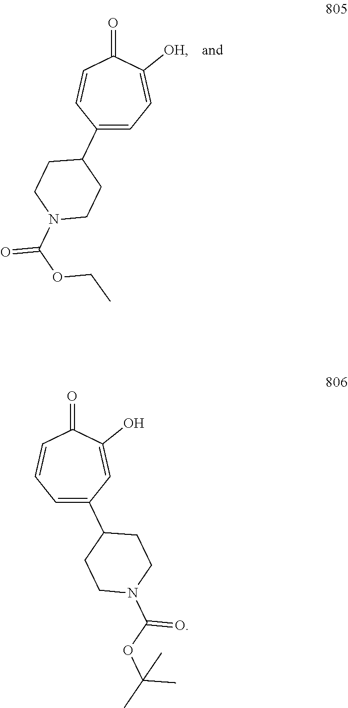 [SMILES: CCOC(=O)N1CCC(c2ccc(O)c(=O)cc2)CC1 ; CC(C)(C)OC(=O)N1CCC(c2cccc(=O)c(O)c2)CC1]

In certain embodiments, in Formula Ia or Ib,
R_{b}, R_{c}, and R_{d} independently represent hydrogen or —OR₃; and
R₃ independently represents (C1-C15)alkyl, 3-12 membered cycloalkyl or bicycloalkyl, 3-12 membered heterocycloalkyl or heterobicylcloalkyl comprises one or two oxygen atoms, one or two nitrogen atoms, one or two sulfur atoms, or any combination of two atoms selected from the group consisting of oxygen, nitrogen, and sulfur atoms.

In certain embodiments, the compound of Formula Ia or Ib is selected from the group consisting of:

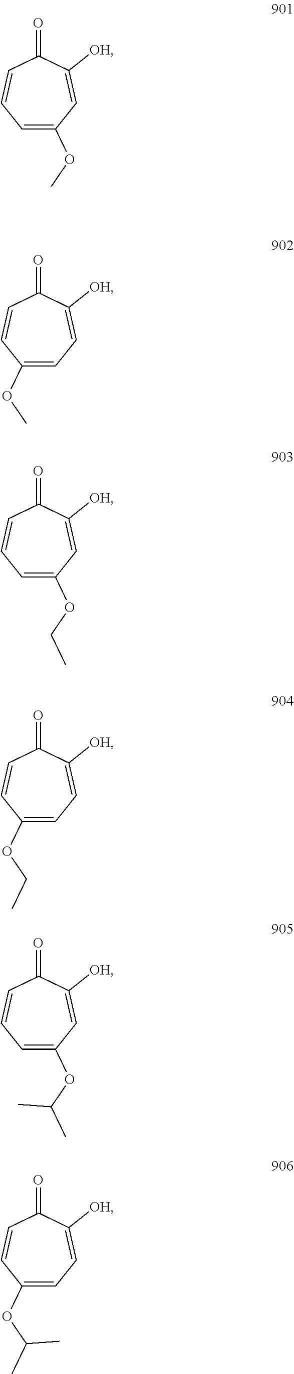

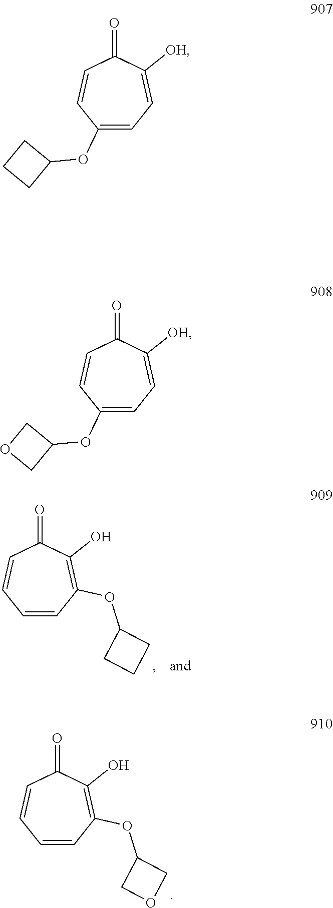

In certain embodiments, in Formula Ia or Ib, each instance of R₃ is optionally substituted with R₄; and R₄ independently represents alkyl, halogen-substituted alkyl, alkoxy, or hydroxy.

In certain embodiments, in Formula Ia or Ib, the compound is selected from the group consisting of:

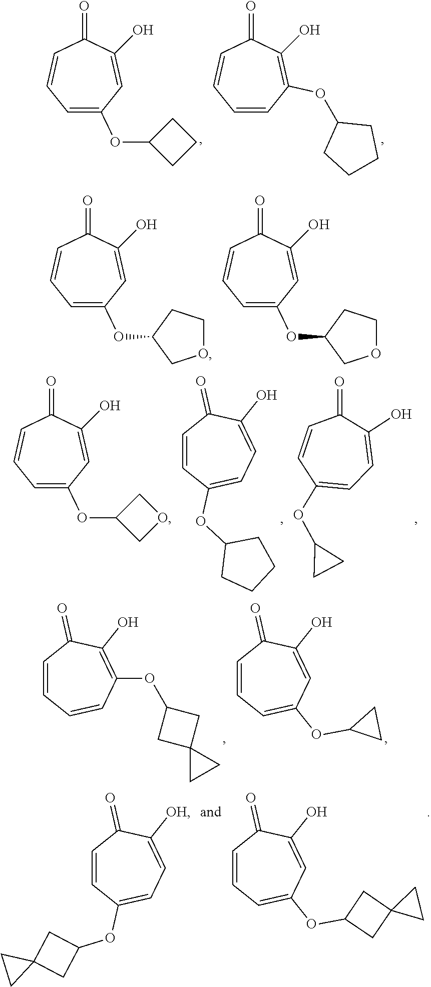

In certain embodiments, in Formula Ia or Ib,
R_{b}, R_{c}, and R_{d} independently represent hydrogen or -alkyl-R₃ or —O-alkyl-R₃; and
R₃ independently represents aryl, substituted aryl, 3-12 membered cycloalkyl or bicycloalkyl, or a 3-12 membered heterocycloalkyl or heterobicylcloalkyl comprising one or two oxygen atoms, one or two nitrogen atoms, one or two sulfur atoms, or any combination of two atoms selected from the group consisting of oxygen, nitrogen, and sulfur atoms.

In certain embodiments, in Formula Ia or Ib, the compound is selected from the group consisting of:

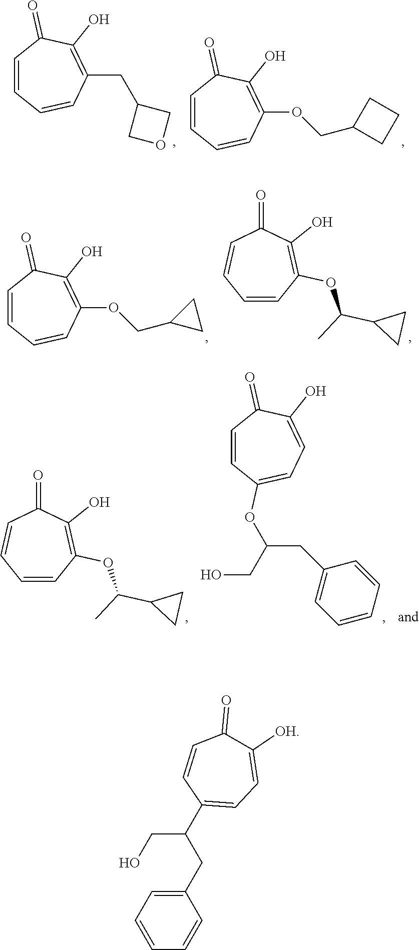

In certain embodiments, in Formula Ia or Ib, wherein Rₐ′ is halo or alkyl.

In certain embodiments, in Formula Ia or Ib, the compound is

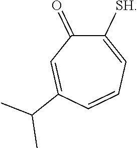

In certain embodiments, in Formula Ia or Ib,
R_{b}, R_{c}, and R_{d} independently represent hydrogen or —OR₃;
R₃ independently represents (C1-C15)alkyl, 3-12 membered cycloalkyl or bicycloalkyl, 3-12 membered heterocycloalkyl or heterobicylcloalkyl comprises one or two oxygen atoms, one or two nitrogen atoms, one or two sulfur atoms, or any combination of two atoms selected from the group consisting of oxygen, nitrogen, and sulfur atoms;
each instance of R₃ is optionally substituted with R₄; and R₄ independently represents alkyl, halogen-substituted alkyl, alkoxy, or hydroxy.

In some aspects, the present disclosure provides a compound or a tautomer thereof, or a pharmaceutically acceptable salt of either, represented by Formula IIa or Formula IIb:

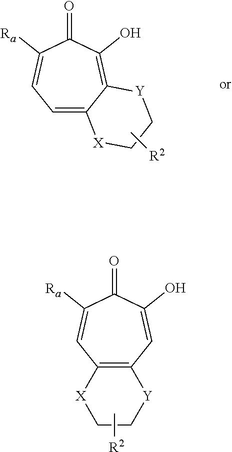

wherein:
   Rₐ represents hydrogen, halo, alkyl, substituted alkyl, heteroalkyl, alkoxy, substituted alkoxy, alkoxyalkyl, substituted alkoxyalkyl, cycloalkyl, substituted cycloalkyl, heterocycloalkyl, substituted heterocycloalkyl, alkenyl, substituted alkenyl, heteroalkenyl, cycloalkenyl, substituted cycloalkenyl, heterocycloalkenyl, substituted heterocycloalkenyl, alkynyl, substituted alkynyl, or heteroalkynyl;
   X and Y independently represent O, S, NH, or CR₅R₆;
   R₂ represents —F, alkyl, haloalkyl, or alkoxy; and
   R₅ and R₆ represent independently for each occurrence H, (C1-C15) alkyl, or substituted (C1-C15)alkyl;
provided the compound is not

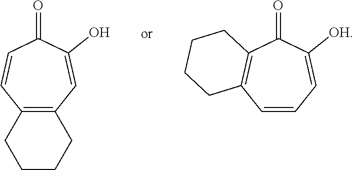 [SMILES: O=c1ccc2c(cc1O)CCCC2 ; O=c1c(O)cccc2c1CCCC2]

In some aspects, the present disclosure provides a compound or a tautomer thereof, or a pharmaceutically acceptable salt of either, represented by Formula IIa, Formula IIb, or Formula IIc:

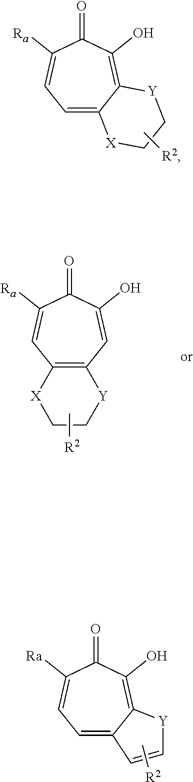

wherein:
   Rₐ represents hydrogen, halo, alkyl, substituted alkyl, heteroalkyl, alkoxy, substituted alkoxy, alkoxyalkyl, substituted alkoxyalkyl, cycloalkyl, substituted cycloalkyl, heterocycloalkyl, substituted heterocycloalkyl, alkenyl, substituted alkenyl, heteroalkenyl, cycloalkenyl, substituted cycloalkenyl, heterocycloalkenyl, substituted heterocycloalkenyl, alkynyl, substituted alkynyl, or heteroalkynyl;
   X and Y independently represent O, S, NH, or CR₅R₆;
   R₂ represents —F, alkyl, haloalkyl, or alkoxy; and
   R₅ and R₆ represent independently for each occurrence H, (C1-C15) alkyl, or substituted (C1-C15)alkyl;
   provided the compound is not

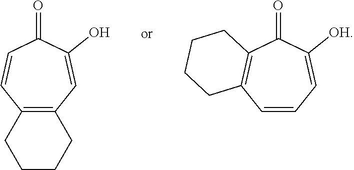 [SMILES: O=c1ccc2c(cc1O)CCCC2 ; O=c1c(O)cccc2c1CCCC2]

In certain embodiments, in Formula IIa and Formula IIb, Rₐ represents hydrogen.

In certain embodiments, the compound of Formula IIa or Formula IIb is selected from the group consisting of:

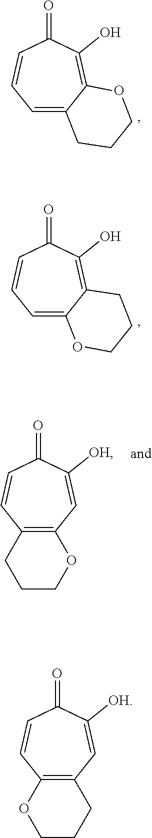 [SMILES: O=c1cccc2c(c1O)OCCC2 ; O=c1cccc2c(c1O)CCCO2 ; O=c1ccc2c(cc1O)OCCC2 ; O=c1ccc2c(cc1O)CCCO2]

In certain embodiments, the compound of Formula IIc is

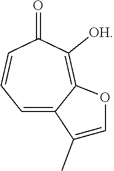

Representative compounds of Formula Ia, Formula Ib, Formula IIa, Formula IIb, and Formula IIc are tropolone derivatives, their tautomers, and pharmaceutically acceptable salts of either.

In certain embodiments, compounds of the present disclosure and tautomers thereof, and pharmaceutically acceptable salts of either, demonstrate desirable Absorption, Distribution, Metabolism, Excretion (ADME) and/or Drug Metabolism and Pharmacokinetics (DMPK) characteristics, demonstrating certain advantages desirable for further drug development. ADME and DMPK characteristics of a compound may be assessed by a variety of different assays that are known to the relevant ordinarily skilled artisan.

Useful such assays include, but are not limited to e.g., those measurements made in PK studies, such as rodent and non-human primate PK studies, including e.g., mouse (such as mouse 6-hour PK studies), rat PK studies, cynomolgus or rhesus PK studies, or the like. Useful measurements obtained in such PK studies include but are not limited to e.g., maximum concentration (Cₘₐₓ) reflecting the “peak” of a drug observed after its administration that can reflect not only the rate but also the extent of absorption, area under the curve (AUC) representing the area under the plot of tissue (e.g., plasma) concentration against time after drug administration which is of particular use in estimating bioavailability of drugs and drug total clearance, half-life (t_{1/2}) or the period of time required for the concentration or amount of drug to be reduced to exactly one-half of a given concentration or amount that indicates the persistence of the drug in its volume of distribution, and the like. Compounds of the present disclosure displayed improved PK characteristics, e.g., as measured by PK assays, including but not limited to e.g., where such compounds have improved PK characteristics as compared to a reference compound, such as hinokitiol.

Further examples of useful assays include metabolic stability assays, such as but not limited to e.g., liver microsomal clearance assays which provide measurement such as, but not limited to e.g., liver microsomal clearance half-life (t_{1/2}), liver microsomal intrinsic clearance (CLᵢₙₜ), and the like. Such measurements are useful in assessing various characteristics of a subject compound, such as e.g., the availability of an intact compound to provide a pharmacological effect. A compound having a longer microsomal clearance half-life (t_{1/2}), e.g., than a reference compound, will provide better exposure of the intact compound and greater availability to produce the relevant pharmacological effect. A compound having a less microsomal clearance, e.g., as measured by liver microsomal intrinsic clearance (CLᵢₙₜ), will similarly result in greater exposure of the intact compound available for an increase in the relevant pharmacological effect, e.g., as compared to that of a reference compound with a higher liver microsomal intrinsic clearance (CLᵢₙₜ). Other useful assays include in vitro hepatocyte metabolic stability assays. Compounds of the present disclosure displayed desirable characteristics in in vitro hepatocyte metabolic stability assays, including e.g., decreased metabolic clearance and increased systemic exposure. Compounds of the present disclosure displayed improved clearance characteristics, e.g., as measured by metabolic stability assays, including but not limited to e.g., where such compounds have improved clearance characteristics as compared to a reference compound, such as hinokitiol.

In certain embodiments, the compound of Formula Ia, Formula Ib, Formula IIa, Formula IIb, and Formula IIc has a human liver microsomal clearance half-life (t_{1/2}) of greater than 9 minutes. In certain embodiments, the compound of Formula Ia, Formula Ib, Formula IIa, Formula IIb, and Formula IIc has a human liver microsomal clearance half-life (t_{1/2}) of greater than 12 minutes. In certain embodiments, the compound of Formula Ia, Formula Ib, Formula IIa, Formula IIb, and Formula IIc has a human liver microsomal clearance half-life (t_{1/2}) of greater than 25 minutes In certain embodiments, the compound of Formula Ia, Formula Ib, Formula IIa, Formula IIb, and Formula IIc has a human liver microsomal clearance half-life (t_{1/2}) of greater than 50 minutes. In certain embodiments, the compound of Formula Ia, Formula Ib, Formula IIa, Formula IIb, and Formula IIc has a human liver microsomal clearance half-life (t_{1/2}) of greater than 100 minutes. In certain embodiments, the compound of Formula Ia, Formula Ib, Formula IIa, Formula IIb, and Formula IIc has a human liver microsomal clearance half-life (t_{1/2}) of greater than 150 minutes. In some instances, a compound of Formula Ia, Formula Ib, Formula IIa, Formula IIb, or Formula IIc of the present disclosure may have a human liver microsomal clearance half-life (t_{1/2}) of greater than 9 minutes, including but not limited to e.g., 10 min. or more, such as e.g., greater than 11 min., greater than 12 min., greater than 13 min., greater than 14 min., greater than 15 min., greater than 20 min., greater than 25 min., greater than 30 min., greater than 35 min., greater than 40 min., greater than 45 min., greater than 50 min., greater than 55 min., greater than 60 min., greater than 65 min., greater than 70 min., greater than 75 min., greater than 80 min., greater than 85 min., greater than 90 min., greater than 95 min., greater than 100 min., greater than 105 min., greater than 110 min., greater than 115 min., greater than 120 min., greater than 125 min., greater than 130 min., greater than 135 min., greater than 140 min., greater than 145 min., or greater than 150 min. or more. In some instances, a compound of Formula Ia, Formula Ib, Formula IIa, Formula IIb, or Formula IIc of the present disclosure may have a human liver microsomal clearance half-life (t_{1/2}) that is greater than a reference compound, such as but not limited to e.g., one or more of the reference compounds described herein, such as but not limited to e.g., hinokitiol.

In certain embodiments, the compound of Formula Ia, Formula Ib, Formula IIa, Formula IIb, and Formula IIc has a human liver microsomal intrinsic clearance (CLᵢₙₜ) of less than 120 μL/min/mg protein. In certain embodiments, the compound of Formula Ia, Formula Ib, Formula IIa, Formula IIb, and Formula IIc has a human liver microsomal intrinsic clearance (CLᵢₙₜ) of less than 50 μL/min/mg protein. In certain embodiments, the compound of Formula Ia, Formula Ib, Formula IIa, Formula IIb, and Formula IIc has a human liver microsomal intrinsic clearance (CLᵢₙₜ) of less than 46 μL/min/mg protein. In certain embodiments, the compound of Formula Ia, Formula Ib, Formula IIa, Formula IIb, and Formula IIc has a human liver microsomal intrinsic clearance (CLᵢₙₜ) of less than 43 μL/min/mg protein. In certain embodiments, the compound of Formula Ia, Formula Ib, Formula IIa, Formula IIb, and Formula IIc has a human liver microsomal intrinsic clearance (CLᵢₙₜ) of less than 25 μL/min/mg protein. In some instances, a compound of Formula Ia, Formula Ib, Formula IIa, Formula Ib, or Formula IIc of the present disclosure may have a human liver microsomal intrinsic clearance (CLᵢₙₜ) of less than 120 μL/min/mg protein, including but not limited to e.g., 119 μL/min/mg protein or less, such as e.g., less than 118 μL/min/mg protein, less than 117 μL/min/mg protein, less than 116 μL/min/mg protein, less than 115 μL/min/mg protein, less than 110 μL/min/mg protein, less than 105 μL/min/mg protein, less than 100 μL/min/mg protein, less than 95 μL/min/mg protein, less than 90 μL/min/mg protein, less than 85 μL/min/mg protein, less than 80 μL/min/mg protein, less than 75 μL/min/mg protein, less than 70 μL/min/mg protein, less than 65 μL/min/mg protein, less than 60 μL/min/mg protein, less than 55 μL/min/mg protein, less than 50 μL/min/mg protein, less than 45 μL/min/mg protein, less than 40 μL/min/mg protein, less than 35 μL/min/mg protein, less than 30 μL/min/mg protein, less than 25 μL/min/mg protein, less than 20 μL/min/mg protein, less than 15 μL/min/mg protein, or less than 10 μL/min/mg protein or less. In some instances, a compound of Formula Ia, Formula Ib, Formula IIa, Formula IIb, or Formula IIc of the present disclosure may have a human liver microsomal intrinsic clearance (CLᵢₙₜ) that is less than a reference compound, such as but not limited to e.g., one or more of the reference compounds described herein, such as but not limited to e.g., hinokitiol.

In certain embodiments, the compound of Formula Ia, Formula Ib, Formula IIa, Formula IIb, and Formula IIc has a 6-hour PK Cmax greater than 1000 ng/mL. In certain embodiments, the compound of Formula Ia, Formula Ib, Formula IIa, Formula IIb, and Formula IIc has a 6-hour PK Cmax greater than 1500 ng/mL. In certain embodiments, the compound of Formula Ia, Formula Ib, Formula IIa, Formula IIb, and Formula IIc has a 6-hour PK Cmax greater than 2000 ng/mL. In certain embodiments, the compound of Formula Ia, Formula Ib, Formula IIa, Formula IIb, and Formula IIc has a 6-hour PK Cmax greater than 2500 ng/mL. In certain embodiments, the compound of Formula Ia, Formula Ib, Formula IIa, Formula IIb, and Formula IIc has a 6-hour PK Cmax greater than 3000 ng/mL. In certain embodiments, the compound of Formula Ia, Formula Ib, Formula IIa, Formula IIb, and Formula IIc has a 6-hour PK Cmax greater than 3500 ng/mL. In certain embodiments, the compound of Formula Ia, Formula Ib, Formula IIa, Formula IIb, and Formula IIc has a 6-hour PK Cmax greater than 4000 ng/mL. In certain embodiments, the compound of Formula Ia, Formula Ib, Formula IIa, Formula IIb, and Formula IIc has a 6-hour PK Cmax greater than 5000 ng/mL. In certain embodiments, the compound of Formula Ia, Formula Ib, Formula IIa, Formula IIb, and Formula IIc has a 6-hour PK Cmax greater than 7500 ng/mL. In certain embodiments, the compound of Formula Ia, Formula Ib, Formula IIa, Formula IIb, and Formula IIc has a 6-hour PK Cmax greater than 10,000 ng/mL. In certain embodiments, the compound of Formula Ia, Formula Ib, Formula IIa, Formula IIb, and Formula IIc has a 6-hour PK Cmax greater than 15,000 ng/mL. In some instances, a compound of Formula Ia, Formula Ib, Formula IIa, Formula IIb, or Formula IIc of the present disclosure may have a 6-hour PK Cmax of greater than 1000 ng/mL, including but not limited to e.g., 1100 ng/mL or more, such as e.g., greater than 1200 ng/mL, greater than 1300 ng/mL, greater than 1400 ng/mL, greater than 1500 ng/mL, greater than 1600 ng/mL, greater than 1700 ng/mL, greater than 1800 ng/mL, greater than 1900 ng/mL, greater than 2000 ng/mL, greater than 2200 ng/mL, greater than 2400 ng/mL, greater than 2600 ng/mL, greater than 2800 ng/mL, greater than 3000 ng/mL, greater than 3200 ng/mL, greater than 3400 ng/mL, greater than 3600 ng/mL, greater than 3800 ng/mL, greater than 4000 ng/mL, greater than 4500 ng/mL, greater than 5000 ng/mL, greater than 5500 ng/mL, greater than 6000 ng/mL, greater than 6500 ng/mL, greater than 7000 ng/mL, greater than 7500 ng/mL, greater than 8000 ng/mL, greater than 8500 ng/mL, greater than 9000 ng/mL, greater than 9500 ng/mL, greater than 10000 ng/mL, greater than 10500 ng/mL, greater than 11000 ng/mL, greater than 11500 ng/mL, greater than 12000 ng/mL, greater than 12500 ng/mL, greater than 13000 ng/mL, greater than 13500 ng/mL, greater than 14000 ng/mL, greater than 14500 ng/mL, or greater than 15000 ng/mL or more. In some instances, a compound of Formula Ia, Formula Ib, Formula IIa, Formula IIb, or Formula IIc of the present disclosure may have a 6-hour PK Cmax that is greater than a reference compound, such as but not limited to e.g., one or more of the reference compounds described herein, such as but not limited to e.g., hinokitiol.

In certain embodiments, the compound of Formula Ia, Formula Ib, Formula IIa, Formula IIb, and Formula IIc has a 6-hour PK AUCₗₐₛₜ 0-6 hr greater than 590 hr*ng/mL. In certain embodiments, the compound of Formula Ia, Formula Ib, Formula IIa, Formula IIb, and Formula IIc has a 6-hour PK AUCₗₐₛₜ 0-6 hr greater than 700 hr*ng/mL. In certain embodiments, the compound of Formula Ia, Formula Ib, Formula IIa, Formula IIb, and Formula IIc has a 6-hour PK AUCₗₐₛₜ 0-6 hr greater than 1000 hr*ng/mL. In certain embodiments, the compound of Formula Ia, Formula Ib, Formula IIa, Formula IIb, and Formula IIc has a 6-hour PK AUCₗₐₛₜ 0-6 hr greater than 1500 hr*ng/mL. In certain embodiments, the compound of Formula Ia, Formula Ib, Formula IIa, Formula IIb, and Formula IIc has a 6-hour PK AUCₗₐₛₜ 0-6 hr greater than 5000 hr*ng/mL. In certain embodiments, the compound of Formula Ia, Formula Ib, Formula IIa, Formula IIb, and Formula IIc has a 6-hour PK AUCₗₐₛₜ 0-6 hr greater than 10,000 hr*ng/mL. In certain embodiments, the compound of Formula Ia, Formula Ib, Formula IIa, Formula IIb, and Formula IIc has a 6-hour PK AUCₗₐₛₜ 0-6 hr greater than 20,000 hr*ng/mL. In certain embodiments, the compound of Formula Ia, Formula Ib, Formula IIa, Formula IIb, and Formula IIc has a 6-hour PK AUCₗₐₛₜ 0-6 hr greater than 45,000 hr*ng/mL. In some instances, a compound of Formula Ia, Formula Ib, Formula IIa, Formula IIb, or Formula IIc of the present disclosure may have a 6-hour PK AUCₗₐₛₜ 0-6 hr of greater than 590 hr*ng/mL, including but not limited to e.g., 600 hr*ng/mL or more, such as e.g., greater than 700 ng/mL, greater than 800 ng/mL, greater than 900 ng/mL, greater than 1000 ng/mL, greater than 1500 ng/mL, greater than 2000 ng/mL, greater than 2500 ng/mL, greater than 3000 ng/mL, greater than 3500 ng/mL, greater than 4000 ng/mL, greater than 4500 ng/mL, greater than 5000 ng/mL, greater than 5500 ng/mL, greater than 6000 ng/mL, greater than 6500 ng/mL, greater than 7000 ng/mL, greater than 7500 ng/mL, greater than 8000 ng/mL, greater than 8500 ng/mL, greater than 9000 ng/mL, greater than 9500 ng/mL, greater than 10000 ng/mL, greater than 12000 ng/mL, greater than 14000 ng/mL, greater than 16000 ng/mL, greater than 18000 ng/mL, greater than 20000 ng/mL, greater than 22000 ng/mL, greater than 24000 ng/mL, greater than 26000 ng/mL, greater than 28000 ng/mL, greater than 30000 ng/mL, greater than 32000 ng/mL, greater than 34000 ng/mL, greater than 36000 ng/mL, greater than 38000 ng/mL, or greater than 40000 ng/mL or more. In some instances, a compound of Formula Ia, Formula Ib, Formula IIa, Formula IIb, or Formula IIc of the present disclosure may have a 6-hour PK AUCₗₐₛₜ 0-6 hr that is greater than a reference compound, such as but not limited to e.g., one or more of the reference compounds described herein, such as but not limited to e.g., hinokitiol.

In certain embodiments, the compound of Formula Ia, Formula Ib, Formula IIa, Formula IIb, and Formula IIc has a 6-hour PK tin greater than 1 hr. In certain embodiments, the compound of Formula Ia, Formula Ib, Formula IIa, Formula IIb, and Formula IIc has a 6-hour PK t_{1/2} greater than 1.3 hr. In certain embodiments, the compound of Formula Ia, Formula Ib, Formula IIa, Formula IIb, and Formula IIc has a 6-hour PK tin greater than 1.5 hr. In certain embodiments, the compound of Formula Ia, Formula Ib, Formula IIa, Formula IIb, and Formula IIc has a 6-hour PK tin greater than 2 hr. In certain embodiments, the compound of Formula Ia, Formula Ib, Formula IIa, Formula IIb, and Formula IIc has a 6-hour PK tin greater than 2.5 hr. In certain embodiments, the compound of Formula Ia, Formula Ib, Formula IIa, Formula IIb, and Formula IIc has a 6-hour PK t_{1/2} greater than 3 hr. In some instances, a compound of Formula Ia, Formula Ib, Formula IIa, Formula IIb, or Formula IIc of the present disclosure may have a 6-hour PK tin of greater than 1 hr, including but not limited to e.g., 1.1 hr or more, such as e.g., greater than 1.2 hr, greater than 1.3 hr, greater than 1.4 hr, greater than 1.5 hr, greater than 1.6 hr, greater than 1.7 hr, greater than 1.8 hr, greater than 1.9 hr, greater than 2 hr, greater than 2.1 hr, greater than 2.2 hr, greater than 2.3 hr, greater than 2.4 hr, greater than 2.5 hr, greater than 2.6 hr, greater than 2.7 hr, greater than 2.8 hr, greater than 2.9 hr, greater than 3 hr, greater than 3.1 hr, greater than 3.2 hr, greater than 3.3 hr, greater than 3.4 hr, greater than 3.5 hr, greater than 3.6 hr, greater than 3.7 hr, greater than 3.8 hr, greater than 3.9 hr, greater than 4 hr, greater than 4.1 hr, greater than 4.2 hr, greater than 4.3 hr, greater than 4.4 hr, greater than 4.5 hr, greater than 4.6 hr, greater than 4.7 hr, greater than 4.8 hr, greater than 4.9 hr, or greater than 5 hr or more. In some instances, a compound of Formula Ia, Formula Ib, Formula IIa, Formula IIb, or Formula IIc of the present disclosure may have a 6-hour PK tin that is greater than a reference compound, such as but not limited to e.g., one or more of the reference compounds described herein, such as but not limited to e.g., hinokitiol.

In certain embodiments, a compound of Formula Ia, Formula Ib, Formula IIa, Formula IIb, or Formula IIc of the present disclosure may have a combination of two or more, including three or more, four or more, etc., of the herein described, including aforementioned, characteristics. For example, a compound of the present disclosure may, in some instances, have two or more, three or more, or four or more, of human liver microsomal clearance half-life (t_{1/2}), human liver microsomal intrinsic clearance (CLᵢₙₜ), 6 hour PK Cmax, 6 hour PK AUCₗₐₛₜ 0-6 hr, and 6-hour PK t_{1/2} greater or less than, as relevant, a threshold value disclosed herein, including above.

### EXEMPLIFICATION

The present disclosure now being generally described, it will be more readily understood by reference to the following, which is included merely for purposes of illustration of certain aspects and embodiments of the present disclosure, and is not intended to limit the present disclosure.

### A. Synthesis of Tropolone Intermediates

### Preparation of building block BB1: 2-(benzyloxy)-7-bromocyclohepta-2,4,6-trien-1-one

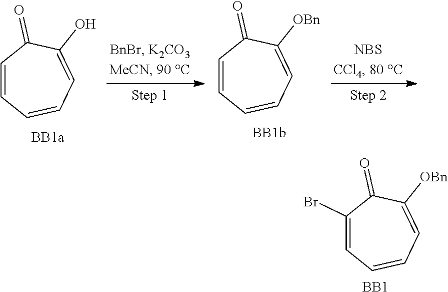

### Step 1:

To a stirred mixture of 2-hydroxycyclohepta-2,4,6-trien-1-one BB1a (20 g, 164 mmol, 1 eq) and K₂CO₃ (90.5 g, 655 mmol, 4 eq) in anhydrous MeCN (500 mL) was added benzyl bromide (42.0 g, 245 mmol, 1.5 eq) dropwise at 25 °C under N₂. The reaction mixture was then heated to reflux for 16 hrs. TLC (CH₂Cl₂:MeOH=10:1) showed the starting material BB1a was consumed completely and a new spot was observed. After cooling, the reaction mixture was filtered, and the filtrate was concentrated to dryness. Water (300 mL) was added to the residue, and the aqueous mixture was extracted with dichloromethane (200 mL×3). The combined organic phases were washed with brine (500 mL×2), dried with anhydrous Na₂SO₄, filtered and concentrated under reduced pressure to dryness. The residue was purified by silica gel column chromatography eluting with Petroleum ether:Ethyl acetate (20:1 to 5:1) to afford crude BB1b (36 g, 80% purity) as a yellow solid.

### Step 2:

To a solution of crude BB1b (Corel_3, 36 g, 132 mmol, 1 eq) in CCl₄ (450 mL) was added 1-bromopyrrolidine-2,5-dione (NBS) (30.5 g, 171 mmol, 1.3 eq) at 25 °C under N₂. The mixture was heated to 80 °C and stirred for 3 hrs. LCMS showed the starting material was almost completely consumed. After cooling, a saturated aqueous sodium thiosulfate solution (500 mL) was added, and the aqueous mixture was extracted with dichloromethane (200 mL×3). The combined organic phases were washed with brine (500 mL), dried over anhydrous Na₂SO₄, filtered and concentrated under reduced pressure to dryness. The residue was purified by silica gel column chromatography (Petroleum ether:Ethyl acetate=20:1 to 5:1) to afford 2-benzyloxy-7-bromo-cyclohepta-2,4,6-trien-1-one BB1 (15 g, 31.6% yield via 2 steps) as a yellow solid; ¹H NMR: 400 MHz CD₃OD, δ ppm 8.39-8.37 (m, 1H), 7.48-7.39 (m, 2H), 7.37-7.26 (m, 5H), 6.89-6.84 (m, 1H), 5.29 (s, 2H).

### Preparation of building block BB2: 4-bromo-7-fluoro-2-hydroxycyclohepta-2,4,6-trien-1-one

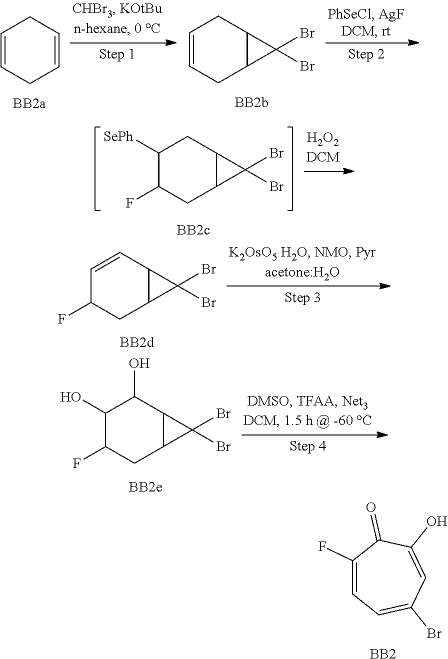

### Step 1:

To a mixture of 1,4-cyclohexene diene BB2a (10 g, 125 mmol, 1.2 eq) and KOtBu (19.8 g, 177 mmol, 1.7 eq) in n-hexane (150 mL) was added bromoform (26.3 g, 104 mmol, 1 eq) dropwise at 0 °C under N₂ atmosphere. The mixture was stirred at 0 °C for 3 h. TLC (100% pure petroleum ether) showed a new spot (Rf=0.8) was formed. The mixture was poured into water (100 mL) and extracted with petroleum ether (100 mL×3). The combined organic phases were washed with brine (100 mL), dried over Na₂SO₄, filtered and concentrated under reduced pressure to dryness. The residue was purified by silica gel column chromatography eluting with pure petroleum ether to afford BB2b (13.5 g, 42.9%) as a white solid.

### Step 2:

To a solution of BB2b (5 g, 19.9 mmol, 1 eq) in dichloromethane (70 mL) was added phenylselenyl chloride (4.56 g, 23.8 mmol, 1.2 eq) and silver fluoride (7.55 g, 59.5 mmol, 3 eq) under N₂ atmosphere. The system was degassed and recharged with nitrogen, repeated three times. The resulting mixture was stirred at 25 °C for 16 h under a N₂ atmosphere. TLC (100% pure petroleum ether) showed BB2b (Rf=0.8) was almost completely consumed. The mixture was filtered. The filtrate was treated with H₂O₂ (5.63 g, 49.61 mmol, 30% purity, 2.5 eq) dropwise at 25 °C. And the resulting mixture was stirred at 25 °C for 4 h. TLC (100% pure petroleum ether) showed a new spot (Rf=0.85) was formed. After filtering, the filtrate was washed with aq. Na₂SO₃ solution (30 mL×2), brine (30 mL), dried over Na₂SO₄, filtered and concentrated under reduced pressure to dryness. The residue was purified by silica gel column chromatography eluting with Petroleum ether:Ethyl acetate (20:1) to give crude BB2d (2 g, 37.4%) as a yellow oil.

### Step 3:

To a solution of NMO (3.34 g, 28.5 mmol, 1.4 eq) in acetone (60 mL) and H₂O (10 mL) was added BB2d (5.5 g, 20.4 mmol, 1 eq) and followed by the addition of K₂OsO₅·2H₂O (150 mg, 408 umol, 0.02 eq) in water (5 mL) under N₂. The mixture was stirred at 20 °C for 16 h under a N₂ balloon. TLC (petroleum ether:ethyl acetate=1:1) showed a new spot (Rf=0.5) was formed. Solid Na₂SO₃ (5 g) was added to quench the reaction at 0 °C. Then the mixture was concentrated to remove acetone. Brine (50 mL) was added to the residue, the aqueous mixture was extracted by EtOAc (50 mL×3). The combined organic layers were washed with brine (50 mL), dried over Na₂SO₄, filtered and concentrated under reduced pressure. The residue was purified by silica gel column chromatography eluting with Petroleum ether:Ethyl acetate (1:1) to give BB2e (3.2 g, 51.6%) as a white solid.

### Step 4:

To a stirred solution of DMSO (12.3 g, 158 mmol, 8 eq) in dichloromethane (50 mL) was added TFAA (33.2 g, 158 mmol, 8 eq) drop-wise under a N₂ atmosphere at −60 °C, the resulting colorless mixture was stirred at −60 °C for 15 min. A solution of BB2e (6 g, 19.7 mmol, 1 eq) in DMSO (30 mL) was added drop-wise at a temperature below −60 °C. The mixture was stirred at −60 °C for another 1.5 hours. And then Et₃N (27.8 g, 275 mmol, 14 eq) was added dropwise. Reaction was continued at −60 °C for 2 hours. The reaction mixture was warmed to 25 °C and stirred for 16 hours. TLC (EtOAc) indicated BB2e was consumed completely, and a new spot was formed. Water (100 mL) was added to the mixture and the aqueous layer was extracted with dichloromethane (100 mL×2). The combined organic phases were washed with brine (50 mL×2), dried over Na₂SO₄ and evaporated to dryness. The residue was purified by silica gel column chromatography eluting with Petroleum ether:Ethyl acetate (20:1 to 3:1) to afford building block BB2 (1.8 g, 41.7%) as a yellow solid.

### Preparation of building block BB14: 4-bromo-2-hydroxycyclohepta-2,4,6-trien-1-one

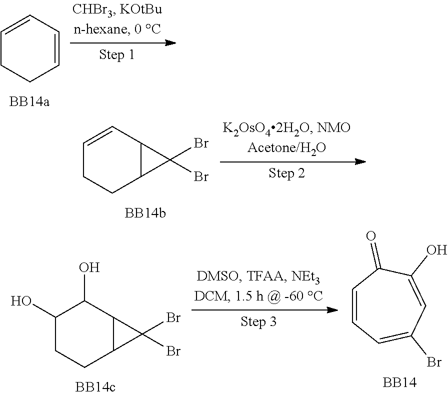 [SMILES: C1=CCCC=C1 ; BrC1(Br)C2C=CCCC21 ; OC1CCC2C(C1O)C2(Br)Br ; "BB14" ; O=c1cccc(Br)cc1O]

### Step 1:

To a solution of 1,3-cyclohexadiene BB14a (76 g, 949.6 mmol, 1.2 eq) and KOtBu (151 g, 1.35 mol, 1.7 eq) in n-hexane (500 mL) was added bromoform (200 g, 791.4 mmol, 1 eq) dropwise at 0 °C. The mixture was stirred at 0 °C for 1 h, and then continued at 25 °C 2 hours. TLC (100% petroleum ether) showed BB14a (Rf=0.9) was consumed completely, and a new spot was observed (Rf=0.8). The mixture was poured into water (500 mL), and the aqueous mixture was extracted with petroleum ether (300 mL×3). The combined organic phases were washed with brine (200 mL×2), dried over Na₂SO₄ and concentrated under reduced pressure to dryness. The residue was purified by silica gel column chromatography eluting with Petroleum ether/Ethyl acetate (100:0 to 100:1) to give BB14b (180 g, 90%) as a colorless oil.

### Step 2:

To a stirred solution of NMO (58.6 g, 500 mmol, 1.4 eq) in acetone (500 mL) and water (100 mL) was added BB14b (90 g, 357 mmol, 1 eq). A solution of K₂OsO₄·2H₂O (500 mg, 1.36 mmol, 0.004 eq) in water (30 mL) was added under N₂. The resulting mixture was stirred at 15 °C for 16 hr under a N₂ balloon. TLC (petroleum ether:EtOAc=1:1) indicated BB14b (Rf=0.9) was consumed completely and a new product spot was observed (Rf=0.3). Na₂SO₃ (15 g) was added to quench the reaction at 0 °C. The mixture was concentrated to remove acetone. Brine (500 mL) was added to the residue, then the aqueous mixture was extracted with EtOAc (200 mL×3). The combined organic layers were washed with brine (200 mL), dried over Na₂SO₄, and concentrated under reduced pressure to dryness. The residue was purified by silica gel column chromatography eluting with Petroleum ether/Ethyl acetate (10:1 to 1:1) to provide BB14c (70 g, 68.5%) as a white solid.

### Step 3:

To a solution of DMSO (26.2 g, 335 mmol, 8 eq) in DCM (300 mL) was added TFAA (70.5 g, 335 mmol, 8 eq) dropwise at −60 °C under N₂, the resulting colorless mixture was stirred at −60 °C for 15 min. A solution of 7,7-dibromonorcarane-2,3-diol BB14c (12 g, 41.9 mmol, 1 eq) in DMSO (10 mL) was added to the above mixture dropwise at a temperature below −60 °C. The mixture was stirred at −60 °C for another 1.5 hours. Et₃N (59.5 g, 588 mmol, 14 eq) was added dropwise, the resulting yellow solution was stirred at −60 °C for another 2 hours, then warmed to 25 °C. Reaction was continued at room temperature for 16 hours. TLC (EtOAc) indicated BB14c (Rf=0.6) was consumed completely, and a new spot was detected (Rf=0.2). Water (500 mL) was added to the mixture and the aqueous layer was extracted with DCM (200 mL×2). The combined organic phases were washed with brine (200 mL×2), dried over Na₂SO₄. and evaporated under vacuo to dryness. The residue was purified by silica gel column chromatography eluting with Petroleum ether/Ethyl acetate (50:1 to 20:1) to afford BB14 (5 g, 59.2%) as a yellow solid; ¹H NMR: 400 MHz CDCl₃, δ ppm 7.70-7.75 (m, 1H), 7.31-7.38 (m, 1H), 7.21-7.25 (m, 1H), 7.08-7.15 (m, 1H).

### Preparation of building block BB3

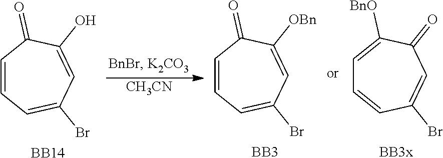

To a mixture of BB14 (10 g, 50 mmol, 1 eq) and K₂CO₃ (17.25 g, 125 mmol, 2.5 eq) in CH₃CN (60 mL) was added benzyl bromide (8.5 g, 75 mmol, 1.5 eq) in one portion at 20 °C. The mixture was heated and stirred at 90 °C for 2 hr. TLC (Petroleum ether:Ethyl acetate=3:1, Rf=0.3) indicated the starting material was consumed and a new spot was found. After cooling and filtering, the mixture was concentrated under reduced pressure to remove the solvent. The residue was purified by silica gel column chromatography eluting with Petroleum ether:Ethyl acetate (10:1 to 3:1) to give an inseparable mixture of BB3 and BB3x (6 g, ~80% purity) as a white solid. Methyl tert-butyl ether (MTBE, 60 mL) was added to the above solid mixture and the suspension was stirred at 50 °C for 30 min. After filtering of the hot mixture, the filter cake was washed with MTBE (20 mL×2) to give pure BB3 & BB3x (3 g, >95% purity), and the combined filtrate and washings were concentrated under reduced pressure to dryness to provide crude BB3 & BB3x (2.0 g, ~90% purity). ¹H NMR (400 MHz, METHANOL-d4) δ ppm 7.66 (d, J=1.98 Hz, 1H) 7.47 (t, J=6.73 Hz, 2H) 7.31-7.43 (m, 4H) 7.25 (dd, J=11.25, 1.54 Hz, 1H) 7.12-7.16 (m, 1H) 5.27 (d, J=5.51 Hz, 2H); LC-MS m/z [M+H]⁺: 290.8, 293.7.

### Preparation of building block BB4: 2-(benzyloxy)-5-bromocyclohepta-2,4,6-trien-1-one

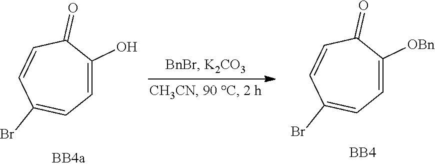

To a mixture of BB4a (commercially available) (10.9 g, 54.2 mmol, 1 eq) and K₂CO₃ (22.5 g, 163 mmol, 3 eq) in MeCN (200 mL) was added benzyl bromide (13.9 g, 81.3 mmol, 1.5 eq) in one portion at 25 °C under N₂. The mixture was stirred at 90 °C for 2 hours. TLC (petroleum ether:EtOAc=3:1) indicated the staring material was consumed completely and one new spot was formed. After cooling, the reaction mixture was filtered, and the filtrate was concentrated under reduced pressure to dryness. The residue was purified by silica gel column chromatography eluting with Petroleum ether/Ethyl acetate (20/1 to 3/1) to give BB4 (8 g, 50.7%) as a yellow solid ¹H NMR (400 MHz, DMSO-d6) δ ppm 7.54-7.44 (m, 2H), 7.43-7.33 (m, 5H), 6.94-6.88 (m, 2H), 5.18 (s, 2H).

### Preparation of building block BB5: 4-bromo-7-oxocyclohepta-1,3,5-trien-1-yl tert-butyl carbonate

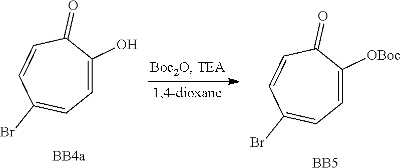

To a stirred solution of BB4a (5 g, 24.9 mmol, 1 eq) in dioxane (10 mL) was added TEA (10.1 g, 99.5 mmol, 4 eq) and Boc₂O (16.3 g, 74.6 mmol, 3 eq) in one portion at 25 °C under N₂. The mixture was heated to 118 °C and stirred for 1 hour. TLC (petroleum ether:EtOAc=5:1) indicated the starting material was consumed completely and one major new spot with lower polarity detected. After cooling, the mixture was concentrated under reduced pressure to dryness. The residue was purified by silica gel column chromatography (petroleum ether:EtOAc=20:1 to 8:1) to give building block BB5 (5 g, 33.4%) as a yellow solid; ¹H NMR (400 MHz, DMSO-d6) δ ppm 7.75-7.55 (m, 2H), 7.35-7.2 (m, 2H), 7.1-6.9 (m, 2H), 1.45 (s, 9H).

### Preparation of building block BB6: 3-bromo-7-oxocyclohepta-1,3,5-trien-1-yl tert-butyl carbonate

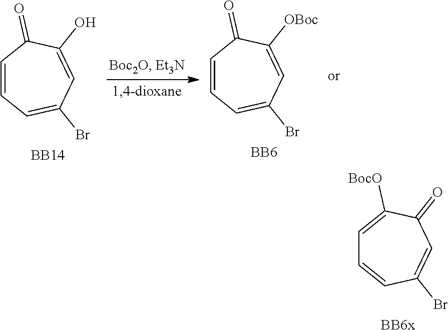

To a solution of BB14 (19 g, 94.5 mmol, 1 eq) in 1,4-dioxane (100 mL) was added Boc₂O (51.6 g, 236 mmol, 2.5 eq) and Et₃N (38.2 g, 378 mmol, 4 eq). The mixture was heated to 100 °C and stirred for 2 hours. TLC (petroleum ether:EtOAc=3:1) indicated BB14 (Rf=0.1) was consumed completely and a new spot (Rf=0.8) was observed. After cooling to room temperature, the mixture was concentrated under reduced pressure to dryness. The residue was purified by silica gel column chromatography (Petroleum ether/Ethyl acetate=20:1 to 3:1) twice to afford building block BB6 (12 g, 40.6%) as a brown oil; ¹H NMR 400 MHz, CD₃OD, 6 ppm 7.709-7.705 (m, 1H), 7.508-7.365 (m, 1H), 7.316-7.262 (m, 1H), 7.02-6.98 (m, 1H), 1.51 (s, 9H).

### Preparation of building block BB7

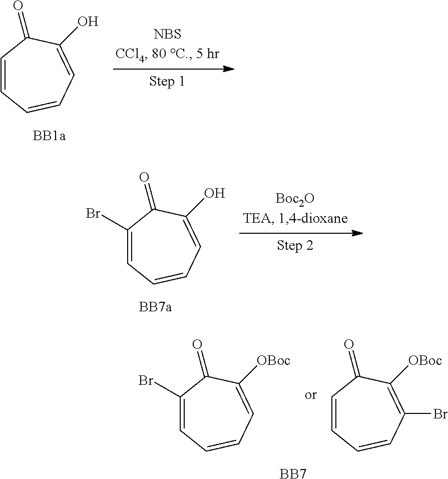

### Step 1:

To a solution of BB1a (20 g, 164 mmol, 1 eq) in CCl₄ (400 mL) was added NBS (26.1 g, 147 mmol, 0.9 eq) in portions at 25 °C under N₂. The mixture was heated and stirred at 80 °C for 5 hrs. LCMS showed the starting material was almost consumed. After cooling, a saturated aq. sodium thiosulfate solution (300 mL) was added drop-wise and the mixture was stirred for another 10 min. The aqueous mixture was extracted with dichloromethane (200 mL×3). The combined organic phases were washed with water (100 mL×2), brine (100 mL), dried over anhydrous Na₂SO₄, filtered and concentrated under reduced pressure to dryness to afford crude BB7a (20 g, crude) as an off-white solid.

### Step 2:

To a solution of crude BB7a (20 g, 100 mmol, 1 eq) in dioxane (200 mL) was added Boc₂O (43.6 g, 200 mmol, 2 eq) and TEA (20.2 g, 200 mmol, 2 eq) at 25 °C under N₂. The mixture was heated and stirred at 100 °C for 2 hr. LCMS showed the starting material was almost consumed and desired product mass was observed. After cooling, water (100 mL) was added and stirred for 10 min. The aqueous mixture was extracted with ethyl acetate (300 mL×3). The combined organic phases were washed with water (200 mL×2), brine (300 mL), dried over anhydrous Na₂SO₄, filtered and concentrated under reduced pressure to dryness. The residue was purified by silica gel column chromatography (Petroleum ether:Ethyl acetate=20:1 to 5:1) twice to afford building block BB7 (10 g, 33.3% yield, 90% purity) as a yellow oil; ¹H NMR (400 MHz, DMSO-d6) δ ppm 8.50-8.48 (m, 1H), 7.64-7.61 (m, 1H), 7.36-7.31 (m, 1H), 7.16-7.11 (m, 1H), 1.47 (s, 9H).

### Preparation of building block BB8: tert-butyl (4-iodo-7-oxocyclohepta-1,3,5-trien-1-yl) carbonate

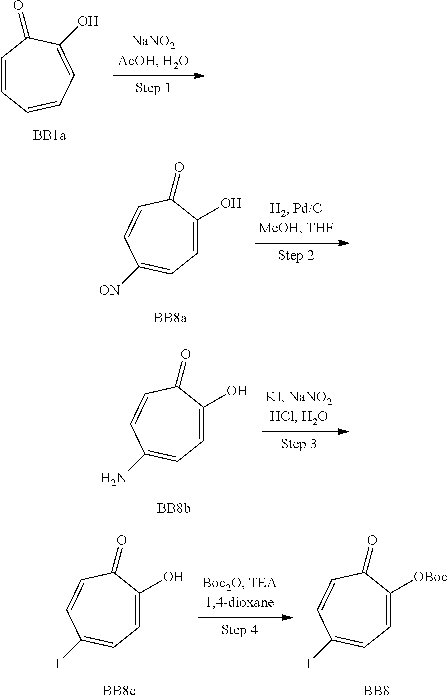

### Step 1:

To a mixture of Tropolone BB1a (100 g, 819 mmol, 1 eq) in AcOH (600 mL) and H₂O (200 mL) was added a solution of NaNO₂ (84.7 g, 1.23 mol, 1.5 eq) in H₂O (400 mL) dropwise at 0 °C. The mixture was stirred at 0 °C for 1 hr. LCMS showed the starting material was consumed. A yellow precipitate was collected by filtration and rinsed with H₂O (200 mL) to give the intermediate BB8a (106 g, 85.7%) as a yellow solid which was used in the next step without further purification

### Step 2:

To a solution of BB8a (10 g, 66.2 mmol, 1 eq) in MeOH (300 mL) and THF (600 mL) was added 10% Pd/C (3.52 g, 0.05 eq) under N₂. The system was degassed and purged with H₂ three times. The mixture was stirred under a hydrogen balloon (15 psi) at 15 °C for 4 hrs. TLC (DCM:MeOH=10:1) showed the starting material was consumed completely. The reaction mixture was filtered through a pad of Celite and the filter cake was washed with MeOH (100 mL×2). The combined filtrates were concentrated under reduced pressure to give BB8b (9 g, crude) as a yellow solid, which was used in the next step directly.

### Step 3:

To a solution of BB8b (30 g, 218 mmol, 1 eq) in H₂O (600 mL) and HCl (600 mL) was added NaNO₂ (30.2 g, 437 mmol, 2 eq) in H₂O (300 mL) drop-wise at 0 °C. The mixture was stirred at 0 °C for 15 min. After that, a solution of KI (109 g, 656 mmol, 3 eq) in H₂O (300 mL) was added to drop-wise at 0 °C. The reaction mixture was stirred at 15 °C for 16 hours. TLC (Ethyl acetate:MeOH=1:1) showed a new product spot was formed. The mixture was filtered, and the filter cake was washed with EtOAc (500 mL). The mixture was separated, the aqueous phase was extracted with EtOAc (1 L×3). The combined organic phase and organic extracts were washed with Sat. NaHSO₃ (1 L×3), water (1 L×3) and brine (1 L×2), dried over Na₂SO₄ and concentrated under reduced pressure to give intermediate BB8c (29 g, crude). The crude material was purified by silica gel column chromatography eluting with petroleum ether:EtOAc (10:1 to 0:1) to give intermediate BB8c (15 g, 27.6%) as a yellow solid.

### Step 4:

To a mixture of BB8c (30 g, 121 mmol, 1 eq) and Et₃N (61.1 g, 605 mmol, 5 eq) in 1,4-dioxane (500 mL) was added Boc₂O (79.1 g, 363 mmol, 3 eq) drop-wise at 20 °C. The mixture was stirred at 110 °C for 2 hrs. TLC (MeOH/Ethyl acetate=1:1) showed the starting material was consumed completely and TLC (Petroleum ether/Ethyl acetate=5:1) showed a new product spot was observed (Rf=0.5). After cooling, the mixture was concentrated under reduced pressure to dryness. The residue was purified by silica gel column chromatography eluting with petroleum ether:EtOAc (50:1 to 10:1) to afford the building block BBB (23 g, 54%) as a yellow solid; ¹H NMR (400 MHz, DMSO-d6) δ ppm 7.84-7.81 (m, 2H), 7.054 (brs, 1H), 6.81 (brs, 1H), 1.45 (s, 9H).

### Preparation of building block BB9: tert-butyl (4-iodo-7-oxocyclohepta-1,3,5-trien-1-yl) carbonate

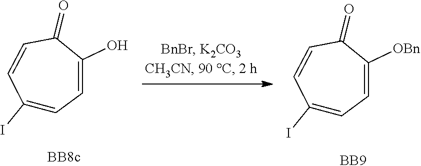

To a solution of iodo-tropolone BB8c (4.9 g, 19.7 mmol, 1 eq) in MeCN (200 mL) was added K₂CO₃ (8.19 g, 59.3 mmol, 3 eq) and benzyl bromide (5.07 g, 29.6 mmol, 1.5 eq) in one portion at 25 °C under N₂. The mixture was heated and stirred at 90 °C for 2 h. TLC (Petroleum ether:EtOAc=3:1) indicated the starting material was consumed completely and one new spot was formed. The reaction mixture was filtered, and the filtrate was concentrated under reduced pressure to dryness. The residue was purified by silica gel column chromatography eluting with Petroleum ether/Ethyl acetate (20/1 to 3/1) to afford the building block BB9 (2 g, 29.9%) as a yellow solid; ¹H NMR 400 MHz, CDCl₃, δ ppm 7.58-7.52 (m, 2H), 7.40-7.37 (m, 5H), 7.27-6.82 (m, 1H), 6.80-6.36 (m, 1H), 5.24 (s, 2H).

### Preparation of building block BB10: tert-butyl (7-oxo-3-(4,4,5,5-tetramethyl-1,3,2-dioxaborolan-2-yl)cyclohepta-1,3,5-trien-1-yl) carbonate

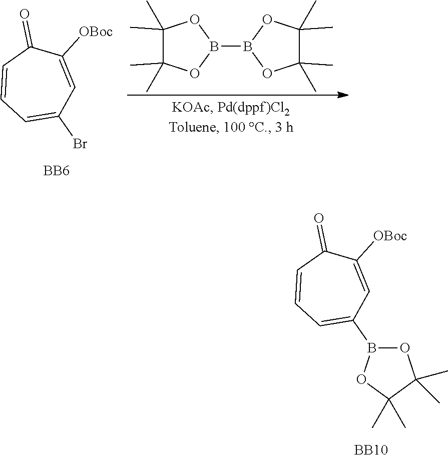

To a solution of building block BB6 (4 g, 13.3 mmol, 1 eq) in toluene (50 mL) was added bis(pinacolato)diboron (Pin2B2, 3.54 g, 13.9 mmol, 1.05 eq), KOAc (1.96 g, 19.9 mmol, 1.5 eq) and Pd(dppf)Cl₂ (972 mg, 1.33 mmol, 0.1 eq) under N₂. The system was degassed and recharged with nitrogen for three times. The mixture was heated and stirred at 100 °C for 3 hours under nitrogen. TLC (Petroleum ether:Ethyl acetate=5:1 and 1:1) indicated starting material was consumed completely and one major new spot with larger polarity was detected. After cooling, the reaction solution was filtered through a pad of Celite and the filter cake was washed with CH₂Cl₂ (50 mL×2). The combined filtrates were washed with brine (20 mL), dried over Na₂SO₄, filtered and concentrated under reduced pressure to give the crude product. The crude product was purified by silica gel column chromatography eluting with Petroleum ether:Ethyl acetate (20:1 to 2:1) to give building block BB10 (2.7 g, 7.75 mmol, 58.4%) as a yellow gum.

### Preparation of building block BB11: tert-butyl (3-hydroxy-7-oxocyclohepta-1,3,5-trien-1-yl) carbonate

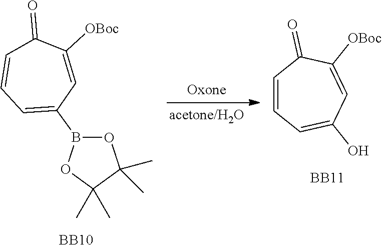

To a solution of building block BB10 (250 mg, 720 umol, 1 eq) in acetone (4 mL) and H₂O (1 mL) was added Oxone (660 mg, 1.08 mmol, 1.5 eq) in one portion at 25 °C. The mixture was stirred at 25 °C for 2 hr. LC-MS showed the starting material was consumed completely and the desired product mass was detected. The result mixture was poured into water (10 mL) and then extracted with ethyl acetate (20 mL×3). The combined solution was washed with aqueous Na₂SO₃ solution (10 mL×2), brine (10 mL), dried over anhydrous Na₂SO₄, filtered and concentrated in vacuo to give the building block BB11 (98 mg, 60%) as a white solid; ¹H NMR 400 MHz, DMSO-d6, δ ppm 7.82 (brs, 1H), 7.75-7.68 (m, 1H), 7.57-7.52 (m, 1H), 7.33 (brd, J=11.4 Hz, 1H), 1.45 (s, 9H).

### Preparation of Building Block BB12

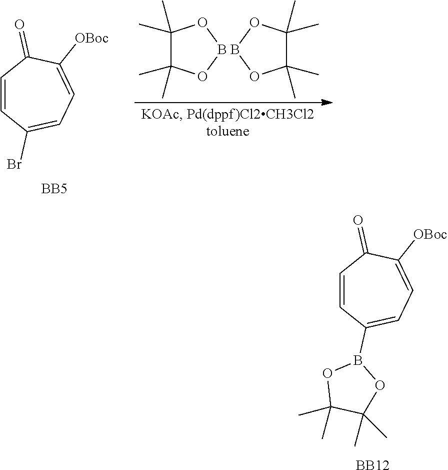

A mixture of building block BB5 (1.00 g, 3.32 mmol, 1.00 eq), 4,4,4′,4′,5,5,5′,5′-octamethyl-2,2′-bi(1,3,2-dioxaborolane) (927 mg, 3.65 mmol, 1.10 eq) and KOAc (651 mg, 6.64 mmol, 2.00 eq) in toluene (20.0 mL) was degassed and purged with N₂. To the mixture was added Pd(dppf)Cl₂·CH₂Cl₂ complex (542 mg, 664 umol, 0.20 eq), and the mixture was stirred at 120 °C for 1 hour under N₂ atmosphere. Reaction progress was monitored by TLC (petroleum ether:EtOAc (3:1, product Rf=0.1). The reaction mixture was filtered and concentrated to dryness under vacuum. The residue was purified by silica gel column chromatography eluting with petroleum ether:EtOAc (100:1 to 9:1) to afford BB12 (1.40 g, crude) as a white solid.

### B. Synthesis of Tropolone Derivatives

In certain embodiments, tropolone intermediates obtained in step a. were further reacted with various reagents to produce tropolone derivatives. The synthesis, purification, and characterization of each representative tropolone derivative are described in detail in the following examples.

### Examples 1 and 2: Preparation of 7-fluoro-2-hydroxy-4-isopropylcyclohepta-2,4,6-trien-1-one (Ex.1) and 3-fluoro-2-hydroxy-4-isopropylcyclohepta-2,4,6-trien-1-one (Ex.2)

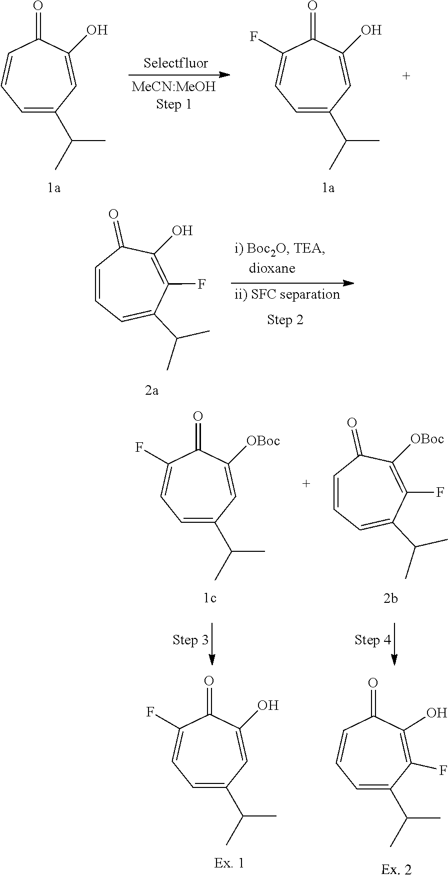

### Step 1:

To a solution of β-thujaplicin 1a (1 g, 6.09 mmol, 1 eq) in MeCN (10 mL) and MeOH (10 mL) was added Selectfluor (3.24 g, 9.14 mmol, 1.50 eq) at 0 °C under a N₂ atmosphere. The reaction mixture was heated to and stirred at 40 °C for 20 hours. The reaction progress was monitored by LCMS which showed some starting material 1a still remained while desired intermediates were formed. After cooling to room temperature, brine (50 mL) was added, and the aqueous mixture was extracted with dichloromethane (50 mL×3). The combined organic layers were washed with brine (20 mL), dried over anhydrous Na₂SO₄, filtered and concentrated under reduced pressure to dryness to provide a crude mixture of fluorinated β-thujaplicin 1b and 2a (2 g, crude) as a yellow gum, which was used in the next step directly without further purification.

### Step 2:

To a mixture of 1b and 2a from step 1 above (2 g, 10.9 mmol, 1 eq) and TEA (1.11 g, 10.9 mmol, 1.00 eq) in dioxane (25 mL) was added Boc₂O (4.79 g, 21.9 mmol, 2.00 eq) at 20 °C under N₂. The resulting mixture was heated to and stirred at 110 °C for 1 h. The reaction progress was monitored by TLC, new product spot was observed (Petroleum ether:Ethyl acetate=5:1, Rf=0.5). After cooling to room temperature, brine (50 mL) was added and the aqueous mixture was extracted with methyl tert-butyl ether (MTBE, 50 mL×3). The combined organic extracts were washed with water (20 mL), brine (20 mL), dried over anhydrous Na₂SO₄, filtered and concentrated under reduced pressure to dryness. The residue was purified by silica gel column chromatography eluting with petroleum ether-ethyl acetate (5:1). Removal of solvents gave a crude product (240 mg), which was further purified and separated by chiral SFC (column: Phenomenex-Cellulose-2 (250 mm*30 mm, 10 um); mobile phase:[Neu-IPA]; B %: 50%-50%, 4 min) to afford intermediate 1c (50 mg) as a yellow gum and intermediate 2b (100 mg) as a yellow solid.

### Step 3:

To a solution of intermediate 1c (50 mg) in dichloromethane (2 mL) was added TFA (0.5 mL) in one portion at 0 °C. The mixture was warmed to and stirred at 25 °C for 1 hr. The reaction mixture was diluted with CH₂Cl₂ (10 mL) and concentrated under reduced pressure to dryness at temperature below 10 °C. The residue was re-dissolved in CH₂Cl₂ (10 mL), treated with Amberlyst A21 (0.1 g) and stirred for another 0.5 hr. The mixture was filtered, and the filter cake was rinsed with CH₂Cl₂ (5 mL×2). The combined filtrates were concentrated under reduced pressure to provide the titled compound Ex.1 (31 mg, 96%) as a yellow gum; ¹H NMR: 400 MHz CD₃OD, n 7.71-7.59 (m, 1H), 7.48 (d, J=1.2 Hz, 1H), 7.08 (br d, J=10.6 Hz, 1H), 2.99 (quin, J=6.8 Hz, 1H), 1.35-1.28 (m, 6H); LC-MS: m/z [M+H]⁺=183.1.

### Step 4:

To a solution of intermediate 2b (100 mg, 354 umol, 1 eq) in dichloromethane (2 mL) was added TFA (0.5 mL) in one portion at 0 °C. The mixture was warmed to and stirred at 25 °C for 1 hr. The reaction mixture was diluted with CH₂Cl₂ (10 mL) and concentrated under reduced pressure to dryness at temperature below 10 °C. The residue was re-dissolved in CH₂Cl₂ (10 mL) and treated with Amberlyst A21 (0.1 g), the mixture was stirred for 0.5 hr and filtered, the filter cake was rinsed with CH₂Cl₂ (5 mL×2). The combined filtrates were concentrated under reduced pressure to provide the titled product Ex.2 (50 mg, 77.4%) as a yellow gum; ¹H NMR: 400 MHz CD₃OD, δ ppm 7.30-7.21 (m, 1H), 7.31-7.20 (m, 1H), 7.06 (s, 1H), 6.42 (br d, J=11.6 Hz, 1H), 2.76 (td, J=13.6 Hz, 2H), 1.23 (d, J=7.2 Hz, 6H); LC-MS: m/z [M+H]⁺=183.1.

### Example 3: Preparation of 2-fluoro-7-hydroxycyclohepta-2,4,6-trien-1-one (Ex.3)

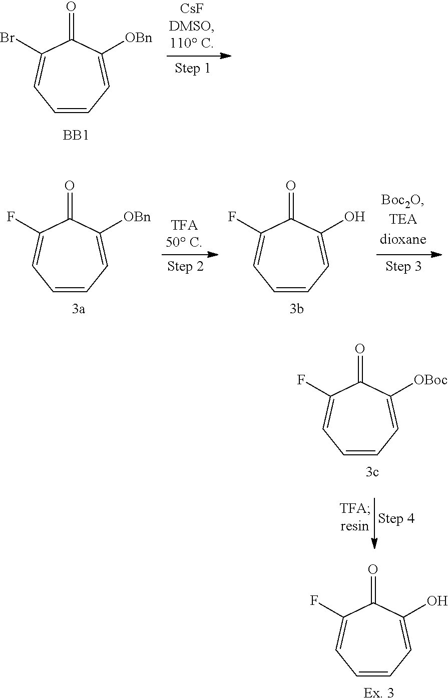

### Step 1:

To a stirred solution of building block BB1 (8 g, 27.4 mmol, 1 eq) in DMSO (100 mL) was added dried CsF (6.25 g, 41.1 mmol, 1.5 eq) under a N₂ atmosphere. The mixture was degassed and then charged with nitrogen, repeated the process three times. The mixture was then heated to and stirred at 110 °C for 5 hours. Reaction progress was monitored by LCMS, ~40% of desired product was formed with some BB1 remained. After cooling to 25 °C, brine (200 mL) was added and the aqueous mixture was extracted with EtOAc (200 mL×2); the combined organic layers were washed with brine (50 mL), dried over anhydrous Na₂SO₄, filtered and concentrated under reduced pressure to dryness. The yellow residue was purified by silica gel column chromatography eluting with petroleum ether:EtOAc (20:1 to 4:1) to give fluoro intermediate 3a (2.1 g, 33.2% yield) as yellow solid.

### Step 2:

Intermediate 3a (2.1 g) from step 1 above was dissolved in TFA (7.5 mL) and the mixture was stirred at 50 °C for 1 h. After cooling, the mixture was concentrated under reduced pressure at a temperature below 5 °C to give crude product 3b (1.6 g, crude) as a yellow gum.

### Step 3:

To a solution of crude 3b (0.1 g, 0.71 mmol, 1 eq) in dioxane (2 mL) was added TEA (140 mg, 1.4 mmol, 2 eq) and Boc₂O (320 mg, 1.4 mmol, 1.64 mL, 2 eq) in one portion at 25 °C under N₂. The mixture was heated to and stirred at 100 °C for 1 hour. Reaction progress was monitored by TLC (petroleum ether:EtOAc=5:1, product Rf=0.5). After cooling, the mixture was concentrated under reduced pressure to dryness and the residue was purified by pre-TLC (petroleum ether:ethyl acetate=5:1) to provide 3c (60 mg, 35%) as a yellow solid.

### Step 4:

To a solution of 3c (60 mg) in CH₂Cl₂ (4 mL) was added TFA (0.2 mL) in one portion at 0 °C. The resulting mixture was warmed to and stirred at 25 °C for 1 hr. The reaction mixture was diluted with CH₂Cl₂ (10 mL) and concentrated under a reduced pressure to dryness at a temperature below 10 °C. The residue was re-dissolved in CH₂Cl₂ (10 mL) and treated with Amberlyst A21 (0.1 g). The mixture was stirred for 0.5 hr, filtered, and the filtering cake was rinsed with with CH₂Cl₂ (5 mL×2). The filtrate was concentrated under reduced pressure to provide the titled product Ex.3 (30 mg, 85%) as a yellow solid; ¹H NMR: 400 MHz CDCl₂, δ ppm 7.56 (dd, J=10.4 Hz, 1H), 7.49-7.43 (m, 1H), 7.41-7.33 (m, 1H), 7.03 (dt, J=10.4 Hz, 1H); LC-MS: m/z [M+H]⁺=141.1.

### Example 4: Preparation of 7-fluoro-2-hydroxy-3-isopropylcyclohepta-2,4,6-trien-1-one (Ex.4)

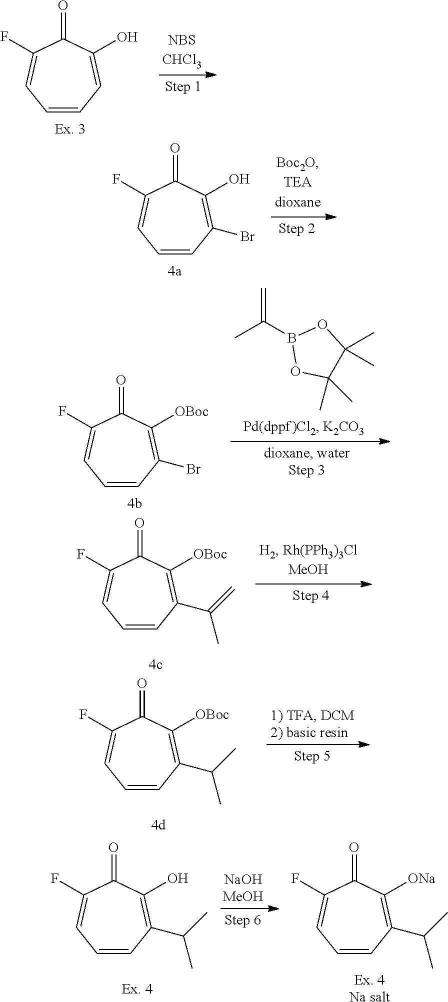

### Step 1:

To a stirred solution of Ex.3 (1.60 g, 11.4 mmol, 1.00 eq) in CHCl₃ (30 mL) was added NBS (2.44 g, 13.7 mmol, 1.20 eq) in portions at 25 °C under N₂. The mixture was heated to and stirred at 80 °C for 1 h. Reaction progress was monitored by LCMS. After cooling, water (30 mL) was added and the aqueous mixture was extracted with dichloromethane (30 mL×3). The combined organic extracts were washed with water (20 mL), brine (50 mL), dried over anhydrous Na₂SO₄, filtered and concentrated under reduced pressure to dryness to give crude fluoro/bromo intermediate 4a (4 g, crude) as a yellow gum, which was used in the next step without purification.

### Step 2:

To a stirred solution of 4a (4 g, 18.2 mmol, 1.00 eq) in dioxane (5 mL) was added Et₃N (5.54 g, 54.7 mmol, 3.00 eq) followed by Boc₂O (7.97 g, 36.5 mmol, 2.00 eq) in one portion at 25 °C under N₂. The resulting mixture was heated to and stirred at 110 °C for 1 h. Reaction progress was monitored by LCMS. After cooling, the mixture was concentrated under reduced pressure to dryness. The residue was purified by silica gel column chromatography eluting with Petroleum ether:Ethyl acetate (20:1 to 5:1, Rf=0.65) to provide 4b (0.9 g, 15.4%) as a colorless gum.

### Step 3:

To a mixture of 4b (150 mg, 470.04 umol, 1 eq) and 4,4,5,5-tetramethyl-2-(prop-1-en-2-yl)-1,3,2-dioxaborolane (144 mg, 940 umol, 2 eq) in dioxane (3 mL) and water (0.6 mL) was added K₂CO₃ (129 mg, 940 umol, 2 eq) and Pd(dppf)Cl₂ (34.3 mg, 47.0 umol, 0.10 eq) in one portion under N₂ atmosphere. The resulting mixture was degassed and then charged with nitrogen, repeating this process three times. The mixture was then heated to and stirred at 118 °C for 30 min. Reaction progress was monitored by TLC (Petroleum ether:Ethyl acetate=10:1, product Rf=0.55). After cooling to room temperature, brine (10 mL) was added, the aqueous mixture was extracted with EtOAc (25 mL×2), and the combined organic extracts were washed with water (10 mL), brine (20 mL), dried over anhydrous Na₂SO₄, filtered and concentrated under reduced pressure to dryness. The residue was purified by prep-TLC (Petroleum ether:Ethyl acetate=10:1, Rf=0.5) to yield 4c (100 mg, 75.9%) as a colorless gum.

### Step 4:

To a stirred solution of 4c (100 mg, 356 umol, 1 eq) in MeOH (4 mL) was added Rh(PPh₃)₃Cl (330 mg, 356 umol, 1 eq) under N₂. The suspension was degassed under vacuum and purged with H₂ three times. The mixture was stirred under H₂ (30 psi) at 25 °C for 0.5 hour. The product was detected by TLC (Petroleum ether:Ethyl acetate=5:1, Rf=0.6). The reaction mixture was filtered through a pad of Celite and the filter cake was washed with MeOH (5 mL×3). The combined filtrates were concentrated under reduced pressure to dryness. The residue was purified by prep-TLC (Petroleum ether:Ethyl acetate=5:1, Rf=0.5) to give 4d (60 mg, 59.5%) as a colorless gum.

### Step 5:

To a solution of 4d (37 mg, 131 umol, 1 eq) in dichloromethane (2 mL) was added TFA (0.5 mL) in one portion at 0 °C. The mixture was warmed to and stirred at 25 °C for 0.5 hr. TLC (petroleum ether:EtOAc=5:1) showed the starting material was consumed completely. The reaction mixture was diluted with CH₂Cl₂ (10 mL) and concentrated under reduced pressure to dryness at a temperature below 10 °C. The residue was re-dissolved in CH₂Cl₂ (10 mL) and treated with Amberlyst A21 (0.1 g). The mixture was stirred for 0.5 hr, filtered, and the filtering cake was rinsed with CH₂Cl₂ (5 mL×2). The combined filtrate was concentrated under reduced pressure to afford Ex.4 (22 mg, 92.1%) as light yellow solid; ¹H NMR: 400 MHz CDCl₃, δ ppm 7.48-7.37 (m, 2H), 6.98 (m, 1H), 3.74 (m, 1H), 1.27 (d, J=7.2 Hz, 6H); LC-MS: m/z [M+H]⁺=183.2.

### Step 6:

To a solution of Ex.4 from step above (51.6 mg, 283 umol, 1 eq) in MeOH (1 mL) was added a solution of NaOH (11.3 mg, 283 umol, 1 eq) in distilled water (0.1 mL) drop-wise at 25 °C. The reaction mixture was stirred at 25 °C for 1 h, and then concentrated under reduced pressure to remove MeOH. The crude product was triturated with acetone at 25 °C and stirred for another 30 min, then filtered. The filtering cake was washed with acetone (5 mL×2) and the precipitate was collected and dried in vacuum to provide sodium salt of Ex.4 (46 mg, 79.5%) as a yellow solid; ¹H NMR: 400 MHz CD₃OD, S ppm 7.27-7.12 (m, 2H), 6.40 (dt, J=10.8 Hz, 1H), 3.83-3.75 (m, 1H), 3.79 (td, J=7.2, 13.6 Hz, 1H), 1.15 (d, J=6.8 Hz, 6H); LC-MS: m/z [M+H]⁺=183.1.

### Example 5: Preparation of 5-(8-oxabicyclo[3.2.1]octan-3-yl)-2-hydroxycyclohepta-2,4,6-trien-1-one (Ex.5)

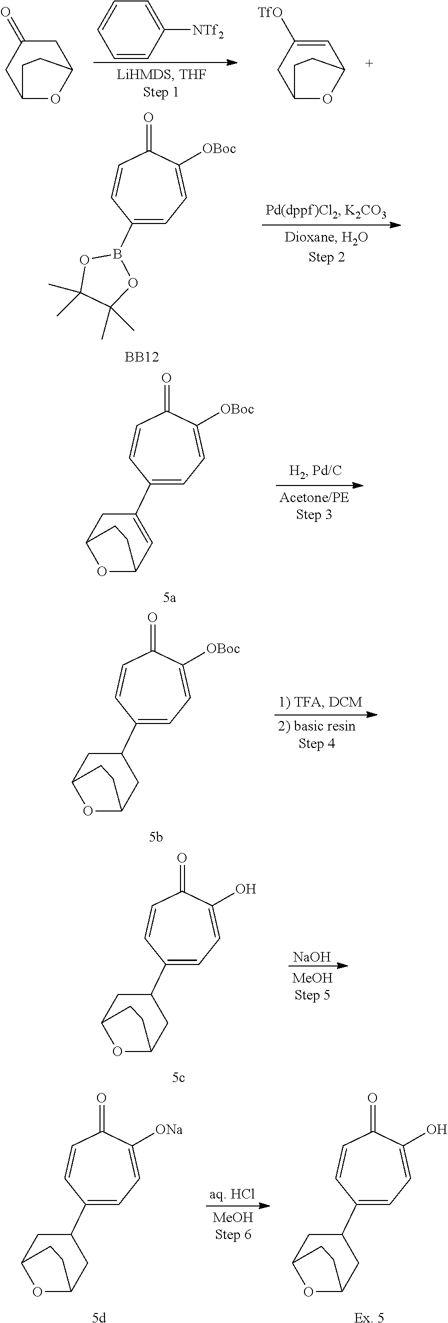

### Step 1:

To a solution of 8-oxabicyclo[3.2.1]octan-3-one (300 mg, 2.38 mmol, 1 eq) in THF (8 mL) was added LiHMDS (1 M, 3.3 mL, 1.4 eq) in one portion at −78 °C under N₂. The mixture was stirred at −78 °C for 30 min, then N,N-bis(trifluoromethylsulphonyl)aniline (1.10 g, 3.09 mmol, 1.3 eq) in THF (8 mL) was added to the mixture drop-wise under N₂ and stirred for 0.5 hours. The mixture was slowly warmed to 25 °C and stirred for another 2 hours. The reaction was monitored by TLC (petroleum ether:EtOAc=5:1). After cooling to 0 °C, the reaction was quenched with H₂O (10 mL) and then extracted with petroleum ether (50 mL×3). The combined organic layers were washed with water (10 mL), brine (10 mL), dried over Na₂SO₄, filtered and concentrated under reduced pressure to give 8-oxabicyclo[3.2.1]oct-2-en-3-yl trifluoromethanesulfonate (600 mg, crude) as a yellow oil.

### Step 2:

To a solution of building block BB12 (674 mg, 1.94 mmol, 1 eq) in dioxane (10 mL) and H₂O (2 mL) was added K₂CO₃ (535 mg, 3.87 mmol, 2 eq), 8-oxabicyclo[3.2.1]oct-2-en-3-yl trifluoromethanesulfonate (0.5 g, 1.94 mmol, 1 eq) and Pd(dppf)Cl₂·CH₂Cl₂ (158 mg, 194 umol, 0.1 eq) in one portion under N₂ atmosphere. The system was degassed and charged with nitrogen three times. The mixture was heated and stirred at 118 °C for 1 hr under N₂ atmosphere. Reaction progress was monitored by TLC (petroleum ether:EtOAc=1:1, product Rf=0.30). After cooling, water (10 mL) was added and then extracted with EtOAc (70 mL×3). The combined organic layers were washed with brine (10 mL), dried over Na₂SO₄, filtered and concentrated under reduced pressure to give a residue. The residue was purified by silica gel column chromatography eluting with petroleum ether:EtOAc (20:1 to 3:1) to afford product 5a (0.16 g, 25.0% yield) as a yellow oil.

### Step 3:

To a solution of 5a (125 mg, 378 umol, 1 eq) in acetone (2 mL) and petroleum ether (0.5 mL) was added Pd/C (30 mg, 10% purity) under N₂. The suspension was degassed under vacuum and purged with H₂ several times. The mixture was stirred under H₂ (15 psi) at 40 °C for 0.5 hour. The reaction was monitored by TLC (petroleum ether:EtOAc=3:1, product Rf=0.32). The reaction mixture was filtered through a pad of Celite and the filter cake was washed with acetone (10 mL×2). The combined filtrates were concentrated under reduced pressure to dryness. The residue was purified by prep-HPLC (column: Welch Xtimate C18 (100*25 mm, 3 um); mobile phase:[water(0.1% TFA)-ACN]; B %: 35%-55%, 12 min) to give product 5b (80 mg, 63.6% yield) as brown oil.

### Step 4:

To a solution of 5b (80 mg, 240 umol, 1 eq) in CH₂Cl₂ (1 mL) was added TFA (0.5 mL) in one portion at 0 °C. The mixture was warmed and stirred at 25 °C for 0.5 hr. The reaction was monitored by TLC (petroleum ether/EtOAc=1:1, product Rf=0.00). The reaction mixture was diluted with CH₂Cl₂ (10 mL) and concentrated under reduced pressure to dryness below 10 °C. Then the mixture was re-dissolved in CH₂Cl₂ (10 mL), treated with Amberlyst A21 (0.1 g) and stirred for another 0.5 hr. After filtering, the cake was washed with CH₂Cl₂ (5 mL×2) and the filtrates were concentrated under reduced pressure to provide product 5c (35 mg, crude) as a yellow solid.

### Step 5:

To a solution of 5c (25 mg, 108 umol, 1 eq) in MeOH (2 mL) was added NaOH (5 M, 21 μL, 1 eq) at 25 °C, and the mixture was stirred at 25 °C for 20 min. The reaction mixture was concentrated under reduced pressure to dryness. The crude product was triturated with acetone (5 mL) at 25 °C and stirred for another 30 min. After filtering, the cake was washed with acetone (5 mL×2), and the precipitate was collected and dried in vacuum to provide product 5d (25 mg, 83.8% yield) as a yellow solid; ¹H NMR: 400 MHz CD₃OD, δ 1.39 (br t, J=11.6 Hz, 2H) 1.70-1.79 (m, 2H) 1.92-2.02 (m, 2H) 2.26-2.37 (m, 2H) 2.76 (br t, J=6.40 Hz, 1H) 4.42 (brd, J=6.8 Hz, 2H) 6.91-7.00 (m, 2H) 7.09 (d, J=11.6 Hz, 2H); LC-MS: m/z [M+H-Na]=233.0.

### Step 6:

To a solution of 5d (25 mg, 98 umol, 1 eq) in MeOH (2 mL) was added HCl (1 M, 0.2 mL, 2 eq) and then the mixture was stirred at 25 °C for 20 min. The reaction mixture was diluted with CH₂Cl₂ (15 mL) and then extracted with CH₂Cl₂ (15 mL×2). The combined organic layers were washed with brine (15 mL), dried over Na₂SO₄, filtered and concentrated under reduced pressure to give Ex.5 (15 mg, 65.4% yield) as a yellow solid; ¹H NMR: 400 MHz CD₃OD, δ 1.32-1.41 (m, 2H) 1.69-1.75 (m, 2H) 1.92-1.98 (m, 2H) 2.24-2.34 (m, 2H) 2.70-2.77 (m, 1H) 4.34-4.45 (m, 2H) 6.93 (d, J=11.6 Hz, 2H) 7.07 (d, J=11.6 Hz, 2H); LC-MS: m/z [M+1]⁺=233.1.

### Example 6: Preparation of 4-(8-oxabicyclo[3.2.1]octan-3-yl)-2-hydroxycyclohepta-2,4,6-trien-1-one (Ex.6)

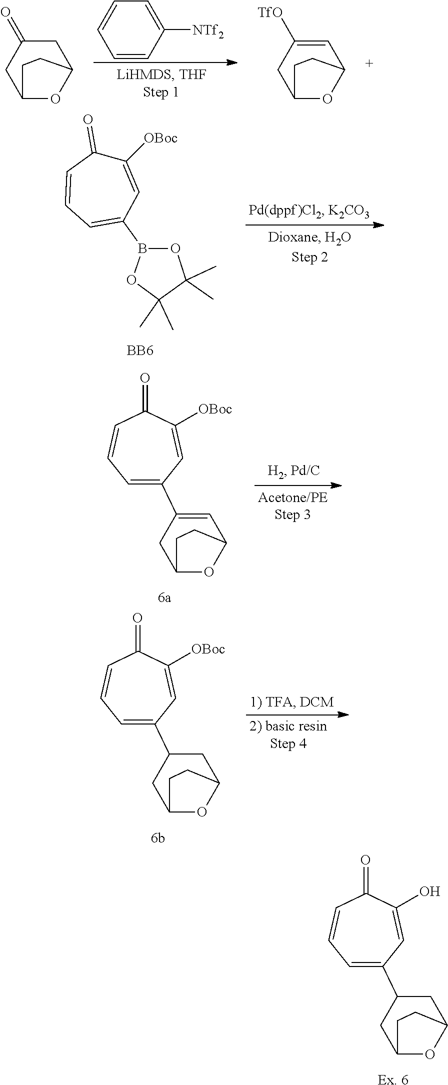

### Step 1:

To a solution of 8-oxabicyclo[3.2.1]octan-3-one (300 mg, 2.38 mmol, 1 eq) in THF (8 mL) was added LiHMDS (1 M, 3.3 mL, 1.4 eq) in one portion at −78 °C under N₂. The mixture was stirred at −78 °C for 30 min, then N,N-bis(trifluoromethylsulphonyl)aniline (1.10 g, 3.09 mmol, 1.3 eq) in THF (8 mL) was added to the mixture drop-wise under N₂ and stirred for 0.5 hours. The mixture was slowly warmed to 25 °C and stirred for another 2 hours. The reaction was monitored by TLC (petroleum ether:EtOAc=5:1). After cooling to 0 °C, the reaction was quenched with H₂O (10 mL) and then extracted with petroleum ether (50 mL×3). The combined organic layers were washed with water (10 mL), brine (10 mL), dried over Na₂SO₄, filtered and concentrated under reduced pressure to give 8-oxabicyclo[3.2.1]oct-2-en-3-yl trifluoromethanesulfonate (600 mg, crude) as a yellow oil.

### Step 2:

To a solution of building block BB6 (413 mg, 1.19 mmol, 1 eq) in dioxane (5 mL) and H₂O (1 mL) was added K₂CO₃ (328 mg, 2.38 mmol, 2 eq), 8-oxabicyclo[3.2.1]oct-2-en-3-yl trifluoromethanesulfonate (307 mg, 1.19 mmol, 1 eq) and Pd(dppf)Cl₂·CH₂Cl₂ (97 mg, 118 umol, 0.1 eq) under N₂ atmosphere. The system was degassed and charged with nitrogen three times. The mixture was heated to and stirred at 118 °C for 30 min under N₂ atmosphere. The reaction was monitored by TLC (petroleum ether:EtOAc=1:1, product Rf=0.30). After cooling to room temperature, water (10 mL) was added, and the mixture was extracted with EtOAc (30 mL×3). The combined organic layers were washed with brine (10 mL), dried over Na₂SO₄, filtered and concentrated under reduced pressure. The residue was purified by silica gel column chromatography eluted with petroleum ether:EtOAc (20:1 to 3:1) to give 6a (190 mg, 48.4% yield) as yellow oil.

### Step 3:

To a solution of 6a (75 mg, 227 umol, 1 eq) in acetone (4 mL) and petroleum ether (1 mL) was added 10% Pd/C (10 mg) under N₂. The suspension was degassed under vacuum and purged with H₂ several times. The mixture was stirred under H₂ (15 psi) at 25 °C for 0.5 hour. The reaction was monitored by LC-MS. The reaction mixture was filtered through a pad of Celite, and the filter cake was washed with acetone (10 mL×2). The combined filtrates were concentrated under reduced pressure to dryness. The residue was purified by prep-HPLC {column: Welch Xtimate C18 (100*25 mm, 3 um); mobile phase: [water (0.1% TFA)-ACN]; B %: 35%-50%, 12 min} to give product 6b (60 mg, 79.5% yield) as a yellow solid.

### Step 4:

To a solution of 6b (20 mg, 60.2 umol, 1 eq) in CH₂Cl₂ (1 mL) was added TFA (0.1 mL) in one portion at 0 °C. The mixture was warmed to room temperature and stirred at 25 °C for 0.5 hr. The reaction was monitored by TLC (petroleum ether:EtOAc=1:1, product Rf=0.00). The reaction mixture was diluted with CH₂Cl₂ (10 mL) and concentrated under reduced pressure to dryness below 10 °C. The mixture was then re-dissolved in CH₂Cl₂ (10 mL), treated with Amberlyst A21 (0.1 g) and stirred for another 0.5 hr. After filtering, the cake was washed with CH₂Cl₂ (5 mL×2) and the combined filtrates were concentrated under reduced pressure to provide Ex. 6 (11 mg, 78.4% yield, 99.6% purity) as a yellow solid; ¹H NMR: 400 MHz CD₃OD, S 1.40-1.50 (m, 2H) 1.74-1.81 (m, 2H) 1.97-2.06 (m, 2H) 2.35-2.45 (m, 2H) 2.87-2.99 (m, 1H) 4.46 (ddd, J=6.4, 4.4, 2.4 Hz, 2H) 7.13 (d, J=9.6 Hz, 1H) 7.22 (d, J=10.8 Hz, 1H) 7.35 (d, J=1.6 Hz, 1H) 7.39-7.47 (m, 1H); LC-MS: m/z [M+1]⁺=233.2.

### Example 7: Preparation of 2-hydroxy-5-(tetrahydro-2H-pyran-4-yl)cyclohepta-2,4,6-trien-1-one (Ex.7)

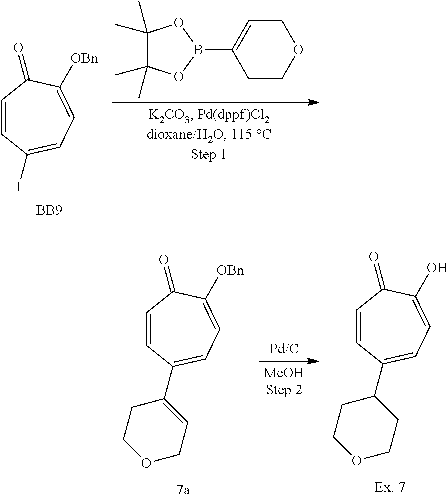

### Step 1:

To a mixture of building block BB9 (500 mg, 1.48 mmol, 1 eq), 2-(3,6-dihydro-2H-pyran-4-yl)-4,4,5,5-tetramethyl-1,3,2-dioxaborolane (465 mg, 2.22 mmol, 1.5 eq) and K₂CO₃ (408 mg, 2.96 mmol, 2 eq) in dioxane (15 mL) and H₂O (3 mL) was added Pd(dppf)Cl₂·CH₂Cl₂ (120 mg, 147 umol, 0.1 eq). The mixture was degassed and charged with nitrogen three times. The mixture was heated to and stirred at 115 °C for 0.5 hour. The reaction was monitored by TLC (petroleum ether:EtOAc=1:1). After cooling to room temperature, the reaction mixture was poured into H₂O (10 mL) and extracted with EtOAc (10 mL×3). The combined organic phases were washed with brine (10 mL), dried over anhydrous Na₂SO₄, filtered and concentrated under reduced pressure to give a residue. The residue was purified by silica gel column chromatography eluting with petroleum ether:EtOAc (8:1 to 1:1) to obtain product 7b (280 mg, 64.3% yield) as a brown solid.

### Step 2:

To a solution of 7a (0.25 g, 849 umol, 1 eq) in MeOH (5 mL) was added Pd/C (10%, 250 mg) under N₂. The suspension was degassed under vacuum and purged with H₂ three times. The mixture was stirred under H₂ (15 psi) at 15 °C for 1 hour. The reaction was monitored by LCMS. The reaction mixture was filtered through a pad of Celite and the filter cake was washed with MeOH (10 mL×2). The combined filtrates were concentrated to dryness under reduced pressure. The residue was purified by prep-HPLC {column: Nano-micro Kromasil C18 (100*30 mm, 5 um); mobile phase: [water (0.1% TFA)-ACN]; B %: 20%-30%, 10 min} to give Ex.7 (32 mg, 18.2% yield) as a white solid; ¹H NMR: 400 MHz CD₃OD, δ 7.40-7.48 (m, 2H), 7.27-7.36 (m, 2H), 4.04 (br d, J=10.4 Hz, 2H), 3.46-3.60 (m, 2H), 2.81 (s, 1H), 1.77 (br s, 3H), 1.67-1.83 (m, 1H); LC-MS: m/z [M+1]⁺=207.1.

### Example 8: Preparation of 2-hydroxy-3-(tetrahydro-2H-pyran-4-yl)cyclohepta-2,4,6-trien-1-one (Ex.8)

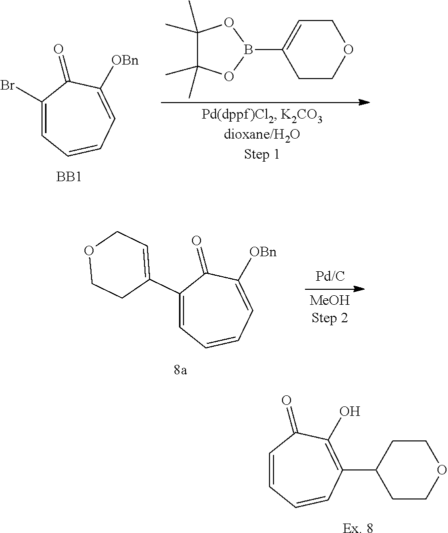

### Step 1

To a mixture of building block BB1 (300 mg, 1.03 mmol, 1 eq) and 2-(3,6-dihydro-2H-pyran-4-yl)-4,4,5,5-tetramethyl-1,3,2-dioxaborolane (324 mg, 1.55 mmol, 1.5 eq) in dioxane (5 mL) and H₂O (1 mL) was added Pd(dppf)Cl₂ (75 mg, 103 umol, 0.1 eq) and K₂CO₃ (427 mg, 3.09 mmol, 3 eq) at 25 °C under N₂. The mixture was degassed and charged with nitrogen three times. The mixture was heated to and stirred at 120 °C for 0.5 hour. The reaction was monitored by LCMS. After cooling, water (10 mL) was added and the mixture extracted with ethyl acetate (10 mL×3). The combined organic phases were washed with brine (20 mL), dried over anhydrous Na₂SO₄, filtered and concentrated under vacuum to dryness. The residue was purified by silica gel column chromatography eluting with petroleum ether:EtOAc (20:1 to 4:1) to obtain product 8a (200 mg, 65.9% yield) as a yellow oil.

### Step 2:

To a solution of 8a (200 mg, 679 umol, 1 eq) in MeOH (10 mL) was added Pd/C (100 mg, 10%) under N₂. The suspension was degassed under vacuum and purged with H₂ three times. The mixture was stirred under H₂ (15 psi) at 25 °C for 1 hr. The reaction was monitored by LCMS. The reaction mixture was filtered through a pad of Celite and the filter cake was washed with MeOH (10 mL×2). The combined filtrates were concentrated to dryness under reduced pressure. The residue was purified by prep-HPLC (column: Nano-micro Kromasil C18 (100*30 mm, 5 um); mobile phase: [water (0.1% TFA)-ACN]; B %: 20%-30%, 10 min) to give Ex. 8 (13 mg, 61.7 umol, 9.09% yield, 98% purity) as a green solid after lyophilization; ¹H NMR: 400 MHz CD₃OD, δ 7.57 (d, J=9.6 Hz, 1H), 7.37-7.24 (m, 2H), 7.13-7.05 (m, 1H), 4.01-3.92 (m, 2H), 3.43-3.53 (m, 3H), 1.74-1.58 (m, 4H); LC-MS: m/z [M+1]⁺=207.1.

### Example 9: Preparation of 2-hydroxy-4-(tetrahydro-2H-pyran-4-yl)cyclohepta-2,4,6-trien-1-one (Ex.9)

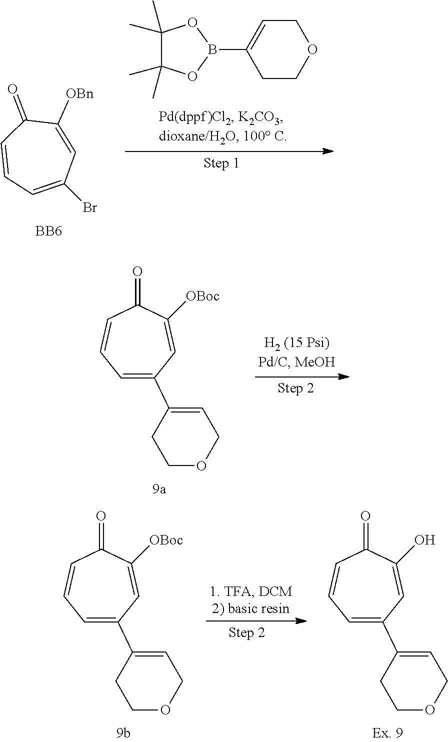

### Step 1:

To a mixture of building block BB6 (526 mg, 1.66 mmol, 1 eq) and 2-(3,6-dihydro-2H-pyran-4-yl)-4,4,5,5-tetramethyl-1,3,2-dioxaborolane (523 mg, 2.49 mmol, 1.5 eq) in dioxane (8 mL) and H₂O (0.5 mL) was added K₂CO₃ (458 mg, 3.32 mmol, 2 eq) in one portion at 20 °C under N₂ Atmosphere. The mixture was degassed and charged with nitrogen three times. The mixture was heated and stirred at 100 °C for 2 hours under N₂. The reaction was monitored by LCMS. After cooling, water (10 mL) was added and the mixture extracted with EtOAc (30 mL×3). The combined organic layers were washed with brine (10 mL), dried over Na₂SO₄, filtered and concentrated under reduced pressure to give a residue. The residue was purified by silica gel column chromatography eluting with petroleum ether:EtOAc (20:1 to 4:1) to afford 9a (380 mg, 73.7% yield) as a yellow solid.

### Step 2:

To a solution of 9a (365 mg, 1.18 mmol, 1 eq) in MeOH (5 mL) was added Pd/C (10%, 50 mg) under N₂. The suspension was degassed under vacuum and purged with H₂ three times. The mixture was stirred under H₂ (15 psi) at 25 °C for 2 hours. The reaction was monitored by LCMS. The reaction mixture was filtered through a pad of Celite and the filter cake was washed with MeOH (10 mL×2). The combined filtrates were concentrated to dryness under reduced pressure. The residue was purified by silica gel column chromatography eluting with petroleum ether:EtOAc (20:1 to 1:1) to give 9b (74 mg, 20.5% yield) as a white solid.

### Step 3:

To a solution of 9b (88 mg, 156 umol, 1 eq) in dichloromethane (4 mL) was added TFA (770 mg) in one portion at 25 °C. The mixture was stirred at 25 °C for 0.5 hour. The reaction was monitored by TLC (petroleum ether:EtOAc=3:1). A drop of water was added, and the solution was concentrated under reduced pressure to dryness. The residue was re-dissolved in dichloromethane (5 mL) and treated with Amberlyst A21 (0.2 g). The suspension was stirred at 25 °C for 30 min and then filtered. The filter cake was washed with dichloromethane (5 mL×2) and the filtrate was concentrated under reduced pressure below 5 °C to obtain Ex. 9 (53 mg, 92.8% yield) as a brown solid; ¹H NMR: 400 MHz CD₃OD, δ 7.41-7.51 (m, 1H), 7.34 (d, J=1.6 Hz, 1H), 7.24 (d, J=10.8 Hz, 1H), 7.11 (dd, J=10.8 Hz, 1H), 3.97-4.13 (m, 2H), 3.48-3.63 (m, 2H), 2.78-2.94 (m, 1H), 1.76-1.89 (m, 4H); LC-MS: m/z [M+1]⁺=207.1.

### Example 10: Preparation of 2-hydroxy-3-(tetrahydro-2H-pyran-2-yl)cyclohepta-2,4,6-trien-1-one (Ex.10)

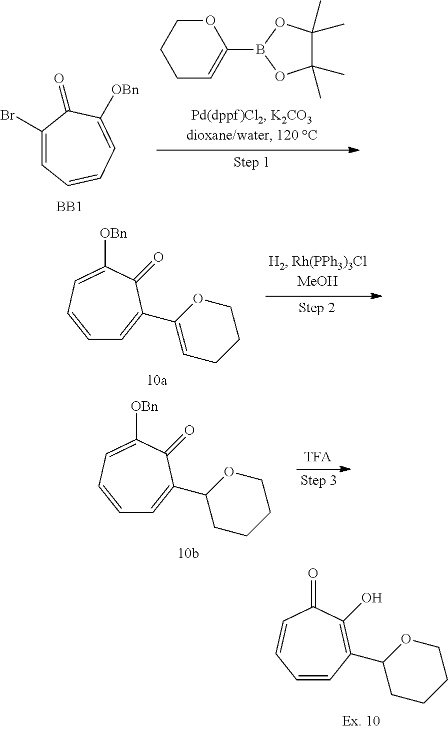

### Step 1:

To a mixture of building block BB1 (1.00 g, 3.43 mmol, 1.00 eq), 2-(3,4-dihydro-2H-pyran-6-yl)-4,4,5,5-tetramethyl-1,3,2-dioxaborolane (1.08 g, 5.15 mmol, 1.50 eq) and K₂CO₃ (949 mg, 6.87 mmol, 2.00 eq) in a mixture of dioxane and water (5:1, 10 mL) was added Pd(dppf)Cl₂·CH₂Cl₂ (280 mg, 343 umol, 0.10 eq) under N₂ atmosphere. The system was degassed and charged with nitrogen three times. The mixture was heated and stirred at 120 °C for 2 hours under N₂ atmosphere. The reaction was monitored by TLC (petroleum ether:EtOAc=1:1, product Rf=0.27). After cooling, the mixture was filtered through a pad of Celite, and the filter cake was washed with CH₂Cl₂ (30 mL×3). The filtrate was concentrated under reduced pressure to dryness. Two equal reactions were carried out in parallel and combined together to be purified by silica gel column chromatography eluting with dichloromethane:MeOH (50:1 to 30:1) to afford 10a (1.50 g, 74.2% yield, 90% purity) as a yellow solid.

### Step 2:

To a solution of 10a (500 mg, 1.70 mmol, 1.00 eq) in MeOH (4 mL) was added Rh(PPh₃)₃Cl (50.0 mg, 0.54 mmol, 0.03 eq) under N₂. The suspension was degassed under vacuum and purged with H₂ three times. The mixture was stirred under H₂ (30 psi) for 1 hour. The reaction was monitored by LCMS. The reaction mixture was filtered through a pad of Celite, and the filter cake was washed with MeOH (5 mL×3). The combined filtrates were concentrated under reduced pressure to dryness. The residue was purified by silica gel column chromatography eluting with petroleum ether:EtOAc (20:1 to 1:1) to give 10b (390 mg, crude) as a yellow oil.

### Step 3:

In two round bottom flasks intermediate 10b (50.0 mg, 169 umol, 1.00 eq) was dissolved in TFA (2 mL) and the mixtures stirred at 50 °C for 1 hour. Reaction progress was monitored by LCMS. The two reactions were then combined, diluted with CH₂Cl₂ (10 mL) and concentrated under reduced pressure to dryness below 10 °C.

The resulting residue was purified by prep-HPLC (column: Welch Xtimate C18 (100*25 mm, 3 um); mobile phase: [water(0.1% TFA)-ACN]; B %: 25%-55%, 10.5 min) to give Ex. 10 (25.0 mg, 35.9% yield, 99.7% purity) as a yellow solid; ¹H NMR: 400 MHz DMSO-d₆, δ 7.74-7.77 (m, 1H), 7.34-7.39 (m, 1H), 7.26-7.28 (m, 1H), 7.12-7.17 (m, 1H), 4.72-4.75 (m, 1H), 4.07-4.09 (m, 1H), 3.57-3.62 (m, 1H), 1.84-1.96 (m, 2H), 1.57-1.65 (m, 3H), 1.13-1.18 (m, 1H); LC-MS: m/z [M+1]⁺=207.0.

### Example 11: Preparation of 25-cyclobutoxy-2-hydroxycyclohepta-2,4,6-trien-1-one (ex.11)

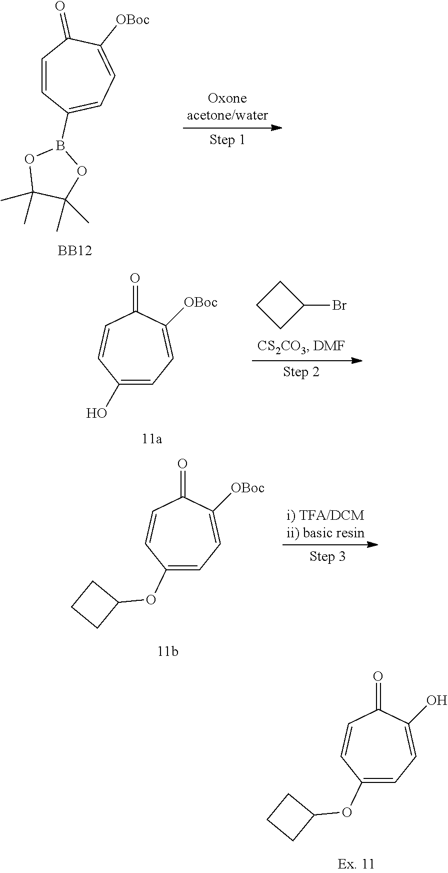

### Step 1:

To a solution of building block BB12 (500 mg, 1.44 mmol, 1.00 eq) in acetone (8.00 mL) and H₂O (1.00 mL) was added oxone (1.32 g, 2.15 mmol, 1.50 eq). The mixture was stirred at 25 °C for 2 hours. Reaction progress was monitored by TLC (petroleum ether:EtOAc=3:1, product Rf=0.1). To the reaction mixture was added aq.NaHSO₃ (50 mL) and the mixture stirred at 20 °C for 0.5 hour. The mixture was then washed with n-hexane (10 mL). Product 11a (0.31 g, crude) was obtained as a yellow solid which was used into the next step without further purification.

### Step 2:

To a solution of alcohol 11a (0.30 g, 1.26 mmol, 1.00 eq) and bromocyclobutane (340 mg, 2.52 mmol, 237 μL, 2.00 eq) in DMF (4.00 mL) was added Cs₂CO₃ (410 mg, 1.26 mmol, 1.00 eq) and Bu₄NI (465 mg, 1.26 mmol, 1.00 eq). The mixture was stirred at 90 °C for 7 hours. The reaction was monitored by LCMS. The mixture was poured into water (50 mL), and the aqueous phase was extracted with ethyl acetate (50 mL×3). The combined organic phases were washed with aq NH₄Cl (50 mL), dried with anhydrous Na₂SO₄, filtered and to dryness concentrated under vacuum. The residue was purified by prep-HPLC (column: Nano-micro Kromasil C18 (100*30 mm, 5 um); mobile phase: [water (0.1% TFA)-ACN]; B %: 20%-30%, 10 min) to afford compound 11b (50.0 mg, 13.5% yield) as a yellow solid.

### Step 3:

To a solution of 11b (40 mg, 136 umol, 1.00 eq) in DCM (1.00 mL) was added TFA (0.1 mL). The mixture was stirred at 25 °C for 0.5 hour. The reaction progress was monitored by TLC (petroleum ether:EtOAc=1:1, product Rf=0.13). Solvents were evaporated under reduced pressure. The residue was re-dissolved in dichloromethane (5 mL), treated with Amberlyst A21 (0.2 g), stirred for another 0.5 h, and filtered. The solid cake was rinsed with DCM. The combined filtrate and washings were concentrated under vacuo to afford the titled product Ex. 11 (41.0 mg, crude) as a yellow solid; ¹H NMR: 400 MHz CD₃OD, δ ppm 7.31 (d, J=12.2 Hz, 2H), 7.08-7.01 (m, 2H), 4.70 (quin, J=7.2 Hz, 1H), 2.55-2.44 (m, 2H), 2.21-2.08 (m, 2H), 1.93-1.70 (m, 2H); LC-MS: m/z [M+1]⁺=193.

### Example 12: Preparation of 2-hydroxy-5-(oxetan-3-yloxy)cyclohepta-2,4,6-trien-1-one (Ex.12)

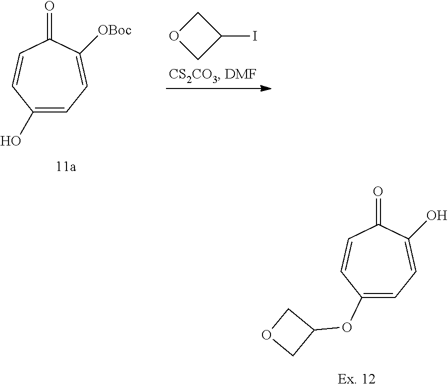

To a solution of alcohol 11a (380 mg, 1.60 mmol, 1 eq) available from preparative example 11 and 3-iodooxetane (381 mg, 2.07 mmol, 1.3 eq) in DMF (3 mL) was added Cs₂CO₃ (519 mg, 1.60 mmol, 1 eq) in one portion under nitrogen. The mixture was heated to and stirred at 120 °C for 7 hours. Reaction progress was monitored by LCMS. After cooling, water (10 mL) was added and the mixture extracted with EtOAc (30 mL×3). The combined organic layers were washed with brine (10 mL), dried over Na₂SO₄, filtered and concentrated under reduced pressure to give a residue. The residue was purified by prep-HPLC (column: Nano-micro Kromasil C18 (100*30 mm, 5 um); mobile phase: [water (0.1% TFA)-ACN]; B %: 20%-30%, 10 min) to obtain Ex. 12 (25 mg, 8.07% yield) as a yellow solid; ¹H NMR: 400 MHz DMSO-d₆, δ 7.14 (d, J=12.4 Hz, 2H), 6.86 (d, J=12.4 Hz, 2H), 5.26 (t, J=5.6 Hz, 1H), 4.92 (t, J=6.8 Hz, 2H), 4.54 (dd, J=5.6, 7.3 Hz, 2H); LC-MS: m/z [M+1]⁺=195.1.

### Example 13: Preparation of 2-hydroxy-5-isopropoxycyclohepta-2,4,6-trien-1-one (Ex.13)

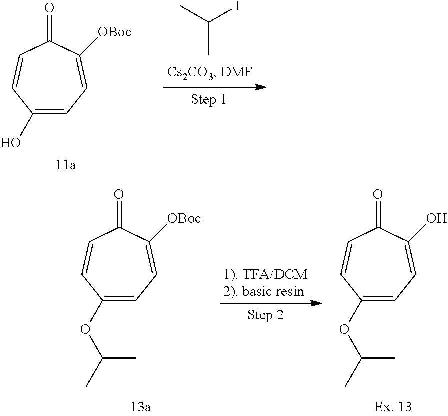

### Step 1:

To a mixture of 11a (50 mg, 209 umol, 1 eq) available from preparative example 11 and Cs₂CO₃ (193 mg, 594 umol, 2.83 eq) in DMF (1 mL) was added 2-iodopropane (53 mg, 314 umol, 1.5 eq) dropwise over 1 min at 0 °C. The mixture was warmed and stirred at 20 °C for 14 hours. The reaction was monitored by LCMS. The mixture was poured into 10% aqueous NH₄Cl solution (5 mL) and then extracted with EtOAc (20 mL×3). The combined organic phases were washed with water (10 mL), brine (10 mL), dried over anhydrous Na₂SO₄, filtered and concentrated under vacuum to dryness. The residue was purified by prep-TLC (petroleum ether:EtOAc=3:1, product Rf=0.4) to afford 13a (35 mg, 59.5% yield) as a light yellow gum.

### Step 2:

To a solution of 13a (30 mg, 107 umol, 1 eq) in dichloromethane (2 mL) was added trifluoroacetic acid (122 mg, 1.07 mmol, 10 eq) dropwise over 1 min at 0 °C. The resulting light yellow solution was warmed and stirred at 25 °C for 30 min. The reaction was monitored by TLC (petroleum ether:EtOAc=3:1). A drop of water was added and the solution was concentrated under reduced pressure to dryness. The residue was re-dissolved in dichloromethane (10 mL) and treated with Amberlyst A21 (0.2 g). The suspension was stirred at 25 °C for 30 min and then filtered. The cake was washed with dichloromethane (5 mL×2) and the filtrate was concentrated under reduced pressure below 5 °C to afford Ex. 13 (18 mg, 93.3% yield) as a yellow solid; ¹H NMR: 400 MHz CDCl₃, δ 7.35-7.33 (m, 1H), 7.32-7.30 (m, 1H), 7.17-7.15 (m, 1H), 7.19-7.15 (m, 1H), 7.14-7.12 (m, 1H), 4.62 (td, J=6.1, 12.0 Hz, 1H), 1.33 (d, J=6.4 Hz, 6H); LC-MS: m/z [M+1]⁺=181.1.

### Example 14: Preparation of 2-hydroxy-5-methoxycyclohepta-2,4,6-trien-1-one

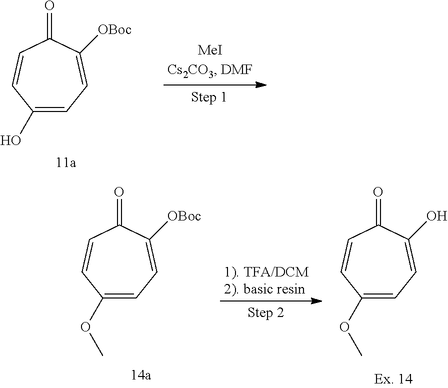

### Step 1:

Two identical reactions were carried out in parallel. To a solution of alcohol 11a (100 mg, 419 umol, 1 eq) in DMF (4 mL) was added Cs₂CO₃ (273 mg, 839 umol, 2 eq) and iodomethane (89 mg, 629.63 umol, 1.5 eq) at 0 °C. The mixture was warmed and stirred at 20 °C for 2 hours. The reaction was monitored by TLC (petroleum ether:ethyl acetate=1:1, product Rf=0.65). The mixture was poured into ice-water (10 mL) and extracted with CH₂Cl₂ (20 mL×3). The combined organic phases were washed with brine (30 mL), dried over anhydrous Na₂SO₄, filtered and concentrated in vacuum to dryness. The residue was purified by prep-TLC (petroleum ether:ethyl acetate=1:1, Rf=0.6) to obtain compound 14a (20 mg, 18.9% yield) as a yellow oil.

### Step 2:

To a solution of 14a (40 mg) in CH₂Cl₂ (2 mL) was added TFA (1 mL) in one portion at 0 °C. The mixture was warmed and stirred at 25 °C for 1 hour. The reaction was monitored by LCMS. A drop of water was added, and the solution was concentrated under reduced pressure to dryness. The residue was re-dissolved in dichloromethane (5 mL) and treated with Amberlyst A21 (0.1 g). The suspension was stirred at 25 °C for 30 min and then filtered. The cake was washed with dichloromethane (5 mL×2) and the filtrate was concentrated under reduced pressure below 5 °C to afford product Ex. 14 (20 mg, 82.9% yield) as a yellow solid; ¹H NMR: 400 MHz CD₃OD, δ 7.36 (s, 1H), 7.37-7.29 (m, 2H), 7.17-7.10 (m, 2H), 3.83 (s, 3H); LC-MS: m/z [M+1]⁺=153.1.

### Example 15: Preparation of 5-ethoxy-2-hydroxycyclohepta-2,4,6-trien-1-one (Ex.15)

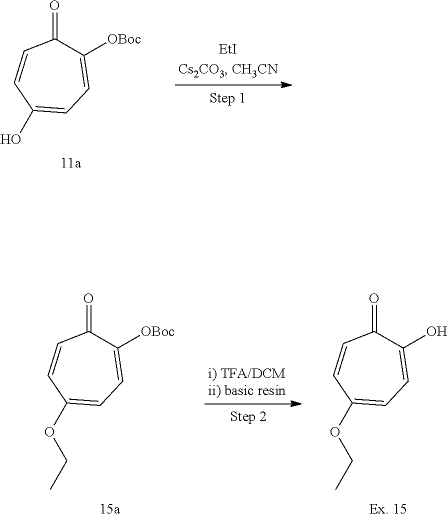

### Step 1:

To a solution of alcohol 11a (100 mg, 419 umol, 1 eq) in CH₃CN (2 mL) was added Cs₂CO₃ (273 mg, 839 umol, 2 eq) and iodoethane (98 mg, 629 umol, 1.5 eq) at 0 °C. The mixture was heated and stirred at 50 °C for 3 hours. The reaction was monitored by TLC (petroleum ether:EtOAc=1:1, product Rf=0.7). After cooling, the reaction mixture was filtered through a pad of Celite and the filter cake was washed with dichloromethane (2 mL×2). The combined organic phases were concentrated under reduced pressure to give a residue that was purified by prep-TLC (petroleum ether:EtOAc=1:1) to give 15a (30 mg, 26.8% yield) as a yellow solid.

### Step 2:

To a solution of 15a (30 mg) in dichloromethane (1 mL) was added trifluoroacetic acid (0.5 mL) in one portion at 0 °C. The mixture was warmed and stirred at 25 °C for 1 hour. The reaction was monitored by TLC (petroleum ether:EtOAc=1:1, product Rf=0.2). A drop of water was added, and the solution was concentrated under reduced pressure to dryness. The residue was re-dissolved in dichloromethane (10 mL) and treated with Amberlyst A21 (0.2 g). The suspension was stirred at 25 °C for 30 min and then filtered. The filter cake was washed with dichloromethane (5 mL×2) and the filtrate was concentrated under reduced pressure below 5 °C to afford compound Ex. 15 (17 mg, 90.9% yield) as a yellow solid; ¹H NMR: 400 MHz DMSO-d₆, δ 7.17-7.19 (m, 1H), 7.14-7.16 (m, 1H), 7.03-7.05 (m, 1H), 7.00-7.02 (m, 1H), 4.01 (q, J=7.02 Hz, 2H), 1.38 (t, J=7.02 Hz, 3H); LC-MS: m/z [M+1]⁺=167.0.

### Example 16: Preparation of 2-hydroxy-4-isopropoxycyclohepta-2,4,6-trien-1-one (Ex.16)

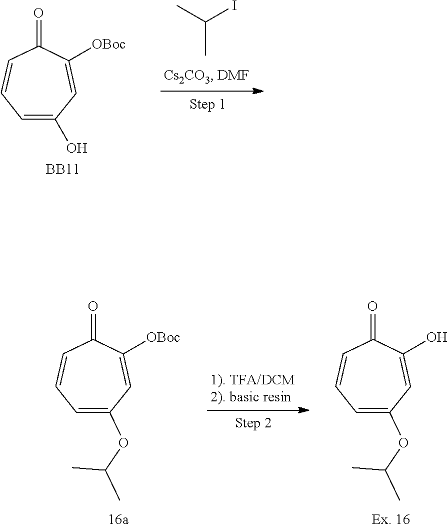

### Step 1:

To a solution of building block BB11 (110 mg, 461 umol, 1 eq) in DMF (4 mL) was added Cs₂CO₃ (300 mg, 923 umol, 2 eq) and 2-iodopropane (117 mg, 693 umol, 1.5 eq) dropwise over 1 min at 0 °C. The mixture was warmed and stirred at 25 °C for 3 hours. The reaction was monitored by TLC (petroleum ether:EtOAc=1:1, (product Rf=0.7). The mixture was poured into 10% aqueous NH₄Cl solution (5 mL) and then extracted with EtOAc (20 mL×3). The combined organic phases were washed with water (10 mL) and brine (10 mL), dried over anhydrous Na₂SO₄, filtered and concentrated under reduced pressure to dryness. The residue was purified prep-TLC (petroleum ether:EtOAc=1:1) to afford compound 16a (18 mg, 13.9% yield) as a yellow solid.

### Step 2:

To a solution of 16a (18 mg) in dichloromethane (1 mL) was added trifluoroacetic acid (0.5 mL) dropwise over 1 min at 0 °C. The mixture was warmed and stirred at 25 °C for 1 hour. The reaction was monitored by TLC (petroleum ether:EtOAc=2:1, product Rf=0.5). A drop of water was added and the solution was concentrated under reduced pressure to dryness. The residue was re-dissolved in dichloromethane (5 mL) and treated with Amberlyst A21 (0.1 g). The suspension was stirred at 25 °C for 30 min and then filtered. The filter cake was washed with dichloromethane (5 mL×2) and the filtrate was concentrated under reduced pressure below 5 °C to afford product Ex. 16 (11 mg, 95% yield) as a yellow solid; ¹H NMR: 400 MHz CD₃OD, δ 7.24-7.32 (m, 1H), 6.99 (d, J=17.6 Hz, 1H), 6.93 (d, J=2.64 Hz, 1H), 6.78 (d, J=11.4 Hz, 1H), 4.69-4.77 (m, 1H), 1.37 (d, J=5.6 Hz, 6H); LC-MS: m/z [M+1]⁺=181.1.

### Example 17: Preparation of 2-hydroxy-4-methoxycyclohepta-2,4,6-trien-1-one (Ex.17)

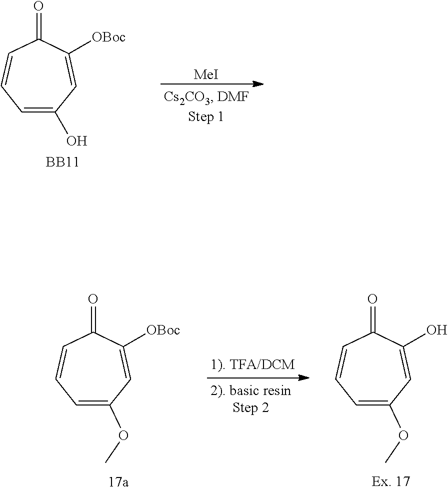

### Step 1:

To a solution of building block BB11 (0.37 g, 1.55 mmol, 1 eq) in DMF (2 mL) was added Cs₂CO₃ (1.01 g, 3.11 mmol, 2 eq) and iodomethane (330 mg, 2.33 mmol, 1.5 eq). The mixture was stirred at 20 °C for 2 hours. TLC (petroleum ether:EtOAc=1:1, product Rf=0.7). The mixture was poured into ice-water (20 mL), and the aqueous phase was extracted with CH₂Cl₂ (20 mL×3). The combined organic phases were washed with brine (20 mL), dried over anhydrous Na₂SO₄, filtered and concentrated under reduced pressure to dryness. The residue was purified by prep-TLC (petroleum ether:EtOAc=1:1) to give compound 17a (60 mg, 15.3% yield) as a yellow oil.

### Step 2:

To a solution of 17a (60 mg) in CH₂Cl₂ (2 mL) was added trifluoroacetic acid (1 mL) dropwise over 1 min at 0 °C. The mixture was warmed and stirred at 20 °C for 0.5 hour. The reaction was monitored by TLC (petroleum ether:EtOAc=1:1). The reaction mixture was diluted with CH₂Cl₂ (10 mL) and concentrated under reduced pressure below 10 °C to dryness. The mixture was re-dissolved in CH₂Cl₂ (10 mL) and treated with Amberlyst A21 (0.1 g) and stirred at 25 °C for another 0.5 hour. After filtering, the cake was washed with CH₂Cl₂ (5 mL×2) and the filtrate was concentrated under reduced pressure to provide compound Ex. 17 (15 mg, 41.4% yield) as a yellow solid; ¹H NMR: 400 MHz CDCl₃, δ 7.18-7.10 (m, 1H), 6.98 (d, J=10 Hz, 1H), 6.92 (d, J=2.8 Hz, 1H), 6.70 (dd, J=11.6 Hz, 1H), 3.86 (s, 3H); LC-MS: m/z [M+1]⁺=153.1.

### Example 18: Preparation of 2-hydroxy-4-ethoxycyclohepta-2,4,6-trien-1-one (Ex.18)

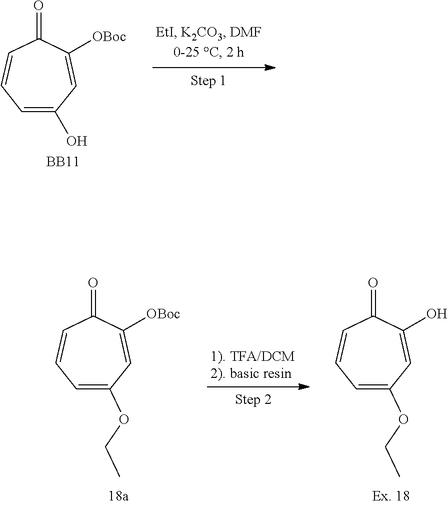

### Step 1:

To a mixture of building block BB11 (100 mg, 419 umol, 1 eq) and K₂CO₃ (116 mg, 839 umol, 2 eq) in DMF (3 mL) was added iodoethane (327 mg, 2.10 mmol, 5 eq) at 0 °C. The mixture was warmed to room temperature and stirred at 25 °C for 2 hours under N₂ atmosphere. The reaction was monitored by LCMS. The reaction mixture was quenched by addition of water (8 mL) and then extracted with ethyl acetate (8 mL×3). The combined organic layers were washed with brine (8 mL), dried over Na₂SO₄, filtered and concentrated under reduced pressure to give a residue. The residue was purified by prep-TLC (petroleum ether:EtOAc=1:1, product Rf=0.6) to obtain 18a (17 mg, 15.2% yield) as a yellow solid.

### Step 2:

To a solution of 18a (17 mg, 63.8 umol, 1 eq) in CH₂Cl₂ (0.5 mL) was added trifluoroacetic acid (0.2 mL) dropwise at 0 °C. The mixture was warmed to room temperature and stirred at 20 °C for 0.5 hour. The reaction was monitored by LCMS. The reaction mixture was diluted with CH₂Cl₂ (10 mL) and concentrated under reduced pressure below 10 °C to dryness. The mixture was re-dissolved in CH₂Cl₂ (5 mL) and treated with Amberlyst A21 (0.1 g) and stirred at 25 °C for another 0.5 hour. After filtering, the filter cake was washed with CH₂Cl₂ (5 mL×2), and the filtrate was concentrated under reduced pressure to provide Ex.18 (10 mg, 92.3% yield, 98% purity) as a yellow solid; ¹H NMR: 400 MHz CDCl₃, δ 7.29 (t, J=11.0 Hz, 1H), 6.99 (d, J=10.5 Hz, 1H), 6.94-6.90 (m, 1H), 6.81-6.74 (m, 1H), 4.10 (q, J=7.0 Hz, 2H), 1.44 (t, J=7.0 Hz, 3H); LC-MS: m/z [M+1]⁺=167.1.

### Example 19: Preparation of 2-hydroxy-3,7-dimethylcyclohepta-2,4,6-trien-1-one (Ex.19)

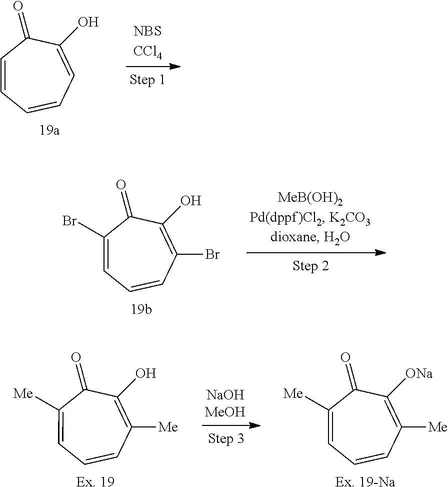

### Step 1:

To a mixture of 2-hydroxy-2,4,6-cycloheptatrien-1-one (19a) (500 mg, 4.09 mmol, 1.00 eq) in CCl₄ (10.0 mL) was added NBS (1.60 g, 9.01 mmol, 2.20 eq) in one portion at 25 °C under N₂. The mixture was heated to 80 °C and stirred for 30 min. The reaction was monitored by LCMS. After cooling, water (30 mL) was added, and the mixture was extracted with dichloromethane (30 mL×3). The combined organic phases were washed with water (20 mL) and brine (20 mL), dried over anhydrous Na₂SO₄, filtered and concentrated under reduced pressure to dryness to give a compound 19b (1.15 g, crude) as a yellow solid which was used to next step without purification.

### Step 2:

To a solution of crude 19b (1.15 g, 4.00 mmol estimated from Step 1, 1.00 eq) in dioxane (10.0 mL) and H₂O (2.00 mL) was added MeB(OH)₂ (6 g, 100 mmol, 25.0 eq), K₂CO₃ (2.21 g, 16 mmol, 4.00 eq) and Pd(dppf)Cl₂·CH₂Cl₂ (660 mg, 800 umol, 0.20 eq) in one portion at 25 °C under N₂ atmosphere. The system was degassed and charged with nitrogen three times. The mixture was heated to and stirred at 118 °C for 30 min. The reaction was monitored by LCMS. After cooling to room temperature, the mixture was filtered through a pad of Celite, and the filter cake was washed with EtOAc (30 mL×3). The filtrate was concentrated under reduced pressure to dryness. The residue was purified by prep-HPLC (column: Nano-micro Kromasil C18 (100*30 mm, 5 um); mobile phase: [water (0.1% TFA)-ACN]; B %: 20%-30/o, 10 min) to give the titled compound Ex.19 (5 mg, 0.8% yield) as black oil.

### Step 3:

To a mixture of Ex.19 (66.0 mg, 439 umol, 1.00 eq) in MeOH (1.50 mL) was added NaOH (5 M, 87.90 uL, 1.00 eq) in one portion at 25 °C. The mixture was stirred at 25 °C for 20 min. The reaction mixture was concentrated under reduced pressure to remove MeOH. The crude product was triturated with acetone (5 mL) at 25 °C and stirred for another 30 min. After filtering, the cake was washed with acetone (5 mL×2) and the precipitate was collected and dried in vacuum to provide the sodium salt of the titled compound Ex.19-Na (60.0 mg, 79.3%) as a yellow solid; ¹H NMR: 400 MHz CD₃OD, δ 7.23 (d, J=10.0 Hz, 2H), 6.38 (t, J=9.6 Hz, 1H), 2.31 (s, 6H); LC-MS: m/z [M+H]⁺=151.2.

### Example 20: Preparation of 7-hydroxy-2,4-dimethylcyclohepta-2,4,6-trien-1-one (Ex.20)

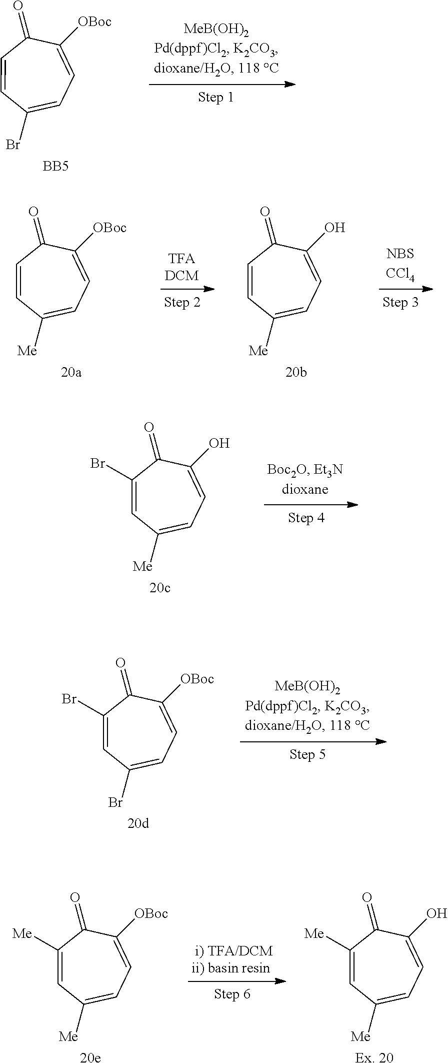

### Step 1:

To a mixture of building block BB5 (500 mg, 1.66 mmol, 1 eq) and K₂CO₃ (458 mg, 3.32 mmol, 2 eq) in dioxane (20 mL) and H₂O (4 mL) was added methylboronic acid (993 mg, 16.6 mmol, 10 eq) and Pd(dppf)Cl₂·CH₂Cl₂ (135 mg, 166 umol, 0.1 eq) in one portion at 25 °C under N₂ atmosphere. The system was degassed and charged with nitrogen three times. The mixture was heated and stirred at 118 °C for 30 min. The reaction was monitored by TLC (petroleum ether:EtOAc=3:1, product Rf=0.13). After cooling to room temperature, the reaction mixture was poured into H₂O (20 mL) and then extracted with EtOAc (30 mL×3). The combined organic phases were washed with brine (30 mL), dried over anhydrous Na₂SO₄, concentrated under vacuum to give a residue. The residue was purified by solica gel column chromatography (petroleum ether:EtOAc=20:1 to 4:1) to afford 20a (110 mg, 28% yield) as a yellow solid.

### Step 2:

To a solution of 20a (600 mg, 2.54 mmol, 1 eq) in dichloromethane (6 mL) was added TFA (2 mL) at 0 °C. The mixture was warmed and stirred at 25 °C for 0.5 hour. The reaction was monitored by TLC (petroleum ether:EtOAc=2:1, product Rf=0.1). The reaction mixture was diluted with CH₂Cl₂ (20 mL) and concentrated under reduced pressure below 10 °C to afford 20b (344 mg, 99.5% yield) as a yellow solid.

### Step 3:

To a mixture of 20b (344 mg, 2.53 mmol, 1 eq) in CCl₄ (5 mL) was added NBS (224 mg, 1.26 mmol, 0.5 eq) in one portion at 25 °C under N₂. The mixture was heated to and stirred at 80 °C for 1 hour. The reaction was monitored by LCMS. The reaction was halted with ~23.9% of desired compound and ~50% of starting material remaining. After cooling, water (30 mL) was added and the mixture extracted with EtOAc (30 mL×3). The combined organic phases were washed with water (20 mL) and brine (50 mL), dried over anhydrous Na₂SO₄, filtered and concentrated under reduced pressure to dryness to give crude 20c (300 mg, crude) as a yellow solid, which was used to next step for further purification.

### Step 4:

To a solution of crude 20c (300 mg, 1.4 mmol, 1 eq) in dioxane (5 mL) was added Et₃N (565 mg, 5.6 mmol, 4 eq) and Boc₂O (916 mg, 4.2 mmol, 3 eq) in one portion at 25 °C under N₂. The mixture was heated and stirred at 110 °C for 0.5 hour. The reaction was monitored by TLC (petroleum ether:EtOAc=3:1, product Rf=0.4). After cooling to room temperature, the mixture was concentrated under reduced pressure to dryness. The residue was purified by silica gel column chromatography (petroleum ether:EtOAc=50:1 to 10:1) to provide compound 20d (181 mg, 22.7% yield from 2 steps) as a yellow solid.

### Step 5:

To a mixture of 20d (181 mg, 574 umol, 1.00 eq) and K₂COI (158 mg, 1.15 mmol, 2 eq) in dioxane (5 mL) and H₂O (1 mL) was added methylboronic acid (343 mg, 5.74 mmol, 10 eq) and Pd(dppf)Cl₂·CH₂Cl₂ (46.9 mg, 57.4 umol, 0.1 eq) in one portion at 25 °C under N₂ atmosphere. The system was degassed and charged with nitrogen three times. The mixture was heated and stirred at 118 °C for 30 min. The reaction was monitored by TLC (petroleum ether:EtOAc=3:1, product Rf=0.6). After cooling to room temperature, the reaction mixture was poured into H₂O (30 mL) and then extracted with ethyl acetate (20 mL×3). The combined organic phases were washed with brine (30 mL), dried over anhydrous Na₂SO₄, concentrated in vacuum to give a residue. The residue was purified by silica gel column chromatography (petroleum ether:EtOAc=20:1 to 4:1) to afford compound 20e (120 mg 83.4%) as a white oil.

### Step 6:

To a solution of 20e (60 mg) in dichloromethane (2 mL) was added TFA (0.5 mL) in one portion at 0 °C. The mixture was warmed and stirred at 25 °C for 30 min. The reaction was monitored by TLC (petroleum ether:EtOAc=3:1, product Rf=0.1). The reaction mixture was diluted with CH₂Cl₂ (10 mL) and concentrated under reduced pressure to dryness below 10 °C. The residue was re-dissolved in CH₂Cl₂ (5 mL) and treated with Amberlyst A21 (0.1 g) and stirred at 25 °C for another 0.5 hour. After filtering, the filter cake was washed with CH₂Cl₂ (5 mL×2), and the filtrate was concentrated under reduced pressure to provide compound Ex. 20 (28 mg, 77.8%) as a yellow solid; ¹H NMR: 400 MHz CD₃OD, δ 7.55 (s, 1H), 7.17-7.22 (m, 1H), 7.12-7.17 (m, 1H), 2.35-2.36 (m, 3H), 2.32 (s, 3H); LC-MS: m/z [M+H]⁺=151.1.

### Example 21: Preparation of 2-hydroxy-4,7-dimethylcyclohepta-2,4,6-trien-1-one (Ex.21)

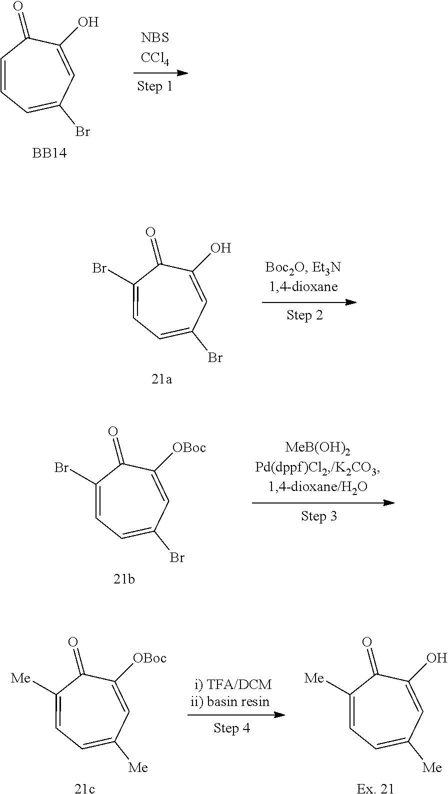

### Step 1:

To a solution of building block BB14 (1 g, 4.97 mmol, 1 eq) in CCl₄ (10 mL) was added NBS (973 mg, 5.47 mmol, 1.1 eq) at 25 °C under N₂. The mixture was heated to and stirred at 80 °C for 1 hour. The reaction was monitored by LCMS, and when its progress reached 36% of the desired peak, the reaction was halted. After cooling to room temperature, water (30 mL) was added and the mixture extracted with ethyl acetate (30 mL×3). The combined organic phases were washed with water (20 mL) and brine (20 mL), dried over anhydrous Na₂SO₄, filtered and concentrated under reduced pressure to dryness to give crude 21a (1.0 g, crude) as a brown solid, which was used to next step for further purification.

### Step 2:

To a solution of 21a (1.30 g, 4.64 mmol, 1 eq) in 1,4-dioxane (20 mL) was added Boc₂O (10.1 g, 46.4 mmol, 10 eq) and Et₃N (4.70 g, 46.4 mmol, 10 eq) in one portion at 25 °C under N₂. The mixture was heated to and stirred at 110 °C for 2 hours. The reaction was monitored by TLC (petroleum ether:EtOAc=3:1, product Rf=0.4). After cooling, the mixture was concentrated under reduced pressure to dryness. The residue was purified by silica gel column chromatography (petroleum ether:EtOAc=100:1 to 3) to get 21b (0.5 g, 1.32 mmol, 28.3% yield) as a yellow oil.

### Step 3:

To a solution of 21b (500 mg, 1.32 mmol, 1 eq) in 1,4-dioxane (20 mL) and water (5 mL) was added methylboronic acid (787 mg, 13.2 mmol, 10 eq), K₂CO₃ (2.47 g, 13.2 mmol, 10 eq) and Pd(dppf)Cl₂·CH₂Cl₂ (107 mg, 131 umol, 0.1 eq) under N₂ atmosphere. The system was degassed and charged with nitrogen three times. The mixture was heated to and stirred at 120 °C for 2 hours under N₂. The reaction was monitored by LCMS and halted when 9% of desired compound was detected. After cooling to 25 °C, water (20 mL) was added and the mixture extracted with CH₂Cl₂ (20 mL×3). The combined organic phases were washed with water (10 mL) and brine (20 mL), dried over anhydrous Na₂SO₄, filtered and concentrated under reduced pressure to dryness. The residue was purified by silica gel column chromatography (petroleum ether:EtOAc=100:1 to 3:1) to get 21c (100 mg, 30.7% yield) as a yellow oil.

### Step 4:

To a solution of 21c (100 mg, 399 umol, 1 eq) in dichloromethane (2 mL) was added TFA (0.5 mL) in one portion at 0 °C. The mixture was warmed to room temperature and stirred at 25 °C for 1 hour. The reaction was monitored by TLC (petroleum ether:EtOAc=1:1). The reaction mixture was diluted with CH₂Cl₂ (10 mL) and concentrated under reduced pressure to dryness below 10 °C. The mixture was re-dissolved in CH₂Cl₂ (5 mL), treated with Amberlyst A21 (0.1 g) and stirred at 25 °C for another 0.5 hour. After filtering, the cake was washed with CH₂Cl₂ (5 mL×2) and the filtrate was concentrated under reduced pressure to provide product Ex. 21 (25 mg, 41.7% yield) as a brown solid; ¹H NMR: 400 MHz CD₃OD, δ 7.49 (br d, J=10.4 Hz, 1H) 7.31 (s, 1H) 6.98 (br d, J=10.4 Hz, 1H) 2.46 (s, 3H) 2.39 (s, 3H); LC-MS: m/z [M+H]⁺=151.1.

### Example 22: Preparation of 4-cyclopropyl-2-hydroxy-7-methylcyclohepta-2,4,6-trien-1-one (Ex.22)

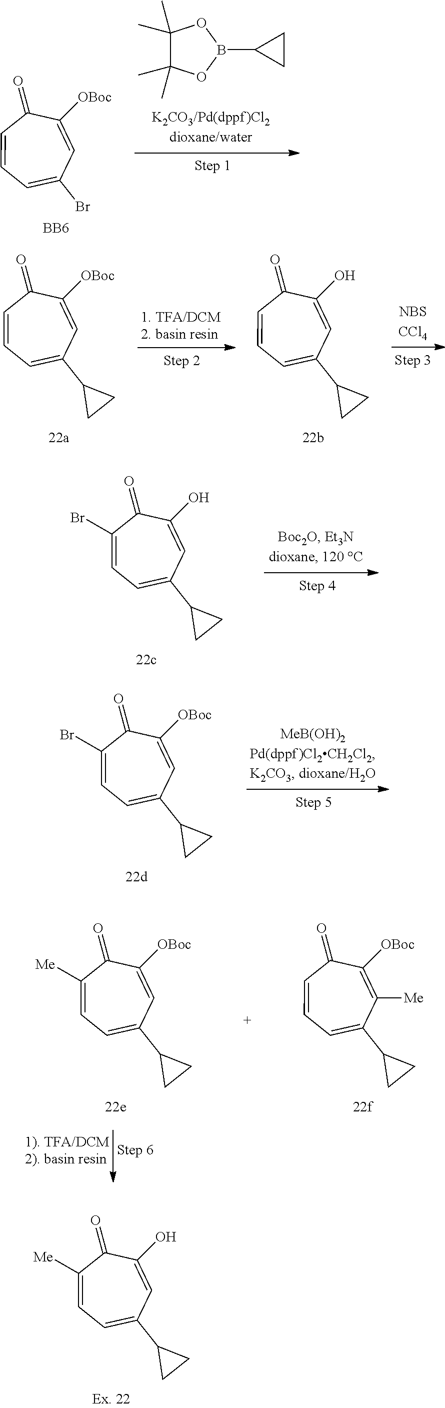

### Step 1:

To a solution of building block BB6 (1 g, 3.32 mmol, 1 eq) in 1,4-dioxane (10 mL) and water (2 mL) was added Pd(dppf)Cl₂CH₂Cl₂ (271 mg, 332 umol, 0.1 eq), K₂CO₃ (5.41 g, 16.6 mmol, 5 eq), and 2-cyclopropyl-4,4,5,5-tetramethyl-1,3,2-dioxaborolane (4.46 g, 26.5 mmol, 8 eq) under N₂ atmosphere. The system was degassed and charged with nitrogen three times. The mixture was heated and stirred at 120 °C for 1 hour under N₂. The reaction was monitored by TLC (petroleum ether:EtOAc=3:1, product Rf=0.3). After cooling, the mixture was filtered through a pad of Celite and the filter cake washed with CH₂Cl₂ (30 mL×3). The filtrate was concentrated under reduced pressure to dryness. The residue was purified by silica gel column chromatography (petroleum ether:EtOAc=100:1 to 3:1) to get 22a (0.8 g, 91.8% yield) as a yellow oil.

### Step 2:

To a solution of 22a (0.8 g) in dichloromethane (8 mL) was added TFA (2 mL) in one portion at 0 °C. The mixture was warmed to room temperature and stirred at 25 °C for 1 hour. The reaction was monitored by TLC (petroleum ether:EtOAc=1:1). The reaction mixture was diluted with CH₂Cl₂ (20 mL) and concentrated under reduced pressure below 10 °C to provide 22b (0.47 g, crude) as yellow oil, which was used for the next step without purification.

### Step 3:

To a solution of 22b (370 mg, 2.28 mmol, 1 eq) in CCl₄ (3 mL) was added NBS (446 mg, 2.51 mmol, 1.1 eq) at 25 °C under N₂. The mixture was heated and stirred at 80 °C for 1 h. The reaction was monitored by LCMS and halted when 27% of the desired MS was detected. After cooling, water (30 mL) was added and the mixture extracted with ethyl acetate (30 mL×3). The combined organic phases were washed with water (20 mL) and brine (50 mL), dried over anhydrous Na₂SO₄, filtered and concentrated under reduced pressure to dryness to give crude 22c (0.5 g) as a brown solid that was a mixture of regioisomers that was taken on to the next step without further purification.

### Step 4:

To a solution of crude 22c (0.60 g, 2.49 mmol, 1 eq) in 1,4-dioxane (6 mL) was added Boc₂O (5.43 g, 24.8 mmol, 10 eq) and Et₃N (2.51 g, 24.9 mmol, 10 eq) in one portion at 25 °C under N₂. The mixture was heated to and stirred at 110 °C for 2 hours. The reaction was monitored by TLC (petroleum ether:EtOAc=2:1, product Rf=0.3). After cooling, the mixture was concentrated under reduced pressure to dryness. The residue was purified by silica gel column chromatography (petroleum ether:EtOAc=100:1 to 2:1) to get the regioisomeric mixture containing 22d (0.27 g, 31.8% yield) a yellow oil.

### Step 5:

To a solution of crude 22d (270 mg, 791 umol, 1 eq) in 1,4-dioxane (10 mL) and water (1 mL) was added methylboronic acid (473 mg, 7.91 mmol, 10 eq), K₂CO₃ (0.27 g, 1.65 mmol, 5 eq) and Pd(dppf)Cl₂CH₂Cl₂ (64.6 mg, 79.1 umol, 0.1 eq) under N₂ atmosphere. The system was degassed and charged with nitrogen three times. The mixture was heated to and stirred at 120 °C for 0.5 hour under N₂. The reaction was monitored by TLC (petroleum ether:EtOAc=1:2, product Rf=0.45). After cooling to room temperature, the mixture was filtered through a pad of Celite, and the filter cake was washed with CH₂Cl₂ (30 mL×3). The filtrate was concentrated under reduced pressure to dryness. The residue was purified by silica gel column chromatography (petroleum ether:EtOAc=100:1 to 3:1) and further purified by prep-HPLC {column: Nano-micro Kromasil C18 (100*30 mm, 5 um); mobile phase: [water (0.1% TFA)-ACN]; B %: 20%-30%, 10 min} to get the compound 22e (50 mg, 22.8% yield) and 22f (70.0 mg, 253 umol, 32.0% yield) as yellow oils. Both were confirmed by ¹HNMR.

### Step 6:

To a solution of 22e (50 mg) in dichloromethane (1 mL) was added TFA (0.2 mL) in one portion at 0 °C. The mixture was warmed to and stirred at 25 °C for 1 hour. The reaction was monitored by TLC (petroleum ether:EtOAc=2:1). The reaction mixture was diluted with CH₂Cl₂ (10 mL) and concentrated under reduced pressure to dryness below 10 °C. The mixture was re-dissolved in CH₂Cl₂ (10 mL) and treated with Amberlyst A21 (0.1 g) and stirred for another 0.5 hour. After filtering, the cake was washed with CH₂Cl₂ (5 mL×2), and the filtrate was concentrated under reduced pressure to provide Ex. 22 (29 mg, 91% yield) as brown oil; ¹H NMR: 400 MHz CDCl₃, δ 7.36 (d, J=10.4 Hz, 1H), 7.07 (d, J=1.6 Hz, 1H), 6.76 (dd, J=10.4, 1.6 Hz, 1H), 2.43 (s, 3H), 1.89-1.98 (m, 1H), 1.07-1.14 (m, 2H), 0.82-0.88 (m, 2H); LC-MS: m/z [M+H]⁺=177.1.

### Example 23: Preparation of sodium 3-cyclopropyl-2-methyl-7-oxocyclohepta-1,3,5-trien-1-olate (Ex.23)

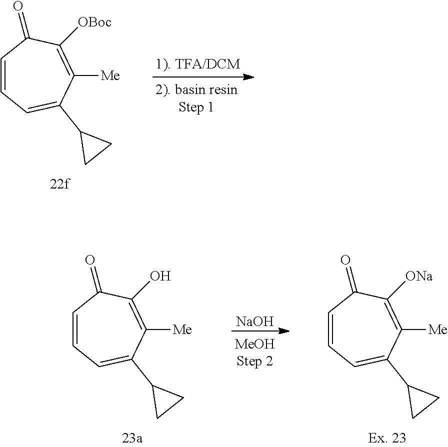

### Step 1:

To a solution of 22f (70.0 mg, available from preparative example 22) in dichloromethane (1 mL) was added TFA (0.3 mL) in one portion at 0 °C. The mixture was warmed and stirred at 25 °C for 1 hour. The reaction was monitored by TLC (petroleum ether:EtOAc=2:1). The reaction mixture was diluted with CH₂Cl₂ (10 mL) and concentrated under reduced pressure to dryness below 10 °C. The mixture was re-dissolved in CH₂Cl₂ (10 mL) and treated with Amberlyst A21 (0.1 g) and stirred for another 0.5 hour. After filtering, the cake was washed with CH₂Cl₂ (5 mL×2), and the filtrate was concentrated under reduced pressure to dryness. The residue was further purified by prep-HPLC {column: Nano-micro Kromasil C18 (100*30 mm, 5 um); mobile phase: [water (0.1% TFA)-ACN]; B %: 20%-30%, 10 min} to get 23a (25 mg, 56% yield) as a yellow oil.

### Step 2:

To a solution of 23a (25 mg, 141 umol, 1.00 eq) in MeOH (5 mL) was added 25% solution of NaOH (6 mg, 141 umol, 1.00 eq) in water (180 uL) at 25 °C. The mixture was stirred at 45 °C for 0.5 hour. After cooling, the reaction mixture was concentrated under reduced pressure to remove MeOH. The crude product was triturated with acetone at 25 °C and stirred for another 30 min. After filtering, the cake was washed with acetone (5 mL×2), and the precipitate was collected and dried in vacuum to get Ex.23 (11.5 mg, 40.9% yield) as a yellow solid; ¹H NMR: 400 MHz CD₃OD, δ 6.87-6.94 (m, 1H) 6.80-6.85 (m, 1H) 6.56 (d, J=10.4 Hz, 1H) 2.52 (s, 3H) 2.01-2.11 (m, 1H) 0.96-1.03 (m, 2H) 0.65 (q, J=6.0 Hz, 2H); LC-MS: m/z [M+H-Na]⁺=177.1.

### Example 24: Preparation of 4-cyclopropyl-7-hydroxy-2-methylcyclohepta-2,4,6-trien-1-one (Ex.24)

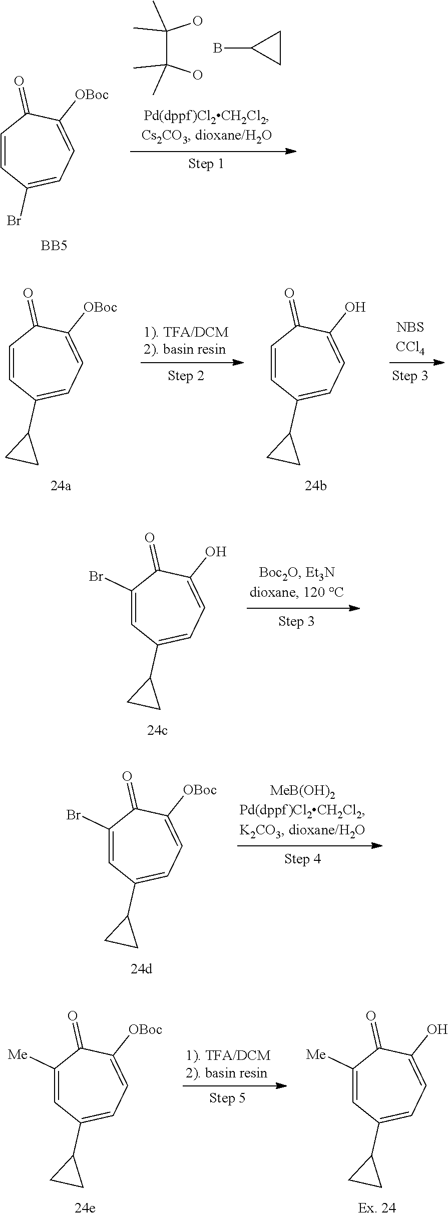

### Step 1:

To a solution of building block BB5 (1.00 g, 3.32 mmol, 1 eq) in dioxane (40 mL) and H₂O (5 mL) added 2-cyclopropyl-4,4,5,5-tetramethyl-1,3,2-dioxaborolane (5.58 g, 33.2 mmol, 10 eq) and Cs₂CO₃ (5.41 g, 16.6 mmol, 5 eq) at 25 °C. The mixture was degassed and purged with N₂ twice, and Pd(dppf)Cl₂·CH₂Cl₂ (542 mg, 664 umol, 0.2 eq) was added under N₂ atmosphere. The system was degassed and charged with nitrogen three more times, and the mixture was heated to and stirred at 120 °C for 2 hours under N₂. After cooling, the mixture was filtered through a pad of Celite and the filter cake washed with CH₂Cl₂ (40 mL×3). The filtrate was concentrated under reduced pressure to dryness. The residue was purified by silica gel column chromatography (petroleum ether:EtOAc=20:1 to 3:1) to afford 24a (670 mg, 76.9% yield) as a yellow solid.

### Step 2:

To a solution of 24a (240 mg) in CH₂Cl₂ (2 mL) was added TFA (1 mL) in one portion at 0 °C. The mixture was warmed and stirred at 25 °C for 1 hr. The reaction was monitored by TLC (petroleum ether:EtOAc=3:1). The reaction mixture was diluted with CH₂Cl₂ (10 mL) and concentrated under reduced pressure to dryness below 10 °C. The residue was purified by prep-HPLC {column: Welch Xtimate C18 (100*25 mm, 3 um); mobile phase: [water(0.1% TFA)-ACN]; B %: 1%-55%, 12 min} to afford 24b (100 mg, 67.4% yield) as a yellow solid.

### Step 3:

To a solution of 24b (440 mg, 2.71 mmol, 1 eq) in CCl₄ (7.00 mL) was added NBS (434 mg, 2.44 mmol, 0.90 eq) at 25 °C under N₂. The mixture was heated to and stirred at 80 °C for 1 hour. The reaction was monitored by LCMS. After cooling to room temperature, the mixture was concentrated under reduced pressure to provide crude 24c as a brown oil. This crude product (654 mg, 2.71 mmol, 1 eq) was dissolved in dioxane (5 mL) and Boc₂O (5.92 g, 27.1 mmol, 6.23 mL, 10 eq) and Et₃N (2.75 g, 27.1 mmol, 3.78 mL, 10 eq) were added. The resulting mixture was heated to and stirred at 120 °C for 1 hour. The reaction was monitored by LCMS. After cooling to room temperature, the mixture was concentrated under reduced pressure to dryness. The residue was purified by silica gel column chromatography (petroleum ether:EtOAc=50:1 to 10:1) to afford 24d (270 mg, 29.1% yield) as a yellow solid.

### Step 4:

To a mixture of 24d (250 mg, 732 umol, 1.00 eq) and K₂CO₃ (202 mg, 1.47 mmol, 2.00 eq) in dioxane (10 mL) and H₂O (2 mL) were added methylboronic acid (438 mg, 7.33 mmol, 10 eq) and Pd(dppf)Cl₂·CH₂Cl₂ (59.8 mg, 73.2 umol, 0.10 eq) in one portion at 25 °C under N₂ atmosphere. The system was degassed and charged with nitrogen three times. The mixture was heated to and stirred at 120 °C for 0.5 hour. After cooling to room temperature, the mixture was filtered through a pad of Celite and the filter cake washed with CH₂Cl₂ (50 mL×3). The filtrate was concentrated under reduced pressure to dryness. The residue was purified by prep-HPLC {column: Nano-micro Kromasil C18 (100*30 mm, 5 um); mobile phase: [water(0.1% TFA)-ACN]; B %: 40%-55%, 10 min} to afford 24e (150 mg, 74.0% yield) as a yellow solid.

### Step 5:

To a solution of 24e (50 mg, 180 umol, 1.00 eq) in dichloromethane (3 mL) was added TFA (1 mL) in one portion at 0 °C. The mixture was warmed and stirred at 25 °C for 1 hour. The reaction was monitored by TLC (petroleum ether:EtOAc=3:1). The reaction mixture was diluted with CH₂Cl₂ (10 mL) and concentrated under reduced pressure to dryness below 10 °C. The mixture was re-dissolved in CH₂Cl₂ (5 mL) and stirred with Amberlyst A21 (0.1 g) at 25 °C for another 0.5 hour. After filtering, the cake was washed with CH₂Cl₂ (10 mL×2) and the filtrate was concentrated under reduced pressure to provide compound Ex. 24 (21 mg, 65.9% yield) as a yellow solid; ¹H NMR: 400 MHz DMSO-d₆, δ 7.39 (s, 1H), 7.19-7.12 (m, 1H), 7.09-7.02 (m, 1H), 2.33 (s, 3H), 1.99 (tt, J=8.4 Hz, 1H), 1.00-0.92 (m, 2H), 0.78-0.71 (m, 2H); LC-MS: m/z [M+H]⁺=177.1.

### Example 25: Preparation of 4-ethyl-7-hydroxy-2-methylcyclohepta-2,4,6-trien-1-one (Ex.25)

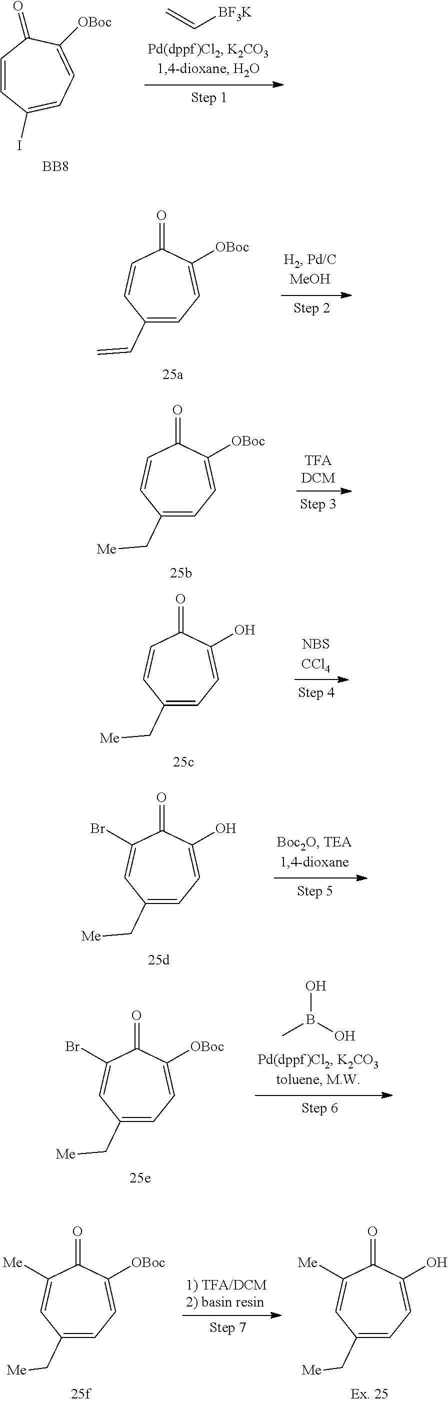

### Step 1:

To a mixture of building block BB8 (2.5 g, 7.18 mmol, 1 eq) and potassium vinyltrifluoroborate (1.44 g, 10.7 mmol, 1.5 eq) in 1,4-dioxane (25 mL) and H₂O (5 mL) were added K₂CO₃ (1.98 g, 14.3 mmol, 2 eq) and Pd(dppf)Cl₂·CH₂Cl₂ (586 mg, 0.72 mmol, 0.1 eq) in one portion at 20 °C under N₂ atmosphere. The system was degassed and charged with nitrogen three times. The mixture was heated to and stirred at 110 °C for 2.5 hours. The reaction was monitored by TLC (petroleum ether:EtOAc=5:1, product Rf=0.45). After cooling to room temperature, the reaction mixture was quenched by water (50 mL) and then extracted with EtOAc (50 mL×3). The combined organic layers were washed with brine (40 mL×3), dried over Na₂SO₄, filtered and concentrated under reduced pressure to give a residue. The residue was purified by silica gel column chromatography (petroleum ether:EtOAc=100:1 to 5:1) to give 25a (1.7 g, 47.7% yield) as a yellow solid.

### Step 2:

To a solution of 25a (0.85 g, 3.42 mmol, 1 eq) in MeOH (5 mL) was added 10% Pd/C (0.3 g) under N₂. The suspension was degassed under vacuum and purged with H₂ several times. The mixture was stirred under H₂ (15 psi) at 20 °C for 30 min. The reaction was monitored by TLC (petroleum ether:EtOAc=4:1). The reaction mixture was filtered through a pad of Celite, and the filter cake was washed with MeOH (10 mL×2). The combined filtrates were concentrated under reduced pressure to dryness. The residue was purified by silica gel column chromatography (petroleum ether:EtOAc=100:1 to 8:1) to give 25b (1.15 g, 67.1% yield) as a yellow solid.

### Step 3:

To a solution of 25b (1.15 g, 4.59 mmol, 1 eq) in CH₂Cl₂ (15 mL) was added TFA (3 mL) in one portion at 0 °C under N₂. The mixture was warmed and stirred at 20 °C for 1 hour. The reaction was monitored by TLC (petroleum ether:EtOAc=4:1). The reaction mixture was diluted with CH₂Cl₂ (20 mL) and concentrated under reduced pressure to dryness. The residue was re-dissolved in CH₂Cl₂ (10 mL) and stirred with Amberlyst A21 (1 g) at 20 °C for 20 min. After filtering, the cake was washed with CH₂Cl₂ (5 mL×2), and the filtrate was concentrated under reduced pressure below 10 °C to give 25c (637 mg, 92.3% yield) as a yellow solid.

### Step 4:

To a solution of 25c (637 mg, 4.24 mmol, 1 eq) in CCl₄ (20 mL) was added NBS (490 mg, 2.76 mmol, 0.65 eq) in one portion at 20 °C under N₂. The mixture was heated to and stirred at 80 °C for 1 hour. The reaction was monitored by HPLC and halted when it showed ~14% of desired compound. After cooling to room temperature, the reaction mixture was concentrated under reduced pressure to give crude 25d (970 mg, crude) as a yellow solid which was used to next step directly.

### Step 5:

To a mixture of 25d (970 mg, 4.24 mmol, 1 eq) and triethylamine (3.43 g, 33.9 mmol, 8 eq) in 1,4-dioxane (15 mL) was added Boc₂O (5.55 g, 25.4 mmol, 6 eq) in one portion at 20 °C under N₂. The mixture was heated to 110 °C and stirred for 1 hour. The reaction was monitored by TLC (petroleum ether:EtOAc=1:1, product Rf=0.45). After cooling to room temperature, the reaction mixture was concentrated under reduced pressure to dryness and residue was purified by silica gel column chromatography (petroleum ether:EtOAc=100:1 to 8:1) to give 25e (0.44 g, 31.5% yield) as a yellow solid.

### Step 6:

To a mixture of 25e (440 mg, 1.34 mmol, 1 eq) and methylboronic acid (800 mg, 13.4 mmol, 10 eq) in toluene (10 mL) were added K₂CO₃ (369 mg, 2.67 mmol, 2 eq) and Pd(dppf)Cl₂·CH₂Cl₂ (327 mg, 0.4 mmol, 0.3 eq) in one portion at 20 °C under N₂ atmosphere. The system was degassed and charged with nitrogen three times. The mixture was heated to and stirred at 120 °C for 2.5 hours in a microwave. The reaction was monitored by LC-MS. After cooling to room temperature, the reaction mixture was quenched with brine (30 mL) and extracted with EtOAc (20 mL×3). The combined organic layers were washed with brine (20 mL×2), dried over Na₂SO₄, filtered and concentrated under reduced pressure to give a residue. The residue was purified by silica gel column chromatography (petroleum ether:EtOAc=100:1 to 8:1) to give 25f (45 mg, 12.7% yield) as a yellow solid.

### Step 7:

To a solution of 25f (45 mg) in CH₂Cl₂ (2 mL) was added trifluoroacetic acid (0.5 mL) in one portion at 0 °C under N₂. The mixture was warmed and stirred at 20 °C for 1 hour. The reaction was monitored by TLC (petroleum ether:EtOAc=5:1). The mixture was diluted with CH₂Cl₂ (10 mL) and concentrated under reduced pressure to dryness. The residue was re-dissolved in CH₂Cl₂ (5 mL) and stirred with Amberlyst A21 (200 mg) at 20 °C for 20 min. After filtering, the cake was washed with CH₂Cl₂ (5 mL×2) and the filtrate was concentrated under reduced pressure below 10 °C to provide Ex. 25 (25 mg, 89.4% yield) as a brown solid; ¹H NMR: 400 MHz CD₃OD, δ 7.59 (s, 1H), 7.27-7.37 (m, 2H), 2.67 (q, J=7.6 Hz, 2H), 2.44 (s, 3H), 1.25 (t, J=7.6 Hz, 3H); LC-MS: m/z [M+H]⁺=165.1.

### Example 26: Preparation of 4-ethyl-2-hydroxy-7-methylcyclohepta-2,4,6-trien-1-one (Ex. 26)

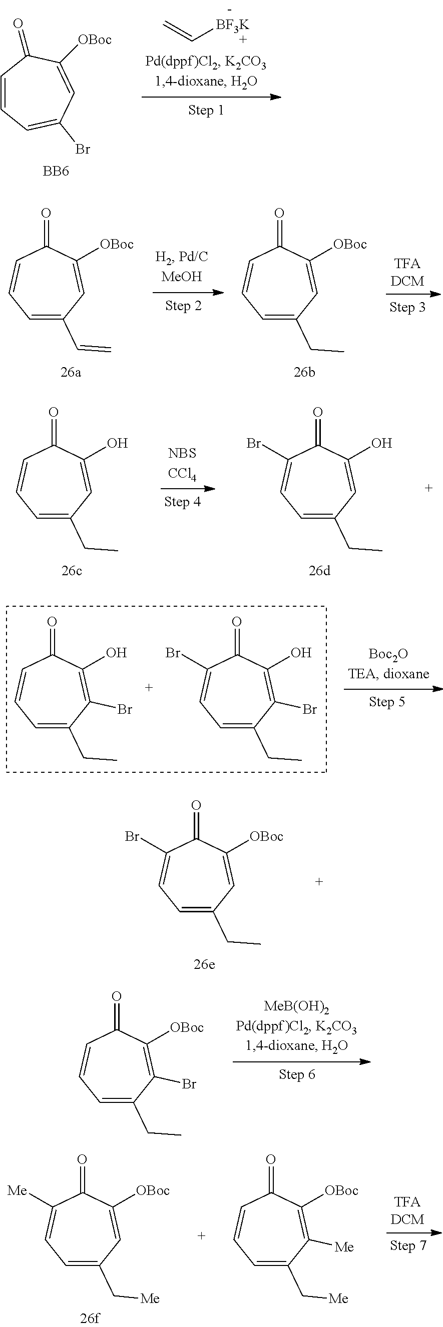

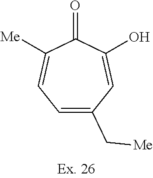 [SMILES: CCc1ccc(C)c(=O)c(O)c1]

### Step 1:

To a mixture of BB6 (8 g, 26.6 mmol, 1 eq) and potassium vinyltrifluoroborate (7.12 g, 53.1 mmol, 2 eq) in 1,4-dioxane (80 mL) and H₂O (16 mL) were added K₂CO₃ (7.34 g, 53.1 mmol, 2 eq) and Pd(dppf)Cl₂·CH₂Cl₂ (1.08 g, 1.33 mmol, 0.05 eq) in one portion at 25 °C under N₂ atmosphere. The system was degassed and charged with nitrogen three times. The mixture was heated to and stirred at 105 °C for 1 hour under N₂. The reaction was monitored by TLC (petroleum ether:EtOAc=5:1, product Rf=0.36). After cooling to 25 °C, water (120 mL) was added and the mixture extracted with EtOAc (120 mL×3). The combined organic phases were washed with water (30 mL) and brine (30 mL), dried over anhydrous Na₂SO₄, filtered and concentrated under reduced pressure to dryness. The residue was purified by silica gel column chromatography (petroleum ether:EtOAc=10:1 to 5:1) to give 26a (5 g, 75.8% yield) as yellow gum.

### Step 2:

To a solution of 26a (2.5 g, 10 mmol, 1 eq) in MeOH (40 mL) was added 10% Pd/C (0.5 g) under N₂ atmosphere. The suspension was degassed under vacuum and purged with H₂ three times. The mixture was stirred under H₂ (15 psi) at 25 °C for 1 hour. The reaction was monitored by TLC and HPLC (petroleum ether:EtOAc=5:1). The reaction mixture was filtered through a pad of Celite and the filter cake was washed with MeOH (10 mL×2). The combined filtrates were concentrated under reduced pressure to dryness. The crude product was purified by silica gel column chromatography (petroleum ether:EtOAc=50:1 to 10:1) to give 26b (1.2 g, 47.6% yield) as a yellow gum.

### Step 3:

To a solution of 26b (1.0 g, 4.00 mmol, 1 eq) in CH₂Cl₂ (10 mL) was added TFA (2 mL) in one portion at 0 °C. The reaction mixture was warmed to room temperature and stirred at 25 °C for 1 hour. The reaction was monitored by TLC (petroleum ether:EtOAc=3:1). The reaction mixture was concentrated under reduced pressure to dryness below 10 °C. The residue was re-dissolved in CH₃CN (1 mL) and distilled H₂O (2 mL) to give crude 26c (0.5 g, 83.3% yield) as a yellow gum after lyophilization.

### Step 4:

To a solution of 26c (0.5 g, 1 eq) in CCl₄ (5 mL) was added NBS (320 mg, 0.5 eq) at 25 °C under N₂. The mixture was heated and stirred at 80 °C for 2 hours. The reaction was monitored by HPLC which showed three regioisomers with desired mass. After cooling to room temperature, water (10 mL) was added and the mixture extracted with ethyl acetate (30 mL×3). The combined organic phases were washed with water (10 mL) and brine (10 mL), dried over anhydrous Na₂SO₄, filtered and concentrated under reduced pressure to dryness to give crude regioisomeric mixture with 26d (0.9 g) as a brown gum.

### Step 5:

To a solution of crude 26d (5.1 g, 22.2 mmol, 1 eq) in 1,4-dioxane (75 mL) was added Et₃N (4.5 g, 44.5 mmol, 2 eq) and Boc₂O (7.3 g, 33.4 mmol, 1.5 eq) in one portion at 25 °C under N₂. The mixture was heated and stirred at 100 °C for 1 hour. The reaction was monitored by TLC (petroleum ether:EtOAc=5:1, product Rf=0.5). After cooling to room temperature, the mixture was concentrated under reduced pressure to dryness. The residue was purified by silica gel column chromatography (petroleum ether:EtOAc=10:1 to 8:1) to give a crude mixture of 26e and one remaining regioisomer (2.3 g) as a yellow gum.

### Step 6:

To a mixture of crude 26e (700 mg, 2.13 mmol, 1 eq) in 1,4-dioxane (7 mL) and H₂O (1 mL) were added methylboronic acid (893 mg, 14.9 mmol, 7 eq), K₂CO₃ (881 mg, 6.39 mmol, 3 eq) and Pd(dppf)Cl₂ (146 mg, 0.2 mmol, 0.1 eq) in one portion at 25 °C under N₂ atmosphere. The system was degassed and charged with nitrogen three times. The reaction mixture was heated to and stirred at 110 °C for 1 hour under N₂ atmosphere. The reaction was monitored by TLC (petroleum ether:EtOAc=5:1). After cooling to room temperature, the mixture was filtered through a pad of Celite and the filter cake washed with CH₂Cl₂ (30 mL×3). The filtrate was concentrated under reduced pressure to dryness. The residue was purified by silica gel column chromatography (petroleum ether:EtOAc=20:1 to 4:1) to afford crude 26f (200 mg), which was further purified by prep-HPLC {column: Nano-micro Kromasil C18 (100*30 mm, 5 um); mobile phase: [water (0.1% TFA)-ACN]; B %: 20%-30%, 10 min} to give a pure mixture of 26f and one other regioisomer (100 mg) as a yellow oil.

### Step 7:

To a solution of 26f and its regioisomer (200 mg, 0.76 mmol, 1 eq) in CH₂Cl₂ (2 mL) was added TFA (0.5 mL) in one portion at 0 °C. The mixture was warmed and stirred at 25 °C for 1 hour. The reaction was monitored by TLC (petroleum ether:EtOAc=5:1). The reaction mixture was diluted with CH₂Cl₂ (10 mL) and concentrated under reduced pressure below 10 °C to afford crude product (120 mg). A total of 160 mg of crude product from two combined batches (40 mg from another batch) was purified by SFC {column: DAICEL CHIRALCEL OD (250 mm*30 mm, 10 um); mobile phase: [Neu-IPA]; B %: 23%-23%, 3 min} to give pure Ex. 26 (52 mg) as a yellow oil; ¹H NMR: 400 MHz CD₃OD, δ 7.56 (d, J=10.4 Hz, 11H), 7.34 (d, J=1.6 Hz, 1H), 7.02 (d, J=10.4 Hz, 1H), 2.68-2.73 (q, J=7.6 Hz, 2H), 2.42 (s, 3H), 1.29 (t, J=7.6 Hz, 3H); LC-MS: m/z [M+H]⁺=165.1.

### Example 27: Preparation of 7-ethyl-2-hydroxy-3-methylcyclohepta-2,4,6-trien-1-one (Ex.27)

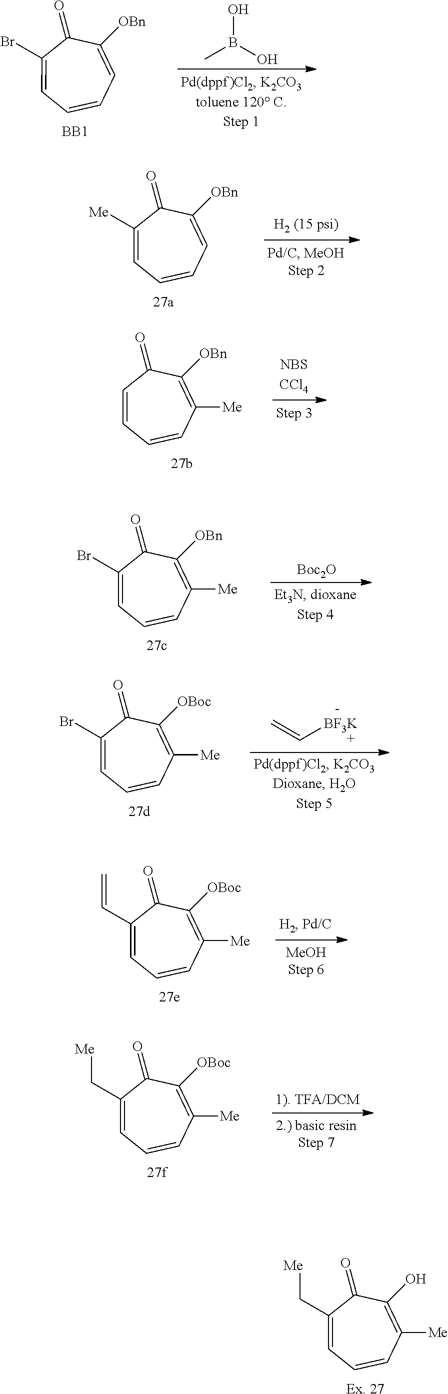

### Step 1:

To a mixture of building block BB1 (10 g, 24.0 mmol, 1 eq) and methylboronic acid (10.1 g, 168 mmol, 7 eq) in dry toluene (100 mL) was added K₂CO₃ (9.97 g, 72.1 mmol, 3 eq) and Pd(dppf)Cl₂ (1.76 g, 2.4 mmol, 0.1 eq) in one portion at 25 °C under N₂ atmosphere. The system was degassed and charged with nitrogen three times. The mixture was heated to and stirred at 120 °C for 3 hours. TLC (Petroleum ether:Ethyl acetate=3:1) showed the starting material was consumed completely and a new spot observed. After cooling, the mixture was filtered through a pad of Celite and the filter cake was washed with CH₂Cl₂ (30 mL×3). The filtrate was concentrated under reduced pressure to dryness. The residue was purified by silica gel chromatography (petroleum ether:EtOAc=20:1 to 4:1) to afford 27a (4 g, 67.6% yield, 92.1% purity) as a yellow oil.

### Step 2:

To a solution of 27a (2 g, 8.84 mmol, 1 eq) in MeOH (80 mL) was added Pd/C (1 g) under N₂. The suspension was degassed under vacuum and purged with H₂ three times. The mixture was stirred under H₂ (15 psi) and heated at 50 °C for 2 hours. The reaction was monitored by TLC (petroleum ether:EtOAc=1:1). After cooling, the reaction mixture was filtered through a pad of Celite, and the filter cake was washed with MeOH (20 mL×2). The combined filtrates were concentrated to dryness under reduced pressure to afford crude 27b (1.5 g, 46.7% yield, 75% purity) as a brown oil.

### Step 3:

To a solution of 27b (1.5 g, 8.26 mmol, 1 eq) in CCl₄ (40 mL) was added NBS (1.32 g, 7.44 mmol, 0.9 eq) at 25 °C under N₂. The mixture was heated and stirred at 80 °C for 5 hours. The reaction was monitored by LCMS. After cooling to room temperature, water (30 mL) was added and the mixture extracted with ethyl acetate (30 mL×3). The combined organic phases were washed with water (20 mL) and brine (50 mL), dried over anhydrous Na₂SO₄, filtered and concentrated under reduced pressure to dryness to give 27c (2 g) as a brown oil, that was taken on without further purification.

### Step 4:

To a solution of 27c (2 g, 1.86 mmol, 1 eq) in 1,4-dioxane (30 mL) were added Et₃N (564 mg, 5.58 mmol, 3 eq) and Boc₂O (2.03 g, 9.3 mmol, 5 eq) in one portion at 25 °C under N₂. The mixture was heated to and stirred at 100 °C for 1 hour. The reaction was monitored by LCMS. After cooling to room temperature, the mixture was concentrated under reduced pressure to dryness. The residue was purified by silica gel column chromatography (petroleum ether:EtOAc=100:1 to 90:1) to afford 27d (0.5 g, 51.2% yield, 60% purity) as a brown oil.

### Step 5:

To a mixture of 27d (500 mg, 951 umol, 1 eq) and potassium vinyltrifluoroborate (255 mg, 1.90 mmol, 2 eq) in 1,4-dioxane (5 mL) and H₂O (1 mL) were added Pd(dppf)Cl₂·CH₂Cl₂ (77 mg, 95.2 umol, 0.1 eq) and K₂CO₃ (394 mg, 2.86 mmol, 3 eq) in one portion at 25 °C under N₂ atmosphere. The system was degassed and charged with nitrogen three times. The mixture was heated to and stirred at 110 °C for 1 hour. The reaction was monitored by LCMS. After cooling to 25 °C, water (20 mL) was added, and the mixture was extracted with CH₂Cl₂ (20 mL×3). The combined organic phases were washed with water (10 mL) and brine (20 mL), dried over anhydrous Na₂SO₄, filtered and concentrated under reduced pressure to dryness. The residue was purified by prep-TLC (petroleum ether:EtOAc=5:1, product Rf=0.6) to afford 27e (200 mg, 40% yield, 50% purity) as a yellow oil.

### Step 6:

To a solution of 27e (100 mg, 382 umol, 1 eq) in MeOH (5 mL) was added Pd/C (100 mg) under N₂. The suspension was degassed under vacuum and purged with H₂ three times. The mixture was stirred under H₂ (15 psi) at 25 °C for 0.5 hour. The reaction was monitored by TLC (petroleum ether:EtOAc=5:1). The reaction mixture was filtered through a pad of Celite and the filter cake was washed with MeOH (5 mL×3). The combined filtrates were concentrated under reduced pressure to dryness. The residue was purified by prep-TLC (petroleum ether:EtOAc=3:1, product Rf=0.4) to give 27f (30 mg, 59.5% yield) as a yellow solid.

### Step 7:

To a solution of 27f (30 mg) in CH₂Cl₂ (2 mL) was added TFA (0.5 mL) in one portion at 0 °C. The mixture was warmed and stirred at 25 °C for 1 hour. The reaction was monitored by TLC (petroleum ether:EtOAc=3:1). The reaction mixture was diluted with CH₂Cl₂ (10 mL) and concentrated under reduced pressure to dryness below 10 °C. The mixture was re-dissolved in CH₂Cl₂ (10 mL) and stirred with Amberlyst A21 (0.1 g) for another 0.5 hour. After filtering, the cake was washed with CH₂Cl₂ (5 mL×2), and the filtrate was concentrated under reduced pressure to provide crude product. The residue was purified by prep-HPLC {column: Nano-micro Kromasil C18 (100*30 mm, 5 um); mobile phase: [water (0.1% TFA)-ACN]; B %: 20%-30%, 10 min} to give Ex. 27, (14 mg, 70.6% yield, 93.4% purity) as a yellow oil after lyophilization; ¹H NMR: 400 MHz CD₃OD, δ 7.51 (dd, J=10.4, 18.4 Hz, 2H), 7.02 (t, J=10.4 Hz, 1H), 2.86 (q, J=7.6 Hz, 2H), 2.44 (s, 3H), 1.24 (t, J=7.6 Hz, 3H); LC-MS: m/z [M+H]⁺=165.1.

### Example 28: Preparation of 7-hydroxy-4-isopropyl-2-methylcyclohepta-2,4,6-trien-1-one (Ex.28)

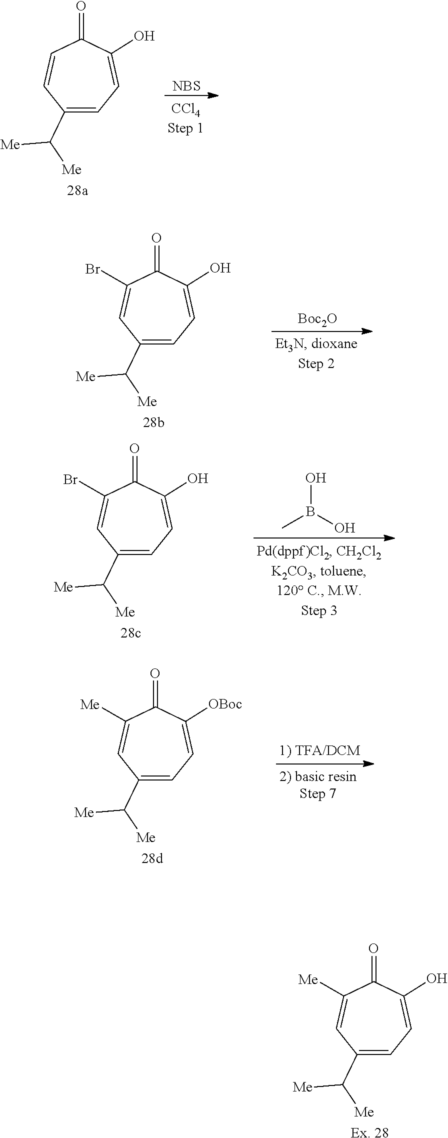

### Step 1:

To a solution of γ-thujaplicin 28a (380 mg, 2.31 mmol, 1 eq) in CCl₄ (15 mL) was added NBS (206 mg, 1.16 mmol, 0.5 eq) in portions at 20 °C under N₂. The mixture was heated to and stirred at 80 °C for 1 hour. The reaction was monitored by HPLC. After cooling to room temperature, the reaction mixture was concentrated under reduced pressure to give crude 28b (561 mg) as a yellow solid, which was used to the next step for further purification. (Three batches were run in parallel and combined for the next step.)

### Step 2:

To a mixture of crude 28b (740 mg, 3.04 mmol, 1 eq) and Et₃N (2.46 g, 24.4 mmol, 8 eq) in 1,4-dioxane (10 mL) was added Boc₂O (4 g, 18.3 mmol, 6 eq) in one portion at 20 °C under N₂. The mixture was heated to 110 °C and stirred for 1 hour. The reaction was monitored by TLC (petroleum ether:EtOAc=1:1). After cooling to room temperature, water (10 mL) was added and the mixture extracted with CH₂Cl₂ (30 mL×3). The combined organic phases were washed with water (10 mL) and brine (10 mL), dried over anhydrous Na₂SO₄, filtered and concentrated under reduced pressure to dryness. The residue was purified by silica gel column chromatography (petroleum ether:EtOAc=20:1 to 8:1) to give 28c (193 mg, 18.4% yield) as a yellow oil. (Three batches were run in parallel and combined for the next step.)

### Step 3:

To a mixture of 28c (400 mg, 1.17 mmol, 1 eq) and methylboronic acid (700 mg, 10 eq) in dry toluene (10 mL) were added K₂CO₃ (320 mg, 2 eq) and Pd(dppf)Cl₂·CH₂Cl₂ (285 mg, 0.3 eq) in one portion at 20 °C under N₂ atmosphere. The system was degassed and charged with nitrogen three times. The mixture was heated to and stirred at 120 °C for 2.5 hours in a microwave. The reaction was monitored by HPLC. After cooling to room temperature, the mixture was filtered through a pad of Celite, and the filter cake was washed with CH₂Cl₂ (20 mL×2). The mixture was concentrated under reduced pressure to dryness. The residue was purified by silica gel column chromatography (petroleum ether:EtOAc=100:1 to 8:1) to give a crude product (200 mg), which was purified by prep-HPLC (column: Waters Xbridge (150*25 mm, 5 um); mobile phase: [water (10 mM NH4HCO3)-ACN]; B %: 50%-50%, 20 min) to give 28d (80 mg, 24.7% yield) as a yellow solid.

### Step 4:

To a solution of 28d (80 mg, 1 eq) in CH₂Cl₂ (4 mL) was added TFA (1 mL) in one portion at 0 °C. The mixture was warmed and stirred at 20 °C for 1 hour. The reaction was monitored by TLC (petroleum ether:EtOAc=5:1). The mixture was concentrated under reduced pressure to dryness and then re-dissolved in CH₂Cl₂ (10 mL). Amberlyst A21 (1 g) was added to above solution and the mixture stirred for another 20 min. After filtering, the cake was washed with CH₂Cl₂ (10 mL×2), and the filtrate was concentrated under reduced pressure to give Ex. 28 (42 mg, 85.9% yield) as a yellow solid ¹H NMR: 400 MHz CD₃OD, δ 7.60 (s, 1H), 7.34 (d, 2H), 2.93 (dt, J=13.74, 6.72 Hz, 1H), 2.45 (s, 3H), 1.27 (d, J=7.04 Hz, 6H); LC-MS: m/z [M+H]⁺=179.1.

### Example 29: Preparation of 2-hydroxy-4-isopropyl-7-methylcyclohepta-2,4,6-trien-1-one (Ex.29)

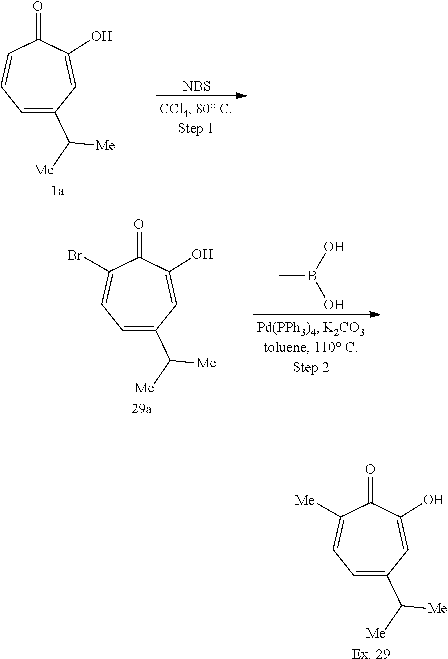

### Step 1:

To a solution of 0-thujaplicin 1a (2 g, 12.2 mmol, 1 eq) in CCl₄ (60 mL) was added NBS (1.84 g, 10.4 mmol, 0.85 eq) in portions at 25 °C under N₂. The mixture was heated and stirred at 90 °C for 4 hours. The reaction was monitored by LCMS. After cooling to room temperature, water (100 mL) was added and the mixture extracted with CH₂Cl₂ (100 mL×3). The combined organic phases were washed with water (50 mL) and brine (50 mL), dried over anhydrous Na₂SO₄, filtered and concentrated under reduced pressure to dryness. The residue was purified by silica gel column chromatography (petroleum ether:EtOAc=10:1 to 4:1) to afford 29a (1.5 g, 50.7% yield) as a yellow oil.

### Step 2:

To a mixture of 29a (1 g, 4.11 mmol, 1 eq) and methylboronic acid (369 mg, 6.17 mmol, 1.5 eq) in toluene (15 mL) were added Pd(dppf)Cl₂·CH₂Cl₂ (167 mg, 205 umol, 0.05 eq) and K₂CO₃ (1.71 g, 12.3 mmol, 3 eq) in one portion at 25 °C under N₂ atmosphere. The system was degassed and charged with nitrogen three times. The mixture was heated and stirred at 110 °C for 4 hours. The reaction was monitored by LCMS. After cooling to room temperature, water (15 mL) was added to above mixture, and the mixture was extracted with CH₂Cl₂ (50 mL×3). The combined organic phases were washed with brine (20 mL), dried over anhydrous Na₂SO₄, filtered and concentrated in vacuum to dryness. The residue was purified by prep-HPLC {column: Phenomenex luna C18 (250*50 mm, 10 um); mobile phase: [water(0.1% TFA)-ACN]; B %: 30%-60%, 20 min} to afford Ex. 29 (200 mg, 27% yield) as a brown oil after lyophilization; ¹H NMR: 400 MHz DMSO-d₆, δ 7.52 (d, J=10.4 Hz, 1H), 7.21 (d, J=1.6 Hz, 1H), 6.93 (d, J=10.4 Hz, 1H), 2.89 (spt, J=6.8 Hz, 1H), 2.30 (s, 3H), 1.20 (d, J=7.2 Hz, 6H); LC-MS: m/z [M+H]⁺=179.1.

### Example 30: Preparation of 2-hydroxy-3-isopropyl-7-methylcyclohepta-2,4,6-trien-1-one (Ex.30)

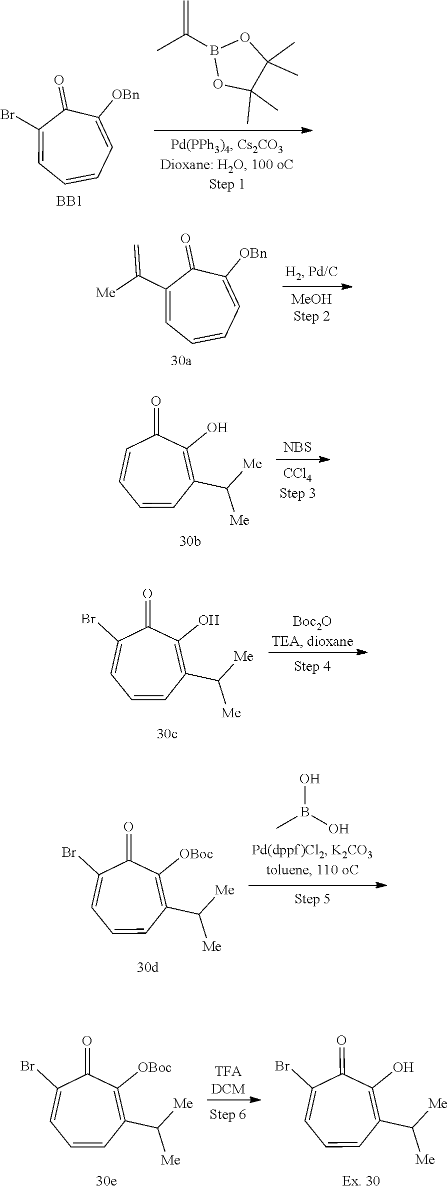

### Step 1:

To a mixture of building block BB1 (7 g, 24.0 mmol, 1 eq) and isopropenylboronic acid pinacol ester (12.1 g, 72.1 mmol, 3 eq) in 1,4-dioxane (80 mL) and water (10 mL) were added Cs₂CO₃ (15.6 g, 48.1 mmol, 2 eq) and Pd(PPh₃)₄ (555 mg, 480.9 umol, 0.02 eq) in one portion at 25 °C under N₂ atmosphere. The system was degassed and charged with nitrogen three times. The mixture was heated to and stirred at 100 °C for 16 hours under N₂ atmosphere. The reaction was monitored by LCMS. After cooling to room temperature, water (100 mL) was added and the mixture extracted with ethyl acetate (100 mL×3). The combined organic phases were washed with water (100 mL) and brine (100 mL), and dried over anhydrous Na₂SO₄. After filtering, the filtrate was concentrated under reduced pressure to dryness. The residue was purified by silica gel column chromatography (petroleum ether:EtOAc=100:1 to 5:1) to afford 30a (2.6 g, 42.86% yield) as a yellow oil.

### Step 2:

To a solution of 30a (2.6 g, 10.3 mmol, 1 eq) in MeOH (60 mL) was added 10% Pd/C (1.3 g) under N₂ atmosphere. The suspension was degassed under vacuum and purged with H₂ three times. The mixture was stirred under H₂ (15 psi) and heated at 50 °C for 4 hours. The reaction was monitored by TLC (petroleum ether:EtOAc=5:1, KMnO₄ as developer). After cooling, the reaction mixture was filtered through a pad of Celite, and the filter cake was washed with MeOH (10 mL×2). The combined filtrates were concentrated to dryness under reduced pressure to provide crude product 30b (1 g, 59.1% yield) as a yellow oil, which was used to next step without further purification.

### Step 3:

To a solution of 30b (1 g, 6.09 mmol, 1 eq) in CCl₄ (20 mL) was added NBS (867 mg, 4.87 mmol, 0.8 eq) in portions at 20 °C under N₂. The mixture was heated and stirred at 80 °C for 3 hours. The reaction was monitored by LCMS. After cooling to room temperature, saturated sodium thiosulfate solution (30 mL) was added drop-wise and the mixture stirred for another 10 min. The aqueous layer was extracted with ethyl acetate (20 mL×3), and the combined organic phases were washed with brine (20 mL), dried over anhydrous Na₂SO₄, filtered and concentrated under reduced pressure to dryness. This afforded crude 30c (1 g, 54% yield, 80% purity) as a brown oil that was used to next step without further purification.

### Step 4:

To a solution of 30c (1 g, 3.29 mmol, 1 eq) in 1,4-dioxane (15 mL) were added Et₃N (2.66 g, 26.3 mmol, 8 eq) and Boc₂O (7.18 g, 32.9 mmol, 10 eq) at 25 °C under N₂. The mixture was heated and stirred at 100 °C for 1 hour. The reaction was monitored by LCMS. After cooling to room temperature, the mixture was concentrated under reduced pressure to dryness. The residue was purified by silica gel column chromatography (petroleum ether:EtOAc=90:10) to give 30d (0.5 g, 22.1% yield) as a brown oil.

### Step 5:

To a mixture of 30d (0.5 g, 0.73 mmol, 1 eq) and methylboronic acid (218 mg, 3.64 mmol, 5 eq) in toluene (5 mL) were added Pd (dppf) Cl₂·CH₂Cl₂ (59 mg, 72.8 umol, 0.1 eq) and K₂CO₃ (302 mg, 2.19 mmol, 3 eq) at 25 °C under N₂ atmosphere. The system was degassed and charged with nitrogen three times. The mixture was heated and stirred at 110 °C for 4 hours. The reaction was monitored by LCMS. After cooling to room temperature, water (20 mL) was added and the mixture extracted with CH₂Cl₂ (10 mL×3). The combined organic phases were washed with brine (20 mL), dried over anhydrous Na₂SO₄, filtered and concentrated under reduced pressure to dryness. The residue was purified by prep-HPLC (column: Nano-micro Kromasil C18 (100*30 mm, 5 um); mobile phase: [water (0.1% TFA)-ACN]; B %: 45%-65%, 10 min) to afford 30e (80 mg, 39.4% yield) as a yellow oil.

### Step 6:

To a solution of 30e (80 mg, 288 umol, 1 eq)) in CH₂Cl₂ (5 mL) was added TFA (1 mL) in one portion at 0 °C. The mixture was warmed and stirred at 25 °C for 1 hour. LCMS showed the reaction was completed and the starting material consumed. The mixture was diluted with CH₂Cl₂ (10 mL) and concentrated under reduced pressure to dryness. The residue was purified by prep-HPLC {column: Nano-micro Kromasil C18 (100*30 mm, 5 um); mobile phase: [water(0.1% TFA)-ACN]; B %: 50%-65%, 10 min} to give Ex. 30 (25 mg, 46.4% yield, 95% purity) as a yellow oil after lyophilization; ¹H NMR: 400 MHz CD₃OD, δ 7.53 (dd, J=4.0, 10.0 Hz, 2H), 7.07 (t, J=10.0 Hz, 1H), 3.71 (quin, J=6.8 Hz, 1H), 2.44 (s, 3H), 1.26 (d, J=6.8 Hz, 6H); LC-MS: m/z [M+H]⁺=179.1.

### Example 31: Preparation of 2-hydroxy-7-methyl-3-(tetrahydro-2H-pyran-2-yl) cyclohepta-2,4,6-trien-1-one (Ex.31)

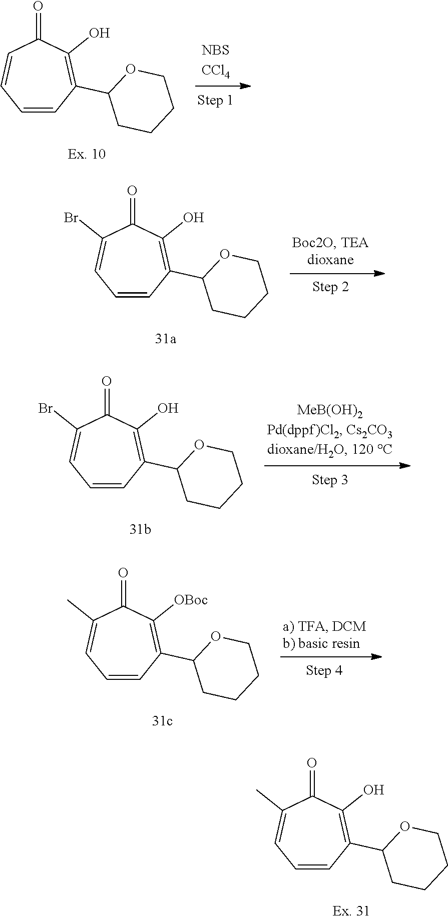

### Step 1:

To a solution of Ex.10 (350 mg, 1.70 mmol, 1.00 eq) in CCl₄ (5 mL) was added NBS (302 mg, 1.70 mmol, 1.00 eq) at 25 °C under N₂. The mixture was heated to and stirred at 80 °C for 1 hour. The reaction was monitored by LCMS. After cooling to room temperature, water (10 mL) was added and the mixture extracted with CH₂Cl₂ (30 mL×3). The combined organic layers were washed with brine (10 mL), dried over Na₂SO₄, filtered and concentrated under reduced pressure to give 31a (860 mg, crude) as a yellow oil that was used in the next step without further purification.

### Step 2:

To a solution of 31a (500 mg, 1.75 mmol, 1.00 eq) in dioxane (5 mL) were added Et₃N (709 mg, 7.01 mmol, 4.00 eq) and Boc₂O (1.15 g, 5.26 mmol, 3.00 eq). The mixture was heated to and stirred at 120 °C for 1 hour under N₂. The reaction was monitored by LCMS. After cooling to room temperature, the mixture was concentrated under reduced pressure to dryness to give a residue. The residue was purified by silica gel column chromatography (petroleum ether:EtOAc=100:1 to 20:1) to obtain 31b (200 mg, 29.6% yield) as a yellow oil.

### Step 3:

To a mixture of 31b (100 mg, 259 umol, 1.00 eq), methylboronic acid (155 mg, 2.60 mmol, 10.0 eq) and Cs₂CO₃ (169 mg, 519 umol, 2.00 eq) in dioxane (8.50 mL) and H₂O (0.50 mL) was added Pd(dppf)Cl₂·CH₂Cl₂ (106 mg, 129 umol, 0.50 eq) under N₂ atmosphere. The system was degassed and charged with nitrogen three times. The mixture was heated to and stirred at 118 °C for 0.5 hour. The reaction was monitored by TLC (petroleum ether:EtOAc=5:1). After cooling to room temperature, water (10 mL) was added and the mixture extracted with EtOAc (30 mL×3). The combined organic layers were washed with brine (10 mL), dried over Na₂SO₄, filtered and concentrated under reduced pressure to give a residue. The residue was purified by silica gel column chromatography (petroleum ether:EtOAc=20:1 to 5:1) to obtain 31c (50 mg, ~80% purity) as a yellow oil.

### Step 4:

To a solution of 31c (60.0 mg, 187 umol, 1.00 eq) in dichloromethane (2 mL) was added TFA (369 mg, 3.24 mmol, 17.3 eq) in one portion at 0 °C. The mixture was warmed to and stirred at 25 °C for 0.5 hour. The reaction was monitored by TLC (petroleum ether:EtOAc=5:1). The reaction mixture was diluted with CH₂Cl₂ (10 mL) and concentrated under reduced pressure to dryness below 10 °C. The residue was re-dissolved in CH₂Cl₂ (10 mL) and stirred with Amberlyst A21 (0.1 g) for another 0.5 hour. After filtering, the cake was washed with CH₂Cl₂ (5 mL×2), and the filtrate was concentrated under reduced pressure to dryness. The residue was purified by prep-HPLC {column: Nano-micro Kromasil C18 (100*30 mm, 5 um); mobile phase: [water (0.1% TFA)-ACN]; B %: 20%-30%, 10 min} give Ex.31 (12 mg, 29.1% yield) as a brown oil; ¹H NMR: 400 MHz DMSO-d₆, δ 7.70-7.73 (m, 1H), 7.32-7.35 (m, 1H), 6.93-6.98 (m, 1H), 4.84-4.88 (m, 1H), 4.09-4.11 (m, 1H), 3.59-3.65 (m, 1H), 1.59-2.01 (m, 5H), 1.18-1.23 (m, 2H); LC-MS: m/z [M+H]⁺=221.1.

### Example 32: Preparation of 7-fluoro-2-hydroxy-3-methylcyclohepta-2,4,6-trien-1-one (Ex.32)

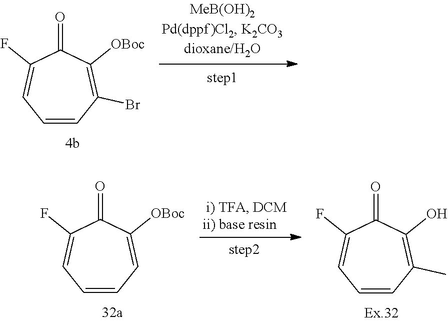

### Step 1:

To a stirred mixture of intermediate 4b (0.2 g, 626 umol, 1 eq), available from preparative example 4, methylboronic acid (375 mg, 6.2 mmol, 10 eq) and K₂CO₃ (173 mg, 1.25 mmol, 2 eq) in dioxane (4 mL) and water (0.8 mL) was added Pd(dppf)Cl₂ (45 mg, 62.67 umol, 0.1 eq) under a N₂ atmosphere. The system was degassed and then charged with nitrogen, the process was repeated three times. The resulting mixture was heated and stirred at 118 °C for 1 hr. Reaction progress was monitored by TLC (product spot was observed, petroleum ether:EtOAc=10:1, Rf=0.55). After cooling, water (10 mL) was added to the mixture, the aqueous mixture was extracted with EtOAc (20 mL×3), and the combined organic layers were washed with brine (10 mL), dried over anhydrous Na₂SO₄, filtered and concentrated under reduced pressure to dryness. The residue was purified by prep-TLC (petroleum ether:EtOAc=10:1, Rf=0.5) to give the methylated intermediate 32a (60 mg, 37.6%) as light yellow gum.

### Step 2:

To a solution of 32a (60 mg) in CH₂Cl₂ (2 mL) was added TFA (1 mL) in one portion at 0 °C. The mixture was warmed to and stirred at 25 °C for 0.5 hr. Reaction progress was monitored by TLC (32a, Petroleum ether:EtOAc=10:1, Rf=0.55, disappearance). The reaction mixture was diluted with CH₂Cl₂ (10 mL) and concentrated under reduced pressure at a temperature below 10 °C to dryness. The residue was re-dissolved in CH₂Cl₂ (10 mL), treated with Amberlyst A21 (0.1 g), and stirred for another 0.5 hr. After filtering, the solid cake was rinsed with CH₂Cl₂ (5 mL×2), the combined filtrate and rinsings were concentrated under reduced pressure to provide the titled product Ex.32 (32 mg, 88%) as light yellow solid; ¹H NMR: 400 MHz CDCl₃, δ 7.49-7.37 (m, 2H), 6.90 (dt, J=10.6 Hz, 1H), 2.50 (s, 3H), 1.25 (s, 1H); LC-MS: m/z [M+H]⁺=155.1.

### Example 33: Preparation of 7-fluoro-2-hydroxy-4-methylcyclohepta-2,4,6-trien-1-one (Ex.33)

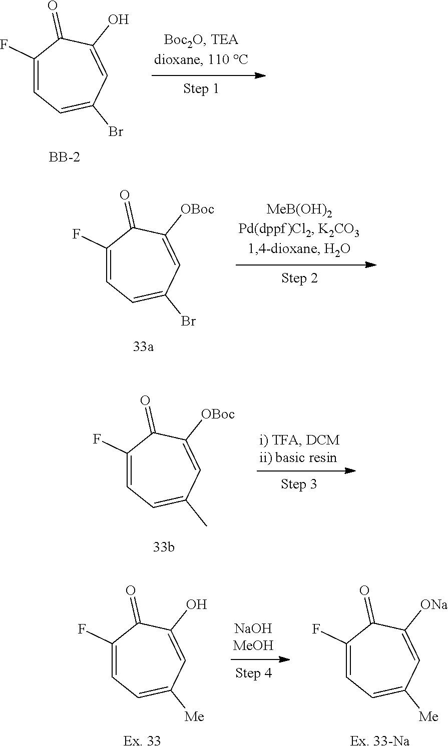

### Step 1:

To a stirred solution of BB2 (1.2 g, 5.5 mmol, 1 eq) in dioxane (5 mL) was added TEA (1.7 g, 16.5 mmol, 3 eq) and Boc₂O (2.4 g, 11.0 mmol, 2 eq) in one portion at 25 °C under N₂. The mixture was heated and stirred at 110 °C for 1 hour. Reaction progress was monitored by TLC (New product, petroleum ether:EtOAc=5:1, Rf=0.4). After cooling, the mixture was concentrated under reduced pressure to dryness. The residue was purified by silica gel column chromatography eluting with Petroleum ether:Ethyl acetate (from 10:1 to 5:1) to provide 33a (0.7 g, 40%) as a yellow gum.

### Step 2:

To a stirred mixture of 33a (250 mg, 783 umol, 1 eq), methylboronic acid (469 mg, 7.83 mmol, 10 eq) and K₂CO₃ (220 mg, 1.57 mmol, 2 eq) in dioxane (3 mL) and water (0.5 mL) was added Pd(dppf)Cl₂·CH₂Cl₂ complex (32 mg, 39.2 umol, 0.05 eq) under N₂ atmosphere. The system was degassed and then charged with nitrogen, the process was repeated three times. The resulting mixture was heated and stirred at 110 °C for 1 hour under a N₂ atmosphere. Reaction progress was monitored by TLC (petroleum ether:EtOAc=5:1, Rf=0.35, new product spot). After cooling, water (20 mL) was added and the aqueous mixture was extracted with EtOAc (20 mL×3). The combined organic extracts were washed with water (10 mL), brine (10 mL), dried over anhydrous Na₂SO₄, filtered and concentrated under reduced pressure to dryness. The residue was purified by prep-TLC (petroleum ether:EtOAc=5:1) to give 33b (45 mg, 22.6%) as a light yellow gum.

### Step 3:

To a stirred solution of 33b (45 mg) in dichloromethane (1 mL) was added TFA (0.2 mL) in one portion at 0 °C. The mixture was warmed and stirred at 25 °C for 0.5 hr. Reaction progress was monitored by TLC. The mixture was diluted with CH₂Cl₂ (10 mL) and concentrated under reduced pressure at a temperature below 10 °C to dryness. The residue was re-dissolved in CH₂Cl₂ (10 mL), treated with Amberlyst A21 (0.1 g), and stirred for another 0.5 hr. After filtering, the filtering cake was washed with CH₂Cl₂ (5 mL×2), and the combined filtrate and washings were concentrated under reduced pressure to afford the titled product Ex.33 (25 mg, 91.6%) as a light yellow solid.

### Step 4:

To a solution of Ex.33 (25 mg, 162 umol, 1 eq) in MeOH (2 mL) was added NaOH (6.5 mg, 162 umol, 1 eq) in water (0.1 mL) drop-wise at 20 °C, and the reaction mixture was stirred at 25 °C for 30 min. The reaction mixture was concentrated under reduced pressure to remove MeOH. The crude product was triturated with acetone (5 mL) at 25 °C and stirred for another 30 min. After filtering, the cake was washed with acetone (5 mL×2) and the precipitate was collected and dried in vacuum to provide the sodium salt of the titled product Ex.33-Na (26 mg, 91%) as a light yellow solid; ¹H NMR: 400 MHz CDCl3, δ ppm 7.56 (dd, J=10.4, Hz, 1H), 7.49-7.43 (m, 1H), 7.41-7.33 (m, 1H), 7.03 (dt, J=10.4 Hz, 1H); LC-MS: m/z [M+H]⁺=155.1.

### Example 34: Preparation of 4-cyclopropyl-7-fluoro-2-hydroxycyclohepta-2,4,6-trien-1-one (Ex.34)

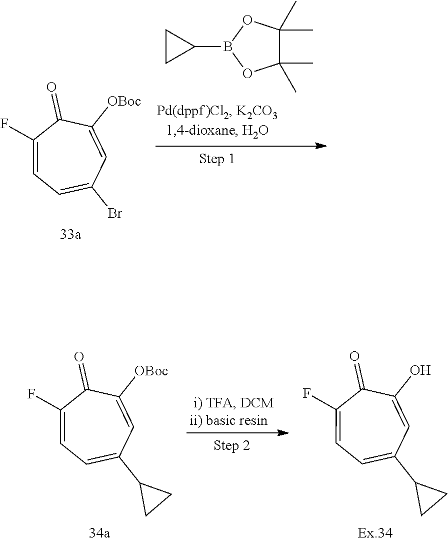

### Step 1:

To a stirred solution of intermediate 33a (300 mg, 940 umol, 1.00 eq) in 1,4-dioxane (7 mL) and H₂O (1 mL) was added cyclopropylboronic acid pinacol ester (1.58 g, 9.40 mmol, 10.0 eq) and Cs₂CO₃ (918 mg, 2.82 mmol, 3.00 eq) in one portion at 20 °C. The mixture was degassed and charged with N₂, the process was repeated three times. The complex Pd(dppf)Cl₂·CH₂Cl₂ (153 mg, 188 umol, 0.20 eq) was added to the mixture a under N₂ atmosphere. The system was degassed again and recharged with nitrogen, repeat three times. The resulting mixture was heated and stirred at 120 °C for 1 hr. Reaction progress was monitored by TLC [Petroleum ether/Ethyl acetate=5/1, Rf (material)=0.55, Rf (product)=0.4], complete conversion was observed. After cooling, the mixture was filtered through a pad of Celite and the filter cake was washed with CH₂Cl₂ (10 mL×3). The filtrate was concentrated under reduced pressure to dryness. The residue was purified by prep-TLC (Petroleum ether/Ethyl acetate=3/1) to give 34a (100 mg, 36.8%) as a yellow solid.

### Step 2:

To a solution of 34a (60 mg) in dichloromethane (1 mL) was added TFA (0.2 mL) in one portion at 0 °C. The mixture was warmed and stirred at 25 °C for 0.5 hr. TLC (Petroleum ether/Ethyl acetate=3/1, Rf (material)=0.55, Rf (product)=0.25) showed the starting material was consumed completely. The reaction mixture was diluted with CH₂Cl₂ (10 mL) and concentrated under reduced pressure at a temperature below 10 °C to dryness. The residue was re-dissolved in CH₂Cl₂ (10 mL), treated with Amberlyst A21 (0.1 g), and stirred for another 0.5 hr. After filtration, the filtering cake was washed with CH₂Cl₂ (5 mL×2), and the combined filtrate and washings were concentrated under reduced pressure to give the titled product Ex.34 (25 mg, 62.9%) as a brown solid; ¹H NMR: 400 MHz CD₃OD, δ ppm 7.53-7.61 (m, 1H), 7.23 (s, 1H), 6.88-6.91 (m, 1H), 2.02-2.07 (m, 1H), 1.13-1.16 (m, 2H), 0.87-0.88 (m, 2H); LC-MS: m/z [M+H]⁺=181.

### Example 35: Preparation of 7-fluoro-2-hydroxy-3-(tetrahydrofuran-2-yl)cyclohepta-2,4,6-trien-1-one (Ex.35)

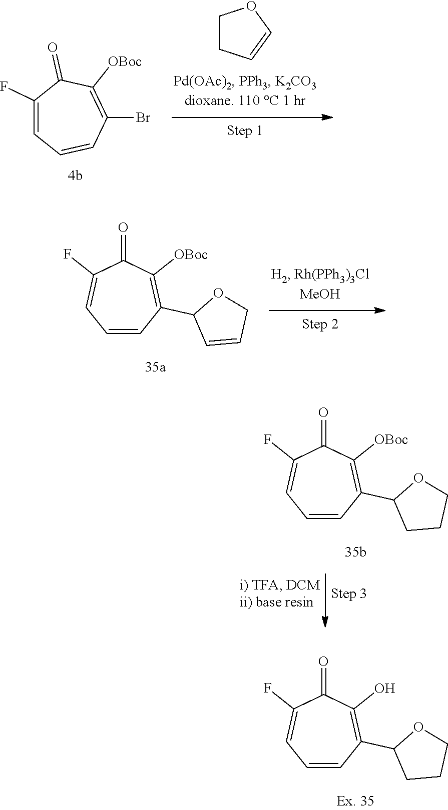

### Step 1:

To a stirring mixture of 4b (400 mg, 1.25 mmol, 1 eq) and 2,3-dihydrofuran (350 mg, 5.00 mmol, 4 eq) in dioxane (8 mL) was added K₂CO₃ (346 mg, 2.51 mmol, 2 eq), PPh₃ (65 mg, 250 umol, 0.2 eq) and Pd(OAc)₂ (28 mg, 125 umol, 0.1 eq) at 25 °C under a N₂ atmosphere. The mixture was degassed and recharged with nitrogen, this process was repeated three times. The resulting mixture was then heated and stirred at 110 °C for 1 h. TLC showed the starting material was consumed completely and a major new product spot was detected. After cooling, water (10 mL) was added and the aqueous mixture was extracted with EtOAc (50 mL×3). The combined organic extracts were dried over Na₂SO₄, filtered and concentrated under reduced pressure to yield a residue which was purified by silica gel column chromatography eluting with Petroleum ether:Ethyl acetate (80:1 to 20:1) to afford 35a (200 mg, 46.5%) as a yellow oil.

### Step 2:

To a solution of 35a (200 mg, 648 umol, 1 eq) in MeOH (5 mL) was added Rh(PPh₃)₃C1 (120 mg, 129 umol, 0.2 eq) under N₂. The resulting suspension was degassed under vacuum and purged with H₂ three times. The mixture was stirred under H₂ (20 psi) at 40 °C for 1.5 hours. After cooling, the mixture was filtered through a pad of Celite and the filter cake was rinsed with MeOH (10 mL×2). The combined filtrate and rinsings were concentrated under reduced pressure to dryness to give a crude product of 35b (300 mg, 40% purity) as a yellow oil.

### Step 3:

To a solution of 35b (300 mg, 966 umol, 1 eq) from step 2 above in dichloromethane (5 mL) was added TFA (1 mL) at 20 °C. The mixture was stirred at 20 °C for 20 min. Reaction progress was monitored by LCMS, and starting material was consumed completely with this period. The reaction mixture was then diluted with CH₂Cl₂ (10 mL) and concentrated under reduced pressure at a temperature below 10 °C to dryness. The residue was re-dissolved in CH₂Cl₂ (10 mL), treated with Amberlyst A21 (0.2 g), and stirred for another 0.5 hr. After filtration, the filter cake was washed with CH₂Cl₂ (5 mL×2), and the combined filtrate and washings were concentrated under reduced pressure to a residue. The residue was purified by prep-HPLC (Nano-micro Kromasil C18 100×30 mm 8 um; mobile phase: [water (0.1% TFA)-ACN]; B %: 20%-30%, 10 min) to afford the titled product Ex.35 (40 mg, 19.4%, 99.0% purity) as a yellow solid; ¹H NMR: 400 MHz CD₃OD, δ ppm 7.83 (d, J=10.4 Hz, 1H), 7.61 (dd, J=10.4 Hz, 1H), 7.20-7.06 (m, 1H), 5.31 (t, J=7.6 Hz, 1H), 4.22-4.08 (m, 1H), 3.98 (q, J=7.6 Hz, 1H), 2.71-2.55 (m, 1H), 2.10-1.88 (m, 2H), 1.70-1.54 (m, 1H); LC-MS: m/z [M+H]⁺=211.1.

### Example 36: Preparation of 7-fluoro-2-hydroxy-3-(tetrahydro-2H-pyran-4-yl)cyclohepta-2,4,6-trien-1-one (Ex.36)

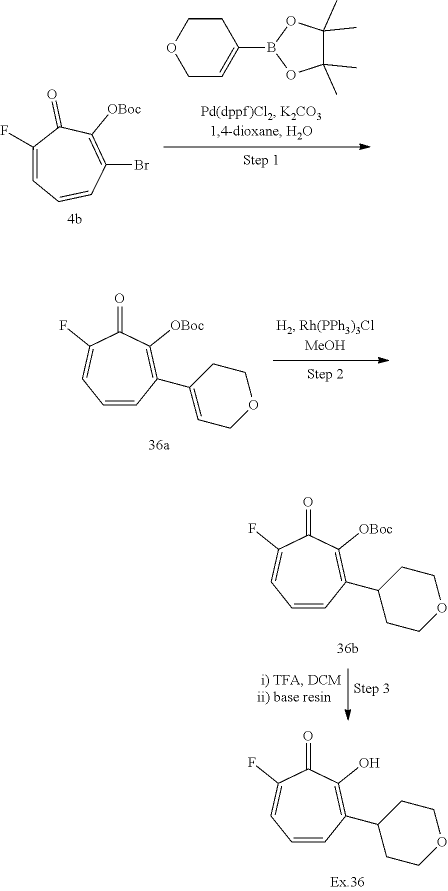

### Step 1:

To a mixture of 4b (0.5 g, 1.57 mmol, 1 eq) available from preparative example 4, K₂CO₃ (434 mg, 3.14 mmol, 2 eq), and 2-(3,6-dihydro-2H-pyran-4-yl)-4,4,5,5-tetramethyl-1,3,2-dioxaborolane (495 mg, 2.36 mmol, 1.5 eq) in dioxane/H₂O=15/1 (10 mL) was added Pd(dppf)Cl₂·CH₂Cl₂ complex (128 mg, 157 umol, 0.1 eq) under N₂ atmosphere. The system was degassed and re-charged with nitrogen, the process was repeated three times. The resulting mixture was heated and stirred at 120 °C for 0.5 hour under a N₂ atmosphere. Reaction progress was monitored by TLC [Petroleum ether/Ethyl acetate=5/1, Rf (material)=0.6, Rf (product)=0.3] which showed the starting material was consumed completely. After cooling, the mixture was filtered through a pad of Celite and the filter cake was washed with CH₂Cl₂ (30 mL×3). The filtrate and washings were combined and concentrated under reduced pressure to dryness. The residue was purified by silica gel column chromatography eluting with Petroleum ether:Ethyl acetate (20:1 to 5:1) to afford a crude product 36a (280 mg, crude) as a yellow oil.

### Step 2:

To a solution of 36a (280 g, 868 umol, 1 eq) in MeOH (5 mL) was added Rh(PPh₃)₃C1 (80 mg, 87 umol, 0.1 eq) under N₂. The suspension was degassed under vacuum and purged with H₂ several times. The mixture was stirred under H₂ (30 psi) at 25 °C for 0.5 hours. TLC (Petroleum ether/Ethyl acetate=3/1, Rf (material)=0.47, Rf (product)=0.43) showed no starting material remained and a new product spot was present. The reaction mixture was filtered through a pad of Celite and the filter cake was washed with MeOH (5 mL×3). The combined filtrates were concentrated under reduced pressure to dryness. The residue was purified by prep-TLC (petroleum ether:EtOAc=3:1, Rf=0.4) to provide 36b (100 mg, 35.5%) as a yellow solid.

### Step 3:

To a solution of 36b (60 mg) in CH₂Cl₂ (2 mL) was added TFA (0.5 mL) in one portion at 0 °C. The mixture was warmed and stirred at 25 °C for 1 hr. TLC (petroleum ether:EtOAc=3:1) showed the starting material was consumed completely. The reaction mixture was diluted with CH₂Cl₂ (10 mL) and concentrated under reduced pressure at a temperature below 10 °C to dryness. The residue was purified by pre-HPLC (TFA) to afford the titled product Ex.36 (12 mg, 28.9%) as a white solid; ¹H NMR: 400 MHz CD₃OD, δ 7.56-7.63 (m, 2H), 7.10-7.15 (m, 2H), 4.05-4.09 (m, 2H), 3.58-3.67 (m, 3H), 1.72-1.90 (m, 4H); LC-MS: m/z [M+H]⁺=225.1.

### Example 37: Preparation of 2-hydroxy-4-methyl-7-(trifluoromethyl)cyclohepta-2,4,6-trien-1-one (Ex.37)

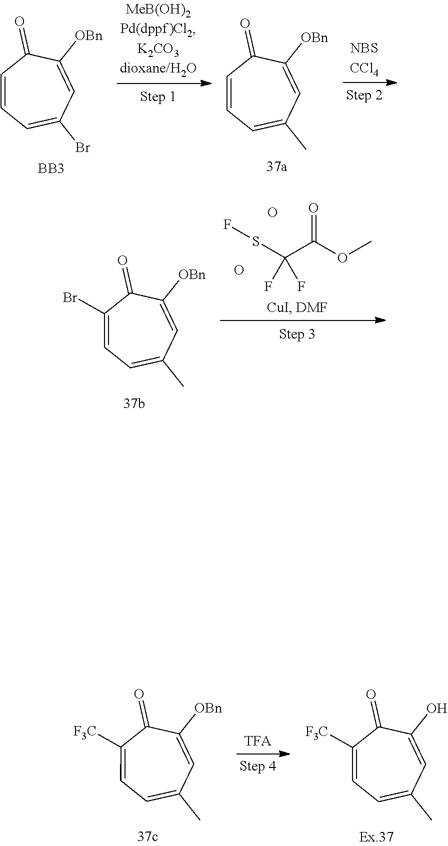

### Step 1:

To a mixture of intermediate BB3 (1.15 g, 3.93 mmol, 1 eq) and K₂CO₃ (1.09 g, 7.87 mmol, 2 eq) in dioxane (20 mL) and H₂O (4 mL) was added methyl boronic acid (2.35 g, 39.33 mmol, 10 eq) and the Pd(dppf)Cl₂·CH₂Cl₂ (321 mg, 39 umol, 0.1 eq) in one portion at 25 °C under N₂ atmosphere. The system was degassed and then recharged with nitrogen, the process was repeated three times. The resulting mixture was heated and stirred at 118 °C for 30 mins. Reaction progress was monitored by TLC [Petroleum ether/Ethyl acetate=3:1, Rf (material)=0.55, Rf (product)=0.3] which showed the starting material was consumed completely and a new product spot was formed. After cooling, the reaction mixture was poured into H₂O (100 mL) and extracted with ethyl acetate (50 mL×3). The combined organic extracts were washed with brine (50 mL×3), dried over anhydrous Na₂SO₄, filtered and concentrated under reduced pressure to dryness. The residue was purified by silica gel column chromatography eluting with Petroleum ether/Ethyl acetate (20/1 to 3/1) to give 37a (803 mg, 90.2%) as a yellow oil.

### Step 2:

To a solution of 37a (1.61 g, 7.10 mmol, 1 eq) in CCl₄ (20 mL) was added NBS (1.64 g, 9.23 mmol, 1.3 eq) in one portion at 25 °C under N₂. The mixture was heated and stirred at 80 °C for 1 hour. The reaction progress was monitored by TLC [Petroleum ether/Ethyl acetate=3:1, Rf (material)=0.3, Rf (product)=0.5] which showed completion of reaction. After cooling, water (30 mL) was added, the aqueous mixture was extracted with ethyl acetate (30 mL×3). The combined organic extracts were washed with water (20 mL), brine (50 mL), dried over anhydrous Na₂SO₄, filtered and concentrated under reduced pressure to dryness. The residue was purified by silica gel column chromatography eluting with Petroleum ether/Ethyl acetate (20/1 to 7/3) to provide 37b (800 mg, 67.3%) as a yellow oil.

### Step 3:

To a mixture of 37b (720 mg, 2.36 mmol, 1 eq) and methyl-2, 2-difluoro-2-fluorosulfonyl-acetate (2.27 g, 11.8 mmol, 5 eq) in DMF (5 mL) was added CuI (2.25 g, 11.8 mmol, 5 eq) in one portion at 25 °C under N₂ atmosphere. The system was degassed and recharged with nitrogen, the process was repeated three times. The resulting mixture was heated and stirred at 110 °C for 8 hours. Reaction progress was monitored by LCMS, and by end of the period all starting material was consumed and the desired product mass was observed. After cooling, the reaction mixture was poured into H₂O (100 mL) and the aqueous mixture was extracted with EtOAc 150 mL (50 mL×3). The combined organic extracts were washed with brine (50 mL), dried over Na₂SO₄, filtered and concentrated under reduced pressure to dryness. The residue was purified by prep-HPLC (TFA condition) to give 37c (139 mg, 20.0%) as a yellow solid.

### Step 4:

A solution of 37c (127 mg) in TFA (5 mL) was heated and stirred at 50 °C for 4 hours. LCMS showed the starting material was consumed and desired product mass was observed at the end of the period. The reaction mixture was diluted with CH₂Cl₂ (10 mL) and concentrated under reduced pressure at a temperature below 10 °C to dryness. The residue was re-dissolved in CH₂Cl₂ (10 mL), treated with Amberlyst A21 (0.1 g), and stirred for another 0.5 hr. After filtering, the solid cake was washed with CH₂Cl₂ (5 mL×2) and the filtrate was concentrated under reduced pressure to dryness. The residue was re-dissolved in CH₃CN (1 mL) and distilled water (2 mL), and then lyophilized to provide the titled product Ex.37 (71 mg, 80.5%) as a yellow solid; ¹H NMR: 400 MHz CD₃OD, δ 2.58 (q, J=3.2 Hz, 3H) 6.95 (br d, J=11.2 Hz, 1H) 7.08 (br d, J=10.0 Hz, 1H) 7.20-7.33 (m, 1H); LC-MS: m/z [M+H]⁺=205.1.

### Example 38: Preparation of 2-hydroxy-4-isopropyl-7-(trifluoromethyl)cyclohepta-2,4,6-trien-1-one (Ex.38)

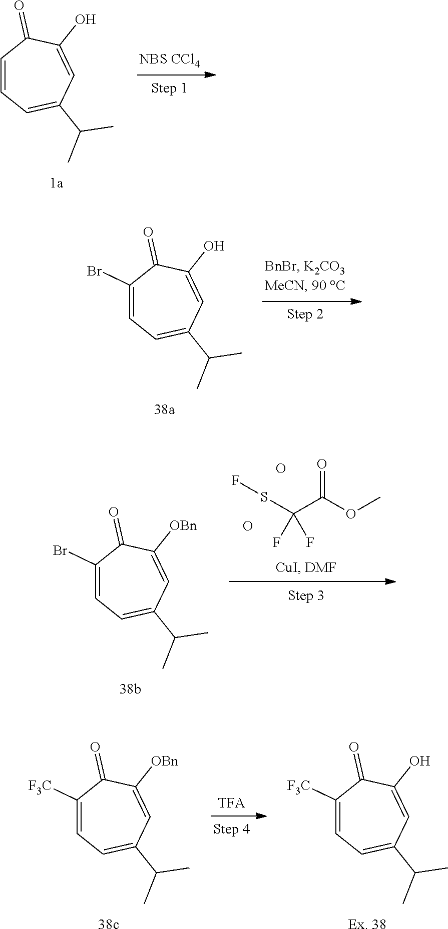

### Step 1:

To a solution of 0-thujaplicin 1a (1 g, 6.09 mmol, 1 eq) in CCl₄ (20 mL) was added NBS (1.19 g, 6.70 mmol, 1.1 eq) in one portion at 25 °C under N₂. The mixture was stirred at 80 °C for 30 min. LCMS showed the starting material was consumed and desired MS observed. After cooling, the reaction mixture was washed with brine (10 mL), dried over Na₂SO₄, filtered and concentrated under reduced pressure to dryness to give 38a (1.58 g, crude) as a white solid, which was used in the next step without purification.

### Step 2:

To a mixture of 38a (1.07 g, 4.42 mmol, 1 eq) in CH₃CN (20 mL) was added K₂CO₃ (1.83 g, 13.25 mmol, 3 eq) and benzyl bromide (1.13 g, 6.63 mmol, 1.5 eq) in one portion at 25 °C under N₂. The mixture was heated and stirred at 90 °C for 1 hour. The reaction progress was monitored by TLC (Petroleum ether/Ethyl acetate=5:1, Rf (material)=0, Rf (product)=0.3) which showed starting material was consumed at the end of the period. After cooling, the reaction mixture was filtered and concentrated under reduced pressure to dryness. The residue was purified by silica gel column chromatography eluting with Petroleum ether/Ethyl acetate (20/1 to 10/1) to give 38b (0.53 g, 36.0%) as a yellow oil.

### Step 3:

To a mixture of 38b (711 mg, 2.13 mmol, 1 eq) and methyl 2, 2-difluoro-2-fluorosulfonyl-acetate (2.05 g, 10.67 mmol, 5 eq) in DMF (9 mL) was added CuI (2.03 g, 10.7 mmol, 5 eq) in one portion at 25 °C under a N₂ atmosphere. The system was degassed and re-charged with nitrogen, the process was repeated three times. The resulting mixture was heated and stirred at 110 °C for 8 hours. Reaction progress was monitored by LCMS which showed consumption of starting material and the appearance of the desired product mass. After cooling, water (50 mL) was added, the aqueous mixture was extracted with EtOAc (50 mL×3). The combined organic extracts were washed with brine (50 mL), dried over Na₂SO₄, filtered and concentrated under reduced pressure to dryness. The residue was purified by prep-HPLC (TFA condition) to give 38c (192 mg, 27.9%) as a green solid.

### Step 4:

A solution of 38c (117 mg) in TFA (5 mL) was heated and stirred at 50 °C for 4 hours. Upon cooling to room temperature, the reaction mixture was diluted with CH₂Cl₂ (10 mL) and concentrated under reduced pressure at a temperature below 10 °C to dryness. The residue was re-dissolved in CH₂Cl₂ (10 mL), treated with Amberlyst A21 (0.1 g), and stirred for another 0.5 hr. After filtering, the solid cake was washed with CH₂Cl₂ (5 mL×2) and the filtrate was concentrated under reduced pressure to dryness. The residue was re-dissolved in CH₃CN (1 mL) and distilled water (2 mL), lyophilized, to provide Ex.38 (72 mg, 85.4%) as a yellow solid; ¹H NMR: 400 MHz CD₃OD, δ 7.56 (dd, J=10.4 Hz, 1H), 7.49-7.43 (m, 1H), 7.41-7.33 (m, 1H), 7.03 (dt, J=10.4 Hz, 1H); LC-MS: m/z [M+H]⁺=233.1.

### Example 39: Preparation of 7-hydroxy-4-methyl-2-(trifluoromethyl)cyclohepta-2,4,6-trien-1-one (Ex.39)

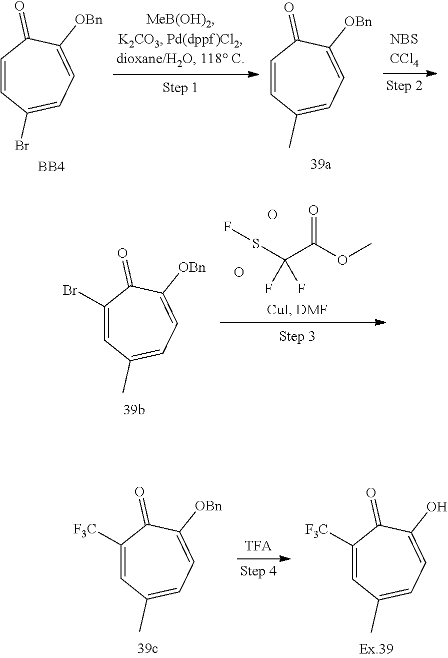

### Step 1:

To a mixture of building block BB4 (0.56 g, 1.93 mmol, 1 eq) and K₂CO₃ (533 mg, 3.86 mmol, 2 eq) in dioxane (20 mL) and H₂O (4 mL) was added methyl boronic acid (1.16 g, 19.3 mmol, 10 eq) and Pd(dppf)Cl₂·CH₂Cl₂ complex (157 mg, 193 umol, 0.1 eq) in one portion at 25 °C under N₂ atmosphere. The system was degassed and then recharged with nitrogen, the process was repeated three times. The resulting mixture was heated and stirred at 118 °C for 30 min. TLC (Petroleum ether/Ethyl acetate=3/1, Rf (material)=0.6, Rf (product)=0.3) showed the starting material was consumed completely and a new spot formed. After cooling, the mixture was filtered through a pad of Celite and the filter cake was washed with CH₂Cl₂ (30 mL×3). The combined filtrates were concentrated under reduced pressure to dryness. The residue was purified by silica gel column chromatography eluting with Petroleum ether/Ethyl acetate (20/1 to 4/1) to give 39a (0.36 g, 82.4%) as a yellow oil.

### Step 2:

To a mixture of 39a (0.36 g, 1.59 mmol, 1 eq) in CCl₄ (6 mL) was added NBS (424 mg, 2.39 mmol, 1.5 eq) in one portion at 25 °C under N₂. The mixture was stirred at 80 °C for 2 hours. Reaction progress was monitored by LCMS which showed the desired product mass peak. After cooling, water (30 mL) was added and the aqueous mixture was extracted with ethyl acetate (30 mL×3). The combined organic extracts were washed with water (20 mL), brine (50 mL), dried over anhydrous Na₂SO₄, filtered and concentrated under reduced pressure to dryness. The residue was purified by silica gel column chromatography eluting with Petroleum ether/Ethyl acetate (20/1 to 4/1) to provide 39b (0.33 g, 67.9%) as a yellow solid.

### Step 3:

To a mixture of 39b (0.16 g, 540 umol, 1 eq) and methyl 2,2-difluoro-2-(fluorosulfonyl)acetate (519 mg, 2.70 mmol, 5 eq) in DMF (3 mL) was added CuI (514 mg, 2.70 mmol, 5 eq) in one portion at 25 °C under N₂ atmosphere. The system was degassed and then recharged with nitrogen, the process was repeated three times. The resulting mixture was heated and stirred at 110 °C for 8 hours. LCMS showed the starting material was almost completely consumed and desired product mass was observed. After cooling to 25 °C, water (20 mL) was added and the aqueous mixture was extracted with EtOAc (50 mL×3). The combined organic extracts were washed with water (10 mL), brine (20 mL), dried over Na₂SO₄, filtered and concentrated under reduced pressure to a residue which was purified by prep-HPLC (TFA condition) to afford 39c (37.0 mg, 23.2%) as a yellow solid.

### Step 4:

A solution of 39c (89 mg, 302 umol, 1 eq) in TFA (3 mL) was heated and stirred at 50 °C for 5 hours. LCMS showed the starting material was almost completely consumed and the desired product mass was observed. After cooling, the reaction mixture was diluted with CH₂Cl₂ (20 mL) and concentrated under reduced pressure to dryness at a temperature below 10 °C. The residue was re-dissolved in CH₂Cl₂ (10 mL), treated with Amberlyst A21 (0.1 g), and stirred for another 0.5 hr. After filtering, the solid cake was washed with CH₂Cl₂ (5 mL×2), and the combined filtrate and washings were concentrated under reduced pressure to afford the titled product Ex.39 (20 mg, 32.4%) as a yellow solid; ¹H NMR: 400 MHz CD₃OD, δ 7.88 (s, 1H), 7.49 (br d, J=10.6 Hz, 1H), 7.27 (d, J=10.6 Hz, 1H), 2.47 (s, 3H); LC-MS: m/z [M+H]⁺=205.1.

### Example 40: Preparation of 2-hydroxy-3-(tetrahydrofuran-2-yl)cyclohepta-2,4,6-trien-1-one (Ex.40)

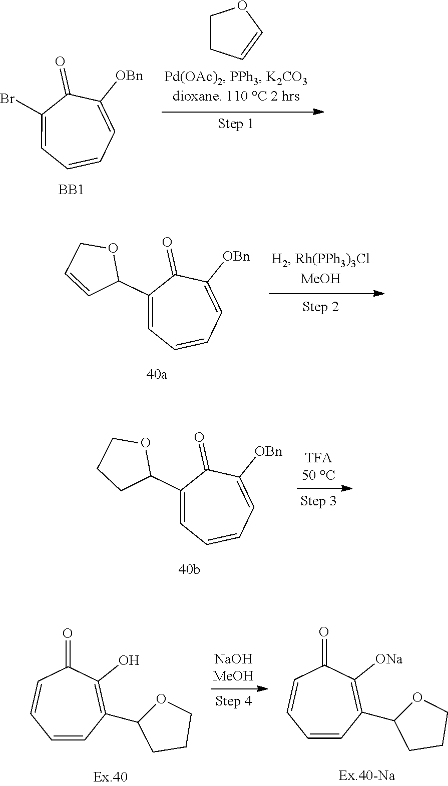

### Step 1:

To a mixture of 2,3-dihydrofuran (3.97 g, 56.6 mmol, 5.00 eq) and building block BB1 (3.30 g, 11.3 mmol, 1.00 eq) in dioxane (50.0 mL) was added K₂CO₃ (3.13 g, 22.6 mmol, 2.00 eq), PPh₃ (594 mg, 2.27 mmol, 0.20 eq) and Pd(OAc)₂ (254 mg, 1.13 mmol, 0.1 eq) in one portion at 20 °C under N₂ atmosphere. The system was degassed and re-charged with nitrogen, the process was repeated three times. The resulting mixture was heated and stirred at 110 °C for 2 hours. LCMS showed the reaction was completed. After cooling, water (50 mL) was added, and the aqueous mixture was extracted with ethyl acetate (50 mL×2). The combined organic extracts were washed with brine (50 mL), dried with anhydrous Na₂SO₄, filtered and concentrated in vacuum. The residue was purified by silica gel column chromatography eluting with petroleum ether:ethyl acetate (20:1 to 8:1) to provide 40a (7.40 g, crude) as a yellow solid.

### Step 2:

To a solution of 40a (2.00 g, 7.13 mmol, 1.00 eq) in MeOH (40 mL) was added Rh(PPh₃)₃C1 (660 mg, 713 umol, 0.10 eq) under N₂. The suspension was degassed under vacuum and purged with H₂ several times. The mixture was stirred under a H₂ (15 psi) atmosphere at 25 °C for 2 hours. LCMS showed the reaction was completed. The reaction mixture was filtered through a pad of Celite and the filter cake was washed with MeOH (20 mL×2). The combined filtrates were concentrated under reduced pressure to dryness to give 40b (3.30 g, crude) as a brown oil.

### Step 3:

Intermediate 40b (3.30 g, 11.6 mmol, 1.00 eq) from the step above was added into TFA (30.0 mL) at 25 °C. The mixture was heated and stirred at 50 °C for 1 hour. LCMS showed the reaction was completed. After cooling, the reaction mixture was concentrated under vacuo to dryness. The residue was purified by prep-HPLC (column: Phenomenex luna C18 250*80 mm*10 um; mobile phase: [water (0.1% TFA)-ACN]; B %: 15%-45%, 20 min) to afford the titled product Ex.40 (1.10 g, 48.9%) as a brown solid.

### Step 4:

To a solution of Ex.40 (0.30 g, 1.56 mmol, 1.00 eq) in MeOH (6.00 mL) was added 5 M NaOH (310 uL, 1.00 eq) at 25 °C. The mixture was stirred at 25 °C for 1 hr, and solvent was removed under reduced pressure. The crude product was triturated with acetone at 25 °C and stirred for another 30 min, then filtered. The solids were washed with acetone (5 mL×2), collected, further dried in vacuum to afford the sodium salt of the titled product Ex.40-Na (252 mg, 74.9%) as a yellow solid; ¹H NMR: 400 MHz D₂O, δ 7.53 (d, J=10.4 Hz, 1H), 7.28-7.21 (m, 1H), 7.03 (d, J=11.2 Hz, 1H), 6.80 (t, J=9.6 Hz, 1H), 5.31 (t, J=6.8 Hz, 1H), 4.18-4.11 (m, 1H), 3.93 (q, J=7.2 Hz, 1H), 2.50-2.39 (m, 1H), 2.05-1.85 (m, 2H), 1.60 (qd, J=12.8 Hz, 1H); LC-MS: m/z [M+H]⁺=193.1.

### Examples 41 and 42: Preparation of (R)-2-hydroxy-3-(tetrahydrofuran-2-yl)cyclohepta-2,4,6-trien-1-one (Ex.41) and (S)-2-hydroxy-3-(tetrahydrofuran-2-yl)cyclohepta-2,4,6-trien-1-one (Ex.42)

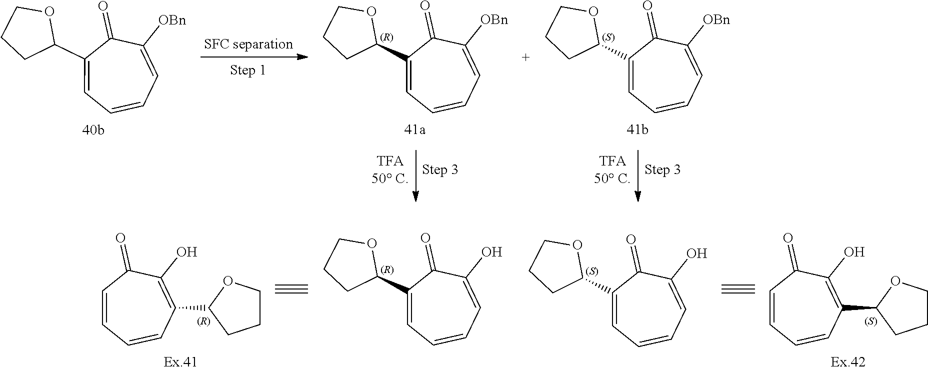

### Step 1:

Intermediate 40b (1 g), prepared from preparative example 40, was separated by SFC (column: DAICEL CHIRALPAK AD (250 mm*30 mm, 10 um); mobile phase: [Neu-IPA]; B %: 40%-40%, min). This separation yielded one enantiomer 41a (0.5 g, 24.8%) and another 41b (0.5 g, 24.8%) both as brown oils. The stereo configuration is arbitrary assigned.

### Step 2:

Isomer 41a from step 1 above (0.3 g, 1.06 mmol, 1 eq) was treated with TFA (6 mL) at 25 °C. The mixture was heated and stirred at 50 °C for 1 hour. LCMS showed the reaction was complete. After cooling, the reaction mixture was concentrated in vacuo to dryness. The residue was purified by prep-HPLC (column: Nano-micro Kromasil C18 100*30 mm 8 um; mobile phase: [water (0.1% TFA)-ACN]; B %: 25%-50%, 10 min) to afford the titled product Ex.41 (145 mg, 70.9%) as a yellow oil; ¹H NMR: 400 MHz CD₃OD, δ 7.88 (d, J=10.0 Hz, 1H), 7.45-7.33 (m, 2H), 7.22-7.14 (m, 1H), 5.26 (t, J=7.2 Hz, 1H), 4.19-4.12 (m, 1H), 3.96 (q, J=7.2 Hz, 1H), 2.66-2.56 (m, 1H), 2.08-1.86 (m, 2H), 1.64-1.53 (m, 1H); LC-MS: m/z [M+H]⁺=193.1

### Step 3:

Isomer 41b from step 1 above (0.3 g, 1.06 mmol, 1 eq) was treated with TFA (6.00 mL) at 25 °C. The mixture was heated and stirred at 50 °C for 1 hour. LCMS showed the reaction was complete. After cooling, the reaction mixture was concentrated in vacuum to dryness. The residue was purified by prep-HPLC (column: Nano-micro Kromasil C18 100*30 mm 8 um; mobile phase: [water (0.1% TFA)-ACN]; B %: 25%-50%, 10 min) to afford the titled pure enantiomer Ex.42 (130 mg, 63.39%) as a yellow oil; ¹H NMR: 400 MHz CD₃OD, δ 7.88 (d, J=10.0 Hz, 1H), 7.45-7.32 (m, 2H), 7.22-7.14 (m, 1H), 5.26 (t, J=7.2 Hz, 1H), 4.18-4.12 (m, 1H), 3.99-3.93 (m, 1H), 2.66-2.56 (m, 1H), 2.08-1.87 (m, 2H), 1.63-1.53 (m, 1H); LC-MS: m/z [M+H]⁺=193.1.

### Example 43: Preparation of 2-hydroxy-3-(tetrahydrofuran-2-yl)cyclohepta-2,4,6-trien-1-one (Ex.43)

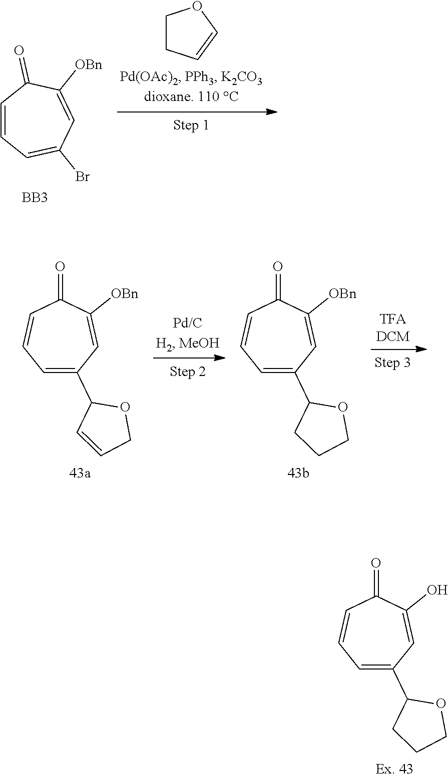

### Step 1:

To a mixture of building block BB3 (0.5 g, 1.72 mmol, 1 eq) and 2,3-dihydrofuran (600 mg, 8.59 mmol, 5 eq) in dioxane (10 mL) was added K₂CO₃ (475 mg, 3.43 mmol, 2 eq), PPh₃ (90 mg, 343.48 umol, 0.2 eq) and Pd(OAc)₂ (38 mg, 171 umol, 0.1 eq) in one portion at 25 °C under a N₂ atmosphere. The system was degassed and recharged with nitrogen, the process was repeated three times. The resulting mixture was heated and stirred at 110 °C for 1 hour. TLC [Petroleum ether/Ethyl acetate=1/1, Rf (material)=0.6, Rf (product)=0.3] showed the starting material was consumed completely and a new product spot appeared. After cooling, the reaction mixture was filtered through a pad of Celite and the filter cake was washed with EtOAc (20 mL×2). The combined filtrates were concentrated to dryness under reduced pressure. The crude product was purified by silica gel column chromatography eluting with Petroleum ether/Ethyl acetate from 10:1 to 1:1 to give 43a (300 mg, 62.3%) as a yellow oil.

### Step 2:

To a solution of 43a (120 mg) in MeOH (8 mL) was added Pd/C (0.20 g, 10/a purity) under N₂. The suspension was degassed under vacuum and purged with H₂ several times. The resulting mixture was stirred under a H₂ (15 psi) atmosphere at 25 °C for 15 min. LC-MS showed the starting materials was consumed completely and the desired product mass peak was detected. After cooling, the reaction mixture was filtered through a pad of Celite and the filtering cake was washed with MeOH (10 mL×2). The combined filtrates were concentrated to dryness under reduced pressure to provide 43b (100 mg, crude) as a yellow oil.

### Step 3:

To a solution of 43b (280 mg) in CH₂Cl₂ (1 mL) was added TFA (5.00 mL). The mixture was heated and stirred at 40 °C for 0.5 hr. TLC (Petroleum ether/Ethyl acetate=1/1, Rf (material)=0.3, Rf (product)=0.00) showed the starting material was consumed completely and a new spot was observed. The reaction mixture was diluted with CH₂Cl₂ (10 mL) and concentrated under reduced pressure to dryness at a temperature below 10 °C. The residue was purified by prep-HPLC (TFA condition, column: Nano-micro Kromasil C18 100*30 mm 8 um; mobile phase: [water (0.1% TFA)-ACN]; B %: 20%-35%, 10 min) to afford the titled product Ex.43 (38 mg, 20%) as a yellow solid; ¹H NMR: 400 MHz CDCl₃, δ 1.73-1.81 (m, 1H) 2.03 (dd, J=7.6, 6.80 Hz, 2H) 2.42 (dd, J=12.8 Hz, 1H) 3.98-4.05 (m, 1H) 4.11-4.18 (m, 1H) 4.85 (t, J=7.6 Hz, 1H) 7.07 (d, J=10.2 Hz, 1H) 7.28 (s, 1H) 7.33-7.42 (m, 2H); LC-MS: m/z [M+H]⁺=193.

### Example 44: Preparation of 2-hydroxy-5-(tetrahydrofuran-2-yl)cyclohepta-2,4,6-trien-1-one (Ex.44)

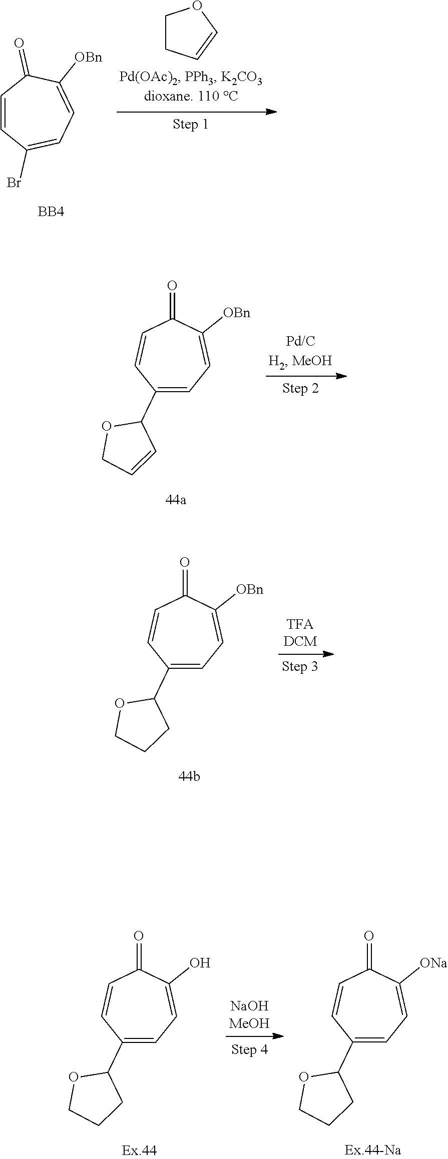

### Step 1:

To a mixture of building block BB4 (500 mg, 1.72 mmol, 1 eq) and 2,3-dihydrofuran (602 mg, 8.59 mmol, 5 eq) in dioxane (10 mL) was added PPh₃ (90 mg, 343 umol, 0.2 eq), K₂CO₃ (474 mg, 3.43 mmol, 2 eq) and Pd(OAc)₂ (39 mg, 171 umol, 0.1 eq) in one portion at 25 °C under N₂ atmosphere. The system was degassed and recharged with nitrogen, the process was repeated two more times. The mixture was heated and stirred at 110 °C for 0.5 hours. LC-MS showed BB4 was consumed completely and a main peak with desired product mass was detected. After cooling, the mixture was filtered through a pad of Celite and the filter cake was washed with CH₂Cl₂ (30 mL×3). The filtrate was concentrated under reduced pressure to dryness. The residue was purified by silica gel column chromatography eluting with Petroleum ether/Ethyl acetate (80/1 to 20/1) to give 44a (250 mg, 51.9%) as a brown solid.

### Step 2:

To a solution of 44a (250 mg, 892 umol, 1 eq) in MeOH (10 mL) was added 10 wt % Pd/C (250 mg) under N₂. The suspension was degassed under vacuum and purged with H₂ several times. The mixture was stirred under H₂ (15 psi) at 25 °C for 30 mins. The reaction progress was monitored by LC-MS which showed 44a was consumed completely and one main peak with desired mass was detected. The reaction mixture was filtered through a pad of Celite and the filter cake was washed with MeOH (5 mL×3). The combined filtrates were concentrated under reduced pressure to dryness to give compound 44b (200 mg, 79.4%) as a blackish solid.

### Step 3:

A solution of 44b (200 mg) in TFA (6 mL) was heated to and stirred for 2 hours at 50 °C under N₂. Reaction progress was monitored by LC-MS which showed a complete consumption of starting material and the detection of desired product mass. After cooling, the reaction mixture was diluted with CH₂Cl₂ (20 mL) and concentrated under reduced pressure to dryness at a temperature below 10 °C. The residue was purified by prep-HPLC (TFA condition) to give the titled product Ex.44 (22.5 mg, 16.5%) as a white solid; ¹H NMR: 400 MHz CD₃OD, δ 1.71 (dq, J=12.4 Hz, 1H) 2.00-2.09 (m, 2H) 2.34-2.42 (m, 1H) 3.92-3.98 (m, 1H) 4.09-4.15 (m, 1H) 4.79 (t, J=7.6 Hz, 1H) 7.34 (d, J=11.6 Hz, 2H) 7.51 (d, J=11.6 Hz, 2H); LC-MS: m/z [M+H]⁺=193.1.

### Step 4:

To a solution of Ex.44 (34 mg, 177 umol, 1 eq) in MeOH (2 mL) was added NaOH (5 M, 35 μL, 1 eq) in one portion at 25 °C. The mixture was stirred at 25 °C for 30 min. Solvent was removed under reduced pressure. The crude product was triturated with acetone (5 mL) at 25 °C and stirred for another 30 min. After filtering, the solids were washed with acetone (5 mL×2) and collected, further dried in vacuum to give the sodium salt of the titled product Ex.44-Na (32 mg, 84.5%) as a yellow solid; ¹H NMR: 400 MHz CD₃OD, δ 1.72 (dq, J=12.28, 8.48 Hz, 1H) 1.96-2.11 (m, 2H) 2.22-2.31 (m, 1H) 3.89 (td, J=7.88, 5.70 Hz, 1H) 4.04-4.11 (m, 1H) 4.66 (dd, J=8.34, 6.58 Hz, 1H) 6.96 (d, J=11.84 Hz, 2H) 7.17 (d, J=11.84 Hz, 2H); LC-MS: m/z [M+H]⁺=193.1.

### Example 45: Preparation of 5-(bicyclo[3.1.0]hexan-3-yl)-2-hydroxycyclohepta-2,4,6-trien-1-one (Ex.45)

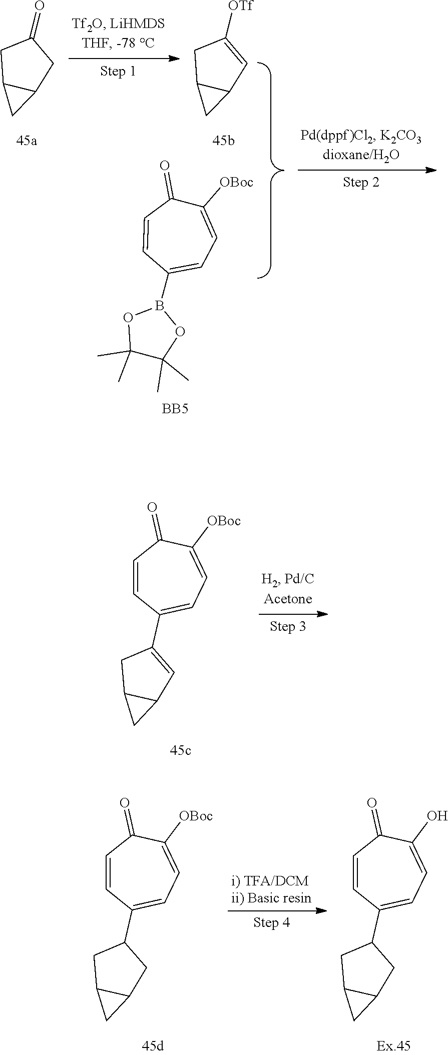

### Step 1:

To a solution of ketone 45a (200 mg, 2.08 mmol, 1 eq) in dry THF (5 mL) was added LiHMDS (1 M, 2.90 mL, 1.4 eq) dropwise at −78 °C under N₂. The mixture was stirred at −78 °C for 30 min, then a solution of Tf₂O (966 mg, 2.70 mmol, 1.3 eq) in dry THF (5 mL) was added dropwise under N₂. The resulting mixture was warmed to and stirred at 25 °C for another 2 h. Reaction progress was monitored by TLC. After cooling to 0 °C, H₂O (10 mL) was added slowly and carefully, and the aqueous mixture was extracted with petroleum ether (20 mL×3). The combined organic extracts were washed with brine (10 mL), dried over Na₂SO₄, filtered and concentrated under reduced pressure to vinyl triflate intermediate 45b (640 mg, crude) as a yellow oil.

### Step 2:

To a mixture of building block BB5 (724 mg, 2.08 mmol, 1 eq) and 45b (474 mg, 2.08 mmol, 1 eq) in dioxane (10 mL) and H₂O (1 mL) was added K₂CO₃ (574 mg, 4.16 mmol, 2 eq) and Pd(dppf)Cl₂·CH₂Cl₂ complex (169 mg, 208 umol, 0.1 eq) in one portion at 25 °C under N₂. The mixture was degassed and recharged with nitrogen, the process was repeated two more times. The resulting mixture was heated and stirred at 118 °C for 30 min. Reaction progress was monitored by TLC (Petroleum ether:Ethyl acetate=3:1) which indicated the starting material was consumed completely and one major new spot was formed. After cooling, water (20 mL) was added and the aqueous mixture was extracted with EtOAc (30 mL×3). The combined organic extracts were washed with brine (20 mL), dried over Na₂SO₄, filtered and concentrated under reduced pressure to give a residue, which was purified by silica gel column chromatography eluting with Petroleum ether:Ethyl acetate (from 100:1 to 5:1) to give 45c (266 mg, 42.58%) as a blackish oil.

### Step 3:

To a solution of 45c (136 mg, 452 umol, 1 eq) in acetone (3 mL) was added 10% Pd/C (20 mg) under N₂. The suspension was degassed under vacuum and purged with H₂ three times. The mixture was stirred under H₂ (15 psi) at 25 °C for 0.5 h. Reaction progress was monitored by TLC (Petroleum ether:Ethyl acetate=3:1) which showed 45c was consumed completely and a new spot was formed. The mixture was filtered through a pad of Celite and the filter cake was washed with acetone (5 mL×2). The combined filtrates were concentrated to dryness under reduced pressure. The residue was purified by prep-TLC (SiO₂, Petroleum ether:Ethyl acetate=3:1) to provide 45d (60 mg, 43.8%) as a yellow oil.

### Step 4:

To a solution of 45d (120 mg, 396 umol, 1 eq) in dichloromethane (3 mL) was added TFA (1 mL) in one portion at 0 °C. The mixture was warmed and stirred at 25 °C for 1 hr. TLC (Petroleum ether:Ethyl acetate=3:1) indicated 45d was consumed completely and one new spot was formed. The reaction mixture was diluted with CH₂Cl₂ (10 mL) and concentrated under reduced pressure to dryness at a temperature below 10 °C. The residue was re-dissolved in CH₂Cl₂ (5 mL), treated with Amberlyst A21 (0.1 g), and stirred at 25 °C for another 0.5 hr. After filtering, the filtration cake was washed with CH₂Cl₂ (5 mL×2). The combined filtrate and washings were concentrated under reduced pressure to afford the titled product Ex.45 (64 mg, 79.7%) as a yellow gum; ¹H NMR: 400 MHz DMSO-d6, δ 0.24-0.44 (m, 1H) 0.78 (td, J=8.4 Hz, 1H) 1.33-1.42 (m, 2H) 1.47 (dd, J=13.6 Hz, 1H) 1.71-2.05 (m, 1H) 2.02 (dd, J=12 Hz, 1H) 2.29-2.38 (m, 2H) 2.64-2.78 (m, 0.4H) 3.53 (quin, J=8.8 Hz, 0.6H) 7.10-7.17 (m, 2H) 7.21-7.35 (m, 2H); LC-MS: m/z [M+H]⁺=203.2.

### Example 46: Preparation of 4-(bicyclo[3.1.0]hexan-3-yl)-2-hydroxycyclohepta-2,4,6-trien-1-one (Ex.46)

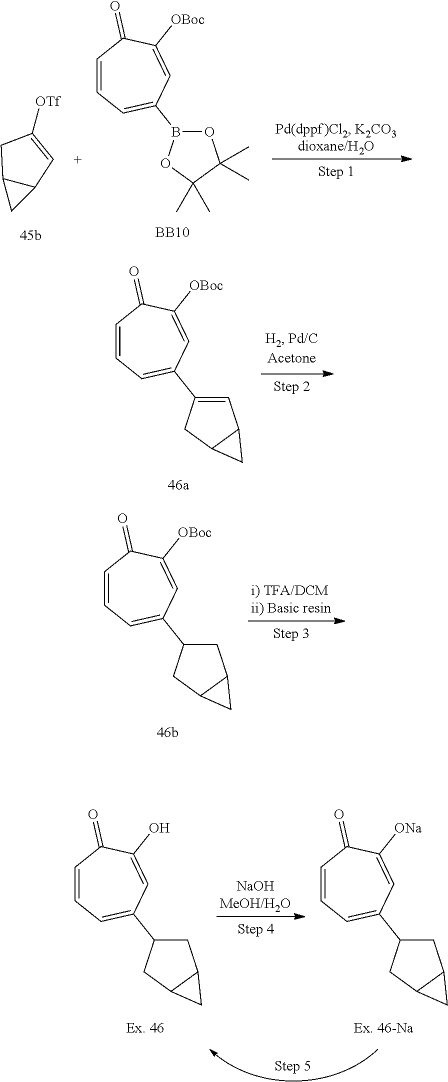

### Step 1:

To a mixture of building block BB10 (427 mg, 1.23 mmol, 1.00 eq) and vinyl triflate 45b (280 mg, 1.23 mmol, 1.00 eq) in dioxane (10 mL) and H₂O (2 mL) was added K₂CO₃ (339 mg, 2.45 mmol, 2.00 eq) and Pd(dppf)Cl₂·CH₂Cl₂ (100 mg, 122 umol, 0.10 eq) in one portion at 25 °C under N₂ atmosphere. The system was degassed and recharged with nitrogen, repeated the process two more times. The resulting mixture was heated and stirred at 118 °C for 30 min. Reaction progress was monitored by LC-MS which showed 45b was consumed completely and one main peak with desired mass was detected. After cooling, the mixture was filtered through a pad of Celite and the filter cake was washed with CH₂Cl₂ (30 mL×3). The filtrate was concentrated under reduced pressure to dryness. The residue was purified by silica gel column chromatography eluting with Petroleum ether/Ethyl acetate (100/1 to 85/15) to give 46a (0.16 g, 43.4%) as a yellow solid.

### Step 2:

To a solution of 46a (0.30 g, 998 umol, 1.00 eq) in petroleum ether (1 mL) and acetone (4 mL) was added 10% Pd/C (0.20 g) under N₂. The suspension was degassed under vacuum and purged with H₂ several times. The mixture was stirred under H₂ (15 psi) at 25 °C for 0.5 hours. TLC (Petroleum ether:Ethyl acetate=5:1) indicated the starting material was consumed completely and one new spot was formed. The reaction mixture was filtered through a pad of Celite and the filter cake was washed with acetone (5 mL×2). The combined filtrates were concentrated to dryness under reduced pressure. The residue was purified by prep-HPLC (neutral condition) to give 46b (68.0 mg, 22.5%) as a white gum.

### Step 3:

To a solution of 46b (67.0 mg) in DCM (5 mL) was added TFA (0.50 mL) in one portion at 0 °C. The mixture was warmed and stirred at 25 °C for 1 hr. TLC (Petroleum ether:Ethyl acetate=3:1) indicated the starting material was consumed completely and one new spot was formed. The reaction mixture was diluted with CH₂Cl₂ (10 mL) and concentrated under reduced pressure to dryness at a temperature below 10 °C. The residue was re-dissolved in CH₂Cl₂ (5 mL), treated with Amberlyst A21 (0.1 g) and stirred at 25 °C for another 0.5 hr. After filtering, the solid cake was washed with CH₂Cl₂ (5 mL×2), and the combined filtrates were concentrated under reduced pressure to provide Ex.46 (38.0 mg, 84.7%) as a yellow gum, trace impurity mixed in.

### Step 4:

To a mixture of Ex.46 (38.0 mg, 187 umol, 1.00 eq) in MeOH (1 mL) was added NaOH (5 M, 37.5 uL, 1.00 eq) in one portion at 25 °C under N₂. The mixture was stirred at 25 °C for 30 min. Solvent was removed under reduced pressure. The crude product was triturated with acetone at 25 °C and stirred for another 30 min. After filtration, the solids were washed with acetone (5 mL×2), collected, and dried in vacuum to provide Ex.46-Na (40.0 mg, 94.9%) as a yellow solid.

### Step 5:

To a solution of Ex.46-Na (27.0 mg, 120 umol, 1.00 eq) in H₂O (2 mL) was added HCl (1 M, 120 uL, 1.00 eq) in one portion at 25 °C under N₂. The mixture was stirred at 25 °C for 30 min. The reaction mixture was extracted with dichloromethane (10 mL×3). The combined organic extracts were washed with brine (10 mL), dried over anhydrous Na₂SO₄, filtered and concentrated under reduced pressure to provide pure titled product Ex.46 (12.0 mg, 49.2%) as a yellow oil; ¹H NMR: 400 MHz DMSO-d6, δ 7.26-7.35 (m, 1H), 7.03-7.17 (m, 2H), 6.88-7.02 (m, 1H), 3.50-3.65 (m, 1H), 2.71-2.83 (m, 1H), 2.38 (td, J=9.2 Hz, 1H), 2.05 (dd, J=12.4, Hz, 1H), 1.77-1.84 (m, 1H), 1.75-1.86 (m, 1H), 1.52 (dd, J=13.6 Hz, 1H), 1.20-1.43 (m, 2H), 0.73-0.89 (m, 1H), 0.28-0.43 (m, 1H); LC-MS: m/z [M+H]⁺=203.2

### Example 47: Preparation of 3-(bicyclo[3.1.0]hexan-3-yl)-2-hydroxycyclohepta-2,4,6-trien-1-one (Ex.47)

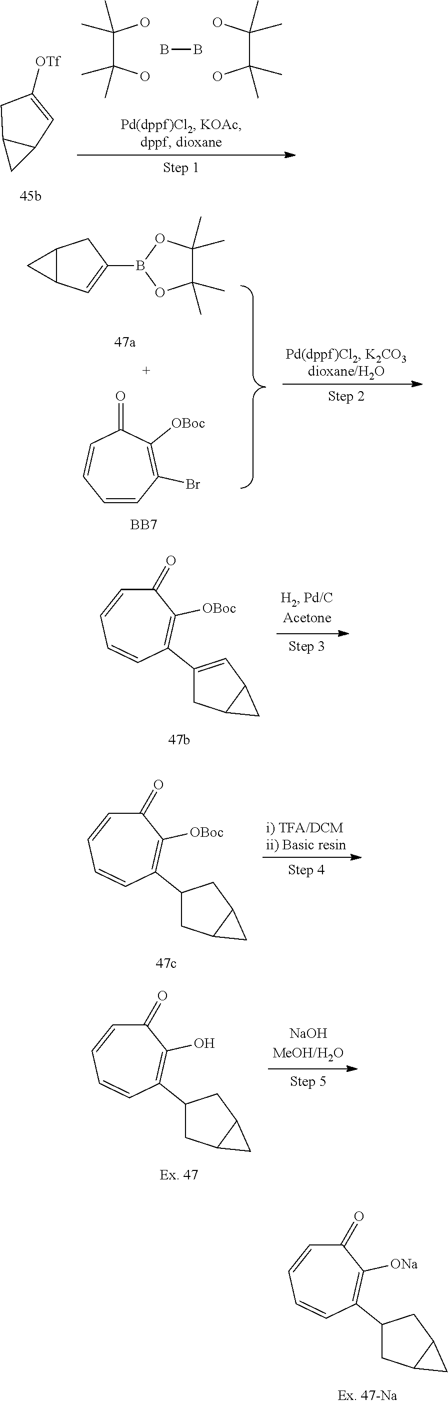

### Step 1:

To a mixture of vinyl triflate 45b (0.25 g, 1.10 mmol, 1.00 eq) and bis(pinacolato)diboron (556 mg, 2.19 mmol, 2.00 eq) in dioxane (5 mL) was added KOAc (430 mg, 4.38 mmol, 4.00 eq) and 1,1′-Bis(diphenylphosphino)ferrocene (dppf, 60.7 mg, 109 umol, 0.10 eq) and the complex Pd(dppf)Cl₂·CH₂Cl₂ (89.5 mg, 109 umol, 0.10 eq) in one portion at 25 °C under N₂ atmosphere. The system was degassed and recharged with nitrogen, repeated two more times. The resulting mixture was heated and stirred at 100 °C for 30 min. TLC (Petroleum ether:Ethyl acetate=10:1) indicated 45b was consumed completely and one new spot was formed. After cooling, the mixture was filtered through a pad of Celite and the filter cake was washed with CH₂Cl₂ (30 mL×3). The combined filtrates were concentrated under reduced pressure to dryness. The residue was purified by silica gel column chromatography eluting with Petroleum ether/Ethyl acetate (100/1 to 10/1) to give 47a1 (85 mg, 37.65%) as a colorless oil.

### Step 2:

To a mixture of 47a (110 mg, 533 umol, 1.00 eq) and BB7 (242 mg, 803 umol, 1.51 eq) in dioxane (5 mL) and H₂O (1 mL) was added K₂CO₃ (147 mg, 1.07 mmol, 2.00 eq) and Pd(dppf)Cl₂·CH₂Cl₂ (43.6 mg, 53.3 umol, 0.10 eq) in one portion at 25 °C under N₂ atmosphere. The system was degassed and recharged with nitrogen, repeated two more times. The resulting mixture was heated and stirred at 118 °C for 30 min. TLC (Petroleum ether:Ethyl acetate=3:1) indicated starting material BB7 was consumed completely and one new spot was formed. After cooling, the mixture was filtered through a pad of Celite and the filter cake was washed with CH₂Cl₂ (30 mL×3). The filtrates were concentrated under reduced pressure to dryness to give 47b (100 mg, 62.4%) as a white solid.

### Step 3:

To a solution of 47b (100 mg, 332 umol, 1.00 eq) in acetone (5 mL) was added 10% Pd/C (40.0 mg) under N₂. The suspension was degassed under vacuum and purged with H₂ several times. The mixture was stirred under H₂ (15 psi) at 25 °C for 0.5 hours. LC-MS showed 47b was consumed completely and one main peak with desired mass was detected. The reaction mixture was filtered through a pad of Celite and the filter cake was washed with acetone (5 mL×2). The combined filtrates were concentrated to dryness under reduced pressure. The residue was purified by prep-HPLC (TFA condition) to give 47c (17.0 mg, 16.9%) as a yellow oil.

### Step 4:

To a solution of 47c (17.0 mg, 56.2 umol, 1.00 eq) in dichloromethane (5 mL) was added TFA (1 mL) in one portion at 0 °C. The mixture was warmed and stirred at 25 °C for 30 min. TLC (Petroleum ether:Ethyl acetate=3:1) indicated 47c was consumed completely and one new spot was formed. The reaction mixture was diluted with CH₂Cl₂ (10 mL) and concentrated under reduced pressure to dryness at a temperature below 10 °C. The residue was re-dissolved in CH₂Cl₂ (5 mL), treated with Amberlyst A21 (0.1 g), and stirred at 25 °C for another 0.5 hr. After filtering, the filter cake was washed with CH₂Cl₂ (5 mL×2), and the combined filtrates and washings were concentrated under reduced pressure to give the titled product Ex.47 (11.0 mg, 96.7%) as a yellow oil.

### Step 5:

To a solution of Ex.47 (11.0 mg, 54.3 umol, 1.00 eq) in MeOH (3 mL) was added aq. NaOH solution (5 M, 11 μL, 1.00 eq) in one portion at 25 °C under N₂. The mixture was stirred at 25 °C for 30 min. The reaction mixture was concentrated under reduced pressure to remove MeOH. The crude product was triturated with acetone (5 mL) at 25 °C and stirred for another 30 min. After filtering, the solids were washed with acetone (5 mL×2), collected, and dried in vacuum to provide sodium salt of the titled product Ex.47-Na (12.0 mg, 98.4%) as a yellow solid; ¹H NMR: 400 MHz CD₃OD, δ 7.18-7.30 (m, 1H), 6.93-7.02 (m, 1H), 6.84-6.92 (m, 1H), 6.51 (dt, J=14.8 Hz, 1H), 4.23 (quin, J=8.4 Hz, 0.5H), 3.60-3.75 (m, 0.5H), 2.39 (br t, J=9.2 Hz, 1H), 2.03 (dd, J=12.0 Hz, 1H), 1.74 (br t, J=12.0 Hz, 1H), 1.51 (br dd, J=13.6 Hz, 1H), 1.27-1.37 (m, 2H), 0.66-0.95 (m, 1H), 0.28-0.50 (m, 1H); LC-MS: m/z [M+H]⁺=203.2.

### Example 48: Preparation of 4-(3,3-dimethylcyclopentyl)-2-hydroxycyclohepta-2,4,6-trien-1-one (Ex.48)

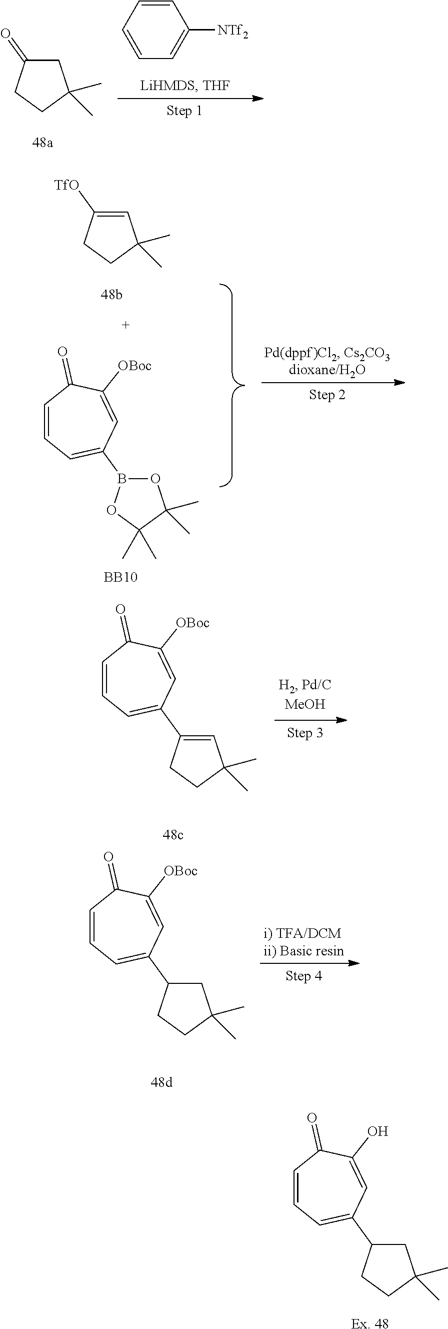

### Step 1:

To a solution 48a (400 mg, 3.57 mmol, 1.00 eq) in anhydrous THF (5 mL) was added LiHMDS (1 M, 5.00 mL, 1.40 eq) slowly at −70 °C under N₂. After addition, the mixture was stirred at −70 °C for 30 min, and a solution of N,N-bis(trifluoromethylsulphonyl)aniline (1.66 g, 4.64 mmol, 1.30 eq) in anhydrous THF (5 mL) was added dropwise. The resulting mixture was stirred at −70 °C for 1 h, and then slowly warmed to 25 °C. Reaction was continued at room temperature for another 2 hrs. TLC (petroleum ether, Rf=0.5) indicated 48a was consumed completely and a major new product spot with less polarity was formed. After cooling to 0 °C, water (10 mL) was added carefully and the aqueous mixture was extracted with EtOAc (20 mL×3). The combined organic extracts were washed with water (10 mL), brine (10 mL), dried over Na₂SO₄, filtered and concentrated under reduced pressure to give the crude intermediate 48b (800 mg, crude) as a brown oil, which was used in the next step without further purification.

### Step 2:

To a solution of BB10 (684 mg, 1.97 mmol, 0.60 eq) in 1,4-dioxane (20 mL) and water (3 mL) was added vinyl triflate intermediate 48b (800 mg, 3.28 mmol, 1.00 eq), Cs₂CO₃ (3.20 g, 9.83 mmol, 3.00 eq) and Pd(dppf)Cl₂ (239 mg, 327 umol, 0.10 eq) under a N₂ atmosphere. The system was degassed and recharged with nitrogen, repeated the process two more times. The resulting mixture was heated and stirred at 110 °C for 2 hours under N₂. TLC (EtOAc:petroleum ether=1:3, Rf=0.3) indicated the complete consumption of starting material BB10 and the formation of a new product spot. After cooling, water (20 mL) was added and the aqueous mixture was extracted with EtOAc (20 mL×3). The combined organic extracts were washed with brine (20 mL), dried over Na₂SO₄, filtered and concentrated under reduced pressure to a residue, which was purified by silica gel column chromatography eluting with Petroleum ether/Ethyl acetate (100/1 to 3/1, Rf=0.3) to afford 48c (0.30 g, 28.9%) as a yellow oil.

### Step 3:

To a solution of 48c (150 mg, 474 umol, 1.00 eq) in EtOAc (5 mL) was added 10% Pd/C (50.0 mg) under N₂. The suspension was degassed under vacuum and purged with H₂ three times. The mixture was stirred under H₂ (15 psi) for 30 min. TLC (Ethyl Acetate:Petroleum Ether=1:3, Rf=0.5) indicated the complete consumption of 48c and formation of a major new product spot. The reaction mixture was filtered through a pad of Celite and the filter cake was washed with EtOAc (10 mL×2). The combined filtrates were concentrated to dryness under reduced pressure to give a crude product. Two batches of crude products were combined and purified by silica gel column chromatography eluting with Petroleum ether:Ethyl acetate (100/1 to 3/1, Rf=0.5) to provide 48d (125 mg, 41.4%) as a colorless oil.

### Step 4:

To a solution of 48d (50.0 mg, 157 umol, 1.00 eq) in dichloromethane (1 mL) was added TFA (0.2 mL) in one portion at 0 °C. The mixture was warmed and stirred at 25 °C for 0.5 hr. TLC (Ethyl Acetate:Petroleum Ether=1:1, Rf=0.2) indicated no starting material 48d remaining and the formation of a major new product spot more polar than 48d. The reaction mixture was diluted with CH₂Cl₂ (10 mL) and concentrated under reduced pressure to dryness at a temperature below 10 °C. The mixture was re-dissolved in CH₂Cl₂ (10 mL), treated with Amberlyst A21 (0.1 g), and stirred for another 0.5 hr. After filtering, the solid cake was washed with CH₂Cl₂ (5 mL×2), and the combined filtrate and washings were concentrated under reduced pressure to afford the titled product Ex.48 (25 mg, 72.9%) as a brownish solid; ¹H NMR: 400 MHz DMSO-d6, δ 7.32 (s, 1H) 7.28-7.33 (m, 1H) 7.19-7.24 (m, 1H) 6.99 (d, J=10.2 Hz, 1H) 3.15-3.27 (m, 1H) 2.10-2.22 (m, 1H) 1.89 (dd, J=12.8 Hz, 1H) 1.75-1.84 (m, 1H) 1.64-1.72 (m, 1H) 1.55-1.62 (m, 1H) 1.47-1.55 (m, 1H) 1.13-1.15 (s, 3H) 1.0 (s, 3H); LC-MS: m/z [M+H]⁺=219.

### Example 49: Preparation of 5-(3,3-dimethylcyclopentyl)-2-hydroxycyclohepta-2,4,6-trien-1-one (Ex.49)

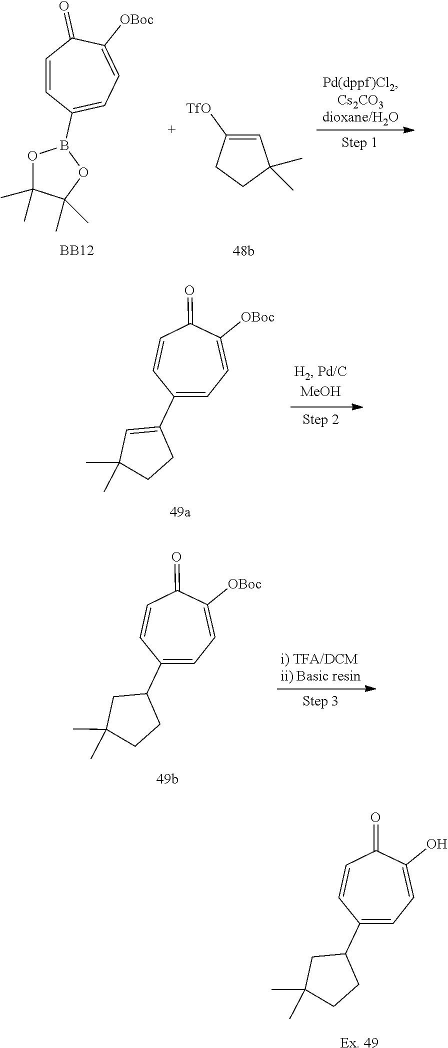

### Step 1:

To a solution of building block BB12 (680 mg, 1.95 mmol, 1.00 eq) in 1,4-dioxane (20 mL) and water (3 mL) was added vinyl triflate 48b (800 mg, 3.28 mmol, 1.00 eq) available from preparative example 48, Cs₂CO₃ (3.21 g, 9.84 mmol, 3.00 eq) and Pd(dppf)Cl₂ (240 mg, 328 umol, 0.10 eq) under N₂ atmosphere. The system was degassed and recharged with nitrogen, the process was repeated two more times. The resulting mixture was heated and stirred at 110 °C for 2 hr under N₂. TLC (EtOAc:petroleum ether=1:3, Rf=0.3) indicated starting material BB12 was consumed completely and a new product spot was formed. After cooling, water (20 mL) was added and the aqueous mixture was extracted with EtOAc (30 mL×3). The combined organic extracts were washed with brine (20 mL), dried over Na₂SO₄, filtered and concentrated under reduced pressure to a residue, which was purified by silica gel column chromatography eluting with Petroleum ether/Ethyl acetate (100/1 to 3:1, Rf=0.3) to give 49a (0.30 g, 28.9%) as a yellow oil.

### Step 2:

To a solution of 49a (250 mg, 790 umol, 1.00 eq) in EtOAc (5 mL) was added 10% Pd/C (100 mg) under N₂. The suspension was degassed under vacuum and purged with H₂ three times. The mixture was stirred under H₂ (15 psi) at 25 °C for 30 min. LCMS showed no starting material 49a remaining and the formation of a new product. The mixture was filtered through a pad of Celite and the filtration cake was washed with EtOAc (10 mL×2). The combined filtrates were concentrated under reduced pressure to dryness to give a crude product, which was purified by silica gel column chromatography eluting with Petroleum ether:Ethyl acetate (100/1 to 3:1, Rf=0.5) to give 49b (100 mg, 39.7%) as colorless oil.

### Step 3:

To a solution of 49b (50.0 mg, 157 umol, 1.00 eq) in dichloromethane (2 mL) was added TFA (0.5 mL) in one portion at 0 °C. The mixture was warmed and stirred at 25 °C for 0.5 hr. TLC (Ethyl Acetate:Petroleum Ether=1:1, Rf=0.2) indicated no starting material 49b remaining and the formation of a new product. The reaction mixture was diluted with CH₂Cl₂ (10 mL) and concentrated under reduced pressure to dryness at a temperature below 10 °C. The residue was re-dissolved in CH₂Cl₂ (10 mL), treated with Amberlyst A21 (0.2 g), and stirred for another 0.5 hr. After filtering, the solid cake was washed with CH₂Cl₂ (5 mL×2) and the combined filtrates were concentrated under reduced pressure to afford the titled product Ex.49 (35.1 mg, 51.1%) as a brown solid; ¹H NMR: 400 MHz DMSO-d6, δ 7.34 (d, J=11.6 Hz, 2H) 7.16 (d, J=11.6 Hz, 2H) 3.15-3.19 (m, 1H) 2.00-2.10 (m, 1H) 1.78 (m, 1H) 1 ppm. 58-1.71 (m, 2H) 1.44-1.54 (m, 1H) 1.39 (t, J=11.6 Hz, 1H) 1.10 (s, 3H) 1.04 (s, 3H); LC-MS: m/z [M+H]⁺=219.

### Example 50: Preparation of 2-hydroxy-4-(spiro[2.5]octan-6-yl)cyclohepta-2,4,6-trien-1-one (Ex.50)

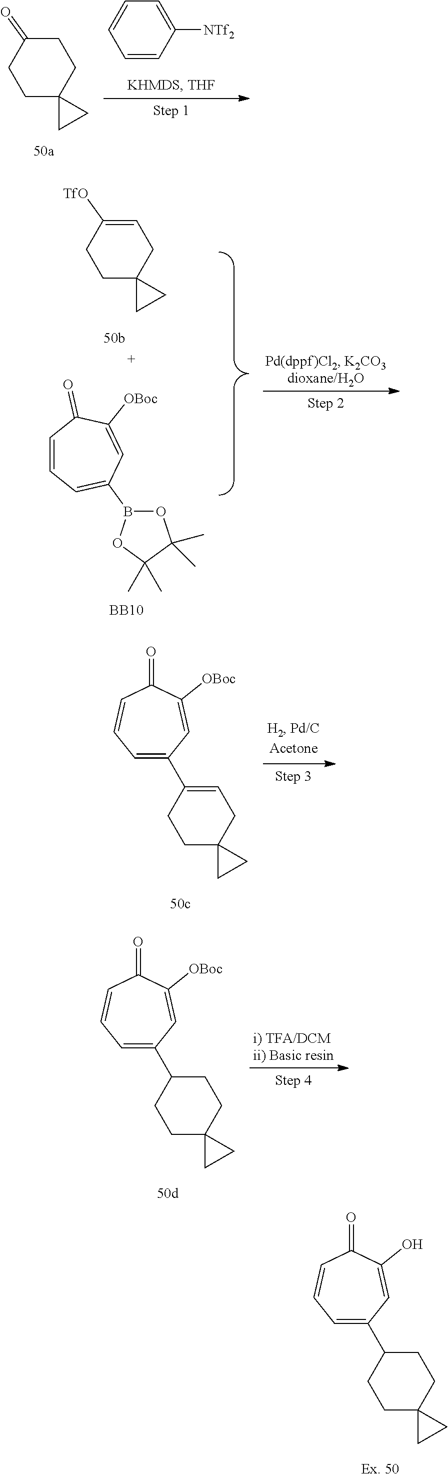

### Step 1:

To a solution of ketone 50a (0.4 g, 3.22 mmol, 1 eq) in anhydrous THF (5 mL) was added KHMDS (1 M, 4.51 mL, 1.40 eq) slowly at −78 °C under N₂. After addition, the mixture was stirred at −78 °C for 30 min, and a solution of N,N-bis(trifluoromethylsulphonyl)aniline (1.50 g, 4.19 mmol, 1.30 eq)) in anhydrous THF (5 mL) was added dropwise to the mixture at −70 °C under N₂. The resulting mixture was stirred at −78 °C for 1 h, and then slowly warmed to 25 °C. Reaction continued at room temperature for another 1 hour. TLC (Petroleum ether, Rf (material)=0, Rf (product)=0.38) showed the completion of reaction at the end of the period. After cooling to 0 °C, H₂O (10 mL) was added, the aqueous mixture was extracted with petroleum ether (30 mL×3). The combined organic extracts were washed with water (10 mL), brine (10 mL), dried over Na₂SO₄, filtered and concentrated under reduced pressure to give the crude vinyl triflate 50b (1.25 g, crude) as a colorless oil.

### Step 2:

To a mixture of building block BB10 (1.12 g, 3.22 mmol, 1.00 eq) and vinyl triflate 50b (0.83 g, 3.22 mmol, 1.00 eq) in dioxane (15 mL) and H₂O (1.5 mL) was added K₂CO₃ (890 mg, 6.44 mmol, 2.00 eq) and Pd(dppf)Cl₂·CH₂Cl₂ (263 mg, 322 umol, 0.10 eq) in one portion at 25 °C under a N₂ atmosphere. The system was degassed and recharged with nitrogen, the process was repeated two more times. The resulting mixture was heated and stirred at 118 °C for 30 mins. LCMS showed completion of the reaction. After cooling, water (10 mL) was added and the aqueous mixture was extracted with EtOAc (20 mL×3). The combined organic extracts were washed with brine (10 mL), dried over Na₂SO₄, filtered and concentrated under reduced pressure to a residue. The residue was purified by silica gel column chromatography eluting with Petroleum ether/Ethyl acetate (100:1 to 80:20) to provide 50c (632 mg, 59.7%) as a yellow oil.

### Step 3:

To a solution of 50c (150 mg, 456 umol, 1.00 eq) in acetone (5 mL) was added 10% Pd/C (150 mg) under N₂. The suspension was degassed under vacuum and purged with H₂ several times. The mixture was stirred under H₂ (15 psi) at 40 °C for 0.5 hour. LC-MS showed the starting material 50c was consumed completely. The reaction mixture was filtered through a pad of Celite and the filtering cake was washed with acetone (10 mL×2). The combined filtrates were concentrated to dryness under reduced pressure to give a crude product, which was purified by silica gel column chromatography eluting with Petroleum ether/Ethyl acetate (100:1 to 80:20) to provide 50d (60 mg, 36.4%) as a yellow oil.

### Step 4:

To a solution of 50d (110 mg) in dichloromethane (2 mL) was added TFA (0.2 mL) in one portion at 0 °C. The mixture was warmed and stirred at 25 °C for 0.5 hr. TLC (Petroleum ether/Ethyl acetate=3:1, Rf (material)=0.36, Rf (product)=0.07) showed the reaction was completed. The reaction mixture was diluted with CH₂Cl₂ (10 mL) and concentrated under reduced pressure to dryness at a temperature below 10 °C. The residue was re-dissolved in CH₂Cl₂ (10 mL), treated with Amberlyst A21 (0.1 g), and stirred for another 0.5 hr. After filtering, the solid cake was washed with CH₂Cl₂ (5 mL×2), and the combined filtrates were concentrated under reduced pressure to afford the titled product Ex.50 (60 mg, 73.0%) as a yellow solid; ¹H NMR: 400 MHz DMSO-d6, δ 7.33 (d, J=10.4 Hz, 1H), 7.17 (s, 1H), 7.10 (d, J=10.8 Hz, 1H), 7.00 (d, J=10.4 Hz, 1H), 2.55 (br t, J=3.2 Hz, 1H), 1.82-1.91 (m, 2H), 1.75 (br d, J=11.6 Hz, 2H), 1.52-1.63 (m, 2H), 0.94 (br d, J=13.2 Hz, 2H), 0.29-0.35 (m, 2H), 0.22-0.28 (m, 2H); LC-MS: m/z [M+H]⁺=231.2.

### Example 51: Preparation of 5-cyclobutyl-2-hydroxycyclohepta-2,4,6-trien-1-one (Ex.51)

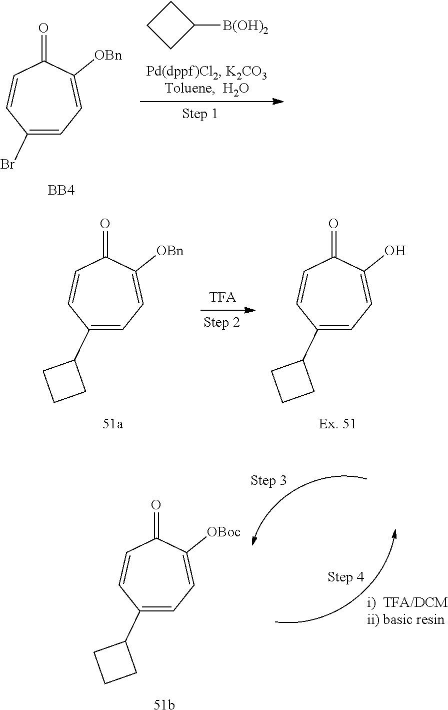

### Step 1:

To a solution of building block BB4 (0.30 g, 1.03 mmol, 1.00 eq) in toluene (24 mL) and H₂O (6 mL) added was added cyclobutyl boronic acid (1.03 g, 10.30 mmol, 10.0 eq) and K₂CO₃ (284 mg, 2.06 mmol, 2.00 eq) in one portion at 20 °C. The mixture was degassed and recharged with N₂, the process was repeated two more times, then Pd(dppf)Cl₂·CH₂Cl₂ complex (168 mg, 206 umol, 0.20 eq) was added under N₂. The system was degassed and recharged with nitrogen again. The resulting mixture was heated and stirred at 100 °C for 1 hr. TLC (Petroleum ether/Ethyl acetate=2/1, Rf (product)=0.43, Rf (material)=0.57) showed the reaction was completed. After cooling, the mixture was filtered through a pad of Celite and the filtering cake was washed with CH₂Cl₂ (30 mL×3). The combined filtrate and washings were concentrated under reduced pressure to dryness. The residue was purified by silica gel column chromatography eluting with Petroleum ether/Ethyl acetate (20/1 to 2/1) to give 51a (0.3 g, 54.6%) as a yellow solid.

### Step 2:

Intermediate 51a (300 mg) was dissolved in TFA (15 mL) and the mixture was stirred at 50 °C for 1 h. LCMS showed the starting material was consumed and the desired product mass was observed. The reaction mixture was diluted with CH₂Cl₂ (50 mL) and concentrated under reduced pressure at a temperature below 10 °C to dryness to give the crude product Ex.51 (200 mg, crude) as a yellow oil, which was used in the next step directly.

### Step 3:

To a solution of crude Ex.51 (150 mg, 851 umol, 1.00 eq) in dioxane (1.00 mL) was added TEA (689 mg, 6.81 mmol, 8.00 eq) and Boc₂O (1.11 g, 5.11 mmol, 6.00 eq) in one portion at 20 °C. The mixture was heated and stirred at 110 °C for 0.5 hr. LCMS showed the reaction was completed. After cooling, the mixture was concentrated under reduced pressure to dryness. The residue was purified by prep-HPLC (column: Welch Xtimate C18 150*25 mm*5 um; mobile phase: [water (10 mM NH4HCO3)-ACN]; B %: 40%-70%, 10.5 min) to give 51b (66 mg, 236 umol, 27.8%, 99% purity) as a yellow oil.

### Step 4:

To a solution of 51b (66.0 mg, 238 umol, 1.00 eq) in dichloromethane (1.00 mL) was added TFA (0.10 mL) in one portion at 0 °C. The mixture was warmed and stirred at 25 °C for 0.5 hr. TLC (Petroleum ether/Ethyl acetate=2/1, Rf (material)=0.45, Rf (product)=0.19) showed no starting material remaining. The reaction mixture was diluted with CH₂Cl₂ (10 mL) and concentrated under reduced pressure at a temperature below 10 °C to dryness. The mixture was re-dissolved in CH₂Cl₂ (10 mL), treated with Amberlyst A21 (0.1 g), and stirred for another 0.5 hr. After filtering, the solid cake was washed with CH₂Cl₂ (5 mL×2), and the combined filtrates were concentrated under reduced pressure to afford the pure product of the titled compound Ex.51 (37.0 mg, 87.0%) as a yellow solid; ¹H NMR: 400 MHz DMSO-d6, δ 7.26-7.28 (m, 2H), 7.16-7.19 (m, 2H), 2.26-2.29 (m, 2H), 2.02-2.07 (m, 4H), 1.77-1.95 (m, 1H); LC-MS: m/z [M+H]⁺=177.

### Example 52: Preparation of 2-hydroxy-5-((1S,2R)-2-hydroxycyclopentyl) cyclohepta-2,4,6-trien-1-one (Ex.52)

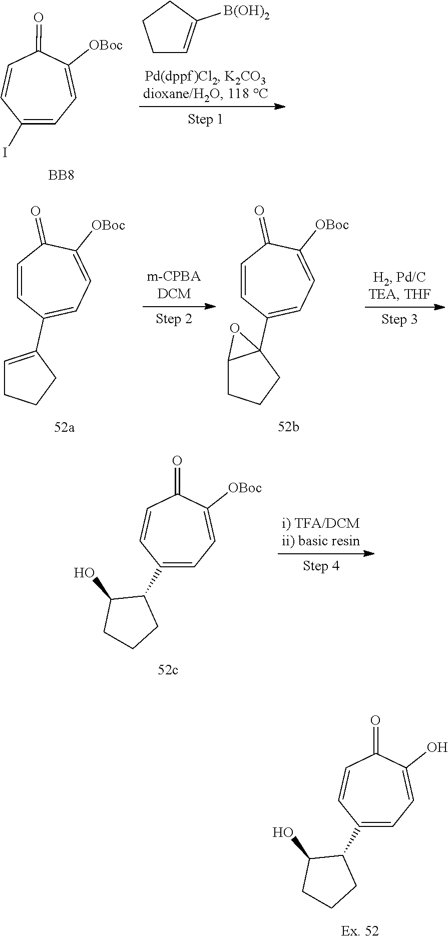

### Step 1:

To a mixture of building block BB4 (500 mg, 1.44 mmol, 1 eq), cyclopenten-1-ylboronic acid (321 mg, 2.87 mmol, 2 eq), and K₂CO₃ (397 mg, 2.87 mmol, 2 eq) in dioxane (10 mL) and H₂O (2 mL) was added Pd(dppf)Cl₂·CH₂Cl₂ complex (50 mg, 61.2 umol, 0.04 eq) under a N₂ atmosphere. The system was degassed and recharged with nitrogen, repeated the process two more times. The resulting mixture was heated and stirred at 118 °C for 0.5 h. TLC (Petroleum ether:Ethyl acetate=5:1) showed the starting material was consumed completely and a new spot was observed. After cooling, the reaction mixture was poured into H₂O (20 mL). The aqueous mixture was extracted with ethyl acetate (25 mL×3); the combined organic extracts were washed with brine (20 mL), dried over anhydrous Na₂SO₄, concentrated in vacuo to a residue. The residue was purified by silica gel column chromatography eluting with Petroleum ether:Ethyl acetate (100:1 to 80:20) to give 52a (317 mg, 1.10 mmol, 76.5%) as a yellow solid.

### Step 2:

To a solution of 52a (150 mg, 520 umol, 1 eq) in dichloromethane (3 mL) was added m-Chloroperbenzoic acid (m-CPBA, 134 mg, 624 umol, 1.2 eq) in portions at 0 °C under N₂. The mixture was stirred at 20 °C for 4 hours. TLC (Petroleum ether:Ethyl acetate=3:1) indicated the starting material 52a was consumed completely and a new spot was formed. The reaction mixture was washed with a 5% Na₂S₂O₃ aqueous solution, 10% NaHCO₃ aqueous solution, and brine successively. The organic layer was dried over Na₂SO₄ and concentrated under reduced pressure to a residue, which was purified by silica gel column chromatography eluting with Petroleum ether:Ethyl acetate (100:1 to 80:20) to afford 52b (130 mg, 427 umol, 82.1%) as a colorless solid.

### Step 3:

To a solution of 52b (50 mg, 164 umol, 1 eq) in THF (3 mL) was added Et₃N (332 mg, 3.28 mmol, 20 eq) and 10% Pd/C (0.03 g) under N₂ atmosphere. The suspension was degassed under vacuum and purged with H₂ several times. The mixture was stirred under H₂ (15 psi) at 20 °C for 15 min. LCMS showed the starting material 52b was consumed completely and desired product mass was observed. The mixture was filtered through a pad of Celite and the filter cake was washed with THF (5 mL×2). The combined filtrates were concentrated under reduced pressure to dryness. The residue was purified by prep-HPLC ((column: Nano-micro Kromasil C18 100×30 mm 5 um; mobile phase: [water (0.1% TFA)-ACN]; B %: 20%-30%, 10 min) to afford 52c (4 mg, 7.95%) as a yellow oil.

### Step 4:

To a mixture of 52c (25 mg) in dichloromethane (2 mL) was added TFA (0.5 mL) in one portion at 0 °C. The mixture was warmed and stirred at 25 °C for 1 hr. LCMS showed the starting material 52c was consumed completely and the desired product mass was observed. The reaction mixture was diluted with CH₂Cl₂ (10 mL) and concentrated under reduced pressure at a temperature below 10 °C to dryness. The residue was re-dissolved in CH₂Cl₂ (10 mL), treated with Amberlyst A21 (0.1 g), and stirred at 25 °C for another 0.5 hr. After filtering, the solid cake was washed with CH₂Cl₂ (5 mL×2), and the combined filtrates were concentrated under reduced pressure to afford the titled product Ex.52 (14 mg, 83.1%) as a yellow solid, the structure is further confirmed by NOESY as trans-configuration; ¹H NMR: 400 MHz CD₃OD, δ 7.43-7.52 (m, 2H), 7.35 (s, 2H), 4.11 (q, J=7.2 Hz, 1H), 2.86 (dt, J=10 Hz, 1H), 2.03-2.21 (m, 2H), 1.78-1.92 (m, 2H), 1.62-1.75 (m, 2H); LC-MS: m/z [M+H]⁺=207.1.

### Example 53: Preparation of 2-hydroxy-7-methyl-3-(tetrahydrofuran-2-yl)cyclohepta-2,4,6-trien-1-one (Ex.53)

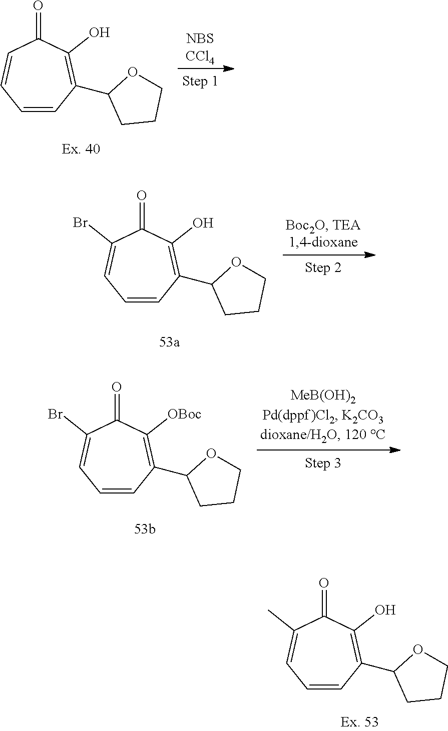

### Step 1:

To a solution of Ex.40 (2.00 g, 10.4 mmol, 1.00 eq) in CCl₄ (30 mL) was added NBS (1.85 g, 10.4 mmol, 1.00 eq) in portions at 25 °C. The mixture was heated and stirred at 80 °C for 4 hours. The reaction was monitored by LCMS. After cooling to room temperature, water (50 mL) was added and the mixture extracted with CH₂Cl₂ (50 mL×3). The combined organic layers were washed with brine (50 mL), dried over Na₂SO₄, filtered and concentrated under reduced pressure to give 53a (3.50 g, crude) as brown oil that was used in next step without further purification.

### Step 2:

To a solution of 53a (3.50 g, 12.9 mmol, 1.00 eq) in dioxane (70 mL) were added Et₃N (5.23 g, 51.6 mmol, 7.19 mL, 4.00 eq) and Boc₂O (13.2 g, 60.6 mmol, 4.70 eq) at 20 °C. The mixture was heated to and stirred at 100 °C for 2 hours. The reaction was monitored by LCMS. After cooling to room temperature, the mixture was concentrated under reduced pressure to dryness. The residue was purified by silica gel column chromatography (petroleum ether:EtOAc=9:1 to 5:1) to afford 53b (0.90 g, 15.0% yield, 80.0% purity) as a yellow oil.

### Step 3:

To a mixture of 53b (0.40 g, 1.08 mmol, 1.00 eq) and methylboronic acid (387 mg, 6.47 mmol, 6.00 eq) in dioxane (10 mL) and H₂O (1 mL) were added Pd(dppf)Cl₂ (78.0 mg, 107 umol, 0.10 eq) and K₂CO₃ (446 mg, 3.23 mmol, 3.00 eq) at 20 °C under N₂ atmosphere. The system was degassed and charged with nitrogen three times. The mixture was heated to and stirred at 120 °C for 2 hours. The reaction was monitored by LCMS. After cooling to room temperature, the mixture was filtered through a pad of Celite, and the filter cake was washed with CH₂Cl₂ (10 mL×3). The filtrate was concentrated under reduced pressure to dryness. The residue was purified by prep-HPLC {column: Welch Xtimate C18 (100*25 mm, 3 um); mobile phase: [water (0.1% TFA)-ACN]; B %: 25%-55%, 12 min} to give Ex. 53 (50 mg, 22.5% yield) as a brown solid; ¹H NMR: 400 MHz CDCl₃, δ 7.77 (d, J=10.4 Hz, 1H), 7.40 (d, J=10.4 Hz, 1H), 7.02 (t, J=10.4 Hz, 1H), 5.33 (t, J=7.2 Hz, 1H), 4.18-4.08 (m 1H), 4.06-3.93 (m, 1H), 2.71-2.59 (m, 1H), 2.48 (s, 3H), 2.08-1.97 (m, 1H), 1.97-1.85 (m, 1H), 1.67-1.54 (m, 1H); LC-MS: m/z [M+H]⁺=207.1.

### Example 54: Preparation of ethyl 4-(4-hydroxy-5-oxocyclohepta-1,3,6-trien-1-yl)piperidine-1-carboxylate (Ex.54)

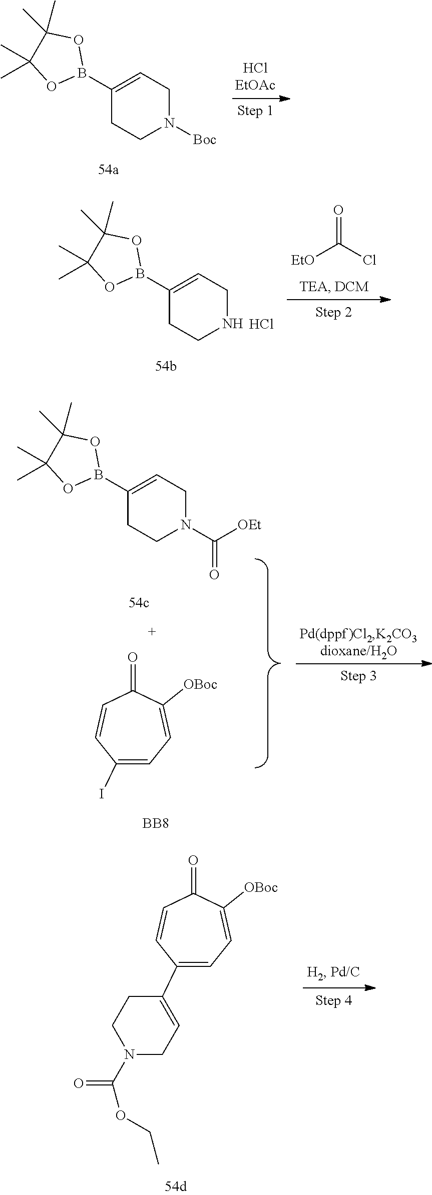

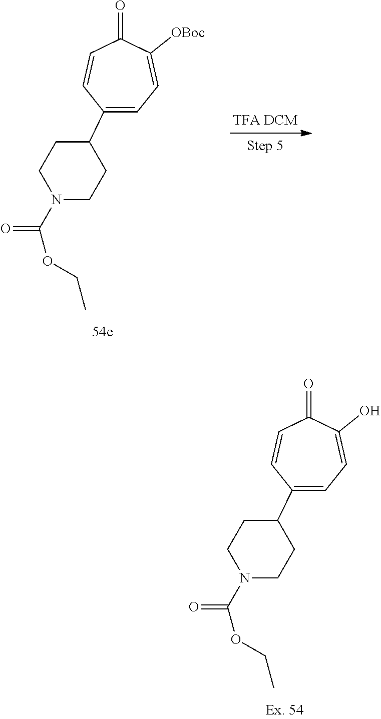

### Step 1:

To a solution of commercially available building block 54a (2 g, 6.47 mmol, 1 eq) in EtOAc (2 mL) was added a 4M solution of HCl gas in EtOAc (4 M, 10 mL) at 0 °C. The mixture was warmed and stirred at 25 °C for 1 hour. TLC (Petroleum ether:Ethyl acetate=3:1) indicated the starting material was consumed completely and a new product spot was formed. The reaction mixture was directly concentrated under reduced pressure to give 54b (1.65 g, crude, HCl salt) as a white solid.

### Step 2:

To a stirred suspension of 54b (3.3 g, 13.4 mmol, 1 eq, HCl) in CH₂Cl₂ (30 mL) was added triethylamine (6.80 g, 67.2 mmol, 5 eq) at 0 °C. After 10 min, ethyl chloroformate (1.75 g, 16.13 mmol, 1.2 eq) in CH₂Cl₂ (5 mL) was added dropwise to the above mixture. The resulting mixture was stirred at 0 °C for another 2 hr. LCMS showed the starting material was consumed completely and one main peak representing the desired product mass was detected. The mixture was poured into water (30 mL) and extracted with CH₂Cl₂ (30 mL×3). The combined organic extracts were washed with a 2N NH₄Cl aqueous solution (10 mL×2), water (20 mL), and brine (20 mL) successively, further dried over anhydrous Na₂SO₄. Removal of solvents under reduced pressure afforded 54c (3.5 g, 92.6%) as a colorless oil.

### Step 3:

To a mixture of 54c (150 mg, 534 umol, 1 eq) and building block BB8 (161 mg, 534 umol, 1 eq) in dioxane (5 mL) and H₂O (1 mL) was added K₂CO₃ (147 mg, 1.07 mmol, 2 eq) and Pd(dppf)Cl₂·CH₂Cl₂ (44 mg, 54 umol, 0.1 eq) in one portion at 15 °C under N₂ atmosphere. The system was degassed and recharged with nitrogen, repeated the process two more times. The mixture was heated and stirred at 118 °C for 1 hr. TLC (Petroleum ether:Ethyl acetate=3:1) indicated the starting material was consumed completely and one new product spot was formed. After cooling, the mixture was filtered through a pad of Celite and the filter cake was washed with CH₂Cl₂ (30 mL×3). The combined filtrates were concentrated under reduced pressure to dryness. The residue was purified by a silica gel column chromatography eluting with Petroleum ether/Ethyl acetate (100/1 to 100/20) to give 54d (97 mg, 48.4%) as a yellow solid.

### Step 4:

To a solution of 54d (97 mg, 258 umol, 1 eq) in acetone (10 mL) was added 10/a Pd/C (0.1 g) under N₂. The suspension was degassed under vacuum and purged with H₂ several times. The mixture was stirred under H₂ (15 psi) at 25 °C for 45 min. TLC (Petroleum ether:Ethyl acetate=1:1) indicated the starting material 54d was consumed completely and one new product spot was formed. The reaction mixture was filtered through a pad of Celite and the filter cake was washed with MeOH (20 mL×2). The combined filtrates were concentrated to dryness under reduced pressure to afford 54e (100 mg, crude) as a yellow oil.

### Step 5:

To a solution of 54e (100 mg, 265 umol, 1 eq) in CH₂Cl₂ (5 mL) was added TFA (0.5 mL) in one portion at 0 °C. The mixture was warmed and stirred at 20 °C for 30 min. TLC (Petroleum ether:Ethyl acetate=1:1) indicated the starting material 54e was consumed completely and one new product spot was formed. The reaction mixture was diluted with CH₂Cl₂ (10 mL) and concentrated under reduced pressure to dryness at a temperature below 10 °C. The residue was purified by prep-HPLC (TFA condition) to afford the titled product Ex.54 (18 mg, 24.5%) as a colorless gum; ¹H NMR: 400 MHz CD₃OD, δ 7.42-7.48 (m, 2H), 7.27-7.36 (m, 2H), 4.28 (br d, J=13.6 Hz, 2H), 4.14 (q, J=7.2 Hz, 2H), 2.92 (br s, 2H), 2.70-2.81 (m, 1H), 1.83 (br d, J=12.8 Hz, 2H), 1.62 (qd, J=12.8 Hz, 2H), 1.27 (t, J=7.2 Hz, 3H); LC-MS: m/z [M+H]⁺=278.1.

### Example 55: Preparation of 5-(1-acetylpiperidin-4-yl)-2-hydroxycyclohepta-2,4,6-trien-1-one (Ex.55)

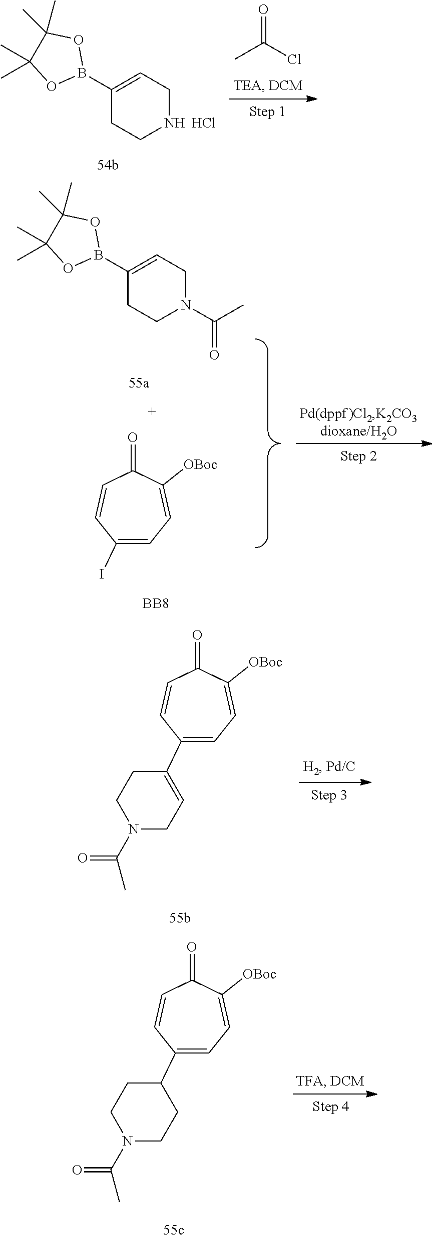

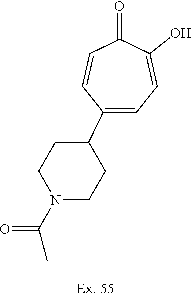 [SMILES: CC(=O)N1CCC(c2ccc(O)c(=O)cc2)CC1]

### Step 1:

To a solution of 54b (1.60 g, 6.52 mmol, 1 eq, HCl salt) in CH₂Cl₂ (20 mL) was added triethylamine (4.62 g, 45.6 mmol, 7 eq) at 0 °C and stirred for 10 min. Acetyl chloride (512 mg, 6.52 mmol, 1 eq) in CH₂Cl₂ (5 mL) was added dropwise to the above mixture at 0 °C. The mixture was stirred at 0 °C for 2 hr. LC-MS showed the starting material 54b was consumed completely and one main peak with desired product mass was detected. The mixture was poured into water (30 mL) and extracted with CH₂Cl₂ (10 mL×3). The combined organic extracts were washed with a 2N NH₄Cl solution (10 mL×2), water (20 mL), brine (20 mL), dried over anhydrous Na₂SO₄, filtered and concentrated under reduced pressure to give 55a (1.4 g, 5.57 mmol, 85.5%) as a colorless oil.

### Step 2:

To a mixture of building block BB8 (0.3 g, 996 umol, 1 eq) and 55a (300 mg, 1.20 mmol, 1.2 eq) in dioxane (15 mL) and H₂O (3 mL) was added K₂CO₃ (275 mg, 1.99 mmol, 2 eq) and Pd(dppf)Cl₂·CH₂Cl₂ (81 mg, 100 umol, 0.1 eq) in one portion at 20 °C under a N₂ atmosphere. The system was degassed and recharged with nitrogen, repeated the process two more times. The resulting mixture was heated and stirred at 118 °C for 30 min. TLC (EtOAc:MeOH=10:1) indicated the starting material was consumed completely and one new spot was formed. After cooling, the reaction mixture was diluted with H₂O (30 mL). The aqueous mixture was extracted with EtOAc (20 mL×3). The combined organic extracts were washed with water (10 mL), brine (10 mL), dried over anhydrous Na₂SO₄, filtered and concentrated under reduced pressure to dryness. The residue was purified by silica gel column chromatography eluting with Ethyl acetate/MeOH (100/1 to 10/1) to provide 55b (0.27 g, 78.5%) as a yellow oil.

### Step 3:

To a solution of 55b (0.27 g, 782 umol, 1 eq) in acetone (10 mL) was added 10% Pd/C (0.2 g) under N₂. The suspension was degassed under vacuum and purged with H₂ several times. The mixture was stirred under H₂ (15 psi) at 25 °C for 45 min. LCMS showed the starting material 55b was consumed completely and one main peak with desired mass was detected. The reaction mixture was filtered through a pad of Celite and the filter cake was washed with MeOH (20 mL×2). The combined filtrates were concentrated to dryness under reduced pressure to provide 55c (0.3 g, crude) as a yellow oil.

### Step 4:

To a solution of 55c (0.3 g, 864 umol, 1 eq) in dichloromethane (5 mL) was added TFA (1 mL) in one portion at 0 °C. The mixture was warmed and stirred at 20 °C for 30 min. TLC (EtOAc:MeOH=10:1) indicated the starting material 55c was consumed completely and one new product spot was formed. The reaction mixture was diluted with CH₂Cl₂ (10 mL) and concentrated under reduced pressure to dryness at a temperature below 10 °C. The residue was purified by prep-HPLC (TFA condition) to afford the titled product Ex.55 (35 mg, 16.4%) as a colorless gum; ¹H NMR: 400 MHz CD₃OD, δ 7.41-7.51 (m, 2H), 7.28-7.36 (m, 2H), 4.58-4.73 (m, 1H), 4.05 (br d, J=11.6 Hz, 1H), 3.15-3.28 (m, 1H), 2.77-2.92 (m, 1H), 2.63-2.76 (m, 1H), 2.14 (s, 3H), 1.83-1.94 (m, 2H), 1.52-1.76 (m, 2H); LC-MS: m/z [M+H]⁺=248.1.

### Example 56: Preparation of 3-cyclobutoxy-2-hydroxycyclohepta-2,4,6-trien-1-one (Ex.56)

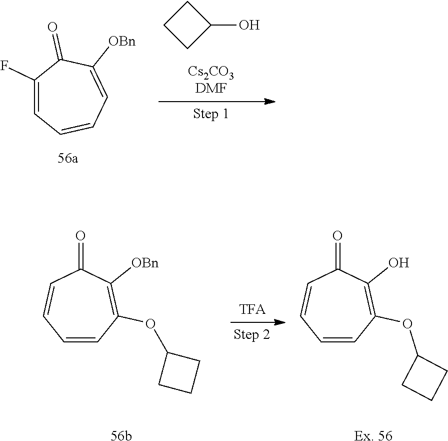 [SMILES: O=c1c(F)ccccc1OCc1ccccc1 ; OC1CCC1 ; O=c1ccccc(OC2CCC2)c1OCc1ccccc1 ; O=c1ccccc(OC2CCC2)c1O ; "56b" ; "Ex. 56"]

### Step 1:

To a mixture of 56a (0.5 g, 2.17 mmol, 1.00 eq) and cyclobutanol (313 mg, 4.34 mmol, 2.00 eq) in DMF (6 mL) Cs₂CO₃ (707 mg, 2.17 mmol, 1.00 eq) was added in one portion at 25 °C under N₂. The mixture was heated and stirred at 60 °C for 3 hrs. LC-MS showed that the reaction was complete. After cooling, water (10 mL) was added to the reaction mixture and extracted with ethyl acetate (10 mL×3). The combined organic phases were washed with brine (10 mL×2), dried over anhydrous Na₂SO₄, filtered and concentrated in vacuum. The residue was purified by pre-TLC (Petroleum ether/Ethyl acetate=2/3, R_{f}=0.4) to give 56b (0.15 g, 24.5% yield) as a yellow oil.

### Step 2:

56b (0.1 g, 354.19 umol, 1.00 eq) was added into TFA (3 mL) at 25 °C. The mixture was stirred at 50 °C for 2 hours. The completed reaction mixture, as indicated by LC-MS, was concentrated in vacuum. The residue was purified by pre-HPLC (column: Xtimate C18 100*30 mm*3 um; mobile phase: [water (0.1% TFA)-ACN]; B %: 15%-45%, 10 min) to afford the titled product Ex.56 (0.061 g, 317.36 umol, 89.60% yield, 100% purity) as a white solid, ¹HNMR: (CDCl₃ 400 MHz) δ 7.40 (d, J=9.7 Hz, 1H), 7.14-7.05 (m, 1H), 7.05-6.98 (m, 2H), 4.84-4.75 (m, 1H), 4.79 (quin, J=7.2 Hz, 1H), 2.60-2.49 (m, 2H), 2.43-2.31 (m, 2H), 1.94 (q, J=10.4 Hz, 1H), 1.82-1.68 (m, 1H); HPLC: MS (M+H): 193.1.

### Example 57: Preparation of 2-hydroxy-3-(oxetan-3-yloxy)cyclohepta-2,4,6-trien-1-one (Ex.57)

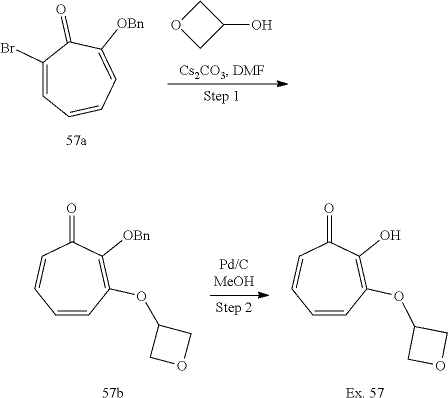 [SMILES: O=c1c(Br)ccccc1OCc1ccccc1 ; OC1COC1 ; O=c1ccccc(OC2COC2)c1OCc1ccccc1 ; O=c1ccccc(OC2COC2)c1O ; "57b" ; "Ex. 57"]

### Step 1:

To a solution of 57a (4 g, 13.7 mmol, 1.00 eq) and oxetan-3-ol (2.04 g, 27.5 mmol, 2.00 eq) in DMF (10 mL) was added Cs₂CO₃ (4.48 g, 13.7 mmol, 1.00 eq). The mixture was heated to 90 °C and stirred for 3 hr. TLC (ethyl acetate, Rt=0.45) showed the starting material was almost consumed completely and new spot was observed. After cooling, water (100 mL) was added to the mixture and extracted with ethyl acetate (100 mL×2). The combined organic phases were washed with brine (100 mL), dried over anhydrous Na₂SO₄, filtered and concentrated in vacuum to dryness. The residue was purified by silica gel chromatography (Petroleum ether/Ethyl acetate=90/10 to 30/70) to give 57b (0.9 g, 20.7% yield, 90% purity) as yellow oil.

### Step 2:

To a solution of 57b (0.5 g, 1.76 mmol, 1.00 eq) in MeOH (3 mL) Pd/C (10%, 0.2 g) was added under N₂. The suspension was degassed under vacuum and purged with H₂ several times. The mixture was stirred under H₂ (15 psi) at 25 °C for 2 hr. LCMS showed the starting material was almost completely consumed and the major desired MS was observed. The mixture was filtered through a pad of Celite and the filter cake was washed with MeOH (10 mL 3×). The filtrate was concentrated under reduced pressure to dryness. The residue was purified by prep-HPLC (column: Nano-micro Kromasil C18 100*30 mm 8 um; mobile phase: [water (0.1% TFA)-ACN]; B %: 5%-30%, 10 min) to afford the titled product Ex.57 (33 mg, 168 umol, 9.57% yield, 99% purity) as a yellow solid. ¹HNMR: 400 MHz; δ ppm 7.36-7.27 (m, 1H), 7.02 (d, J=10.4 Hz, 1H), 6.70-6.60 (m, 2H), 5.36 (quin, J=5.6 Hz, 1H), 5.04 (t, J=6.8 Hz, 2H), 4.70 (dd, J=5.2, 7.6 Hz, 2H); HPLC: MS (M+H): 195.1.

### Example 58: Preparation of 4-cyclobutoxy-2-hydroxycyclohepta-2,4,6-trien-1-one (Ex.58)

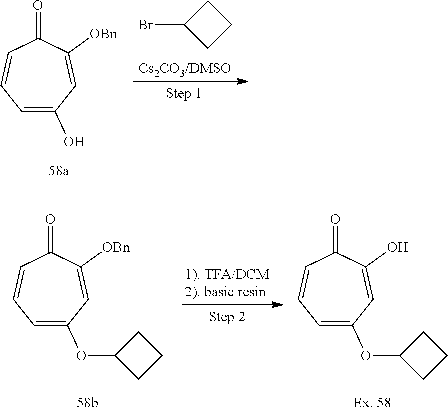 [SMILES: O=c1cccc(O)cc1OCc1ccccc1 ; BrC1CCC1 ; O=c1cccc(OC2CCC2)cc1OCc1ccccc1 ; O=c1cccc(OC2CCC2)cc1O]

### Step 1:

To a solution of 58a (1.70 g, 7.45 mmol, 1.00 eq) in DMF (10 mL) was added bromocyclobutane (2.01 g, 14.9 mmol, 1.41 mL, 2.00 eq), Cs₂CO₃ (4.85 g, 14.9 mmol, 2.00 eq) and tetrabutylammonium iodide (2.75 g, 7.45 mmol, 1.00 eq) in one portion at 25 °C. The mixture was then heated and stirred at 90 °C for 0.5 h. TLC (Ethyl Acetate/Petroleum Ether=1/1, R_{f}=0.3) showed the Reactant 58a was consumed and a new spot, with large polarity, was found. After cooling to room temperature, water (30 mL) was added and then extracted with ethyl acetate (30 mL×3). The combined organic phases were washed with water (20 mL), brine (20 mL), and dried over anhydrous Na₂SO₄. After filtering, the filtrate was concentrated under reduced pressure to dryness. The residue was purified by silica gel chromatography (Petroleum ether/Ethyl acetate=100/1 to 1/1, R_{f}=0.3) to get 58b (210 mg, 743 umol, 9.99% yield) as a yellow brown solid.

### Step 2:

To a solution of 58b (200 mg, 708 umol, 1.00 eq) TFA (3.00 mL) was added in one portion at 20 °C. The mixture was heated and stirred at 75 °C for 1 hour. LCMS showed the Reactant 58b was consumed and the desired MS was detected. The reaction mixture was diluted with CH₂Cl₂ (10 mL) and concentrated under reduced pressure to dryness to give crude product. Then the mixture was re-dissolved in CH₂Cl₂ (5 mL) and Amberlyst A21 (0.1 g) was added and stirred at 25 °C for another 0.5 hr. After filtering, the cake was washed with CH₂Cl₂ (5 mL×2) and the filtrate was concentrated under reduced pressure to afford the titled product Ex.58 (19.0 mg, 98.8 umol, 13.9% yield) as a brown oil. ¹H NMR: (400 MHz, DMSO-d6); δ ppm 7.20 (br dd, J=11.6, 10.8 Hz, 1H) 6.87 (d, J=10.6 Hz, 1H) 6.59-6.64 (m, 2H) 4.77 (m, J=7.14 Hz, 1H) 2.40-2.47 (m, 2H) 2.01-2.12 (m, 2H) 1.76-1.85 (m, 1H) 1.62-1.72 (m, 1H). HPLC: (RT=2.51 min); LCMS: (RT=1.03 min): [M+1]: 193.

### Example 59: Preparation of 2-hydroxy-3-(tetrahydrofuran-3-yl)cyclohepta-2,4,6-trien-1-one (Ex.59)

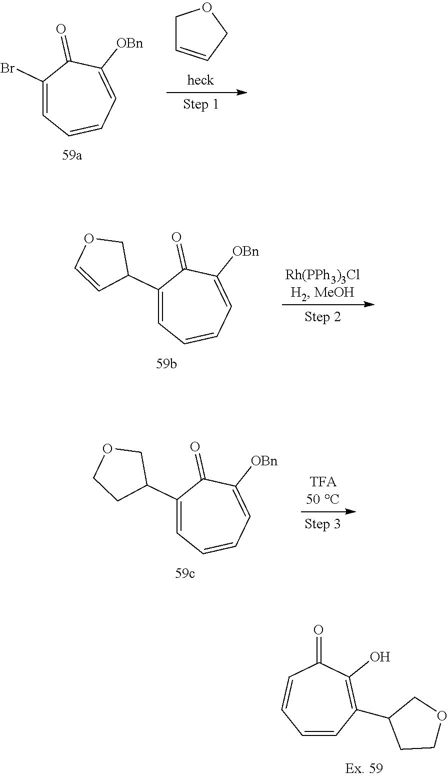

### Step 1:

To a mixture of 2,5-dihydrofuran (1.20 g, 17.2 mmol, 5.00 eq) and 59a (1 g, 3.43 mmol, 1.00 eq) in dioxane (20.0 mL) was added K₂CO₃ (949 mg, 6.87 mmol, 2.00 eq), PPh₃ (180 mg, 687 umol, 0.20 eq) and Pd(OAc)₂ (77.0 mg, 343 umol, 0.10 eq) at 20 °C under N₂ atmosphere. The system was degassed and then charged with nitrogen three times. The mixture was heated and stirred at 110 °C for 2 hrs. TLC (Petroleum ether/Ethyl acetate=3/1, R_{f}=0.42. UV as developer) showed the starting material was consumed and new spot observed. After cooling, the mixture was filtered through a pad of Celite and the filter cake was washed with CH₂Cl₂ (30 mL×3). The filtrate was concentrated under reduced pressure to dryness. The residue was purified by silica gel chromatography (Petroleum ether/Ethyl acetate=100/1 to 3/1) to give 59b (930 mg, 88.4% yield) as a yellow solid.

### Step 2:

To a solution of 59b (0.93 g, 3.32 mmol, 1.00 eq) in MeOH (20 mL) was added Rh(PPh₃)₃Cl (307 mg, 332 umol, 0.10 eq) under N₂. The suspension was degassed under vacuum and purged with H₂ several times. The mixture was stirred under H₂ (15 psi) at 50 °C for 1 hr. TLC (Petroleum ether/Ethyl acetate=1/1, Rt=0.34) showed the reaction was complete. After cooling, the mixture was filtered through a pad of Celite and the filter cake was washed with MeOH (10 mL×3). The filtrate was concentrated under reduced pressure to dryness. The residue was purified by silica gel chromatography (Petroleum ether/Ethyl acetate=10/1 to 2/1) to give 59c (0.82 g, 82.9% yield) as a brown oil.

### Step 3:

59c (300 mg, 1.06 mmol, 1.00 eq) was added into TFA (6.00 mL) at 25 °C. The mixture was heated and stirred at 50 °C for 1 hr. LCMS showed the reaction was complete. The reaction mixture was diluted with DCM (30 mL) and concentrated in vacuo to dryness. The residue was purified by prep-HPLC (column: Nano-micro Kromasil C18 100*30 mm 8 um; mobile phase: [water (0.1% TFA)-ACN]; B %: 1%-25%, 10 min) to afford the titled product Ex.59 (140 mg, 67.9% yield) as an off-white solid. ¹HNMR: 400 MHz; δ 7.36-7.22 (m, 2H), 7.07-6.92 (m, 2H), 4.65 (t, J=9.6 Hz, 1H), 4.41 (dd, J=6.4, 9.6 Hz, 1H), 3.71-3.60 (m, 1H), 3.56-3.47 (m, 2H), 1.90 (dtd, J=4.0, 6.4, 13.2 Hz, 1H), 1.73-1.63 (m, 1H); HPLC: MS: 193.1.

### Example 60: Preparation of 2-hydroxy-4-(oxetan-3-yloxy)cyclohepta-2,4,6-trien-1-one (Ex.60)

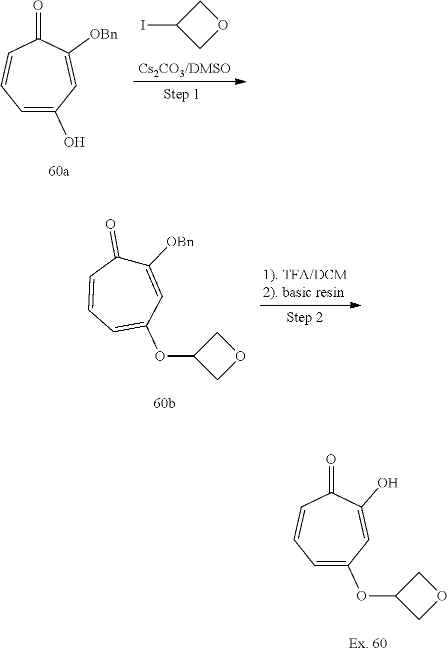 [SMILES: O=c1cccc(O)cc1OCc1ccccc1 ; IC1COC1 ; O=c1cccc(OC2COC2)cc1OCc1ccccc1 ; O=c1cccc(OC2COC2)cc1O]

### Step 1:

To a solution of 60a (800 mg, 3.51 mmol, 1.00 eq) in DMF (10.0 mL) was added 3-iodooxetane (1.93 g, 10.5 mmol, 3.00 eq) and Cs₂CO₃ (1.71 g, 5.26 mmol, 1.50 eq) in one portion at 25 °C. Then the mixture was heated and stirred at 120 °C for 0.5 h. TLC (Ethyl Acetate/Petroleum Ether=2/1, R_{f}=0.4) showed that 60a was consumed and a new spot with large polarity was observed. After cooling to room temperature, water (20 mL) was added and then extracted with ethyl acetate (20 mL×3). The combined organic phases were washed with water (20 mL), brine (20 mL), and dried over anhydrous Na₂SO₄. After filtering, the filtrate was concentrated under reduced pressure to dryness. The residue was purified by silica gel chromatography (Petroleum ether/Ethyl acetate=100/1 to 2/1, R_{f}=0.4) to get 60b (280 mg, 984 umol, 28.1% yield) as a yellow solid.

### Step 2:

To a solution of 60b (150 mg, 527 umol, 1.00 eq) TFA (3.00 mL) was added in one portion at 0 °C. The mixture was heated and stirred at 75 °C for 1 hour. LCMS showed the Reactant 60b was consumed and the desired MS was detected. After cooling, the reaction mixture was concentrated under reduced pressure to remove solvent and then washed two times with CH₂Cl₂ (5 mL) to give crude product. Then the mixture was re-dissolved in CH₂Cl₂ (5.00 mL) and Amberlyst A21 (0.1 g) was added and stirred for another 0.5 hr. After filtering, the cake was washed with CH₂Cl₂ (5 mL×2) and the filtrate was concentrated under reduced pressure to afford the titled product Ex.60 (50 mg, 257 umol, 48.8% yield) as a brown solid. ¹H NMR (400 MHz, CDCl₃-d6); δ ppm 7.15-7.22 (m, 1H) 7.02 (d, J=10.4 Hz, 1H) 6.62 (dd, J=11.6, 2.8 Hz, 1H) 6.49 (d, J=2.8 Hz, 1H) 5.21-5.27 (m, 1H) 5.03 (t, J=6.8 Hz, 2H) 4.76 (dd, J=7.6, 5.2 Hz, 2H). HPLC: (RT=2.13 in); LCMS: (RT=2.12: [M+1]: 193.

### Example 61: Preparation of 2-fluoro-7-hydroxy-4-methylcyclohepta-2,4,6-trien-1-one (Ex.61)

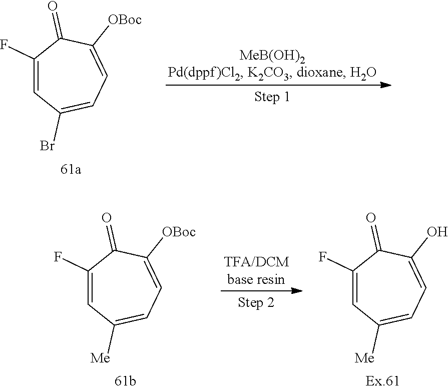

### Step 1:

To a mixture of 61a, methylboronic acid (375 mg, 6.27 mmol, 10.0 eq), K₂CO₃ (173 mg, 1.25 mmol, 2.00 eq) in dioxane (1.5 mL) and water (0.3 mL) was added Pd(dppf)Cl₂·CH₂Cl₂ (102 mg, 125 umol, 0.20 eq) under N₂ atmosphere. The system was degassed and then charged with nitrogen three times. The mixture was heated and stirred at 118 °C for 30 min. A new spot was observed on TLC (PE/EtOAc=100/25, R_{f}=0.35), and 61a was consumed on TLC (Rt=0.55). After cooling, the yellow solution was filtered and the filtrate was concentrated under reduced pressure to dryness. The residue was purified by pre-TLC (PE/EtOAc=5/1, R_{f}=0.35) to give 61b (50.0 mg, 196 umol, 31.3% yield) as a light yellow gum.

### Step 2:

The solution of 61b (31.5 mg, 123 umol, 1.00 eq) and TFA (141 mg, 1.24 mmol, 91.7 uL, 10.0 eq) in DCM (1.00 mL) was stirred at 25 °C for 20 min. The material was consumed and the desired MS was observed on LCMS. The reaction mixture was diluted with CH₂Cl₂ (10 mL) and concentrated under reduced pressure to dryness. Then the mixture was re-dissolved in CH₂Cl₂ (5 mL) and added Amberlyst A21 (0.1 g) and stirred at 25 °C for another 0.5 hr. After filtering, the cake was washed with CH₂Cl₂ (5 mL×2) and the filtrate was concentrated under reduced pressure to afford the titled product Ex.61 (18.0 mg, 116 umol, 94.1% yield) as a white solid. ¹HNMR: 400 MHz) δ 7.65-7.58 (m, 1H), 7.39-7.30 (m, 2H), 2.43 (s, 3H); HPLC: MS: 155.1.

### Example 62: Preparation of (R)-2-hydroxy-4-((tetrahydrofuran-3-yl)oxy)cyclohepta-2,4,6-trien-1-one (Ex.62)

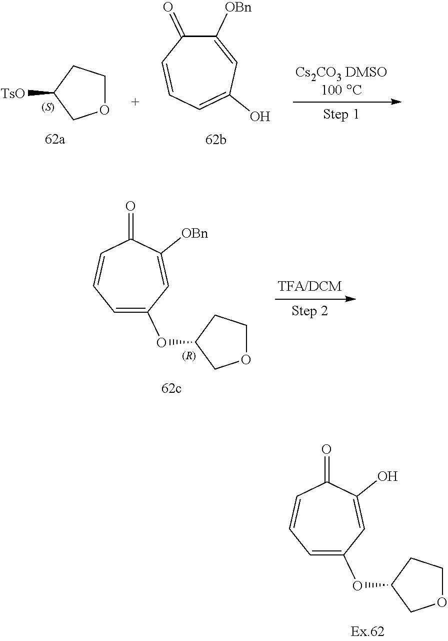

### Step 1:

A mixture of 62b (500 mg, 2.19 mmol, 1.00 eq), 62a (796 mg, 3.29 mmol, 1.50 eq) and Cs₂CO₃ (2.14 g, 6.57 mmol, 3.00 eq) in DMF (5 mL) was stirred at 110 °C for 2 h. LCMS 62c was detected. After cooling, the reaction was quenched by water (40 mL) and extracted with EtOAc (30 mL×3). The combined organic layers were dried over Na₂SO₄, filtered and concentrated under reduced pressure. The residue was purified by prep-HPLC (neutral condition, column: Phenomenex Gemini-NX C18 75*30 mm*3 um; mobile phase: [water(10 mM NH₄HCO₃)-ACN]; B %: 26%-46%, 10.5 min) to provide 62c (500 mg, 1.68 mmol, 76.5% yield) as a brown oil.

### Step 2:

A mixture of 62c (250 mg, 837.99 umol, 1.00 eq) and TFA (4 mL) was heated and stirred at 70 °C for 0.5 h. LCMS showed Ex.62 was detected. The solution was concentrated under reduced pressure to afford the titled product Ex.62 (150 mg, 85.9% yield) as a brown oil.

### Example 63: Preparation of (S)-2-hydroxy-4-((tetrahydrofuran-3-yl)oxy)cyclohepta-2,4,6-trien-1-one (Ex.63)

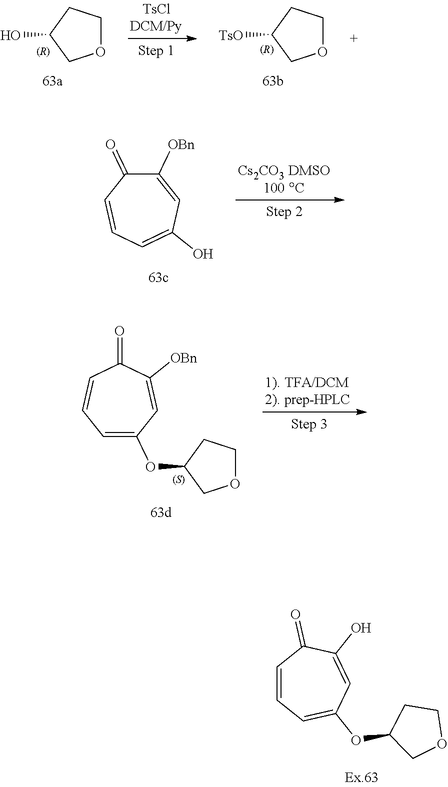

### Step 1:

To a solution of 63a (1.00 g, 11.4 mmol, 1.00 eq) and Py (4.94 g, 62.4 mmol, 5.04 mL, 5.50 eq) in DCM (20 mL) TosCl (3.25 g, 17.0 mmol, 1.50 eq) was added at 0 °C. The mixture was stirred at 25 °C for 16 hrs. TLC (petroleum ether:ethyl acetate=5:1) showed 63b (Rf=0.43) was present. The solution was quenched by water (20 mL) and then extracted with DCM (20 mL×3). The combined organic layers were washed with HCl (1M, 10 mL) and water (10 mL), dried over Na₂SO₄, filtered and concentrated under reduced pressure. The residue was purified by column chromatography (SiO₂, petroleum ether:ethyl acetate=4:1) to provide compound 63b (1.4 g, 5.78 mmol, 50.9% yield) as a colourless oil.

### Step 2:

A mixture of 63c (650 mg, 2.85 mmol, 1 eq), 63b (1.04 g, 4.27 mmol, 1.5 eq) and Cs₂CO₃ (2.78 g, 8.54 mmol, 3 eq) in DMF (10 mL) was stirred at 110 °C for 2 h. LCMS showed that 63d was present. The solution was quenched by water (20 mL) and extracted with EtOAc (20 mL×3). The combined organic layers were washed with brine (10 mL), dried over Na₂SO₄, filtered and concentrated under reduced pressure. The residue was purified by prep-HPLC (neutral condition, column: Phenomenex Gemini-NX C18 75*30 mm*3 um; mobile phase: [water(10 mM NH₄HCO₃)-ACN]; B %: 26%-46%, 10.5 min) to afford the titled product Ex.63 (140 mg, 16.5% yield) as a white solid.

### Example 64: Preparation of 2-mercapto-3-(tetrahydrofuran-2-yl)cyclohepta-2,4,6-trien-1-one (Ex.64)

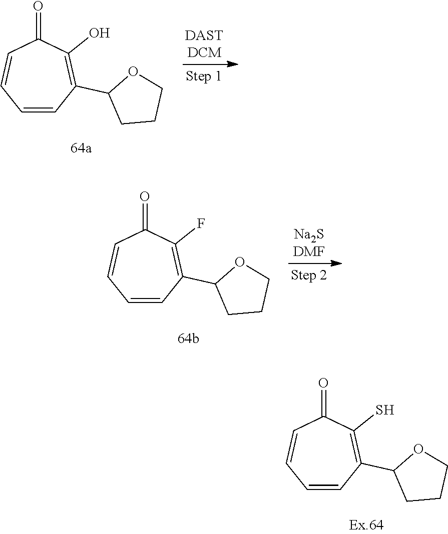 [SMILES: O=c1ccccc(C2CCCO2)c1O ; O=c1ccccc(C2CCCO2)c1F ; O=c1ccccc(C2CCCO2)c1S]

### Step 1:

To a solution of 64a (0.9 g, 4.68 mmol, 1.00 eq) in DCM (10 mL) DAST (1.51 g, 9.36 mmol, 2.00 eq) was added at 0 °C. The mixture was warmed and stirred at 25 °C for 16 h. LCMS showed the desired MS was present. The solution was quenched by sat. NaHCO₃ solution until pH>7 and then extracted with EtOAc (10 mL×3). The combined organic layers were washed with brine (10 mL), dried over anhydrous Na₂SO₄, filtered and concentrated under reduced pressure. The residue was purified by prep-HPLC (TFA condition, column: Phenomenex Luna C18 100*30 mm*5 um; mobile phase: [water(0.1% TFA)-ACN]; B %: 6%-36%, 10 min) to provide 64b (350 mg, 1.80 mmol, 38.5% yield) as a yellow oil.

### Step 2:

A mixture of 64b (100 mg, 515 umol, 1.00 eq) and Na₂S (44 mg, 566 umol, 1.10 eq) in DMF (1.5 mL) was stirred at 20 °C for 2 h. LCMS showed the desired MS (M+1=209.1) was present. The solution was quenched by HCl (1M) until pH<5 and then extracted with EtOAc (3 mL×3). The combined organic layers were washed with (10 mL), dried over anhydrous Na₂SO₄, filtered and concentrated under reduced pressure. The residue was purified by prep-HPLC (TFA condition, column: Nano-micro Kromasil C18 100*30 mm 8 um; mobile phase: [water(0.1% TFA)-ACN]; B %: 40%-70%, 10 min) to afford the titled product Ex.64 (15 mg, 14.0% yield) as a red oil. ¹HNMR: 400 MHz; δ11.34-11.54 (m, 1H), 7.96-8.02 (m, 1H), 7.80 (d, J=2.0 Hz, 2H), 7.65-7.73 (m, 1H), 5.56-5.63 (m, 1H), 4.17-4.24 (m, 1H), 3.89-3.97 (m, 1H), 2.68-2.78 (m, 1H), 1.92-2.04 (m, 1H), 1.76-1.89 (m, 1H), 1.47-1.57 (m, 1H); HPLC: MS: 208.1.

### Example 65: Preparation of 3-(cyclopentyloxy)-2-hydroxycyclohepta-2,4,6-trien-1-one (Ex.65)

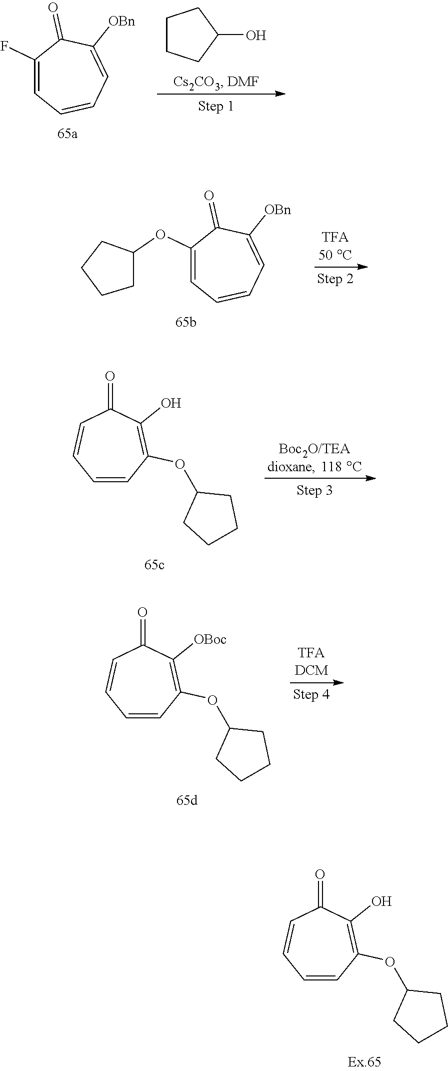

### Step 1:

To a mixture of 65a (500 mg, 2.17 mmol, 1.00 eq) and cyclopentanol (374 mg, 4.34 mmol, 2.00 eq) in DMF (6 mL) Cs₂CO₃ (707 mg, 2.17 mmol, 1.00 eq) was added in one portion at 25 °C under N₂. The mixture was heated to 80 °C and stirred for 3 hours. LC-MS showed 65a was consumed completely and one main peak with desired mass was detected. The reaction mixture was quenched by addition of H₂O (5 mL) at 25 °C, extracted with EtOAc (20 mL×4). The combined organic layers were washed with brine (10 mL), dried over Na₂SO₄, filtered and concentrated under reduced pressure to give a residue. The residue was purified by prep-TLC (SiO₂, EtOAc:PE=1:1) to give 65b (240 mg, 37.3% yield) as a yellow oil.

### Step 2:

A mixture of 65b (240 mg) in TFA (2 mL) was heated to 50 °C and stirred for 5 hours. LC-MS showed 65b was consumed completely and one main peak with desired mass was detected. The reaction mixture was diluted with CH₂Cl₂ (10 mL) and concentrated under reduced pressure to dryness to give 65c (150 mg, 89.8% yield) as a yellow oil.

### Step 3:

To a mixture of 65c (100 mg, 485 umol, 1.00 eq) and tert-butoxycarbonyl tert-butyl carbonate (317 mg, 1.45 mmol, 3.00 eq) in dioxane (2 mL) was added TEA (98 mg, 970 umol, 2 eq) in one portion at 25 °C under N₂. The mixture was heated to 118 °C and stirred for 0.5 hours. TLC (petroleum ether:ethyl acetate=1:1, R_{f}=0.5) showed 65c was consumed completely and desired mass was detected. After cooling, the reaction mixture was concentrated under reduced pressure to give a residue. The residue was purified by column chromatography (SiO₂, Petroleum ether/Ethyl acetate=100/0 to 25/1) to afford 65d (78 mg, 52.5% yield) as yellow oil.

### Step 4:

A mixture of 65d (78 mg) in TFA (0.8 mL) was stirred at 25 °C for 30 mins. LC-MS showed 65d was consumed completely and one main peak with desired mass was detected. The reaction mixture was diluted with CH₂Cl₂ (10 mL) and concentrated under reduced pressure to dryness below 10 °C. Then the mixture was re-dissolved in CH₂Cl₂ (5 mL) and Amberlyst A21 (0.1 g) was added and stirred at 25 °C for another 0.5 hr. After filtering, the cake was washed with CH₂Cl₂ (5 mL×2) and the filtrate was concentrated under reduced pressure to afford the titled product Ex.65 (20 mg, 38.1% yield) as a brown solid. ¹H NMR: 400 MHz; 1H NMR (400 MHz, METHANOL-d4) δ ppm 1.60-1.74 (m, 2H) 1.81-1.97 (m, 4H) 1.99-2.12 (m, 2H) 5.03 (br s, 1H) 7.12-7.25 (m, 2H) 7.39-7.48 (m, 2H); HPLC: MS: 207.2.

### Example 66: Preparation of 2-hydroxy-3-(tetrahydrothiophen-2-yl)cyclohepta-2,4,6-trien-1-one (Ex.66)

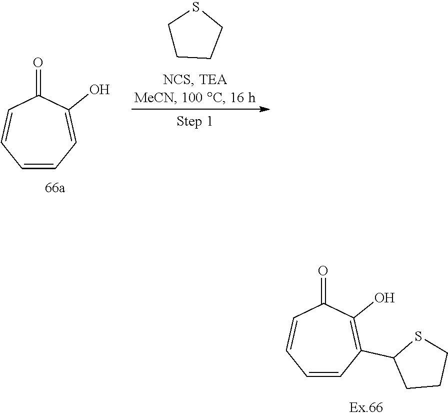

### Step 1:

To a solution of 66a (0.3 g, 2.46 mmol, 1.00 eq) in DCM (7 mL) NCS (328 mg, 2.46 mmol, 1.00 eq) was added in one portion at −60 °C under N₂. The mixture was stirred at −60 °C for 5 min, then tetrahydrothiophene (650 mg, 7.37 mmol, 3.00 eq) was added to the mixture and stirred at −60 °C for 4 hours. TEA (436 mg, 4.31 mmol, 1.75 eq) was added to the mixture and warmed to 20 °C. Then the mixture was concentrated under reduced pressure to remove solvent, TEA (363 mg, 3.59 mmol, 1.46 eq) and MeCN (5 mL) was added and the resulting mixture was heated and stirred at 100 °C for 16 hr. LC-MS showed ~57% of 66a remained. Several new peaks were observed on LC-MS and ~10% of the desired compound was detected. The reaction mixture was quenched by addition H₂O (20 mL) at 20 °C and extracted with Ethyl acetate (10 mL×3). The combined organic layers were dried over Na₂SO₄, filtered and concentrated under reduced pressure to dryness. The reside was purified by prep-HPLC (TFA condition, column: Nano-micro Kromasil C18 100*40 mm 10 um; mobile phase: [water(0.1% TFA)-ACN]; B %: 15%-60%, 8 min) to afford the titled product Ex.66 (11 mg, 53 umol, 2.2% yield) as a yellow solid. ¹H NMR: 400 MHz; δ: 1.79-1.92 (m, 1H) 1.94-2.05 (m, 1H) 2.11 (br d, J=6.2 Hz, 1H) 2.22-2.38 (m, 1H) 2.85-2.97 (m, 1H) 3.04 (dt, J=10.0, 6.8 Hz, 1H) 5.04 (s, 1H) 7.11 (t, J=10.0 Hz, 1H) 7.21-7.28 (m, 1H) 7.31-7.41 (m, 1H) 7.92 (d, J=10.0 Hz, 1H); HPLC: MS (M+H): 209.1.

### Example 67 and 68: Preparation of 4-isopropyl-2-mercaptocyclohepta-2,4,6-trien-1-one (Ex.67) and 6-isopropyl-2-mercaptocyclohepta-2,4,6-trien-1-one (Ex.68)

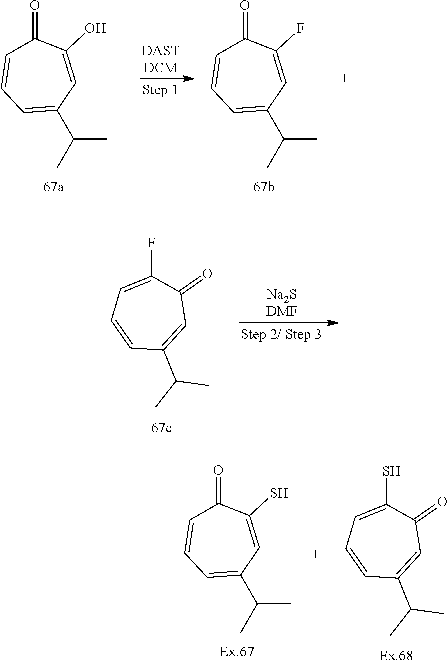

### Step 1:

To a solution of 67a (1 g, 6.09 mmol, 1.00 eq) in DCM (10 mL) DAST (1.28 g, 7.92 mmol, 1.30 eq) was added drop-wise at 0 °C under N₂. The mixture was warmed and stirred at 20 °C for 16 hr. LC-MS showed 67a was consumed completely and one main peak with desired mass was detected. The reaction mixture was quenched by addition of H₂O (10 mL), and then extracted with DCM (10 mL×3). The combined organic layers were washed with brine (10 mL), dried over anhydrous Na₂SO₄, filtered and concentrated under reduced pressure to dryness. The residue was purified by prep-HPLC (TFA condition, column: Phenomenex luna C18 (250*70 mm, 15 um); mobile phase: [water(0.1% TFA)-ACN]; B %: 25%-35%, 20 min) to give 67b (0.24 g, 23.7% yield) as a yellow oil, and 67c (0.24 g, 23.7% yield) as a yellow oil.

### Step 2:

To a solution of 67b (0.17 g, 1.02 mmol, 1.00 eq) in DMF (2 mL) was added Na₂S (160 mg, 2.05 mmol, 2.00 eq) in one portion at 15 °C under N₂. The mixture was stirred at 15 °C for 30 min. LC-MS showed 67b was consumed completely and one main peak with desired mass was detected. The reaction mixture was quenched by addition of H₂O (30 mL) at 15 °C, and then extracted with ethyl acetate (30 mL×3). The combined organic layers were washed with H₂O (10 mL×2), brine (10 mL), dried over anhydrous Na₂SO₄, filtered and concentrated under reduced pressure to dryness. The residue was purified by prep-HPLC (TFA condition, column: Phenomenex Synergi C18 150*25*10 um; mobile phase: [water(0.1% TFA)-ACN]; B %: 40%-75%, 10 min) to afford the titled product Ex.67 (15 mg, 8.13% yield) as a yellow solid. ¹H NMR: 400 MHz; δ: 1.28 (d, J=6.8 Hz, 6H) 2.92 (spt, J=6.8 Hz, 1H) 7.15-7.23 (m, 2H) 7.61 (s, 1H) 8.53-8.63 (m, 1H); HPLC: MS (M+H): 181.1.

### Step 3:

To a solution of 67c (0.17 g, 1.02 mmol, 1.00 eq) in DMF (2 mL) Na₂S (160 mg, 2.05 mmol, 2.00 eq) was added in one portion at 15 °C under N₂. The mixture was stirred at 15 °C for 30 min. LC-MS showed 67c was consumed completely and one main peak with desired mass was detected. The reaction mixture was quenched by addition of H₂O (30 mL) at 15 °C, and then extracted with ethyl acetate (30 mL×3). The combined organic layers were washed with H₂O (10 mL×2), brine (10 mL), dried over anhydrous Na₂SO₄, filtered and concentrated under reduced pressure, purified by prep-HPLC (TFA condition, column: Phenomenex Synergi C18 150*25*10 um; mobile phase: [water(0.1% TFA)-ACN]; B %: 50%-75%, 10 min) to afford the titled product Ex.68 (11 mg, 5.97% yield) as a yellow solid.

¹H NMR: CDCl₃ 400 MHz; δ: 1.32 (d, J=6.8 Hz, 6H) 2.87-3.01 (m, 1H) 7.18 (br d, J=8.4 Hz, 1H) 7.41-7.51 (m, 2H) 8.73 (d, J=1.6 Hz, 1H); HPLC: MS (M+H-Na): 181.1.

### Example 69: Preparation of 3-cyclobutoxy-7-fluoro-2-hydroxycyclohepta-2,4,6-trien-1-one (Ex.69)

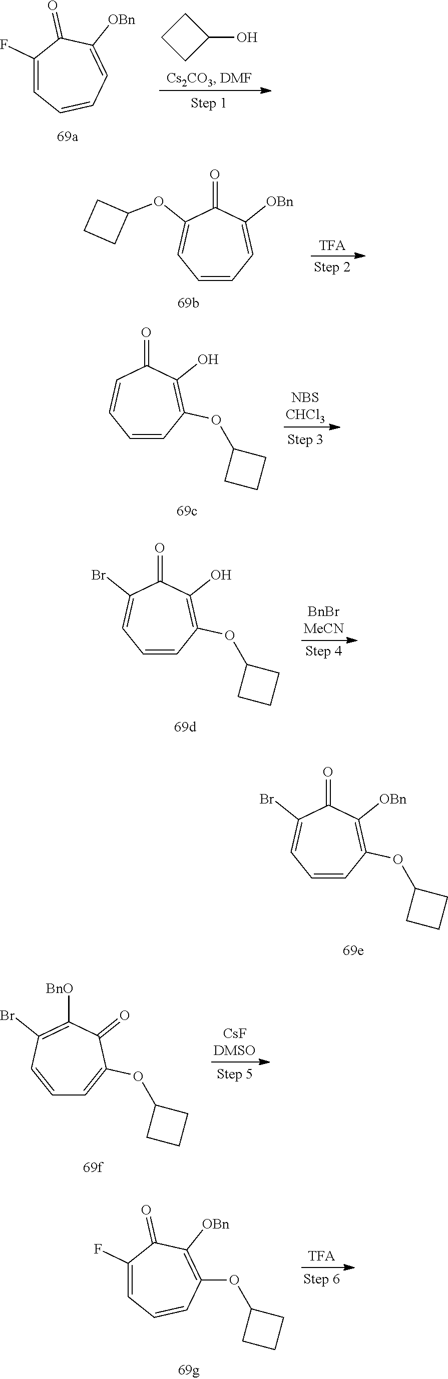

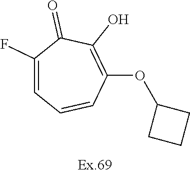 [SMILES: O=c1c(F)cccc(OC2CCC2)c1O]

### Step 1:

A mixture of 69a (6.5 g, 28.2 mmol, 1.00 eq), cyclobutanol (4.07 g, 56.5 mmol, 2.00 eq), Cs₂CO₃ (9.20 g, 28.2 mmol, 1.00 eq) in DMF (30 mL) was degassed and purged with N₂ 3 times, and then the mixture was stirred at 60 °C for 3 hr under N₂ atmosphere. LC-MS showed 69a was consumed completely and one main peak with desired mass was detected. The reaction mixture was quenched by addition of H₂O (5 mL) at 25 °C, extracted with EtOAc (50 mL×4). The combined organic layers were washed with brine (10 mL), dried over anhydrous Na₂SO₄, filtered and concentrated under reduced pressure to give a residue. The residue was purified by column chromatography (SiO₂, Petroleum ether/Ethyl acetate=100/1 to 17/1) to give 69b (13.230 g, crude) as brown oil.

### Step 2:

A mixture of 69b (13.2 g, 46.9 mmol, 1.00 eq) in TFA (0.5 mL) was heated and stirred at 50 °C for 3 hr under N₂ atmosphere. TLC (petroleum ether:ethyl acetate=1:1, R_{f}=0.55) showed 69b was consumed completely and desired mass was detected. The reaction mixture was diluted with CH₂Cl₂ (10 mL) and concentrated under reduced pressure to dryness to give 69c (7 g, crude) as a brown solid. ¹H NMR:

### Step 3:

To a solution of 69c (2 g, 10.4 mmol, 1.00 eq) in CHCl₃ (20 mL) NBS (1.76 g, 9.88 mmol, 0.95 eq) was added in one portion at 20 °C under N₂. The reaction mixture was heated to 60 °C and stirred for 1 hour. HPLC showed 69c was consumed completely and one main peak was detected. After cooling, water (50 mL) was added and then extracted with ethyl acetate (200 mL×5). The combined organic phases were washed with brine (50 mL), dried over anhydrous Na₂SO₄, filtered and concentrated in vacuum to give compound 69d (3.25 g, crude) as a black solid.

### Step 4:

To a solution of 69d (6.47 g, 23.9 mmol, 1.00 eq) in MeCN (80 mL) K₂CO₃ (9.90 g, 71.6 mmol, 3.00 eq) and BnBr (8.17 g, 48 mmol, 2.00 eq) in one portion were added at 20 °C under N₂. The reaction mixture was heated to 90 °C and stirred for 1 hour. LCMS showed 69d was consumed completely and one main peak with desired mass was detected. After cooling, water (50 mL) was added and then extracted with ethyl acetate (100 mL×3). The combined organic phases were washed with brine (50 mL), dried over anhydrous Na₂SO₄, filtered and concentrated under reduced pressure to dryness. The residue was purified by column chromatography (SiO₂, Petroleum ether/Ethyl acetate=72/1 to 28/1) to give 69e (2.88 g, 33.4% yield) as brown oil.

### Step 5:

To a solution of 69e (1.3 g, 3.60 mmol, 1.00 eq) in DMSO (30 mL) CsF (820 mg, 5.40 mmol, 1.50 eq) was added in one portion at 25 °C under N₂. The mixture was heated to 110 °C and stirred for 0.5 hour. LC-MS showed 69e was consumed completely and one main peak with desired mass was detected. The residue was poured into brine (100 mL). The aqueous phase was extracted with ethyl acetate (100 mL×3). The combined organic phases were dried with anhydrous Na₂SO₄, filtered and concentrated in vacuum. The residue was purified by column chromatography (SiO₂, Petroleum ether/Ethyl acetate=72/1 to 28/1) to give 69 g (230 mg, 21.3% yield) as brown oil.

### Step 6:

A mixture of 69 g (180 mg) in TFA (2 mL) was heated to 50 °C and stirred for 2 hours. LC-MS showed 69 g was consumed completely and one main peak with desired mass was detected. The mixture was diluted with DCM (10 mL) and concentrated under reduced pressure to give a residue. The crude product was triturated with petroleum ether twice to afford the titled product Ex.69 (35 mg, 27.8% yield) as a brown solid. ¹H NMR: 400 MHz; HPLC: MS (M+H): 211.1.

### Example 70: Preparation of 5-(cyclopentyloxy)-2-hydroxycyclohepta-2,4,6-trien-1-one (Ex.70)

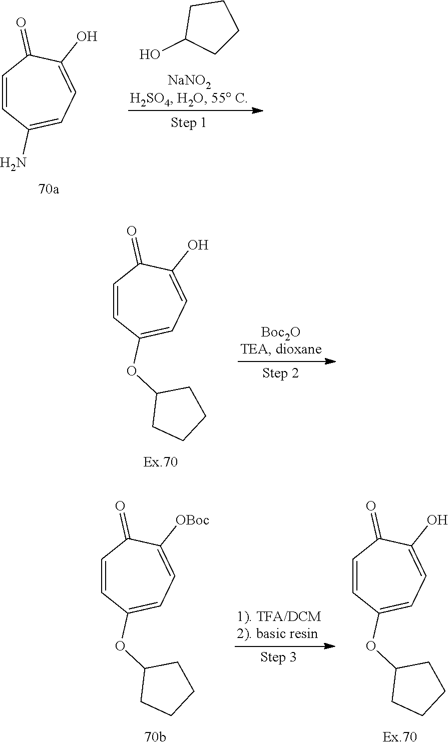

### Step 1:

To a solution of 70a (0.50 g, 3.65 mmol, 1.00 eq) in cyclopentanol (9.49 g, 110 mmol, 10 mL, 30.2 eq) H₂SO₄ (3.68 g, 38.0 mmol, 2 mL, 10.00 eq) was added in one portion at 15 °C under N₂. The mixture was heated to 50 °C and stirred for 5 min, then NaNO₂ (277 mg, 4.02 mmol, 1.10 eq) in H₂O (2 mL) was added drop wise at 55 °C and then stirred for another 90 min. LC-MS showed 70a was consumed completely and one main peak with desired mass was detected. After cooling, the reaction mixture was quenched by addition of H₂O (20 mL) at 15 °C, and then extracted with EtOAc (10 mL×3). The combined organic layers were washed with aq. NaHCO₃ (10 mL×3), brine (10 mL), dried over Na₂SO₄, filtered and concentrated under reduced pressure to give crude Ex.70 (770 mg, crude) as a black oil.

### Step 2:

To a solution of crude Ex.70 (550 mg, 2.67 mmol, 1.00 eq) in dioxane (10 mL) Boc₂O (1.75 g, 8.00 mmol, 3.00 eq) and TEA (1.08 g, 10.6 mmol, 4.00 eq) were added in one portion at 15 °C under N₂. The mixture was heated to 118 °C and stirred for 0.5 hours. TLC (Petroleum ether:Ethyl acetate=3:1) indicated crude Ex.70 was consumed completely and one new spot formed. After cooling, the reaction mixture was concentrated under reduced pressure to dryness and the residue was purified by column chromatography (SiO₂, Petroleum ether/Ethyl acetate=100/1 to 20/1) to give 70b (90.0 mg, 11.0% yield) as yellow oil.

### Step 3:

To a solution of 70b (90.0 mg, 294 umol, 1.00 eq) in DCM (3 mL) TFA (0.5 mL) was added in one portion at 15 °C. The mixture was stirred at 15 °C for 1 hr. TLC (Petroleum ether:Ethyl acetate=3:1) indicated 70b was consumed completely, and one new spot was formed. The reaction mixture was diluted with CH₂Cl₂ (10 mL) and concentrated under reduced pressure to dryness. Then the mixture was re-dissolved in CH₂Cl₂ (5 mL) and Amberlyst A21 (0.1 g) was added and stirred at 25 °C for another 0.5 hr. After filtering, the cake was washed with CH₂Cl₂ (5 mL×2) and the filtrate was concentrated under reduced pressure to afford the titled product Ex.70 (15.0 mg, 24.8% yield) as a yellow solid. ¹H NMR: 400 MHz; δ: 1.60-1.72 (m, 2H) 1.74-1.88 (m, 4H) 1.92-2.07 (m, 2H) 4.80-4.84 (m, 1H) 7.12 (d, J=12.0 Hz, 2H) 7.32 (d, J=12.0 Hz, 2H); HPLC: MS (M+H): 207.1.

### Example 71: Preparation of 9-hydroxy-3,4-dihydrocyclohepta[b]pyran-8(2H)-one (Ex.71)

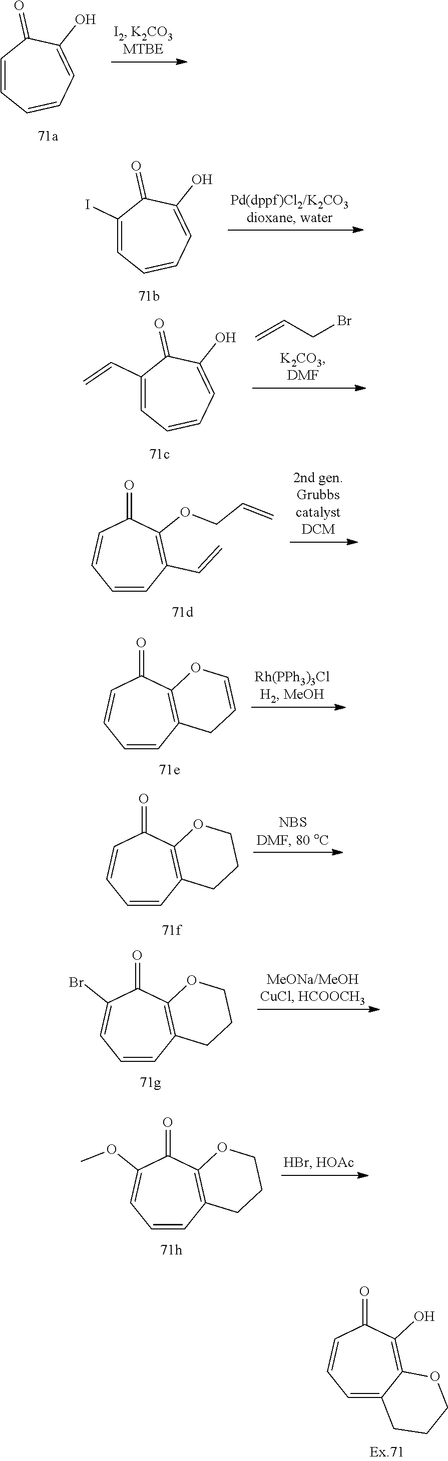

### Step 1:

To a solution of 71a (20 g, 164 mmol, 1.00 eq) in methyl tert-butyl ether (MTBE, 400 mL) K₂CO₃ (52.1 g, 377 mmol, 2.30 eq) and 2 (54.0 g, 213 mmol, 1.30 eq) were added at 20 °C. The mixture was stirred at 20 °C for 20 hr. The reaction mixture was filtered and the filtration was washed with water (50 mL), brine (50 mL), dried over anhydrous Na₂SO₄, filtered and concentrated under reduced pressure to give a crude 71b (40 g, 59.1% yield, 60% purity) as a black solid.

### Step 2:

To a mixture of 71b (40 g, 161 mmol, 1.00 eq) and 4,4,5,5-tetramethyl-2-vinyl-1,3,2-dioxaborolane (44.7 g, 290 mmol, 1.80 eq) in dioxane (500 mL) and H₂O (30 mL) Pd(dppf)Cl₂·CH₂Cl₂ (1.98 g, 2.42 mmol, 15% eq) and K₂CO₃ (44.6 g, 323 mmol, 2.00 eq) were added at 20 °C under N₂. The mixture was heated and stirred at 120 °C for 1 hr. LCMS showed the starting material was almost consumed completely and the major desired MS was observed. After cooling, water (300 mL) was added and then extracted with ethyl acetate (300 mL×3). The combined organic phases were washed with brine (500 mL), dried over anhydrous Na₂SO₄. After filtering, the filtrate was concentrated under reduced pressure to dryness to give 71c (20 g, crude) as a brown oil.

### Step 3:

To a mixture of 71c (20 g, 135 mmol, 1.00 eq) and 3-bromoprop-1-ene (40.8 g, 337 mmol, 2.50 eq) in DMF (200 mL) K₂CO₃ (37.3 g, 270 mmol, 2.00 eq) was added at 20 °C. The mixture was heated and stirred at 70 °C for 1 hr. LCMS showed the starting material was consumed completely and the desired MS was observed. After cooling, the mixture was diluted with EtOAc (500 mL), and the washed with water (10 mL), brine (10 mL), dried over anhydrous Na₂SO₄, filtered and concentrated under reduced pressure to give a residue. The residue was purified by silica gel chromatography (Petroleum ether/Ethyl acetate=95/5 to 80/20) to obtain 71d (0.9 g, 2.83% yield, 80% purity) as a yellow solid.

### Step 4:

To a solution of 71d (0.9 g, 4.78 mmol, 1.00 eq) in DCM (20 mL) benzylidene-[1,3-bis(2,4,6-trimethylphenyl)imidazolidin-2-ylidene]-dichloro-ruthenium tricyclohexylphosphane (2° d generation Grubbs catalyst, 405 mg, 478 umol, 0.1 eq) was added at 25 °C under N₂. The mixture was stirred at 25 °C for 4 hr. TLC (Petroleum ether:Ethyl acetate=3:1, R_{f}=0.1) showed the starting material was consumed and the new spot was observed. After cooling, the mixture was filtered through a pad of Celite and the filter cake was washed with CH₂Cl₂ (30 mL×3). The filtrate was concentrated under reduced pressure to dryness. The residue was purified by silica gel chromatography (Petroleum ether/Ethyl acetate=90/10 to 20/80) to obtain 71e (0.55 g, 64.6% yield, 90% purity) as a brown oil.

### Step 5:

To a solution of 71e (0.55 g, 3.43 mmol, 1.00 eq) in MeOH (12 mL) Rh(PPh₃)₃C1 (222 mg, 240 umol, 0.07 eq) was added under N₂. The suspension was degassed under vacuum and purged with H₂ several times. The mixture was stirred under H₂ (20 psi) at 50 °C for 2 hr. LCMS showed the starting material was consumed completely and desired MS observed. After cooling, the mixture was filtered through a pad of Celite and the filter cake was washed with MeOH (10 mL×2). The filtrate was concentrated under reduced pressure to dryness to get crude 71f (0.6 g, 90% purity) as a brown oil

### Step 6:

To a solution of 71f (0.6 g, 3.70 mmol, 1.00 eq) in DMF (10 mL) NBS (987 mg, 5.55 mmol, 1.50 eq) was added at 25 °C. The mixture was heated and stirred at 80 °C for 1 hr. LCMS showed the starting material was consumed completely and the desired MS was observed. After cooling, saturated sodium thiosulfate (10 mL) was added to the mixture and extracted with DCM (10 mL×2). The combined organic phases were washed with brine (20 mL), dried over anhydrous Na₂SO₄, filtered and concentrated in vacuum to provide 71 g (0.4 g, 31.4% yield, 70% purity) as a brown oil.

### Step 7:

A Teflon-lined autoclave (30 mL) was charged with sodium methanolate (89 mg, 1.66 mmol, 2.00 eq), MeOH (4 mL), chlorocopper (3.29 mg, 33.2 umol, 0.04 eq), methyl formate (19.9 mg, 332 umol, 0.40 eq) and 71 g (0.2 g, 830 umol, 1 eq) under N₂. The resulting mixture was heated and stirred at 115 °C for 2 hr. LCMS showed the starting material was consumed completely and the major desired MS was observed. After cooling, the mixture was filtered through a pad of Celite and the filter cake was washed with CH₂Cl₂ (10 mL×3). The filtrate was concentrated under reduced pressure to dryness to give 71h (0.12 g, 75.3% yield) as a brown oil.

### Step 8:

HBr (2 mL) and AcOH (1 mL) were added into a mixture with 71h (0.1 g, 520 umol, 1.00 eq) at 20 °C. The mixture was heated and stirred at 100 °C for 7 hr. LCMS showed the starting material was consumed completely and the desired MS was observed. After cooling, the reaction mixture was concentrated in vacuum to dryness. The residue was purified by prep-HPLC (column: Welch Xtimate C18 100*25 mm*3 um; mobile phase: [water (0.1% TFA)-ACN]; B %: 10%-40%, 7 min) to afford the titled product Ex.71 (0.02 g, 21.6% yield) as a yellow solid. ¹H NMR: 400 MHz; δ ppm 7.30 (dd, J=1.2, 9.2 Hz, 1H), 7.08-6.95 (m, 2H), 4.43-4.28 (m, 2H), 3.01 (t, J=6.4 Hz, 2H), 2.15-1.97 (m, 2H); HPLC: MS (M+H): 179.

### Example 72: Preparation of 3-cyclopropyl-7-fluoro-2-hydroxycyclohepta-2,4,6-trien-1-one (Ex.72)

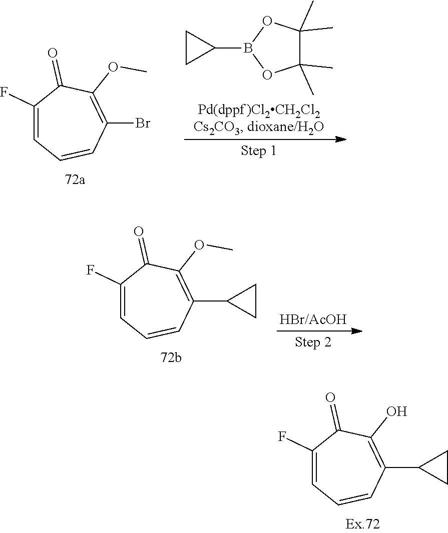

### Step 1:

To a mixture of 72a (0.50 g, 2.15 mmol, 1.00 eq) and 2-cyclopropyl-4,4,5,5-tetramethyl-1,3,2-dioxaborolane (4.33 g, 25.7 mmol, 12.0 eq) in dioxane (6.7 mL) and H₂O (1 mL) Pd(dppf)Cl₂·CH₂Cl₂ (350 mg, 429 umol, 0.20 eq) and Cs₂CO₃ (2.10 g, 6.44 mmol, 3.00 eq) were added in one portion at 15 °C under N₂ atmosphere. The system was degassed and then charged with nitrogen three times. The mixture was heated and stirred at 110 °C for 1 hour. LC-MS showed 72a was consumed completely and one main peak with desired mass was detected. After cooling, the mixture was filtered through a pad of Celite and the filter cake was washed with CH₂Cl₂ (30 mL×3). The filtrate was concentrated under reduced pressure to dryness. The residue was purified by column chromatography (SiO₂, Petroleum ether/Ethyl acetate=72/1 to 28/1) to give compound 72b (630 mg, 75.6% yield) as a brown oil.

### Step 2:

A mixture of 72b (400 mg, 2.06 mmol, 1.00 eq) in HBr (3 mL) and AcOH (1.5 mL) was degassed and purged with N₂ for 3 times, and then the mixture was stirred at 80 °C for 30 min under N₂ atmosphere. LC-MS showed 72b was consumed completely and one main peak with desired mass was detected. After cooling, the reaction mixture was quenched by addition H₂O (10 mL) at 15 °C, and then extracted with EtOAc 50 mL (10 mL×5). The combined organic layers were washed with brine (10 mL), dried over Na₂SO₄, filtered and concentrated under reduced pressure to give a residue. The crude product was triturated with n-hexane (10 mL) at 15 °C twice, and then filtered and filter cake was washed with cold n-hexane (10 mL×2). The precipitate was collected and dried in vacuo to afford the tittled product Ex.72 (68.0 mg, 18.3% yield) as an off white solid. ¹H NMR: 400 MHz; δ 0.75-0.87 (m, 2H) 1.10-1.24 (m, 2H) 2.59 (tt, 0.1=8.4, 5.2 Hz, 1H) 6.86-7.02 (m, 2H) 7.39 (dd, J=18.0, 10.0 Hz, 1H); HPLC: MS: 181.1

### Example 73: Preparation of 2-hydroxy-3-(2-methoxycyclopentyl)cyclohepta-2,4,6-trien-1-one (Ex.73)

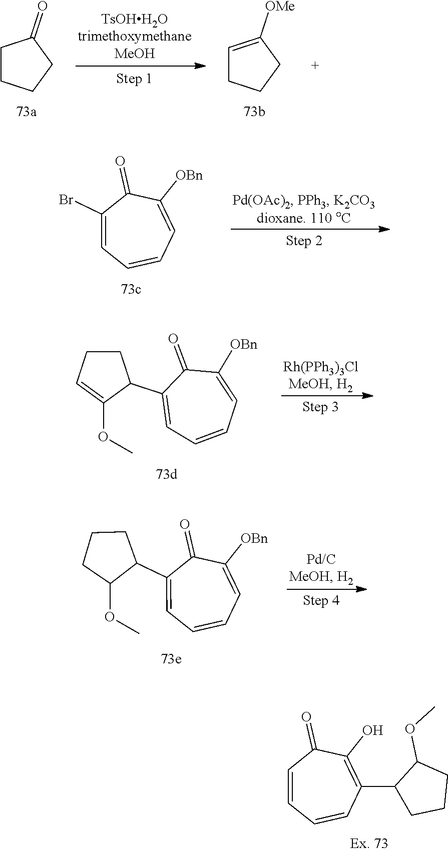 [SMILES: O=C1CCCC1 ; COC1=CCCC1 ; O=c1c(Br)ccccc1OCc1ccccc1 ; COC1=CCCC1c1ccccc(OCc2ccccc2)c1=O ; COC1CCCC1c1ccccc(OCc2ccccc2)c1=O ; COC1CCCC1c1ccccc(=O)c1O]

### Step 1:

To a mixture of 73a (6 g, 71.3 mmol, 6.32 mL, 1.00 eq) and TsOH·H₂O (68 mg, 356 umol, 5% eq) in MeOH (6 mL) trimethoxymethane (7.95 g, 74.9 mmol, 1.05 eq) was added at 5 °C under N₂. The mixture was stirred at 5 °C for 30 min, and then heated to 110 °C and stirred for 19 hrs under N₂. TLC (petroleum ether:ethyl acetate=5:1, phosphomolybdic acid as developer) showed the starting material was consumed and a new spot was observed. The reaction mixture was concentrated in vacuum to give 73b (8 g, crude) as a black solid.

### Step 2:

To a mixture of crude 73b (5.06 g, 51.5 mmol, 5.00 eq) and 73c (3 g, 10.3 mmol, 1.00 eq) in dioxane (15 mL) K₂CO₃ (2.85 g, 20.6 mmol, 2.00 eq), PPh₃ (540.5 mg, 2.06 mmol, 0.20 eq) and Pd(OAc)₂ (231.34 mg, 1.03 mmol, 0.10 eq) were added at 20 °C under N₂ atmosphere. The system was degassed and then charged with nitrogen three times. The mixture was heated and stirred at 110 °C for 1.5 hrs under N₂. TLC (petroleum ether:ethyl acetate=3:1, UV as developer) showed the starting material was consumed and a new spot was observed. After cooling, the mixture was filtered through a pad of Celite and the filter cake was washed with CH₂Cl₂ (30 mL×3). The filtrate was concentrated under reduced pressure to dryness. The residue was purified by by silica gel chromatography (Petroleum ether/Ethyl acetate=100/1 to 100/20) to give 73d (2.4 g, 6.38 mmol, 61.9% yield, 82% purity) as a brown oil.

### Step 3:

To a solution of 73d (0.4 g, 1.30 mmol, 1.00 eq) in MeOH (5 mL) Rh(PPh3)3Cl (120 mg, 130 umol, 0.10 eq) was added under N2 at 25 °C. The suspension was degassed under vacuum and purged with H2 several times. The mixture was stirred under H2 (30 psi) at 40 °C for 3 hours. LCMS showed the starting material was consumed completely and the desired MS was observed. After cooling, the mixture was filtered through a pad of Celite and the filter cake was washed with MeOH (10 mL×2). The filtrate was concentrated under reduced pressure to dryness. The residue was purified by silica gel chromatography (Petroleum ether/Ethyl acetate=100/1 to 100/15) to give 73e (0.20 g, 43.7% yield, 88% purity) as s yellow oil.

### Step 4:

To a solution of 73e (0.1 g, 322 umol, 1.00 eq) in MeOH (4 mL) Pd/C (338 mg, 322 umol, 10% purity) was added under N₂. The suspension was degassed under vacuum and purged with H₂ several times. The mixture was stirred under H₂ (15 psi) at 20 °C for 1 hr. LCMS showed the starting material was consumed completely and the desired MS observed. The mixture was filtered through a pad of Celite and the filter cake was washed with MeOH (10 mL×2). The filtrate was concentrated under reduced pressure to dryness. The residue was purified by prep-HPLC (column: 3_Phenomenex Luna C18 75*30 mm*3 um; mobile phase: [water(0.2% FA)-ACN]; B %: 25%-55%, 7 min) to afford the titled product Ex.73 (10 mg, 13.8% yield, 98% purity) as a yellow gum. ¹HNMR: (CDCl₃, 400 MHz); HPLC: MS: 221.2

### Example 74: Preparation of 8-hydroxy-3-methyl-7H-cyclohepta[b]furan-7-one (Ex.74)

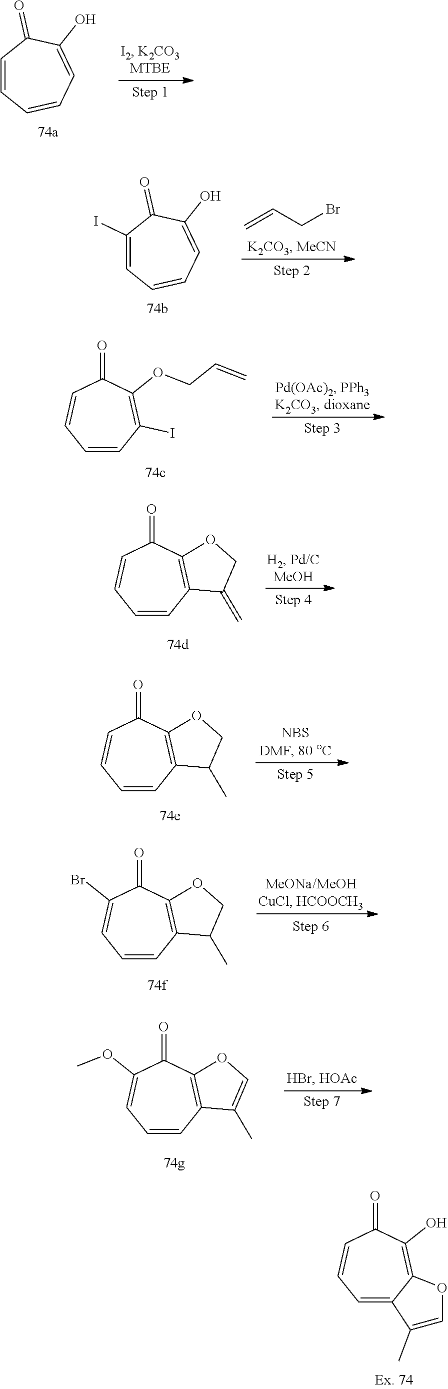

### Step 1:

To a solution of 74a (20 g, 164 mmol, 1.00 eq) in methyl tert-butyl ether (MTBE, 400 mL) K₂CO₃ (52.1 g, 377 mmol, 2.30 eq) and 12 (54.0 g, 213 mmol, 1.30 eq) were added at 20 °C. The mixture was stirred at 20 °C for 20 hr. The reaction mixture was filtered and the filtration was washed with water (50 mL), brine (50 mL), dried over anhydrous Na₂SO₄, filtered and concentrated under reduced pressure to give a crude 74b (40 g, 59.1% yield, 60% purity) as a black solid.

### Step 2:

To a mixture of 74b (30 g, 121 mmol, 1.00 eq) and 3-bromoprop-1-ene (29.3 g, 242 mmol, 2.00 eq) in DMF (300 mL) K₂CO₃ (33.4 g, 242 mmol, 2.00 eq) was added at 20 °C. The mixture was heated and stirred at 70 °C for 1 hr. LCMS showed the starting material was consumed completely and desired MS observed. After cooling, the mixture was filtered through a pad of Celite and the filter cake was washed with CH₂Cl₂ (30 mL×3). The filtrate was concentrated under reduced pressure to dryness. The residue was purified by silica gel chromatography (Petroleum ether/Ethyl acetate=95/5 to 80/20) to give 74c (6.4 g, 17.5% yield, 95% purity) as a yellow oil.

### Step 3:

To a solution of 74c (3.20 g, 11. mmol, 1 eq) in dioxane (250 mL) PPh₃ (874 mg, 3.33 mmol, 0.30 eq), Pd(OAc)₂ (498 mg, 2.22 mmol, 0.20 eq) and K₂CO₃ (3.07 g, 22.2 mmol, 2.00 eq) were added at 20 °C under N₂. The suspension was degassed under vacuum and purged with N₂ several times. The mixture was heated and stirred at 50 °C for 10 hr. TLC (Petroleum ether:Ethyl acetate=3:1, R_{f}=0.1) showed the starting material was consumed and a new spot was observed. After cooling, the mixture was filtered through a pad of Celite and the filter cake was washed with CH₂Cl₂ (30 mL×3). The filtrate was concentrated under reduced pressure to dryness. The residue was purified by silica gel chromatography (Petroleum ether/Ethyl acetate=50/50 to 20/80) to get crude 74d (0.7 g, 50% purity) as a yellow oil.

### Step 4:

To a solution of 74d (0.9 g, 4.50 mmol, 1.00 eq) in MeOH (15 mL) Rh(PPh₃)₃C1 (249 mg, 269.72 umol, 0.060 eq) was added under N₂. The suspension was degassed under vacuum and purged with H₂ several times. The mixture was stirred under H₂ (20 psi) at 50 °C for 3 hr. LCMS showed the starting material was consumed completely and desired MS observed. After cooling, the mixture was filtered through a pad of Celite and the filter cake was washed with MeOH (10 mL×3). The filtrate was concentrated under reduced pressure to dryness. The residue was purified by silica gel chromatography (Petroleum ether/Ethyl acetate=90/10 to 50/50) to obtain crude 74e (0.4 g) as a brown oil.

### Step 5:

To a solution of 74e (400 mg, 1.23 mmol, 1.00 eq) in DMF (4 mL) NBS (263 mg, 1.48 mmol, 1.20 eq) was added at 20 °C. The mixture was heated and stirred at 80 °C for 2 hr. LCMS showed the starting material was consumed completely and desired MS observed. A small amount of water (0.5 mL) was added and the mixture solvent was concentrated in vacuum to dryness. The residue was purified by silica gel chromatography (Petroleum ether/Ethyl acetate=20/80 to 50/50) to give 74f (0.15 g, 35.3% yield, 70% purity) as a brown oil.

### Step 6:

A Teflon-lined autoclave (30 mL) was charged with sodium;methanolate (67 mg, 1.24 mmol, 2.00 eq), MeOH (3 mL), chlorocopper (2.46 mg, 24.9 umol, 0.04 eq), methyl formate (14.9 mg, 248 umol, 0.40 eq) and 74f (0.15 g, 622 umol, 1 eq) under N₂. The reaction mixture was heated and stirred at 115 °C for 2 hr. LCMS showed the starting material was consumed completely and desired MS observed. After cooling, the mixture was filtered through a pad of Celite and the filter cake was washed with CH₂Cl₂ (30 mL×3). The filtrate was concentrated under reduced pressure to dryness. The residue was purified by silica gel chromatography (100% ethyl acetate as eluent) to get 74g (0.06 g, 45.6% yield, 90% purity) as a yellow solid.

### Step 7:

HBr (2 mL) and HOAc (1 mL) was added into a solution of 74g (0.03 g, 158 umol, 1.00 eq) at 20 °C. The mixture was heated and stirred at 100 °C for 3 hr. LCMS showed the starting material was consumed completely and desired MS observed. After cooling, the reaction mixture was concentrated in vacuum to dryness. The residue was purified by prep-HPLC (column: Phenomenex Synergi C18 150*25*10 um; mobile phase: [water (0.1% TFA)-ACN]; B %: 5%-30%, 10 min) to afford the titled product Ex.74 (9 mg, 32.4% yield) as a white solid. ¹H NMR: 400 MHz; δ ppm 7.98 (d, J=1.2 Hz, 1H), 7.52-7.33 (m, 3H), 2.31 (d, J=1.2 Hz, 3H); HPLC: MS (M+H): 177.0

### Example 75: Preparation of 3-(cyclopentyloxy)-7-fluoro-2-hydroxycyclohepta-2,4,6-trien-1-one (Ex. #75)

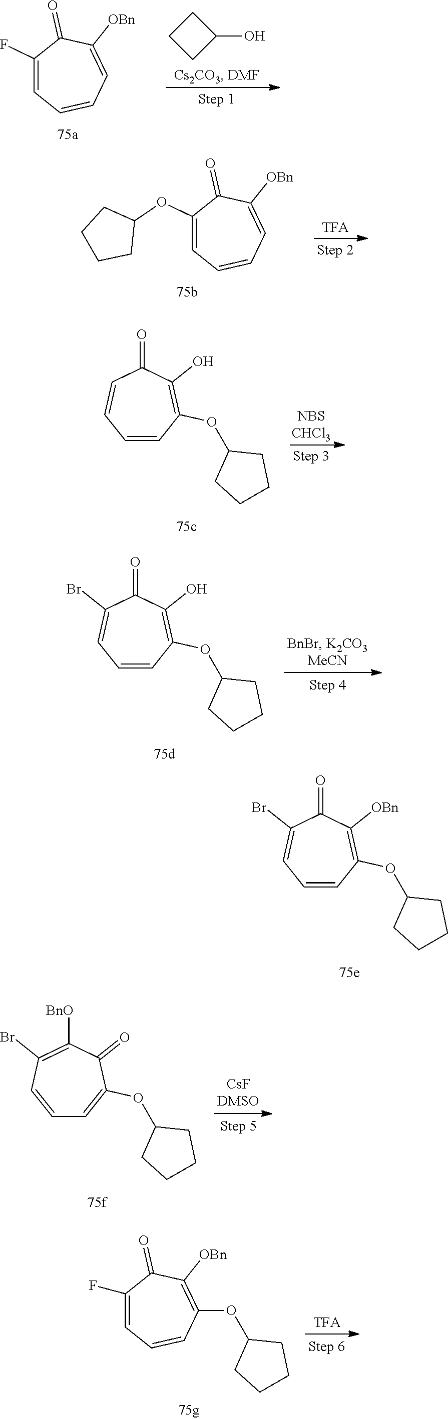

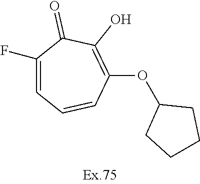 [SMILES: O=c1c(F)cccc(OC2CCCC2)c1O]

### Step 1:

To a mixture of 75a (2.75 g, 11.9 mmol, 1.00 eq) and cyclopentanol (2.06 g, 23.9 mmol, 2.17 mL, 2.00 eq) in DMF (40 mL) Cs₂CO₃ (7.78 g, 23.9 mmol, 2.00 eq) was added in one portion at 25 °C under N₂. The mixture was heated to 80 °C and stirred for 3 hours. LC-MS showed 75a was consumed completely and one main peak with desired mass was detected. After cooling, the reaction mixture was quenched by addition H₂O (10 mL) at 25 °C, and then extracted with EtOAc (20 mL×5). The combined organic layers were washed with brine (10 mL), dried over Na₂SO₄, filtered and concentrated under reduced pressure to give a residue. The residue was purified by column chromatography (SiO₂, Petroleum ether/Ethyl acetate=75/1 to 25/1) to give compound 75b (6.30 g, 21.3 mmol, 88.9% yield) as a yellow oil.

### Step 2:

A solution of 75b (3.00 g, 10.1 mmol, 1.00 eq) in TFA (2 mL) was heated to 50 °C and stirred for 5 hours. LC-MS showed 75b was consumed completely and one main peak with desired mass was detected. The reaction mixture was diluted with DCM (10 mL) and concentrated under reduced pressure to give crude 75c (3.20 g, crude) as a yellow oil, which was used in the next step.

### Step 3:

To a solution of 75c (3.20 g, 15.5 mmol, 1.00 eq) in CHCl₃ (13 mL) NBS (2.62 g, 14.7 mmol, 0.95 eq) was added in one portion at 15 °C under N₂. The mixture was heated to 60 °C and stirred for 20 min. LC-MS showed ~12% of 75c remained. Several new peaks were shown on HPLC and ~48% of desired compound was detected. The reaction mixture was quenched by addition of H₂O (10 mL) at 15 °C, and then extracted with EtOAc (20 mL×5). The combined organic layers were washed with H₂O (10 mL), brine (10 mL), dried over Na₂SO₄, filtered and concentrated under reduced pressure to give crude compound 75c (4.47 g, crude) as a brown oil.

### Step 4:

To a solution of 75d (4.47 g, 15.7 mmol, 1.00 eq) in MeCN (8 mL) K₂CO₃ (6.50 g, 47.0 mmol, 3.00 eq) and BnBr (5.36 g, 31.3 mmol, 3.72 mL, 2.00 eq) were added in one portion at 15 °C under N₂. The mixture was stirred at 15 °C for 2 min, then heated to 90 °C and stirred for 1 hour. HPLC showed 75d was consumed completely and two main peaks with desired mass detected. After cooling, the reaction mixture was quenched by addition of H₂O (10 mL), and then extracted with EtOAc (20 mL×3). The combined organic layers were washed with H₂O (10 mL), brine (10 mL), dried over Na₂SO₄, filtered and concentrated under reduced pressure to give a residue. The residue was purified by column chromatography (SiO₂, Petroleum ether/Ethyl acetate=72/1 to 28/1) to give compound 75e (2.00 g, 5.33 mmol, 34.0% yield) as brown oil and isomer 75f.

### Step 5:

A mixture of 75e (1.80 g, 4.79 mmol, 1.00 eq) and dried CsF (2.18 g, 14.4 mmol, 3.00 eq) in DMSO (8 mL) was degassed and purged with N₂ for 3 times, and then the mixture was heated and stirred at 110 °C for 15 min under N₂ atmosphere. LC-MS showed 75e was consumed completely and one main peak with desired mass was detected. After cooling, the reaction mixture was quenched by addition of H₂O (20 mL), and then extracted with EtOAc (30 mL×4). The combined organic layers were washed with H₂O (20 mL), brine (20 mL), dried over Na₂SO₄, filtered and concentrated under reduced pressure to give a residue. The residue was purified by pre-HPLC (TFA condition) to give compound 75g (153 mg, 10.1% yield) as a brown oil.

### Step 6:

A solution of 75g (153 mg, 486 umol, 1.00 eq) in TFA (2 mL) was degassed and purged with N₂ for 3 times, and then the mixture was stirred at 50 °C for 2 hr under N₂ atmosphere. LC-MS showed 75g was consumed completely and one main peak with desired mass was detected. The reaction mixture was concentrated under reduced pressure to give a residue. The residue was purified by prep-HPLC (FA condition) to afford the titled product Ex.75 (60.0 mg, 54.9% yield) as a yellow oil. ¹H NMR: 400 MHz; S 1.61-1.76 (m, 2H) 1.80-1.97 (m, 4H) 1.98-2.10 (m, 2H) 4.99-5.06 (m, 1H) 7.03-7.18 (m, 1H) 7.24-7.45 (m, 2H); HPLC: MS: 225.1

### Example 76: Preparation of 7-fluoro-2-hydroxy-3-(tetrahydro-2H-pyran-2-yl)cyclohepta-2,4,6-trien-1-one (Ex.76)

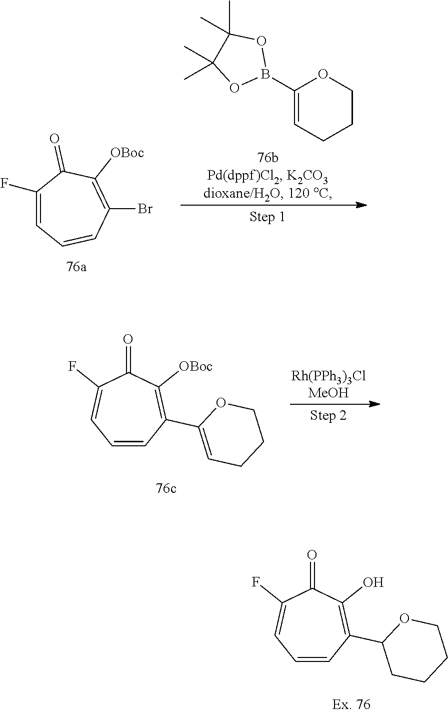

### Step 1:

To a solution of 76a (1.00 g, 3.13 mmol, 1.00 eq) and 76b (789 mg, 3.76 mmol, 1.20 eq) in dioxane (10.0 mL) and water (2.00 mL) K₂CO₃ (866 mg, 6.27 mmol, 2.00 eq) and Pd(dppf)Cl₂·CH₂Cl₂ (255 mg, 313 umol, 0.10 eq) were added at 20 °C under N₂ atmosphere. The system was degassed and then charged with nitrogen three times. The mixture was heated and stirred at 110 °C for 1 hr. LC-MS showed the starting material was consumed and desired MS observed. After cooling, the mixture was filtered through a pad of Celite and the filter cake was washed with CH₂Cl₂ (30 mL×3). The filtrate was concentrated under reduced pressure to dryness. The residue was purified by silica gel chromatography (Petroleum ether/Ethyl acetate=90/10 to 80/20) to get 76c (0.20 g, 620 umol, 19.8% yield) as a yellow oil.

### Step 2:

To a solution of 76c (0.20 g, 620 umol, 1.00 eq) in MeOH (5.00 mL) Rh(PPh₃)₃C1 (57.0 mg, 62.0 umol, 0.10 eq) was added under N₂. The suspension was degassed under vacuum and purged with H₂ several times. The mixture was stirred under H₂ (20 psi) at 50 °C for 1 hr. LCMS showed the starting material was consumed and desired MS observed. The mixture was filtered and the filtrate was concentrated in vacuum to dryness. The residue was purified by prep-HPLC (column: Phenomenex Gemini-NX 150*30 mm*5 um; mobile phase: [water (0.225% FA)-ACN]; B %: 30%-60%, 8 min) to afford the titled product Ex. 76 (85.0 mg, 371 umol, 59.8% yield, 98% purity) as a yellow solid. ¹H NMR: 400 MHz; δ ppm 7.76-7.57 (m, 2H), 7.10 (dt, J=4.0, 10.8 Hz, 1H), 4.77 (d, J=9.6 Hz, 1H), 4.07 (br d, J=11.2 Hz, 1H), 3.64-3.51 (m, 1H), 1.87 (br t, J=11.2 Hz, 2H), 1.72-1.51 (m, 3H), 1.29-1.13 (m, 1H)

### Example 77: Preparation of 4-cyclopropyl-2-fluoro-7-hydroxycyclohepta-2,4,6-trien-1-one (Ex.77)

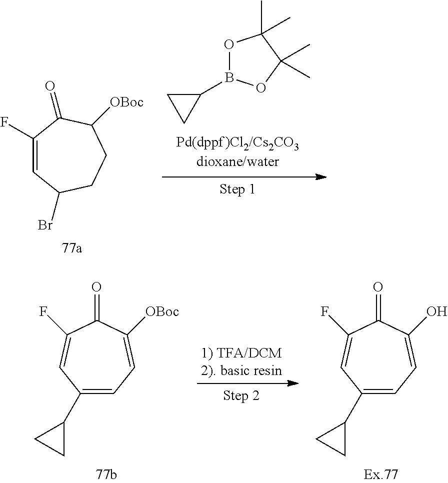

### Step 1:

To a mixture of 77a and Cs₂CO₃ (827 mg, 2.54 mmol, 3.00 eq) in dioxane (7.00 mL) and H₂O (1.00 mL) 2-cyclopropyl-4,4,5,5-tetramethyl-1,3,2-dioxaborolane (1.42 g, 8.46 mmol, 10.0 eq) was added in one portion at 25 °C under N₂. Then Pd(dppf)Cl₂·CH₂Cl₂ (138 mg, 169.2 umol, 0.20 eq) was added to the mixture. The system was degassed and then charged with nitrogen three times. The mixture was heated and stirred at 118 °C for 50 mins. TLC (Petroleum ether/Ethyl acetate=3/1, R_{f} (material)=0.40, R_{f}(product)=0.15) showed the reaction was completed. The mixture was extracted with ethyl acetate (50 mL×3). The combined organic phases were washed with brine (50 mL), dried over anhydrous Na₂SO₄, filtered and concentrated under reduced pressure to give a residue. The residue was purified by column chromatography (SiO₂, Petroleum ether/Ethyl acetate=100/0 to 90/10) to give compound 77b (0.13 g, 463 umol, 54.8% yield) as a yellow oil.

### Step 2:

To a solution of 77b (77.0 mg, 274 umol, 1.00 eq) in DCM (2.00 mL) TFA (31.3 mg, 274 umol, 1.00 eq) was added in one portion at 20 °C for 20 mins. HPLC showed the starting material was consumed completely. The reaction mixture was diluted with CH₂Cl₂ (10 mL) and concentrated under reduced pressure to dryness below 10 °C. Then the mixture was re-dissolved in CH₂Cl₂ (5 mL) and added Amberlyst A21 (0.1 g) and stirred at 25 °C for another 0.5 hr. After filtering, the cake was washed with CH₂Cl₂ (5 mL×2) and the filtrate was concentrated under reduced pressure to afford the titled product Ex.77 (41.0 mg, 227 umol, 82.8% yield) as a yellow solid. ¹H NMR: 400 MHz; S 0.76-0.86 (m, 2H) 1.05-1.14 (m, 2H) 2.04 (tt, J=8.4, 5.11 Hz, 1H) 7.25 (dd, J=10.8, 1.88 Hz, 1H) 7.39 (d, J=10.8 Hz, 1H) 7.42-7.50 (m, 1H); HPLC: MS: (M+1): 181.1

### Example 78: Preparation of 3-ethyl-7-fluoro-2-hydroxycyclohepta-2,4,6-trien-1-one (Ex.78)

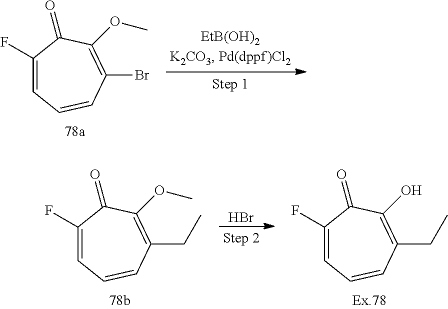

### Step 1:

To a mixture of 78a (600 mg, 2.57 mmol, 1 eq), methylboronic acid (1.90 g, 25.7 mmol, 10 eq) and K₂CO₃ (889 mg, 6.44 mmol, 2.5 eq) in toluene (5 mL) Pd(dppf)Cl₂·CH₂Cl₂ (210 mg, 257 umol, 0.1 eq) was added under N₂ atmosphere. The system was degassed and then charged with nitrogen three times. The mixture was heated and stirred at 110 °C for 30 min under N₂ atmosphere. TLC (petroleum ether:ethyl acetate=3:1) indicated 78a was consumed completely and one new spot formed. The reaction was confirmed by TLC. After cooling, the reaction mixture was quenched by addition of H₂O (10 mL) at 15 °C and extracted with EtOAc (20 mL×4). The combined organic layers were washed with H₂O (10 mL), dried over Na₂SO₄, filtered and concentrated under reduced pressure to give a residue. The residue was purified by column chromatography (SiO₂, Petroleum ether:Ethyl acetate=85:1 to 15:1) to give compound 78b (391 mg, 2.15 mmol, 83.3% yield) as a yellow oil.

### Step 2:

A mixture of 78b (300 mg, 1.65 mmol, 1 eq) in aq.HBr (3 mL) was heated and stirred at 80 °C for 30 min under N₂ atmosphere. LC-MS showed 78b was consumed completely and one main peak with desired mass was detected. After cooling, the reaction mixture was quenched by addition H₂O (2 mL) at 15 °C, and then extracted with DCM/MeOH (10:1, 20 mL×4). The combined organic layers were washed with brine (10 mL), dried over Na₂SO₄, filtered and concentrated under reduced pressure to give a residue. The residue was triturated by EtOAc (2 mL) and then filtered, the filter cake was washed with n-hexane and dried in vacuo to afford the titled product Ex.78 (100 mg, 36.1% yield) as an off white solid. ¹H NMR: 400 MHz; δ: 1.27 (t, 0.1=7.6 Hz, 3H) 2.89 (q, J=7.6 Hz, 2H) 6.95 (td, J=10.4, 4.0 Hz, 1H) 7.34-7.52 (m, 2H); HPLC: MS (M+H): 169.2

### Example 79: Preparation of 5-fluoro-2-hydroxycyclohepta-2,4,6-trien-1-one (Ex.79)

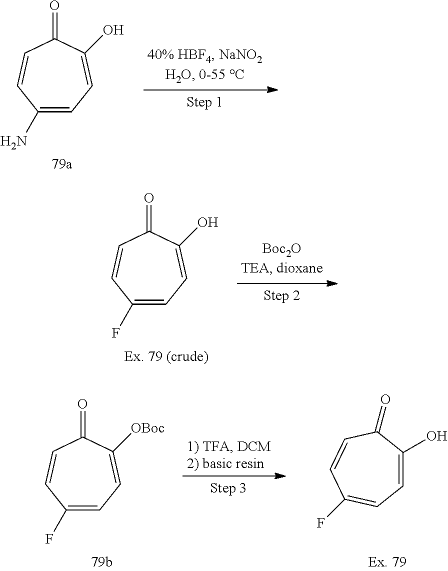

### Step 1:

To a solution of 79a (5 g, 36.5 mmol, 1.00 eq) in trifluoroborane;hydrofluoride (70.5 g, 321 mmol, 50 mL, 40% purity, 8.81 eq) NaNO₂ (2.77 g, 40.1 mmol, 1.10 eq) in H₂O (5 mL) was added drop wise at 0 °C under N₂. The mixture was stirred at 0 °C for 0.5 hr and heated to 55 °C for 60 min. LC-MS showed 79a was consumed completely and one main peak with desired mass detected. After cooling, the reaction mixture was quenched by addition of aq. NaHCO₃ (10 mL) at 25 °C, and then extracted with EtOAc (10 mL×3). The combined organic layers were washed with brine (10 mL), dried over Na₂SO₄, filtered and concentrated under reduced pressure to give Ex.79 (1.7 g, crude) as a yellow solid.

### Step 2:

To a solution of Ex.79 (1.15 g, 8.21 mmol, 1.00 eq) an in dioxane (15 mL) TEA (4.98 g, 49.3 mmol, 6.85 mL, 6 eq) and Boc₂O (7.17 g, 32.8 mmol, 4 eq) were added in one portion at 25 °C under N₂. The mixture was heated and stirred at 118 °C for 30 min. TLC (Petroleum ether:Ethyl acetate=3:1) indicated Ex.79 was consumed completely and one new spot formed. After cooling, the reaction mixture was concentrated under reduced pressure to give a residue. The residue was purified by column chromatography (SiO2, Petroleum ether/Ethyl acetate=100/1 to 4/1) to give 79b (0.5 g, 2.08 mmol, 25.4% yield) as a yellow solid.

### Step 3:

To a solution of 79b (0.14 g, 583 umol, 1.00 eq) in DCM (5 mL) TFA (6.75 mmol, 0.5 mL, 11.6 eq) was added in one portion at 15 °C under N₂. The mixture was stirred at 15 °C for 30 min. TLC (Petroleum ether:Ethyl acetate=1:1) indicated 79b was consumed completely and one new spot formed. The reaction mixture was diluted with CH₂Cl₂ (10 mL) and concentrated under reduced pressure to dryness below 10 °C. Then the mixture was re-dissolved in CH₂Cl₂ (5 mL) and added Amberlyst A21 (0.1 g) and stirred at 25 °C for another 0.5 hr. After filtering, the cake was washed with CH₂Cl₂ (5 mL×2) and the filtrate was concentrated under reduced pressure to afford the titled product Ex.79 (50 mg, 61.2% yield) as a yellow solid. ¹H NMR: 400 MHz; S 7.25-7.33 (m, 2H) 7.34-7.45 (m, 2H); HPLC: MS: 141.1

### Example 80: Preparation of 5-cyclopropoxy-2-hydroxycyclohepta-2,4,6-trien-1-one (Ex.80)

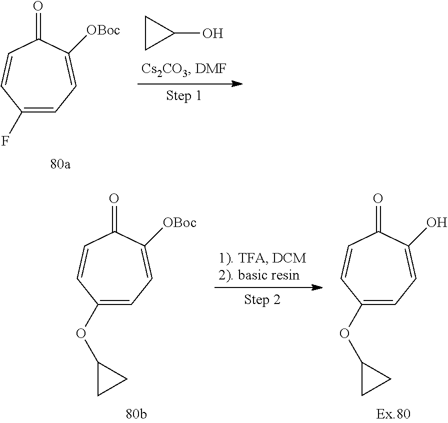

### Step 1:

To a mixture of 80a (0.25 g, 1.04 mmol, 1.00 eq) and cyclopropanol (302 mg, 5.20 mmol, 5.00 eq) in DMF (5 mL) Cs₂CO₃ (1.02 g, 3.12 mmol, 3.00 eq) was added in one portion at 15 °C under N₂. The mixture was heated and stirred at 60 °C for 0.5 hr. TLC (Petroleum ether:Ethyl acetate=1:1) 80a was consumed completely and one new spot formed. The reaction mixture was quenched by addition of H₂O (20 mL) at 15 °C, and then extracted with EtOAc (10 mL×3). The combined organic layers were washed with H₂O (20 mL), brine (20 mL), dried over Na₂SO₄, filtered and concentrated under reduced pressure to give a residue. The residue was purified by column chromatography (SiO₂, Petroleum ether/Ethyl acetate=100/1 to 3/1) to give 80b (140 mg, 503 umol, 48.3% yield) as a yellow oil.

### Step 2:

To a solution of 80b (0.14 g, 503 umol, 1.00 eq) in DCM (5 mL) TFA (0.5 mL) was added in one portion at 15 °C under N₂. The mixture was stirred at 15 °C for 60 min. TLC (Petroleum ether:Ethyl acetate=1:1) indicated 80b was consumed completely and one new spot formed. The reaction mixture was diluted with CH₂Cl₂ (10 mL) and concentrated under reduced pressure to dryness below 10 °C. Then the mixture was re-dissolved in CH₂Cl₂ (5 mL) and added Amberlyst A21 (0.1 g) and stirred at 25 °C for another 0.5 hr. After filtering, the cake was washed with CH₂Cl₂ (5 mL×2) and the filtrate was concentrated under reduced pressure to afford the titled product Ex.80 (80.0 mg, 89.3% yield) as a brown solid. ¹H NMR: 400 MHz; δ: 0.71-0.80 (m, 2H) 0.80-0.91 (m, 2H) 3.81 (tt, J=6.0, 3.2 Hz, 1H) 7.28-7.39 (m, 4H); HPLC: MS (M+H): 179.1

### Example 81: Preparation of 2-hydroxy-3-(oxetan-3-ylmethyl)cyclohepta-2,4,6-trien-1-one (Ex.81)

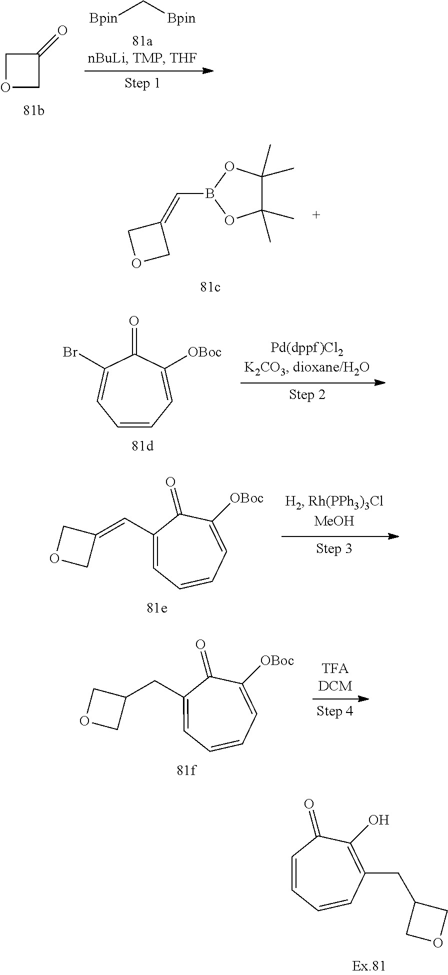

### Step 1:

To a solution of TMP (1.96 g, 13.8 mmol, 2.36 mL, 1.00 eq) in THF (50 mL) n-BuLi (2.5 M, 5.55 mL, 1.00 eq) was added dropwise at −30 °C under N₂, and the mixture was stirred at −30 °C for 30 min. Then the reaction was cooled to −78 °C, and a solution of 81a (3.16 g, 11.8 mmol, 0.85 eq) in THF (2.00 mL) was added drop-wise to the mixture. After stirring for 30 min at −78 °C, another solution of 81b (3.00 g, 41.6 mmol, 3.00 eq) in THF (3 mL) was added drop-wise at −78 °C. The reaction mixture was allowed to slowly warm up to 15 °C and stirred for 10 hrs. A new spot was observed on TLC (PE/EtOAc=1/1, R_{f}=0.45). The mixture was cooled to 0 °C and aq. NH₄Cl (25 mL) was added drop-wise to quench the reaction. After additional stirring for 1 h, the resulting mixture was filtered and the filtrate was rotary evaporated to remove THF. H₂O (30 mL) was added to the obtained residue, and the aqueous layer was extracted with EtOAc (40 mL×4). The combined organic phases were washed with brine (25 mL), dried over anhydrous Na₂SO₄ and concentrated in vacuo to give 81c (2.5 g, crude) as a yellow gum. Which was used in the next step.

### Step 2:

To a mixture of 81d (1.00 g, 3.32 mmol, 1.00 eq), 81c (976 mg, 4.98 mmol, 1.50 eq) and K₂CO₃ (917 mg, 6.64 mmol, 2.00 eq) in dioxane (10 mL) and water (1 mL) Pd(dppf)Cl₂·CH₂Cl₂ (542 mg, 664 umol, 0.20 eq) was added under N₂ atmosphere. The system was degassed and then charged with nitrogen three times. The mixture was heated and stirred at 118 °C for 1.5 hr. A new spot was observed on TLC (PE/EtOAc=1/1, Rf=0.3). After cooling, water (25 mL) was added to the reaction mixture and extracted with ethyl acetate (25 mL×3). The combined organic phases were washed with brine (50 mL), dried over anhydrous Na₂SO₄, filtered and concentrated in vacuum. The residue was purified by silica gel chromatography (PE/EtOAc=3/1, R_{f}=0.26) to give crude product 81e (400 mg, 1.38 mmol, 41.4% yield) as a light-yellow solid.

### Step 3:

A mixture of 81e (400 mg, 1.38 mmol, 1.00 eq) and Rh(PPh₃)₃Cl (1.27 g, 1.38 mmol, 1.00 eq) in MeOH (2.00 mL) was stirred under H₂ (30 psi) at 25 °C for 1 hr. A new was spot was observed on TLC (PE/EtOAc=1/1, R_{f}=0.4). The mixture was filtered through a pad of Celite and the filter cake was washed with MeOH (10 mL×2). The filtrate was concentrated under reduced pressure to dryness. The residue was purified by silica column (PE/EtOAc=2/1) to give the desired 81f (200 mg, 49.6% yield) as a white solid.

### Step 4:

To a solution of 81f (150 mg, 513 umol, 1.00 eq) in water (2.00 mL) HOAc (900 mg, 14.9 mmol, 857 μL, 29.2 eq) was added at 15 °C. The reaction mixture was heated and stirred at 45 °C for 30 min. 81f was disappeared on TLC (PE/EtOAc=10/1, R_{f}=0.3), desired MS observed on LCMS. The yellow solution was concentrated to give a yellow gum. The solid was purified by pre-HPLC (column: Xtimate C18 100*30 mm*3 um; mobile phase: [water (0.2% FA)-ACN]; B %: 1%-30%, 8 min) to afford the titled product Ex.81 (60.0 mg, 312 umol, 60.8% yield) as a white solid. ¹HNMR: 400 MHz; δ 7.20-7.10 (m, 2H), 6.95 (dd, J=8.4, 11.0 Hz, 1H), 4.50-4.43 (m, 1H), 4.06 (dd, J=8.4, 10.8 Hz, 1H), 3.66-3.60 (m, 1H), 3.59-3.52 (m, 1H), 3.00-2.91 (m, 1H), 2.81-2.72 (m, 1H), 2.31-2.15 (m, 1H); HPLC: MS: 193.2

### Example 82: Preparation of 2-hydroxy-3-(spiro[2.3]hexan-5-yloxy)cyclohepta-2,4,6-trien-1-one (Ex.82)

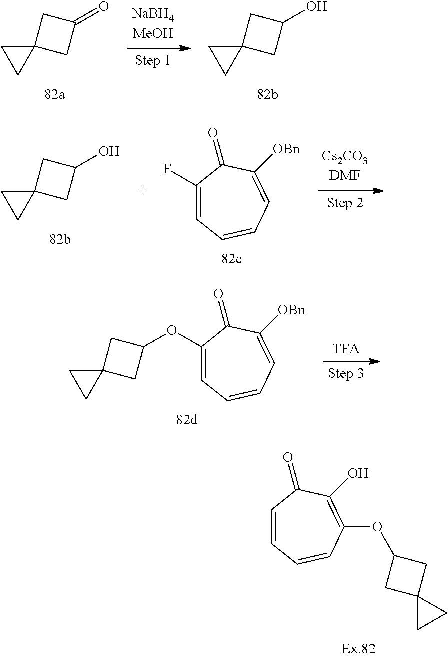 [SMILES: O=C1CC2(CC2)C1 ; OC1CC2(CC2)C1 ; OC1CC2(CC2)C1 ; O=c1c(F)ccccc1OCc1ccccc1 ; O=c1c(OCc2ccccc2)ccccc1OC1CC2(CC2)C1 ; O=c1ccccc(OC2CC3(CC3)C2)c1O]

### Step 1:

To a solution of 82a (0.2 g, 2.08 mmol, 1 eq) in MeOH (3 mL) NaBH₄ (39 mg, 1.04 mmol, 0.5 eq) was added at 0 °C under N₂. The mixture was stirred at 20 °C for 0.5 hr. TLC (petroleum ether:ethyl acetate=3:1, KMnO₄ as developer) showed the starting material was consumed and a new spot observed. The reaction mixture was diluted with CH₂Cl₂ (10 mL), filtered and concentrated under reduced pressure to give crude 82b (0.210 g, crude) as a yellow oil.

### Step 2:

To a mixture of 82c (0.5 g, 2.17 mmol, 1 eq) and 82b (192 mg, 1.95 mmol, 0.9 eq) in DMF (5 mL) Cs₂CO₃ (1.42 g, 4.34 mmol, 2 eq) was added in one portion at 25 °C under N₂. The mixture was heated and stirred at 60 °C for 2 hrs. LCMS showed the starting materials was consumed and the desired MS was detected. After cooling to room temperature, water (15 mL) was added to the reaction mixture and extracted with ethyl acetate (15 mL×2). The combined organic phases were washed with brine (10 mL×2), dried over anhydrous Na₂SO₄, filtered and concentrated under reduced pressure. The residue was purified by silica gel chromatography (Petroleum ether:Ethyl acetate=100:1 to 100:28) to give 82d (0.53 g, 70.4% yield, 89% purity) as a yellow oil.

### Step 3:

A solution of 82d (0.9 g, 2.92 mmol, 1 eq) in TFA (5 mL) was heated and stirred at 50 °C for 1.5 hrs. LCMS showed the starting materials was consumed and the desired MS was detected. After cooling, the mixture was diluted with DCM (20 mL) and concentrated in vacuo to dryness. The residue was purified by prep-HPLC (column: Phenomenex Luna C 18 200*40 mm*10 um; mobile phase: [water (0.2% FA)-ACN]; B %: 25%-55%, 8 min) to afford the titled product Ex.82 (0.210 g, 32.9% yield, 100% purity) as a yellow solid.

### Example 83: Preparation of 3-(cyclopropylmethoxy)-2-hydroxycyclohepta-2,4,6-trien-1-one (Ex.83)

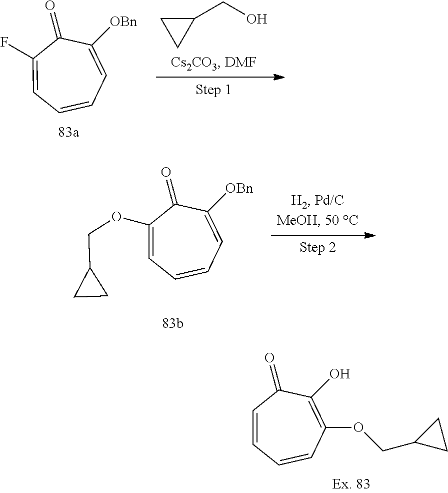

### Step 1:

To a mixture of 83a (1.00 g, 4.34 mmol, 1.00 eq) and Cs₂CO₃ (2.83 g, 8.69 mmol, 2 eq) in DMF (5.00 mL) cyclopropylmethanol (469 mg, 6.52 mmol, 1.5 eq) was added at 20 °C. The reaction mixture was heated and stirred at 60 °C for 1 hr. LCMS showed the starting material was consumed completely. After cooling, the mixture was diluted with H₂O (10 mL), and then extract with EtOAc (20 mL×3). The combined organic layers were washed with brine (10 mL), dried over Na₂SO₄, filtered and concentrated under reduced pressure to dryness. The residue was purified by column chromatography (SiO₂, Petroleum ether:Ethyl acetate=100:0 to 20:1) to give 83b (0.90 g, crude) as a white solid.

### Step 2:

To a solution of 83b (0.60 g, 2.13 mmol, 1.00 eq) in MeOH (15.0 mL) Pd/C (0.30 g, 2.13 mmol, 10% purity) was added under N₂ atmosphere. The system was degassed and then charged with H₂ three times. The reaction mixture was heated and stirred under H₂ (15 psi) at 50 °C for 0.5 hr. LCMS showed the starting material was consumed completely. After cooling, the mixture was filtered through a pad of Celite carefully and the filter cake was washed with MeOH (10 mL×2). The filtrate was concentrated under reduced pressure to dryness. The residue was purified by pre-HPLC (column: Phenomenex Luna C18 200*40 mm*10 um; mobile phase: [water(0.2% FA)-ACN]; B %: 1%-50%, 8 min) to afford the titled product Ex.83 (0.27 g, 67.3% yield) as a yellow oil.

### Example 84: Preparation of 3,7-difluoro-2-hydroxy-4-isopropylcyclohepta-2,4,6-trien-1-one (Ex.84)

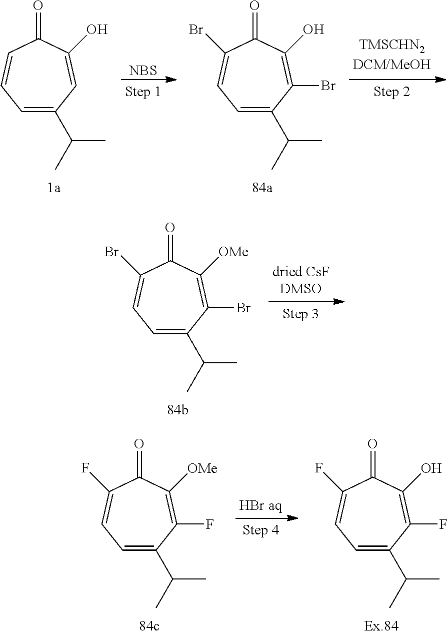

### Step 1:

To a solution of 1a (5.00 g, 30.4 mmol, 1.00 eq) in CHCl₃ (50.0 mL) NBS (11.3 g, 63.9 mmol, 2.10 eq) was added in one portion at 20 °C, and then the mixture was heated and stirred at 90 °C for 2 hr. LCMS showed no material remained. After cooling, the mixture was filtered and the filtrate was concentrated under reduced pressure to give 84a (17.0 g, crude) as a yellow oil, which was used in the next step.

### Step 2:

To a solution of 84a (17.0 g, 52.8 mmol, 1.00 eq) in DCM (170 mL) and MeOH (19.0 mL) DIPEA (10.2 g, 79.1 mmol, 13.7 mL, 1.50 eq) and TMSCHN₂ (2.00 M, 39.6 mL, 1.50 eq) were added at 0 °C. The mixture was warmed and stirred at 20 °C for 12 hrs. TLC (Petroleum ether/Ethyl acetate=5/1, R_{f} (product)=0.45, R_{f} (material)=0.15) showed no material remained. The mixture was quenched with water (20 mL) and extracted with DCM (30 mL×2). The combined organic phases were washed with brine (10 mL), dried over anhydrous Na₂SO₄, filtered and concentrated in vacuum to dryness. The residue was purified by column chromatography (SiO₂, Petroleum ether/Ethyl acetate=10/1 to 5/1) to get 84b (1.70 g, 5.06 mmol, 9.58% yield) as a yellow solid.

### Step 3:

To a solution of 84b (1.60 g, 4.76 mmol, 1.00 eq) in DMSO (16.0 mL) dried CsF (1.45 g, 9.52 mmol, 2.00 eq) was added in one portion at 25 °C under N₂. The mixture was heated to 100 °C and stirred for 2 hours. TLC (Petroleum ether/Ethyl acetate=1/1, R_{f}(material)=0.7, R_{f} (product)=0.4) showed 20% material remained. The residue was diluted with H₂O (20 mL) and extracted with EtOAc (50 mL×2). The combined organic layers were dried over Na₂SO₄, filtered and concentrated under reduced pressure to give a residue. The residue was purified by pre-HPLC (column: Phenomenex Luna C18 100*30 mm*5 um; mobile phase: [water(0.2% FA)-ACN]; B %: 50%-50%, 9 min) to give 84c (120 mg, 560 umol, 11.7% yield) as a yellow solid.

### Step 4:

The mixture of 84c (120 mg, 560 umol, 1.00 eq) in HBr (2.00 mL, 50% purity), was heated and stirred at 80 °C for 15 min. TLC (Petroleum ether/Ethyl acetate=1/1, R_{f} (material)=0.6, R_{f} (product)=0.25) showed no material remained. The mixture was concentrated under reduced pressure to give a residue. The crude product was purified by pre-HPLC (column Phenomenex Luna C18 100*30 mm*5 um; mobile phase: [water(0.2% FA)-ACN]; B %: 20%-50%, 9 min) to afford the titled product Ex.84 (19.0 mg, 94.4 umol, 16.8% yield, 99.5% purity) as a white solid. ¹HNMR: 400 MHz; S 7.37-7.45 (m, 1H), 6.96-7.00 (m, 1H), 3.48-3.58 (m, 1H), 1.30-1.32 (d, J=8.00 Hz, 1H); HPLC: MS+1: 201.2

### Example 85: Preparation of 7-chloro-2-hydroxy-3-methylcyclohepta-2,4,6-trien-1-one (Ex.85)

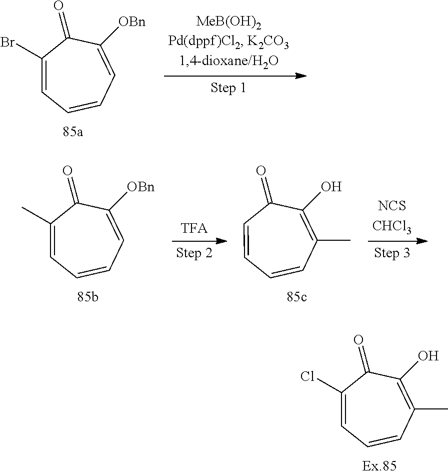

### Step 1:

To a mixture of 85a (5 g, 17.1 mmol, 1 eq), K₂CO₃ (4.75 g, 34.3 mmol, 2 eq) and methylboronic acid (10.3 g, 171 mmol, 10 eq) in dioxane (50 mL) and H₂O (10 mL) Pd(dppf)Cl₂ (1.26 g, 1.72 mmol, 0.1 eq) was added at 20 °C under N₂ atmosphere. The system was degassed and then charged with nitrogen three times. The mixture was heated and stirred at 120 °C for 0.5 hr. LCMS showed the starting material was consumed and the desired MS was detected. After cooling, water (30 mL) was added to the reaction mixture and then extracted with ethyl acetate (40 mL×2). The combined organic phases were washed with brine (50 mL), dried over anhydrous Na₂SO₄, filtered and concentrated in vacuum to dryness. The residue was purified by silica gel chromatography (Petroleum ether/Ethyl acetate=100/1 to 100/26) to give 85b (3.1 g, 39.9% yield) as a yellow solid.

### Step 2:

A solution of 85b (1.5 g, 6.63 mmol, 1 eq) in TFA (5 mL) was heated and stirred at 50 °C for 5 hr. LCMS showed the starting material was consumed and the desired MS was detected. The mixture was diluted with DCM (20 mL) and concentrated in vacuum to give crude product 85c (0.95 g, crude) as yellow oil.

### Step 3:

To a solution of 85c (900 mg, 6.61 mmol, 1 eq) in CHCl₃ (10 mL) NCS (882 mg, 6.61 mmol, 1 eq) was added in one portion at 25 °C. The mixture was heated to 65 °C and stirred for 10 hr. LCMS showed the starting material was consumed and the desired MS was detected. After cooling, saturated sodium thiosulfate (10 mL) was added to the mixture and extracted with DCM (10 mL×3). The combined organic phases were washed with brine (20 mL), dried over anhydrous Na₂SO₄, filtered and concentrated in vacuum. The residue was purified pre-HPLC (column: Phenomenex luna C18 250*50 mm*15 um; mobile phase: [water (0.2% FA)-ACN]; B %: 15%-45%, 20 min) to afford the titled product Ex.85 (0.23 g, 20.40% yield) as a yellow solid. ¹H NMR: 400 MHz; δ: 7.88 (d, J=10.5 Hz, 1H), 7.58 (d, J=10.4 Hz, 1H), 6.95 (t, J=10.4 Hz, 1H), 2.46 (s, 3H); HPLC: MS: 171.1

### Example 86: Preparation of 4-ethyl-2-fluoro-7-hydroxycyclohepta-2,4,6-trien-1-one (Ex.86)

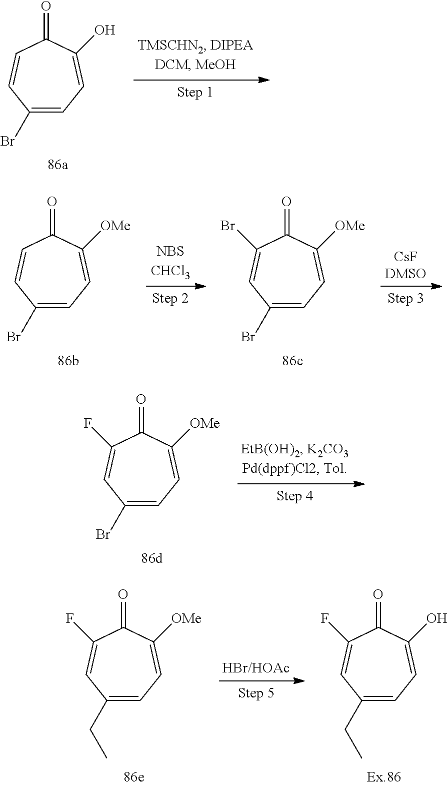

### Step 1:

To a solution of 86a (20 g, 99.4 mmol, 1 eq) in DCM (200 mL) and MeOH (22 mL) DIPEA (19.3 g, 149 mmol, 1.5 eq) was added and diazomethyl(trimethyl)silane (2 M, 99.5 mL, 2 eq) at 0 °C. The reaction mixture was stirred at 25 °C for 16 hr. TLC (Petroleum ether:Ethyl acetate=0:1) showed the starting material was consumed completely and a new spot was observed. The reaction mixture was quenched by addition of H₂O (50 mL) at 0 °C, and then extracted with DCM (50 mL×3). The combined organic phases were washed with brine (50 mL), dried over anhydrous Na₂SO₄, filtered and concentrated under reduced pressure to give a residue. The residue was purified by column chromatography (SiO₂, Petroleum ether:Ethyl acetate=100:0 to 1:1) to give 86b (13 g, 60.4 mmol, 60.8% yield) as a yellow solid.

### Step 2:

To a solution of 86b (6.5 g, 30.2 mmol, 1 eq) in CHCl₃ (200 mL) NBS (43.04 g, 242 mmol, 8 eq) was added at 20 °C. The system was degassed and charged with nitrogen three times and then heated to 100 °C and stirred for 16 hrs. TLC (Petroleum ether:Ethyl acetate=3:1) showed the starting material was consumed completely and a new spot observed. After cooling, water (100 mL) was added and then extracted with CHCl₃ (100 mL×2). The combined organic phases were washed with brine (100 mL), dried over anhydrous Na₂SO₄, filtered and concentrated under reduced pressure to dryness to give 86c (2 g, 22.5% yield) as a yellow solid.

### Step 3:

To a solution of 86c (1.5 g, 5.10 mmol, 1 eq) in DMSO (5 mL) dried CsF (1.55 g, 10.21 mmol, 2 eq) was added. The system was degassed and charged with nitrogen three times and then heated to 100 °C and stirred for 0.5 hr. TLC (Petroleum ether:Ethyl acetate=3:1) showed the starting material was consumed completely and a new spot observed. After cooling, water (50 mL) was added and then extracted with ethyl acetate (100 mL×3). The combined organic phases were washed with brine (50 mL), dried over anhydrous Na₂SO₄, filtered and concentrated under reduced pressure to dryness. The residue was purified by column chromatography (SiO₂, Petroleum ether:Ethyl acetate=1:0 to 3:1) to give (500 mg, 2.15 mmol, 42.0% yield) as a yellow solid.

### Step 4:

To a solution of 86d (100 mg, 429 umol, 1 eq) in toluene (2 mL) K₂CO₃ (148 mg, 1.07 mmol, 2.5 eq), ethylboronic acid (317 mg, 4.29 mmol, 10 eq) and Pd(dppf)Cl₂·CH₂Cl₂ (70 mg, 85 umol, 0.2 eq) were added under N₂. The system was degassed and charged with nitrogen three times. The reaction mixture was heated and stirred at 100 °C for 0.5 hr. TLC (Petroleum ether:Ethyl acetate=3:1) showed the starting material was consumed completely and a new spot was observed. After cooling, the reaction mixture was filtered and concentrated under reduced pressure to give a residue. The residue was purified by column chromatography (SiO₂, Petroleum ether:Ethyl acetate=1:0 to 1:1) to give 86e (55 mg, 823 umol, 38.4% yield) as a yellow solid.

### Step 5:

A solution of 86e (0.05 g, 274 umol, 1 eq) in HBr/HAc (1.5 mL) was heated and stirred at 100 °C for 0.5 hr. TLC (Petroleum ether:Ethyl acetate=3:1) showed the starting material was consumed completely and a new spot was observed. After cooling, the reaction mixture was filtered and concentrated under reduced pressure to give a residue. The residue was purified by prep-HPLC (FA condition; column: 3_Phenomenex Luna C18 75*30 mm*3 um; mobile phase: [water(0.2% FA)-ACN]; B %: 15%-35%, 7 min) to afford the titled product Ex.86 (16 mg, 34.6% yield) as a white solid. ¹HNMR: 400 MHz; HPLC: MS (M+1):169

### Examples 87 and 88: Preparation of 2-fluoro-7-hydroxy-4-(1-methylcyclopropyl)cyclohepta-2,4,6-trien-1-one (Ex.87) and 2-hydroxy-5-(1-methylcyclopropyl)cyclohepta-2,4,6-trien-1-one (Ex.88)

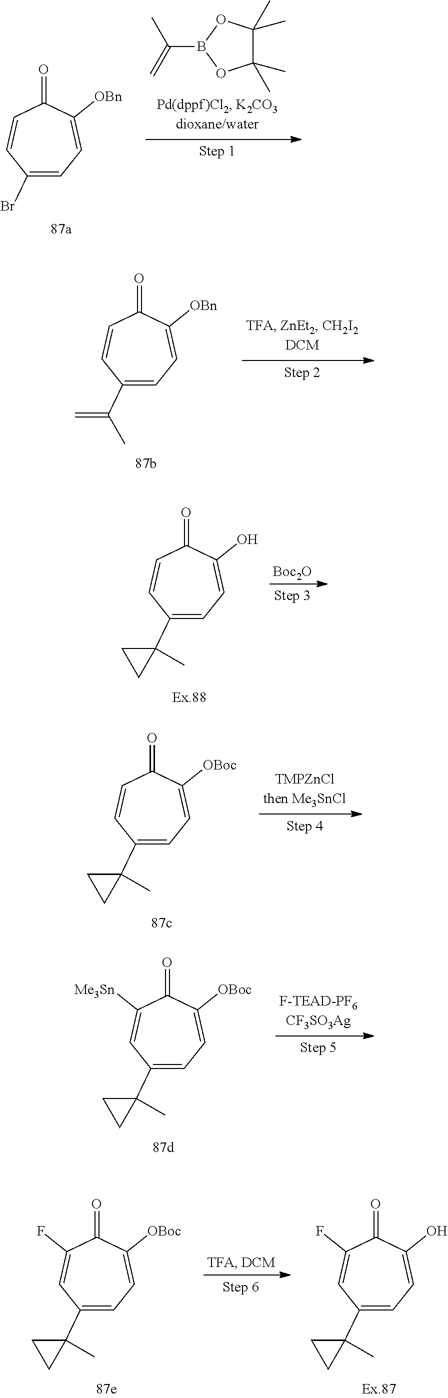

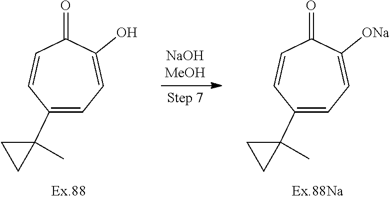

### Step 1:

To a mixture of 87a (10.0 g, 34.3 mmol, 1.00 eq) and 2-isopropenyl-4,4,5,5-tetramethyl-1,3,2-dioxaborolane (8.66 g, 51.5 mmol, 1.50 eq) in dioxane (80 mL) and H₂O (15 mL) Pd(dppf)Cl₂ (1.12 g, 1.37 mmol, 0.04 eq) and K₂CO₃ (9.97 g, 72.1 mmol, 2.10 eq) were added at 20 °C under N₂ atmosphere. The system was degassed and then charged with nitrogen three times. The mixture was heated and stirred at 120 °C for 2 hr. TLC showed the starting material was consumed completely and a new spot was observed. After cooling, the mixture was filtered through a pad of Celite and the filter cake was washed with CH₂Cl₂ (30 mL×3). The filtrate was concentrated under reduced pressure to dryness. The residue was purified by silica gel chromatography (Petroleum ether/Ethyl acetate=90/1 to 80/20) to give 87b (5.30 g, 48.9% yield, 80.0% purity) as a yellow solid.

### Step 2:

A solution of ZnEt₂ (1 M in hexane, 31.0 mL, 3.00 eq) in DCM (30 mL) a solution of TFA (3.52 g, 30.9 mmol, 2.29 mL, 3.00 eq) in DCM (5 mL) was added slowly and carefully at 0 °C. After stirring for 20 min, a solution of CH₂I₂ (8.28 g, 30.9 mmol, 2.49 mL, 3.00 eq) in DCM (5 mL) was added to the above mixture and stirred for another 20 min. Then the resulting mixture was treated with a solution of 87b (2.60 g, 10.3 mmol, 1.00 eq) in DCM (10 mL) at 0 °C. The reaction mixture was allowed to slowly warm to 15 °C and stirred for 20 hr. TLC showed the starting material was consumed completely and new spot was observed. Water (50 mL) was added into and then extracted with DCM (30 mL×3). The combined organic phases were washed with brine (100 mL), dried over anhydrous Na₂SO₄, filtered and concentrated under reduced pressure to give a residue. The residue was purified by prep-HPLC (column: Phenomenex Luna C18 200*40 mm*10 um; mobile phase: [water (0.2% FA)-ACN]; B %: 40%-80%, 8 min) to give Ex.88 (0.40 g, 22% yield) as brown oil.

### Step 3:

To a mixture of Ex.88 (0.40 g, 2.27 mmol, 1.00 eq) and Boc₂O (1.49 g, 6.81 mmol, 1.56 mL, 3.00 eq) in dioxane (5 mL) was added TEA (1.15 g, 11.3 mmol, 1.58 mL, 5.00 eq) at 20 °C. The mixture was heated and stirred at 110 °C for 2 hr. TLC showed the starting material was consumed completely and new spot was observed. The mixture was concentrated under reduced pressure to give a residue. The residue was purified by silica gel chromatography (Petroleum ether/Ethyl acetate=100/0-90/10) to give 87c (0.40 g, 51.0% yield, 80% purity) as brown oil.

### Step 4:

To a solution of 87c (0.30 g, 1.09 mmol, 1.00 eq) in THF (7 mL) chloro(trimethyl)stannane (324 mg, 1.63 mmol, 1.50 eq) and TMPZnCl·LiCl (0.4 M in THF, 8.14 mL, 3.00 eq) was added at 0 °C under N₂. The reaction mixture was heated and stirred at 50 °C for 2 hr. TLC showed the starting material was consumed completely and a new spot was observed. After cooling, water (10 mL) was added and then extracted with ethyl acetate (10 mL×3). The combined organic phases were washed with brine (10 mL), dried over anhydrous Na₂SO₄, filtered and concentrated under reduced pressure to dryness. The residue was purified by silica gel chromatography (Petroleum ether/Ethyl acetate=100/0-95/5) to give 87d (0.18 g, 30.2% yield) as a yellow oil.

### Step 5:

To a solution of 87d (0.15 g, 341 umol, 1.00 eq) in CH₃CN (3 mL) CF₃SO₃Ag (175 mg, 683 umol, 2.00 eq) and F-TEAD-PF₆ (605 mg, 1.71 mmol, 5.00 eq) were added at 0 °C under N₂. The mixture was stirred at 20 °C for 1 hr. TLC showed the starting material was consumed completely and new spot was observed. The mixture was poured into water (10 mL) and extracted with ethyl acetate (10 mL×3). The combined organic phases were washed with brine (20 mL), dried over anhydrous Na₂SO₄, filtered and concentrated under reduced pressure to give a residue. The residue was purified by prep-TLC (Petroleum ether/Ethyl acetate=3:1 Rf=0.5) to give 87e (10 mg, 9.55% yield, 96% purity) as a yellow oil.

### Step 6:

To a solution of 87e (0.01 g, 33.9 umol, 1.00 eq) in DCM (1 mL) TFA (385 mg, 3.38 mmol, 0.25 mL, 99.4 eq) was added at 15 °C. The mixture was stirred at 15 °C for 0.5 hr. TLC showed the starting material was consumed completely and new spot was observed. The reaction mixture was diluted with CH₂Cl₂ (10 mL) and concentrated under reduced pressure to dryness below 10 °C. Then the mixture was re-dissolved in CH₂Cl₂ (5 mL), Amberlyst A21 (0.1 g) was added and the mixture was stirred at 25 °C for another 0.5 hr. After filtering, the cake was washed with CH₂Cl₂ (5 mL×2) and the filtrate was concentrated under reduced pressure to afford the titled product Ex.87 (4.00 mg, 59.2% yield, 97.7% purity) as a yellow solid

### Step 7:

To a solution of Ex.88 (15.0 mg, 85.1 umol, 1.00 eq) in MeOH (1 mL) NaOH (5 M, 17.0 uL, 1.00 eq) was slowly added. Then the solution was stirred at 20 °C for 1 hr. The reaction mixture was concentrated under reduced pressure to remove MeOH. The crude product was triturated with acetone (5 mL) at 25 °C and stirred for another 30 min. After filtering, the cake was washed with acetone (5 mL×2) and the precipitate was collected and dried in vacuum to afford the titled product Ex.88Na (13.0 mg, 75.5% yield, 98% purity) as a yellow solid. ¹H NMR: 400 MHz; δ ppm 7.23 (d, J=12.0 Hz, 2H), 6.97-6.90 (m, 2H), 1.30 (s, 3H), 0.77-0.71 (m, 2H), 0.65 (s, 2H)

### Example 89: Preparation of 4-fluoro-2-hydroxy-7-isopropylcyclohepta-2,4,6-trien-1-one (Ex.89)

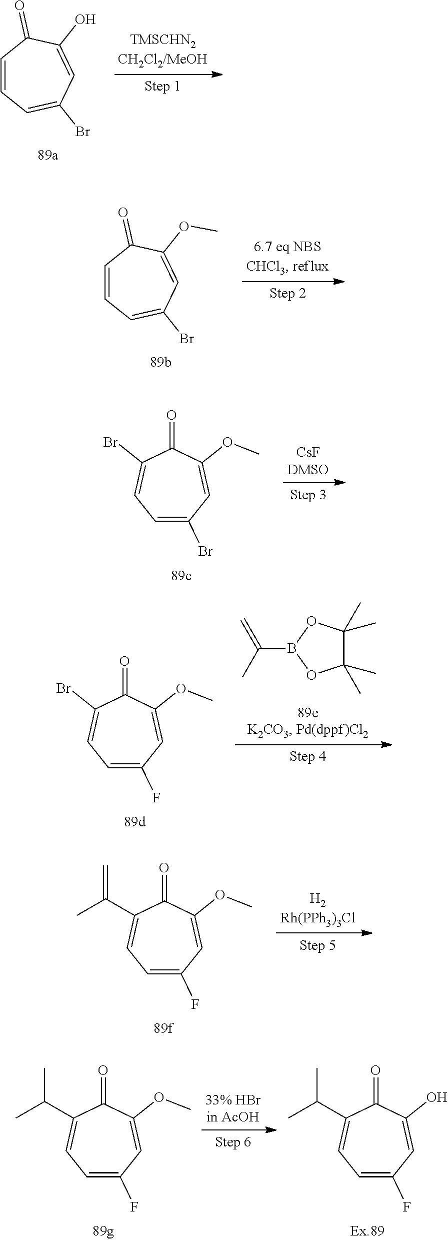

### Step 1:

To a solution of 89a (10.0 g, 49.7 mmol, 1.00 eq) in DCM (100 mL) and MeOH (12.0 mL) DIPEA (9.64 g, 74.6 mmol, 13.0 mL, 1.50 eq) and TMSCHN₂ (2 M, 54.7 mL, 2.20 eq) were added in one portion at 0 °C. The mixture was warmed and stirred at 20 °C for 5 hours. LCMS showed the reaction was completed. The mixture was quenched with water (350 mL) and extracted with DCM (150 mL×3). The combined organic phases were washed with brine (100 mL), dried over anhydrous Na₂SO₄, filtered and concentrated under reduced pressure to give a residue. The crude product was purified by column chromatography (SiO₂, Petroleum ether/Ethyl acetate=100/0 to 61/39) to give compound 89b (3.67 g, 17.1 mmol, 34.2% yield) as a yellow solid.

### Step 2:

To a mixture of 89b (2.00 g, 9.30 mmol, 1.00 eq) and in CHCl₃ (20.0 mL) NBS (11.1 g, 62.3 mmol, 6.70 eq) was added in one portion at 25 °C under N₂. The mixture was heated and stirred at 75 °C for 13 hours. LCMS showed the reaction was completed. The mixture was concentrated under reduced pressure to give a residue. The residue was purified by column chromatography (SiO₂, Petroleum ether/Ethyl acetate=100/0 to 86/14) to give compound 89c (2.30 g, 7.82 mmol, 84.1% yield) as a yellow solid.

### Step 3:

To a solution of 89c (4.70 g, 15.9 mmol, 1.00 eq) in DMSO (30 mL) was added to dried CsF (4.86 g, 31.9 mmol, 2.00 eq) in one portion at 25 °C under N₂. The mixture was heated to 100 °C and stirred for 40 mins. TLC (Petroleum ether/Ethyl acetate=1/1, R_{f} (material)=0.45, R_{f} (product)=0.35) showed the reaction was completed. The reaction mixture was extracted with Ethyl acetate 450 mL (150 mL×3). The combined organic layers were dried over Na₂SO₄, filtered and concentrated under reduced pressure to give a residue. The residue was purified by column chromatography (SiO₂, Petroleum ether/Ethyl acetate=100/0 to 80/20) to give compound 89d (2.70 g, 11.6 mmol, 72.4% yield) as a yellow solid.

### Step 4:

To a mixture of 89d (2.70 g, 11.6 mmol, 1.00 eq) and K₂CO₃ (3.20 g, 23.1 mmol, 2.00 eq) in dioxane (25 mL) and H₂O (5 mL) 89e (3.89 g, 23.2 mmol, 2.00 eq) was added in one portion at 20 °C under N₂. Then Pd(dppf)Cl₂·CH₂Cl₂ (1.89 g, 2.32 mmol, 0.20 eq) was added to the mixture. The system was degassed and then charged with nitrogen three times. The mixture was heated and stirred at 118 °C for 90 mins. TLC (Petroleum ether/Ethyl acetate=1/1, R_{f} (material)=0.55, R_{f} (product)=0.60) showed the reaction was completed. After cooling, the mixture was extracted with ethyl acetate (100 mL×3). The combined organic phases were washed with brine (50 mL), dried over anhydrous Na₂SO₄, filtered and concentrated under reduced pressure to give a residue. The residue was purified by column chromatography (SiO₂, Petroleum ether/Ethyl acetate=100/0 to 80/20) to give compound 89f (854 mg, 4.40 mmol, 37.9% yield) as a yellow oil.

### Step 5:

To a solution of 89f (854 mg, 4.40 mmol, 1.00 eq) in MeOH (8.00 mL) Rh(PPh₃)₃C1 (533 mg, 577 umol, 0.13 eq) was added. The suspension was degassed under vacuum and purged with H₂ several times. The mixture was stirred under H₂ (30 psi) at 30 °C for 40 mins. TLC (Petroleum ether/Ethyl acetate=3/1, R_{f} (material)=0.30, R_{f} (product)=0.40) showed the starting material was consumed completely. The mixture was filtered and the filtrate was concentrated under reduce pressure to dryness. The residue was purified by column chromatography (SiO₂, Petroleum ether/Ethyl acetate=100/0 to 80/20) to give compound 89g (0.20 g, 1.02 mmol, 23.1% yield) as yellow oil.

### Step 6:

89g (200 mg, 1.02 mmol, 1.00 eq) was added to hydrogen bromide (1.49 g, 18.4 mmol, 1.00 mL, 18.1 eq) in one portion at 25 °C. The mixture was stirred at 100 °C for 30 mins. LCMS showed the reaction was completed. MTBE (3 mL) was added to the mixture and then concentrated under reduced pressure to dryness. The residue was triturated with MTBE (2 mL) to afford the titled product Ex.89 (101 mg, 554 umol, 54.4% yield) as a yellow solid. ¹H NMR: 400 MHz; S 1.17 (d, J=6.90 Hz, 6H) 3.46-3.62 (m, 1H) 6.94-7.08 (m, 1H) 7.17 (dd, J=18.51, 2.95 Hz, 1H) 7.53 (dd, J=11.23, 4.33 Hz, 1H); HPLC: MS: (M+1): 183.0

### Example 90: Preparation of 5-fluoro-2-hydroxy-3-methylcyclohepta-2,4,6-trien-1-one (Ex.90)

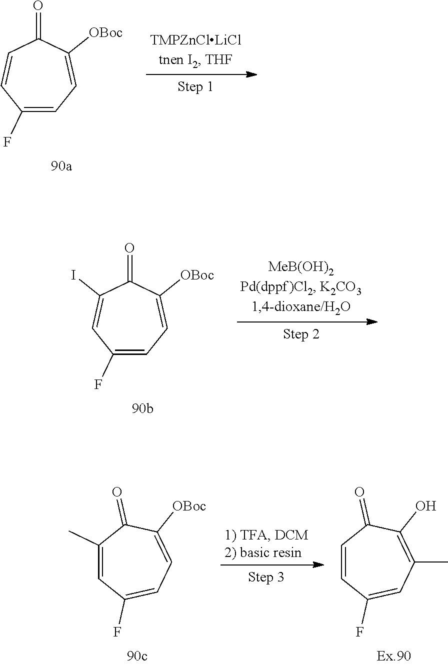

### Step 1:

To a solution of 90a (500 mg, 2.08 mmol, 1 eq) in THF (5 mL) chloro-(2,2,6,6-tetramethyl-1-piperidyl)zinc (0.4 M in THF, 13 mL, 2.5 eq) was added at 0 °C under N₂. After 30 min, I₂ (792 mg, 3.12 mmol, 1.5 eq) in THF (1 mL) was added to above mixture and then stirred at 0 °C for another 30 min under N₂ atmosphere. LC-MS showed 90a was consumed completely and one main peak with desired mass was detected. The reaction mixture was quenched by addition of H₂O (10 mL) at 15 °C, and then extracted with EtOAc (20 mL×4). The combined organic layers were washed with brine (10 mL), dried over Na₂SO₄, filtered and concentrated under reduced pressure to give a residue. The residue was purified by column chromatography (SiO₂, Petroleum ether:Ethyl acetate=85:1 to 15:1) to give compound 90b (731 mg, 95.9% yield) as a yellow solid.

### Step 2:

To a mixture of Core3_7F (300 mg, 819 umol, 1 eq), methylboronic acid (490 mg, 8.19 mmol, 10 eq), K₂CO₃ (283 mg, 2.05 mmol, 2.5 eq) in dioxane (3 mL) and H₂O (0.4 mL) Pd(dppf)Cl₂·CH₂Cl₂ (67 mg, 81.9 umol, 0.1 eq) was added under N₂ atmosphere. The system was degassed and then charged with nitrogen three times. The reaction mixture was heated and stirred at at 110 °C for 30 min under N₂ atmosphere. LC-MS showed 90b was consumed completely and one main peak with desired mass was detected. After cooling, the reaction mixture was quenched by addition H₂O (10 mL) and then extracted with EtOAc (20 mL×4). The combined organic layers were washed with H₂O (10 mL), brine (10 mL), dried over Na₂SO₄, filtered and concentrated under reduced pressure to give a residue. The residue was purified by prep-HPLC (FA condition) to give compound 90c (40 mg, 19.2% yield) as a yellow solid.

### Step 3:

To a solution of 90c (40 mg, 157 umol, 1 eq) in CH₂Cl₂ (1 mL) TFA (0.2 mL, 1 eq) was added in one portion at 25 °C. The reaction mixture was stirred at 25 °C for 30 min. LC-MS showed 90c was consumed completely and one main peak with desired mass was detected. The reaction mixture was diluted with CH₂Cl₂ (10 mL) and concentrated under reduced pressure to dryness below 10 °C. Then the mixture was re-dissolved in CH₂Cl₂ (5 mL) and added Amberlyst A21 (0.1 g) and stirred at 25 °C for another 0.5 hr. After filtering, the cake was washed with CH₂Cl₂ (5 mL×2) and the filtrate was concentrated under reduced pressure to afford the titled product Ex.90 (10 mg, 62% yield) as a yellow solid. ¹H NMR: 400 MHz; δ: 2.44 (s, 3H) 7.15-7.38 (m, 2H) 7.62 (dd, J=17.2, 2.4 Hz, 1H); HPLC: MS (M+H): 155.1

### Example 91: Preparation of 5-fluoro-2-hydroxy-3-(tetrahydrofuran-2-yl)cyclohepta-2,4,6-trien-1-one (Ex.91)

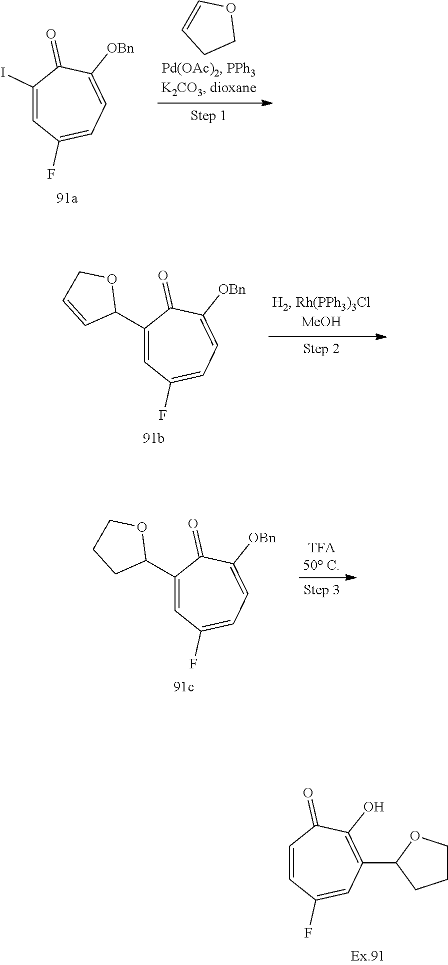 [SMILES: O=c1c(I)cc(F)ccc1OCc1ccccc1 ; C1=COCC1 ; O=c1c(OCc2ccccc2)ccc(F)cc1C1C=CCO1 ; O=c1c(OCc2ccccc2)ccc(F)cc1C1CCCO1 ; O=c1ccc(F)cc(C2CCCO2)c1O]

### Step 1:

To a mixture of 91a (0.31 g, 873 umol, 1 eq), K₂CO₃ (24 mg, 1.75 mmol, 2 eq) and 2,3-dihydrofuran (244 mg, 3.49 mmol, 4 eq) in dioxane (7 mL) PPh₃ (46 mg, 175 umol, 0.2 eq) and Pd(OAc)₂ (19 mg, 87 umol, 0.1 eq) were added in one portion at 25 °C under under N₂ atmosphere. The system was degassed and then charged with nitrogen three times. The mixture was heated and stirred at 110 °C for 30 min. LC-MS showed 91a was consumed completely and one main peak with desired mass was detected. After cooling, the reaction mixture was quenched by addition of H₂O (10 mL) at 25 °C, and then extracted with EtOAc (10 mL×3). The combined organic layers were washed with brine (10 mL), dried over Na₂SO₄, filtered and concentrated under reduced pressure to give a residue. The residue was purified by column chromatography (SiO₂, Petroleum ether/Ethyl acetate=100/1 to 10/1) to give 91b (100 mg, 38.4% yield) as a yellow oil.

### Step 2:

To a solution of 91b (100 mg, 335 umol, 1 eq) in MeOH (5 mL) Rh(PPh₃)₃C1 (0.05 g, 54 umol, 0.16 eq) was added under N₂. The suspension was degassed under vacuum and purged with H₂ several times. The mixture was stirred under H₂ (15 psi) at 30 °C for 1 hr. LC-MS showed 91b was consumed completely and one main peak with desired mass was detected. The mixture was filtered through a pad of Celite and the filter cake was washed with MeOH (5 mL×2). The filtrate was concentrated under reduced pressure to dryness. The residue was purified by prep-TLC (SiO₂, Petroleum ether/Ethyl acetate=3/1) to give 91c (53 mg, 52.6% yield) as a yellow oil.

### Step 3:

A solution of 91c (53 mg, 176 umol, 1 eq) in TFA (1 mL) was heated and stirred at 50 °C for 1 hr. LC-MS showed 91c was consumed completely and one main peak with desired mass was detected. The reaction mixture was diluted with DCM (10 mL) and concentrated under reduced pressure to give a reside, which was purified by prep-HPLC (FA condition column: 3_Phenomenex Luna C18 75*30 mm*3 um; mobile phase: [water(0.2% FA)-ACN]; B %: 20%-40%, 7 min) to afford the titled product Ex.91 (25 mg, 67.4% yield) as a yellow solid. ¹H NMR: 400 MHz; δ: 1.51-1.67 (m, 1H) 1.85-2.09 (m, 2H) 2.56-2.71 (m, 1H) 3.90-4.03 (m, 1H) 4.10-4.20 (m, 1H) 5.20 (t, J=7.2 Hz, 1H) 7.17-7.33 (m, 2H) 7.77 (dd, J=18.0, 2.8 Hz, 1H); HPLC: MS: 211.1

### Example 92 and 93: Preparation of 2-hydroxy-3-((2S.5R)-5-methyltetrahydrofuran-2-yl)cyclohepta-2,4,6-trien-1-one (Ex.92) and 2-hydroxy-3-((2R.5R)-5-methyltetrahydrofuran-2-yl)cyclohepta-2,4,6-trien-1-one (Ex.93)

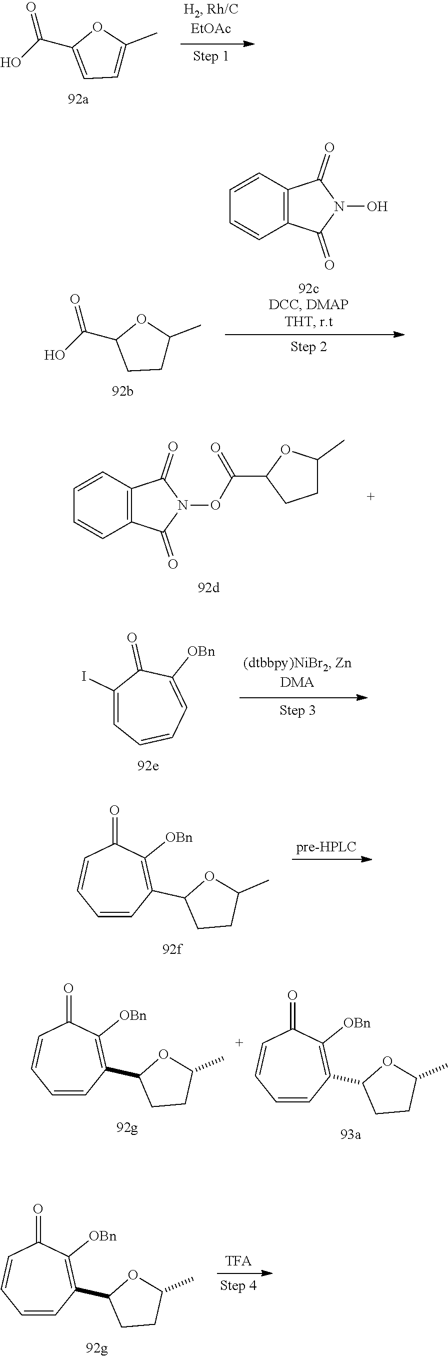

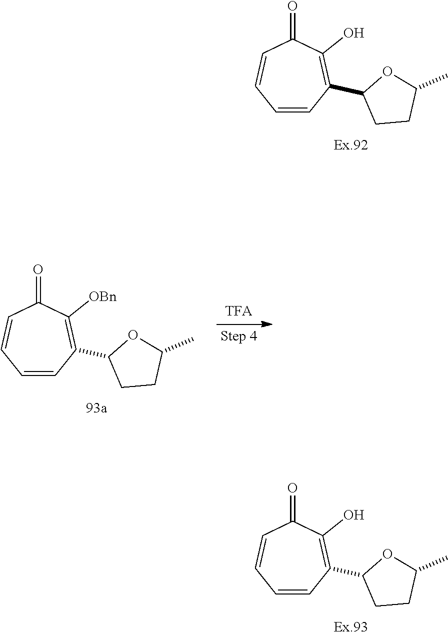

### Step 1:

To a solution of 92a (2.00 g, 15.8 mmol, 1.00 eq) in EtOAc (25 mL) Rh/C (1.87 g, 15.8 mmol, 1.00 eq) was added under N₂ atmosphere. The system was degassed and then charged with H₂ three times. The reaction mixture was stirred under H₂ (30 psi) at 25 °C for 12 hours. New spot was observed on TLC (EtOAc, R_{f}=0.15). The mixture was filtered through a pad of Celite and the filter cake was washed with EtOAc (30 mL×3). The combined filtrates were concentrated in vacuo to give the compound 92b (1.20 g, 9.22 mmol, 58.1% yield) as colorless gum.

### Step 2:

To solution of 92b (700 mg, 4.29 mmol, 1.10 eq) in dry THF (10 mL) DCC (965 mg, 4.68 mmol, 1.20 eq) and DMAP (47.6 mg, 390 umol, 0.10 eq) were added at 25 °C. After stirring for 5 min, 92c (507 mg, 3.90 mmol, 1.00 eq) was added and the reaction mixture was stirred at 25 °C for 24 h. A new spot was observed on TLC (Petroleum ether:Ethyl acetate=5:1, R_{f}=0.35). The precipitate was filtered off and the filtration was concentrated by evaporation of the solvent to dryness. The reside was purified by flash silica column chromatography (Petroleum ether:Ethyl acetate=10:1 to 5:1) to afford the desired product 92d (600 mg, 55.8% yield) as a white solid.

### Step 3:

To a mixture of 92e (200 mg, 590 umol, 1.00 eq) and 92d (195 mg, 708 umol, 1.20 eq) in DMA (4.00 mL) Zn (77.2 mg, 1.18 mmol, 2.00 eq) and (dtbbpy)NiBr₂ (80.0 mg, 41.4 umol, 0.07 eq) were added at 0 °C under N₂ atmosphere. The system was degassed and then charged with nitrogen three times. The mixture was warmed and stirred at 25 °C for 1.5 hours. Desired MS was observed on LCMS. Brine (10 mL) was added to the mixture and extracted with EtOAc (20 mL×2), the combined original layers were concentrated under reduced pressure to give a yellow gum. The residue was purified by silica column chromatography (Petroleum ether:Ethyl acetate=10/1) to give 92f (10 mg, 5.70% yield) as a light yellow gum. 10 parallel reactions were carried out to give ~100 mg racemic product 92f and then separated by pre-HPLC (TFA condition) to obtain 40 mg of 92g and 25 mg of 93a respectively.

### Step 4:

Taking 92g as an example: A solution of 92f (60.0 mg) in TFA (2.00 mL) was stirred at 45 °C for 1 hr. Desired MS was observed on LCMS. After diluting with DCM (10 mL), the yellow solution was concentrated under reduced pressure to dryness. The residue was purified by pre-HPLC (column: 3_Phenomenex Luna C18 75*30 mm*3 um; mobile phase: [water (0.2% FA)-ACN]; B %: 20%-40%, 7 min) to afford the titled product Ex.92 (13.0 mg, 31.1% yield) as yellow solid. ¹HNMR: 400 MHz; HPLC: MS: 207.2. The titled product Ex.93 was similarly produced.

### Example 94: Preparation of 3-ethyl-5-fluoro-2-hydroxycyclohepta-2,4,6-trien-1-one (Ex.94)

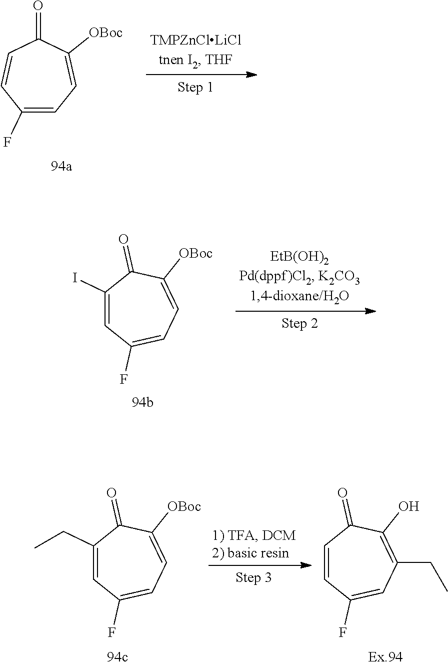

### Step 1:

To a solution of 94a (500 mg, 2.08 mmol, 1 eq) in THF (5 mL) chloro-(2,2,6,6-tetramethyl-1-piperidyl)zinc (0.4 M in THF, 13 mL, 2.5 eq) was added at 0 °C under N₂. After 30 min, I₂ (792 mg, 3.12 mmol, 1.5 eq) in THF (1 mL) was added to above mixture and then stirred at 0 °C for another 30 min under N₂ atmosphere. LC-MS showed 94a was consumed completely and one main peak with desired mass was detected. The reaction mixture was quenched by addition of H₂O (10 mL) at 15 °C, and then extracted with EtOAc (20 mL×4). The combined organic layers were washed with brine (10 mL), dried over Na₂SO₄, filtered and concentrated under reduced pressure to give a residue. The residue was purified by column chromatography (SiO₂, Petroleum ether:Ethyl acetate=85:1 to 15:1) to give compound 94b (731 mg, 95.9% yield) as a yellow solid.

### Step 2:

To a mixture of 94b (0.17 g, 464 umol, 1 eq), ethylboronic acid (343 mg, 4.64 mmol, 10 eq) and K₂CO₃ (128 mg, 928 umol, 2 eq) in toluene (2 mL) Pd(dppf)Cl₂·CH₂Cl₂ (37.9 mg, 46.4 umol, 0.1 eq) was added under N₂ atmosphere. The system was degassed and then charged with nitrogen three times. The reaction mixture was heated and stirred at 110 °C for 30 min under N₂ atmosphere. LC-MS showed 94b was consumed completely and one main peak with desired mass was detected. After cooling, the reaction mixture was quenched by addition H₂O (10 mL) and then extracted with EtOAc (20 mL×4). The combined organic layers were washed with H₂O (10 mL), brine (10 mL), dried over Na₂SO₄, filtered and concentrated under reduced pressure to give a residue. The residue was purified by prep-HPLC (TFA condition) to give compound 94c (80 mg, 64.2% yield) as a yellow solid.

### Step 3:

To a solution of 94c (30 mg, 112 umol, 1 eq) in CH₂Cl₂ (1 mL) TFA (0.2 mL) was added in one portion at 25 °C. The reaction mixture was stirred at 25 °C for 30 min. LC-MS showed 94c was consumed completely and one main peak with desired mass was detected. The reaction mixture was diluted with CH₂Cl₂ (10 mL) and concentrated under reduced pressure to dryness below 10 °C. Then the mixture was re-dissolved in CH₂Cl₂ (5 mL) and added Amberlyst A21 (0.1 g) and stirred at 25 °C for another 0.5 hr. After filtering, the cake was washed with CH₂Cl₂ (5 mL×2) and the filtrate was concentrated under reduced pressure to afford the titled product Ex.94 (13 mg, 69.1% yield) as a yellow solid. ¹H NMR: 400 MHz; δ: 1.24 (t, J=7.6 Hz, 3H) 2.85 (q, J=7.6 Hz, 2H) 7.14-7.34 (m, 2H) 7.55 (dd, J=17.6, 2.4 Hz, 1H); HPLC: MS (M+H): 169.1

### Example 95: Preparation of 7-chloro-3-ethyl-2-hydroxycyclohepta-2,4,6-trien-1-one (Ex.95)

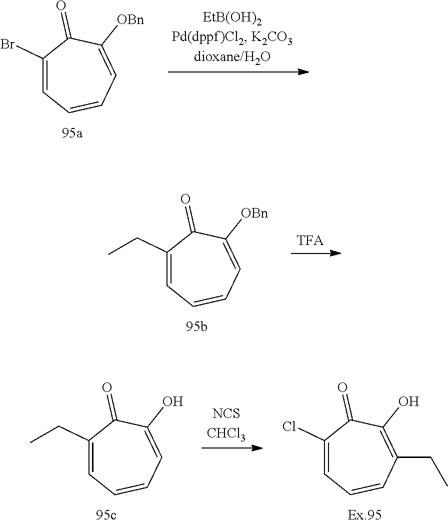 [SMILES: O=c1c(Br)ccccc1OCc1ccccc1 ; CCc1ccccc(OCc2ccccc2)c1=O ; CCc1ccccc(O)c1=O ; CCc1cccc(Cl)c(=O)c1O]

### Step 1:

To a mixture of 95a (3.00 g, 10.3 mmol, 1.00 eq), ethylboronic acid (7.61 g, 103 mmol, 10.0 eq) and K₂CO₃ (3.56 g, 25.76 mmol, 2.50 eq) in toluene (30 mL) Pd(dppf)Cl₂·CH₂Cl₂ (841 mg, 1.03 mmol, 0.10 eq) was added at 20 °C under N₂. The system was degassed and then charged with nitrogen for three times. Then the mixture was heated and stirred at 110 °C for 30 min under N₂ atmosphere. LC-MS showed 95a was consumed completely and one main peak with desired mass was detected. After cooling to the room temperature, the reaction mixture was quenched by addition of H₂O (10 mL), and then extracted with EtOAc (20 mL×4). The combined organic layers were washed with H₂O (10 mL), brine (10 mL), dried over Na₂SO₄, filtered and concentrated under reduced pressure to give a residue. The residue was purified by column chromatography (SiO₂, Petroleum ether/Ethyl acetate=85/1 to 15/1). Compound 95b (2.00 g, 80.8% yield) was obtained as a yellow oil.

### Step 2:

A solution of 95b (1.00 g, 4.16 mmol, 1.00 eq) in TFA (3 mL) was heated and stirred at 50 °C for 5 hr. TLC (petroleum ether:ethyl acetate=3:1, R_{f} (product)=0.5) indicated 95b was consumed completely and one new spot formed. The reaction mixture was diluted with DCM (10 mL) and concentrated under reduced pressure to give crude compound 95c (600 mg, crude) as a yellow oil.

### Step 3:

To a solution of 95c (600 mg, 4.00 mmol, 1.00 eq) in CHCl₃ (6 mL) was added NCS (1.60 g, 12.0 mmol, 3.00 eq) in one portion at 15 °C under N₂. The mixture was heated to 70 °C and stirred for 30 min. LC-MS showed 95c was consumed completely and one main peak with desired mass was detected. After cooling to the room temperature, the reaction mixture was quenched by addition of H₂O (10 mL), and then extracted with EtOAc (20 mL×4). The combined organic layers were washed with brine (10 mL), dried over Na₂SO₄, filtered and concentrated under reduced pressure to give a residue. The residue was purified by prep-HPLC (TFA condition) to afford the titled product Ex.95 (120 mg, 16.3% yield) as a yellow solid. ¹H NMR: 400 MHz; δ 1.24 (t, J=7.6 Hz, 3H) 2.87 (q, J=7.6 Hz, 2H) 4.89 (s, 2H) 6.98 (t, J=10.4 Hz, 1H) 7.55 (d, J=10.4 Hz, 1H) 7.89 (dd, J=10.4, 0.68 Hz, 1H); HPLC: MS (M+H): 185.1

### Example 96: Preparation of 4-cyclobutoxy-2-fluoro-7-hydroxycyclohepta-2,4,6-trien-1-one (Ex.96)

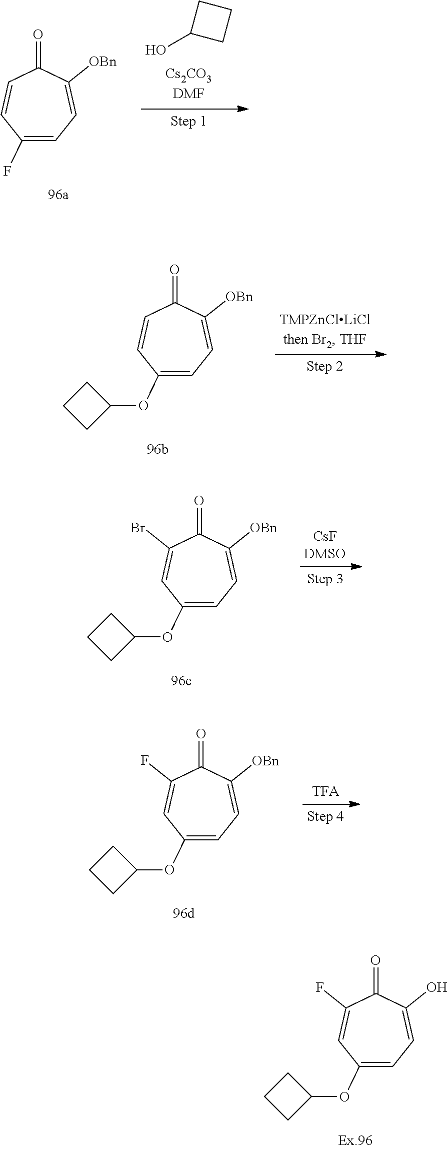 [SMILES: O=c1ccc(F)ccc1OCc1ccccc1 ; OC1CCC1 ; O=c1ccc(OC2CCC2)ccc1OCc1ccccc1 ; O=c1c(Br)cc(OC2CCC2)ccc1OCc1ccccc1 ; O=c1c(F)cc(OC2CCC2)ccc1OCc1ccccc1 ; O=c1c(O)ccc(OC2CCC2)cc1F]

### Step 1:

To a mixture of 96a (0.90 g, 3.91 mmol, 1.00 eq) and cyclobutanol (1.41 g, 20.0 mmol, 5.00 eq) in DMF (10 mL) Cs₂CO₃ (3.82 g, 11.7 mmol, 3.00 eq) was added in one portion at 15 °C under N₂. The mixture was heated and stirred at 60 °C for 2 hr. TLC (Petroleum ether:Ethyl acetate=1:1) indicated 96a was consumed completely and one new spot formed. After cooling, the reaction mixture was quenched by addition of brine (10 mL) at 15 °C and extracted with EtOAc (30 mL×3). The combined organic layers were washed with brine (10 mL), dried over Na₂SO₄, filtered and concentrated under reduced pressure to dryness. The residue was purified by column chromatography (SiO₂, Petroleum ether/Ethyl acetate=100/1 to 9/1) to give compound 96b (0.52 g, 1.84 mmol, 47.1% yield) as a yellow solid.

### Step 2:

To a solution 96b (520 mg, 1.84 mmol, 1.00 eq) in THF (5 mL) TMPZnCl·LiCl (0.48 M in THF, 7.67 mL, 2.00 eq) was added drop wise at 0 °C and stirred for 10 min, then Br₂ (353 mg, 2.21 mmol, 1.20 eq) in DCM (3 mL) was added to the above mixture drop wise at 0 °C under N₂. The reaction mixture was stirred at 0 °C for 10 min. TLC (Petroleum ether:Ethyl acetate=1:1) indicated 96b was consumed completely and one new spot formed. The reaction mixture was quenched by addition of Na₂S₂O₃ aq. (10 mL) at 15 °C, and then extracted with EtOAc (10 mL×3). The combined organic layers were washed with brine (10 mL), dried over Na₂SO₄, filtered and concentrated under reduced pressure to dryness. The residue was purified by column chromatography (SiO₂, Petroleum ether/Ethyl acetate=100/1 to 3/1) to give compound 96c (0.15 g, 22.6% yield) as a yellow solid.

### Step 3:

To a mixture of dried CsF (189 mg, 1.25 mmol, 3.00 eq) and 96c (0.15 g, 415 umol, 1.00 eq) in DMSO (3 mL) was heated and stirred at 110 °C for 1.5 hr under N₂. LC-MS showed 96c was consumed completely and one main peak with desired mass was detected. After cooling, the reaction mixture was quenched by addition of H₂O (20 mL) at 15 °C, and then extracted with EtOAc (10 mL×3). The combined organic layers were washed with H₂O (10 mL×2), dried over Na₂SO₄, filtered and concentrated under reduced pressure to dryness. The residue was purified by prep-TLC (SiO₂, Petroleum ether:Ethyl acetate=1:1) to give 96d (109 mg, 363 umol, 87.4% yield) as a yellow solid.

### Step 4:

A reaction solution of 96d (109 mg, 363 umol, 1.00 eq) in TFA (2 mL) was heated and stirred at 50 °C for 1 hr. LC-MS showed 96d was consumed completely and one main peak with desired mass was detected. After cooling, the mixture was diluted with DCM (10 mL) and concentrated under reduced pressure to give a residue. The residue was purified by prep-HPLC (FA condition column: 3_Phenomenex Luna C18 75*30 mm*3 um; mobile phase: [water (0.2% FA)-ACN]; B %: 20%-50%, 9 min) to afford the titled product Ex.96 (35 mg, 166 umol, 45.9% yield) as a yellow solid. ¹H NMR: 400 MHz; δ: 1.72-1.83 (m, 1H) 1.84-1.95 (m, 1H) 2.10-2.23 (m, 2H) 2.46-2.58 (m, 2H) 4.75 (quin, J=7.2 Hz, 1H) 6.93 (dd, J=11.6, 2.8 Hz, 1H) 7.27-7.36 (m, 1H) 7.42 (d, J=11.6 Hz, 1H); HPLC: MS (M+H): 211.1

### Example 97: Preparation of 4-(cyclopentyloxy)-2-fluoro-7-hydroxycyclohepta-2,4,6-trien-1-one (Ex.97)

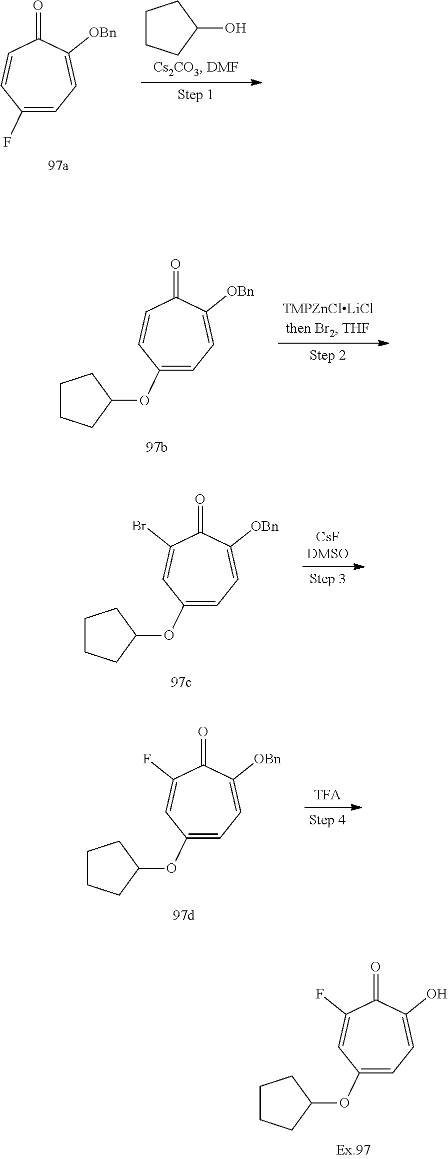 [SMILES: O=c1ccc(F)ccc1OCc1ccccc1 ; OC1CCCC1 ; O=c1ccc(OC2CCCC2)ccc1OCc1ccccc1 ; O=c1c(Br)cc(OC2CCCC2)ccc1OCc1ccccc1 ; O=c1c(F)cc(OC2CCCC2)ccc1OCc1ccccc1 ; O=c1c(O)ccc(OC2CCCC2)cc1F]

### Step 1:

To a mixture of 97a (0.60 g, 2.61 mmol, 1.00 eq) and cyclopentanol (1.12 g, 13 mmol, 1.18 mL, 5.00 eq) in DMF (3 mL) Cs₂CO₃ (2.55 g, 7.80 mmol, 3.00 eq) was added in one portion at 15 °C under N₂. The mixture was heated and stirred at 60 °C for 2 hr. TLC (Petroleum ether:Ethyl acetate=1:1) indicated 97a was consumed completely and one new spot formed. The reaction mixture was quenched by addition of water (10 mL) at 15 °C and extracted with EtOAc (20 mL×3). The combined organic layers were washed with brine (10 mL), dried over Na₂SO₄, filtered and concentrated under reduced pressure to dryness. The residue was purified by column chromatography (SiO₂, Petroleum ether/Ethyl acetate=100/1 to 9/1) to give compound 97b (0.67 g, 86.7% yield) as a yellow solid.

### Step 2:

To a solution of 97b (0.57 g, 1.92 mmol, 1.00 eq) in THF (10 mL) chloro-(2,2,6,6-tetramethyl-1-piperidyl)zinc (0.40 M in THF, 14.4 mL, 3.00 eq) was added drop wise at 0 °C and stirred for 30 min. Then Br₂ (369 mg, 2.31 mmol, 120 μL, 1.20 eq) in DCM (3 mL) was added to the above mixture drop-wise at 0 °C under N₂. The mixture was stirred at 0 °C for another 20 min. TLC (Petroleum ether:Ethyl acetate=1:1) indicated 97b was consumed completely and one new spot formed. The reaction mixture was quenched by addition of aq. Na₂S₂O₃ (10 mL) at 15 °C, and then extracted with EtOAc (20 mL×3). The combined organic layers were washed with water (10 mL), brine (10 mL), dried over Na₂SO₄, filtered and concentrated under reduced pressure to dryness. The residue was purified by column chromatography (SiO₂, Petroleum ether/Ethyl acetate=100/1 to 3/1) to give compound 97c (0.30 g, 41.5% yield) as a yellow solid.

### Step 3:

To a solution of dried CsF (303 mg, 2.00 mmol, 3.00 eq) in DMSO (3 mL) 97c (0.25 g, 666 umol, 1.00 eq) was added in one portion at 15 °C under N₂. The mixture was heated and stirred at 110 °C for 1.5 hr. LC-MS showed 97c was consumed completely and one main peak with desired mass was detected. The reaction mixture was quenched by addition of H₂O (20 mL) at 15 °C, and then extracted with EtOAc (10 mL×3). The combined organic layers were washed with H₂O (10 mL×2), dried over Na₂SO₄, filtered and concentrated under reduced pressure to dryness. The residue was purified by prep-TLC (SiO₂, Petroleum ether:Ethyl acetate=1:1) to give 97d (87.0 mg, 41.5% yield) as a yellow solid.

### Step 4:

A solution of 97d (87.0 mg, 277 umol, 1.00 eq) in TFA (0.5 mL) was heated and stirred at 50 °C for 2.5 hr. LC-MS showed 97d was consumed completely and one main peak with desired mass was detected. After cooling the reaction mixture was concentrated under reduced pressure to dryness. The residue was purified by prep-HPLC (FA condition column: Phenomenex Luna C18 200*40 mm*10 um; mobile phase: [water (0.2% FA)-ACN]; B %: 40%-80%, 8 min) to afford the titled product Ex.97 (30.0 mg, 48.3% yield) as a yellow solid. ¹H NMR: 400 MHz; δ: 1.60-1.74 (m, 2H) 1.75-1.90 (m, 4H) 1.94-2.07 (m, 2H) 4.86-4.92 (m, 1H) 7.05 (dd, J=11.6, 2.8 Hz, 1H) 7.32-7.40 (m, 1H) 7.44 (d, J=11.6 Hz, 1H); HPLC: MS (M+H): 225.1

### Example 98: Preparation of 3-(3,3-difluorocyclobutoxy)-2-hydroxycyclohepta-2,4,6-trien-1-one (Ex.98)

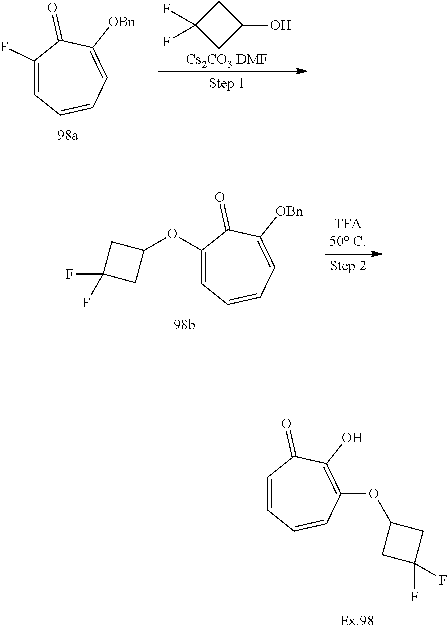

### Step 1:

To a mixture of 98a (0.50 g, 2.17 mmol, 1 eq) and 3,3-difluorocyclobutanol (469 mg, 4.34 mmol, 2 eq) in DMF (4 mL) Cs₂CO₃ (1.42 g, 4.34 mmol, 2 eq) was added at 25 °C. The mixture was heated and stirred at 60 °C for 3 hrs. LCMS showed the starting materials was consumed and the desired MS was detected. After cooling, water (20 mL) was added to the reaction mixture and then extracted with ethyl acetate (20 mL×3). The combined organic phases were washed with brine (10 mL×2), dried over anhydrous Na₂SO₄, filtered and concentrated in vacuum to dryness. The residue was purified by silica gel chromatography (petroleum ether:ethyl acetate=100:1 to 100:25) to give 98b (0.58 g, 74.6% yield) as a yellow oil.

### Step 2:

A solution of 98b (0.50 g, 1.57 mmol, 1 eq) in TFA (2 mL) was heated to 50 °C for 1 hr. LCMS showed the starting materials was consumed and the desired MS was detected. The mixture was diluted with DCM (10 mL) and concentrated in vacuum to dryness. The residue was purified by pre-HPLC (column: 3_Phenomenex Luna C18 75*30 mm*3 um; mobile phase: [water (0.2% FA)-ACN]; B %: 18%-48%, 7 min) to afford the titled product Ex.98 (0.28 g, 77.3% yield, 99% purity) as a white solid. ¹HNMR: 400 MHz; δ: 9.93 (br s, 1H), 7.30 (d, J=9.6 Hz, 1H), 7.23 (d, J=10.0 Hz, 1H), 7.15 (t, J=10.0 Hz, 1H), 7.06-6.99 (m, 1H), 4.94-4.79 (m, 1H), 3.28-3.19 (m, 2H), 2.87-2.70 (m, 2H); HPLC: MS: 229.1

### Example 99: Preparation of 4-cyclopropoxy-2-hydroxycyclohepta-2,4,6-trien-1-one (Ex.99)

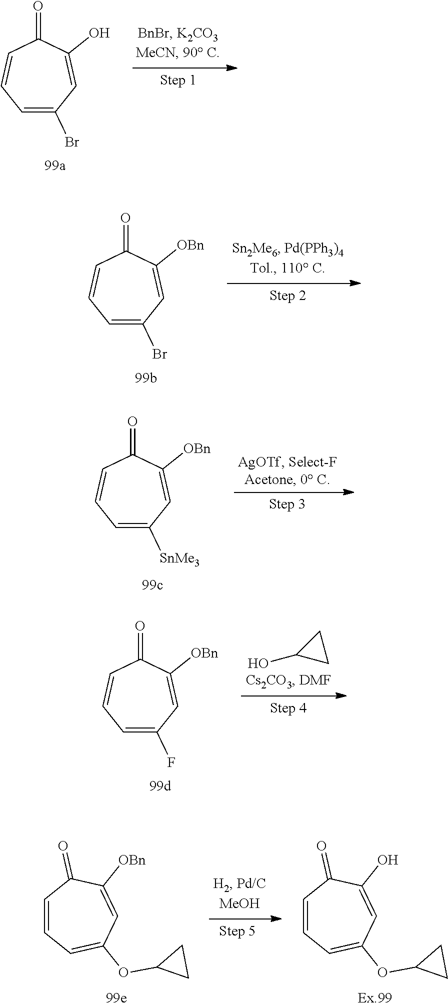

### Step 1:

To a mixture of 99a (5.00 g, 24.8 mmol, 1.00 eq) and K₂CO₃ (6.88 g, 49.7 mmol, 2.00 eq) in CH₃CN (50.0 mL) bromomethylbenzene (37.3 mmol, 4.43 mL, 1.50 eq) was added drop wise at 25 °C. The reaction mixture was heated and stirred at 90 °C for 4 hrs. TLC (petroleum ether/ethyl acetate=3/1, R_{f}=0.5) indicated 99a was consumed completely and one new spot formed. The reaction was clean according to TLC. After cooling to the room temperature, the mixture was added water (200 mL) and then extracted with EtOAc (150 mL×3). The combined organic phases were washed with brine (50 mL×2), dried over anhydrous Na₂SO₄, filtered and concentrated under reduced pressure to give a residue. The residue was purified by column chromatography (SiO₂, Petroleum ether/Ethyl acetate=85/1 to 15/1) to give compound 99b (3.70 g, 12.7 mmol, 51.0% yield) as a yellow solid.

### Step 2:

To a solution of 99b (2.10 g, 7.21 mmol, 1.00 eq) in toluene (45.0 mL Pd(PPh₃)₄ (833 mg, 721 umol, 0.10 eq) and trimethyl(trimethylstannyl)stannane (2.84 g, 8.66 mmol, 1.79 mL, 1.20 eq) were added in one portion at 20 °C under N₂. The system was degassed and then charged with nitrogen three times. The mixture was heated to 110 °C and stirred for 1.5 hours. LCMS showed the reactant was consumed completely and one main peak with desired mass was detected. After cooling to the room temperature, the reaction mixture was quenched by addition water (20 mL), and then extracted with EtOAc (50 mL×4). The combined organic layers were washed with H₂O (20 mL×2), brine (20 mL×2), dried over Na₂SO₄, filtered and concentrated under reduced pressure to give a residue. The residue was purified by column chromatography (SiO₂, Petroleum ether/Ethyl acetate=85/1 to 15/1) to give compound 99c (2.30 g, 85.0% yield) as a yellow solid.

### Step 3:

To a solution of 99c (1.77 g, 4.72 mmol, 1.00 eq) in Acetone (15.0 mL) AgOTf (2.43 g, 9.44 mmol, 2.00 eq) and Select F (2.01 g, 5.66 mmol, 1.20 eq) were added in one portion at 0 °C under N₂. The mixture was stirred at 0 °C for 25 min. LCMS showed 99c was consumed completely and one main peak with desired mass was detected. The reaction mixture was quenched by addition of H₂O (10 mL) at 15 °C and extracted with DCM (20 mL×4). The combined organic layers were washed with H₂O (10 mL), brine (10 mL), dried over Na₂SO₄, filtered and concentrated under reduced pressure to give a residue. The residue was purified by column chromatography (SiO₂, Petroleum ether/Ethyl acetate=85/1 to 15/1) to give compound 99d (0.500 g, 2.17 mmol, 46.0% yield) as a brown solid.

### Step 4:

A mixture of 99d (0.30 g, 1.30 mmol, 1.00 eq), cyclopropanol (68.1 mg, 1.17 mmol, 0.90 eq) and Cs₂CO₃ (636 mg, 1.95 mmol, 1.50 eq) in DMF (3.00 mL) was degassed and purged with N₂ for 3 times, and then the reaction mixture was stirred at 20 °C for 12 hr under N₂ atmosphere. LCMS showed 99d was consumed completely and one main peak with desired mass was detected. The reaction mixture was quenched by addition of H₂O (5 mL) at 25 °C, and extracted with EtOAc (20 mL×3). The combined organic layers were washed with brine (10 mL), dried over Na₂SO₄, filtered and concentrated under reduced pressure to give a residue. The residue was purified by column chromatography (SiO₂, Petroleum ether/Ethyl acetate=83/1 to 17/1) to give compound 99e (130 mg, crude) as a brown oil.

### Step 5:

To a solution of 99e (110 mg, 409 umol, 1.00 eq) in MeOH (1.00 mL) was added Pd/C (10%, 2.00 g) under H₂. The suspension was degassed under vacuum and purged with H₂ several times. The mixture was stirred under H₂ (15 psi) at 50 °C for 0.5 hours. LCMS showed 99e was consumed completely and one main peak with desired mass was detected. After cooling, the mixture was filtered through a pad of Celite and the filter cake was washed with MeOH (5 mL×3). The filtrate was concentrated under reduced pressure to dryness. The residue was purified by prep-HPLC (FA condition) to afford the titled product Ex.99 (25.0 mg, 140 umol, 34.2% yield) as a yellow solid. ¹H NMR: 400 MHz; δ ppm 0.75-0.82 (m, 2H) 0.88-0.95 (m, 2H) 3.82-3.91 (m, 1H) 6.88 (dd, J=11.2, 2.63 Hz, 1H) 7.02 (d, J=10.4 Hz, 1H) 7.24 (d, J=2.8 Hz, 1H) 7.27-7.35 (m, 1H)

### Example 100: Preparation of 4-fluoro-2-hydroxycyclohepta-2,4,6-trien-1-one (Ex.100)

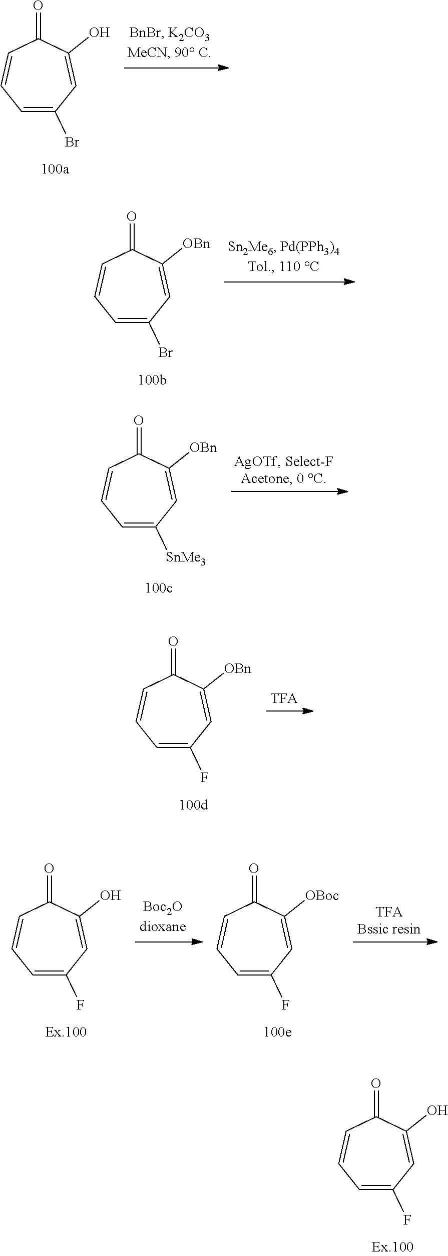

### Step 1:

To a mixture of 100a (5.00 g, 24.9 mmol, 1 eq), K₂CO₃ (6.88 g, 49.7 mmol, 2.00 eq) in CH₃CN (50 mL) bromomethylbenzene (4.43 mL, 37.3 mmol, 1.50 eq) was added drop wise at 25 °C. The mixture was heated and stirred at 90 °C for 4 hrs. TLC (petroleum ether:ethyl acetate=3:1, Rf=0.5. UV 254 nm as developer) indicated 100a was consumed completely and one new spot formed. The reaction was clean according to TLC. After cooling, the mixture was added water (200 mL) and then extracted with EtOAc (150 mL×3). The combined organic phases were washed with brine (50 mL), dried over anhydrous Na₂SO₄, filtered and concentrated in vacuum to dryness. The residue was purified by column chromatography (SiO₂, Petroleum ether/Ethyl acetate=85/1 to 15/1) to give 100b (3.70 g, 51.1% yield) as a yellow solid.

### Step 2:

To a solution of 100b (2.1 g, 7.21 mmol, 1.00 eq) in toluene (45 mL) Pd(PPh₃)₄ (833 mg, 721.30 umol, 0.1 eq) and trimethyl(trimethylstannyl)stannane (2.84 g, 8.66 mmol, 1.79 mL, 1.2 eq) were added in one portion at 20 °C under N₂. The system was degassed and then charged with nitrogen three times. The mixture was heated and stirred at 110 °C for 1.5 hrs. LC-MS showed 100b was consumed completely and one main peak with desired mass was detected. After cooling, the reaction mixture was quenched by addition of MeOH (20 mL) and then extracted with EtOAc (50 mL×4). The combined organic layers were washed with H₂O (10 mL×2), brine, dried over Na₂SO₄, filtered and concentrated under reduced pressure to give a residue. The residue was purified by column chromatography (SiO₂, Petroleum ether/Ethyl acetate=85/1 to 15/1) to give 100c (2.3 g, 85.02% yield) as a yellow solid.

### Step 3:

To a solution of 100c (1.77 g, 4.72 mmol, 1.00 eq) in acetone (15 mL) silver trifluoromethanesulfonate (2.43 g, 9.44 mmol, 2.00 eq) and Select-F (2.01 g, 5.66 mmol, 1.20 eq) were added in one portion at 0 °C under N₂. The mixture was stirred at 0 °C for 30 min. LC-MS showed 100c was consumed completely and one main peak with desired mass was detected. The reaction mixture was quenched by addition H₂O (10 mL) at 15 °C and extracted with DCM (20 mL×4). The combined organic layers were washed with brine (10 mL), dried over Na₂SO₄, filtered and concentrated under reduced pressure to give a residue. The residue was purified by column chromatography (SiO₂, Petroleum ether/Ethyl acetate=85/1 to 15/1) to give 100d (500 mg, 46.02% yield) as a brown solid.

### Step 4:

A mixture of 100d (300 mg, 1.30 mmol, 1.00 eq) in TFA (1.30 mmol, 96.5 uL, 1.00 eq) was degassed and purged with N₂ for 3 times, and then the mixture was heated and stirred at 50 °C for 2 hrs under N₂ atmosphere. LC-MS showed 100d was consumed completely and one main peak with desired mass detected. After cooling, the reaction mixture was diluted with CH₂Cl₂ (10 mL) and concentrated under reduced pressure to dryness to give Ex.100 (170 mg, crude) as a yellow oil.

### Step 5:

A mixture of Ex.100 (0.21 g, 1.50 mmol, 1 eq), Boc₂O (981 mg, 4.50 mmol, 3.00 eq), TEA (6.00 mmol, 834 μL, 4.00 eq) in dioxane (2 mL) was degassed and purged with N₂ for 3 times, and then the mixture was heated and stirred at 118 °C for 0.5 hr under N₂ atmosphere. LC-MS showed Ex.100 was consumed completely and one main peak with desired mass detected. After cooling, the reaction mixture was concentrated under reduced pressure to give a residue. The residue was purified by column chromatography (SiO₂, Petroleum ether/Ethyl acetate=100/1 to 30/1) to give 100e (180 mg, 52.5% yield) as a yellow solid.

### Step 6:

To a solution of 100e (90 mg, 374.65 umol, 1 eq) in CH₂Cl₂ (0.5 mL) TFA (28 uL, 375 umol, 1.00 eq) was added in one portion at 0 °C. The mixture was warmed and stirred at 25 °C for 20 min. LC-MS showed 100e was consumed completely and one main peak with desired mass detected. The reaction mixture was diluted with CH₂Cl₂ (10 mL) and concentrated under reduced pressure to dryness below 10 °C. Then the mixture was re-dissolved in CH₂Cl₂ (5 mL) and Amberlyst A21 (0.1 g) was added and stirred at 25 °C for another 0.5 hr. After filtering, the cake was washed with CH₂Cl₂ (5 mL×2) and the filtrate was concentrated under reduced pressure to afford the titled product Ex.100 (25 mg, 47.6% yield) as a yellow solid. ¹H NMR: 400 MHz; δ ppm 6.93-7.03 (m, 1H) 7.08-7.18 (m, 1H) 7.22 (d, 0.1=10.8 Hz, 1H) 7.46 (td, 0.1=10.8, 4.49 Hz, 1H); HPLC: MS: 141.1

### Example 101: Preparation of 4-ethyl-7-fluoro-2-hydroxycyclohepta-2,4,6-trien-1-one (Ex.101)

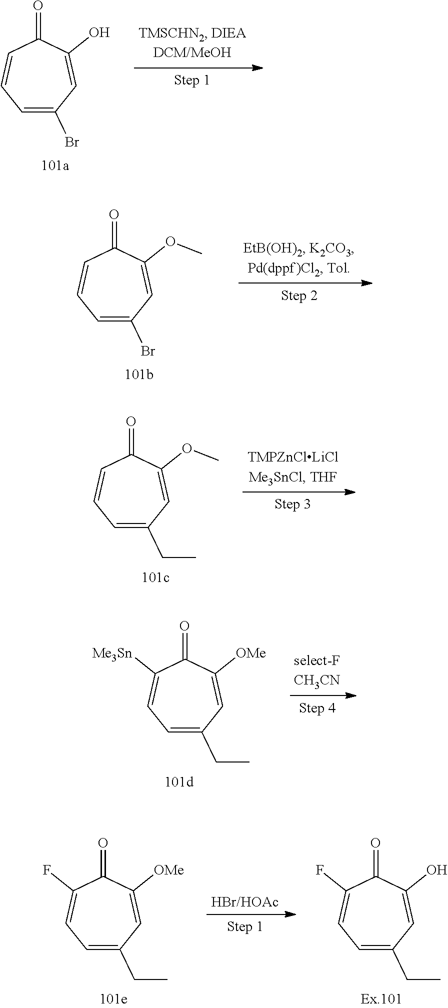

### Step 1:

To a solution of 101a (10 g, 49.7 mmol, 1.00 eq) in DCM (100 mL) and MeOH (12 mL) DIPEA (9.64 g, 74.6 mmol, 13 mL, 1.5 eq) and diazomethyl(trimethyl)silane (2 M, 54 mL, 2.2 eq) were added in one portion at 0 °C. The mixture was stirred at 20 °C for 5 hours. LCMS showed the reaction was completed. The mixture was quenched with water (350 mL) and extracted with DCM (150 mL×3). The combined organic phases were dried over anhydrous Na₂SO₄, filtered and concentrated under reduced pressure to give a residue. The crude product was purified by column chromatography (SiO₂, Petroleum ether:Ethyl acetate=100:0 to 61:39) to give compound 101a (4.5 g, 42.0% yield) as a yellow solid.

### Step 2:

To a mixture of 101b (3.00 g, 13.9 mmol, 1.00 eq) and K₂CO₃ (4.82 g, 34.8 mmol, 2.5 eq) in toluene (30 mL) ethylboronic acid (10.3 g, 139 mmol, 10 eq) was added in one portion at 20 °C under N₂. Then Pd(dppf)Cl₂·CH₂Cl₂ (2.28 g, 2.79 mmol, 0.2 eq) was added to the mixture. The system was degassed and then charged with nitrogen three times. The mixture was heated and stirred at 110 °C for 30 min. LCMS showed the reaction was completed. After cooling, the mixture was filtered through a pad of Celite and the filter cake was washed with EtOAc (100 mL×3). The combined organic phases were washed with water (100 mL), brine (100 mL), dried over anhydrous Na₂SO₄, filtered and concentrated under reduced pressure to give a residue. The residue was purified by column chromatography (SiO₂, Petroleum ether:Ethyl acetate=100:0 to 71:29) to give compound 101c (2 g, 12.2 mmol, 87.3% yield) as a yellow oil.

### Step 3:

To a solution of 101c (1.60 g, 9.74 mmol, 1.00 eq) in THF (16 mL) chloro-(2,2,6,6-tetramethyl-1-piperidyl)zinc (TMPZnCl·LiCl, 0.4 M in THF, 73.1 mL, 3 eq) was added in one portion at −10 °C under N₂. After 10 min, chloro(trimethyl)stannane (2.91 g, 14.6 mmol, 1.5 eq) was added to the above mixture and the reaction mixture was heated and stirred at 50 °C for 4 hours. LC-MS showed the reaction was completed. After cooling, the mixture was diluted with water (50 mL) and then extracted with ethyl acetate (70 mL×3). The combined organic layers were washed with brine (50 mL), dried over Na₂SO₄, filtered and concentrated under reduced pressure to give a residue. The residue was purified by column chromatography (SiO₂, Petroleum ether:Ethyl acetate=100:0 to 84:16) to give compound 101d (778 mg, 24.4% yield) as yellow oil.

### Step 4:

A mixture of 101d (0.77 g, 2.35 mmol, 1 eq) and Select-F (2.50 g, 7.06 mmol, 3 eq) in CH₃CN (6 mL) was stirred at 25 °C for 20 h. LCMS showed the reaction was completed. Then the mixture was quenched with water (50 mL), and then extracted with ethyl acetate (50 mL×3). The combined organic layers were washed with brine (50 mL), dried over anhydrous Na₂SO₄, filtered and concentrated under reduced pressure to give a residue. The residue was purified by column chromatography (SiO₂, Petroleum ether:Ethyl acetate=100:0 to 79:21) to give compound 101e (69 mg, 16.1% yield) as a yellow oil.

### Step 5:

101e (0.06 g, 329 umol, 1 eq) was added to hydrogen bromide (481 mg, 5.95 mmol, 323 uL, 18 eq) in one portion at 25 °C. The mixture was heated and stirred at 100 °C for 30 min. LCMS showed the reaction was completed. After cooling, MTBE (3 mL) was added to the mixture and stirred for 10 min, then filtered and the filter cake was washed with MTBE (2 mL×2). The precipitate was collected and dried in vacuo to afford the titled product Ex.101 (52 mg, 93.9% yield) as an off-white solid. ¹H NMR: 400 MHz; δ: 1.28 (t, J=7.6 Hz, 3H) 2.73 (q, J=7.6 Hz, 2H) 7.00-7.08 (m, 1H) 7.46 (d, J=1.6 Hz, 1H) 7.55-7.69 (m, 1H); HPLC: MS: (M+1): 169.0

### Example 102: Preparation of 5-fluoro-2-hydroxy-3-isopropylcyclohepta-2,4,6-trien-1-one (Ex.102)

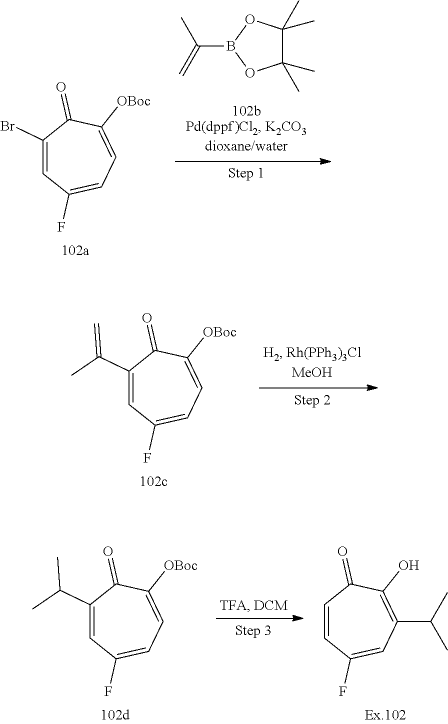

### Step 1:

To a mixture of 102a (400 mg, 1.25 mmol, 1.00 eq) and 102b (421 mg, 2.51 mmol, 2.00 eq) in dioxane (6 mL) and H₂O (0.6 mL) K₂CO₃ (346 mg, 2.51 mmol, 2.00 eq) and Pd(dppf)Cl₂·CH₂Cl₂ (102 mg, 125 umol, 0.10 eq) were added in one portion at 15 °C under N₂. The system was degassed and then charged with nitrogen for three times. Then the mixture was heated and stirred at 118 °C for 30 min. TLC (Petroleum ether/Ethyl acetate=3/1) indicated 102a was consumed completely and one new spot formed. After cooling, the reaction mixture was quenched by addition H₂O (10 mL) at 15 °C and then extracted with EtOAc (10 mL×3). The combined organic layers were washed with brine (10 mL), dried over Na₂SO₄, filtered and concentrated under reduced pressure to dryness. The residue was purified by column chromatography (SiO₂, Petroleum ether/Ethyl acetate=100/1 to 3/1) to give 102c (130 mg, 463 umol, 37.0% yield) as a yellow solid.

### Step 2:

To a solution of 102c (120 mg, 428 umol, 1.00 eq) in MeOH (5 mL) chlororhodium triphenylphosphane (74 mg, 80 umol, 0.18 eq) was added under N₂. The suspension was degassed under vacuum and purged with H₂ several times. The reaction mixture was stirred under H₂ (15 psi) at 30 °C for 1 hr. TLC (Petroleum ether/Ethyl acetate=5/1) indicated 102c was consumed completely and one new spot formed. After cooling, the mixture was filtered through a pad of Celite and the filter cake was washed with MeOH (10 mL×3). The filtrate was concentrated under reduced pressure to dryness. The residue was purified by prep-TLC (SiO₂, Petroleum ether/Ethyl acetate=5/1) to give 102d (73 mg, 60.4% yield) as a white solid.

### Step 3:

To a solution of 102d (73 mg, 258 umol, 1.00 eq) in DCM (3 mL) TFA (1 mL) was added in one portion at 20 °C. The mixture was stirred at 20 °C for 30 min. TLC (Petroleum ether/Ethyl acetate=3/1) indicated 102d was consumed completely and one new spot formed. The reaction mixture was diluted with CH₂Cl₂ (10 mL) and concentrated under reduced pressure to dryness below 10 °C. The residue was purified by prep-HPLC (FA condition column: 3_Phenomenex Luna C18 75*30 mm*3 um; mobile phase: [water (0.2% FA)-ACN]; B %: 30%-50%, 10 min) to afford the titled product Ex.102 (14 mg, 29.7% yield) as a yellow solid. ¹H NMR: 400 MHz; δ: 1.25 (d, J=6.8 Hz, 6H) 3.70 (dt, J=13.6, 6.8 Hz, 1H) 7.17-7.33 (m, 2H) 7.49 (dd, J=18.4, 2.8 Hz, 1H); HPLC: MS (M+H): 183.2

### Example 103: Preparation of 5-fluoro-2-hydroxy-3-(tetrahydro-2H-pyran-2-yl)cyclohepta-2,4,6-trien-1-one (E.103)

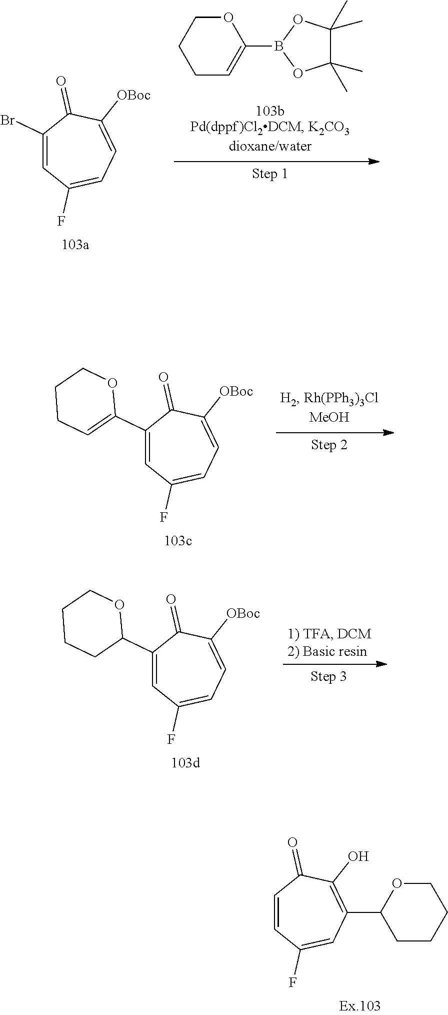

### Step 1:

To a mixture of 103a (340 mg, 1.07 mmol, 1.00 eq) and 103b (447 mg, 2.13 mmol, 2.00 eq) in dioxane (7 mL) and H₂O (0.7 mL) Pd(dppf)Cl₂·CH₂Cl₂ (87 mg, 106 umol, 0.1 eq) and K₂CO₃ (294 mg, 2.13 mmol, 2.00 eq) were added in one portion at 20 °C under N₂. The system was degassed and then charged with nitrogen for three times. Then the reaction mixture was heated and stirred at 118 °C for 1 hr. TLC (Petroleum ether/Ethyl acetate=5/1) indicated 103a was consumed completely and one new spot formed. The reaction mixture was quenched by addition of H₂O (10 mL) at 20 °C and extracted with EtOAc (10 mL×3). The combined organic layers were washed with H₂O (10 mL), brine (10 mL), dried over Na₂SO₄, filtered and concentrated under reduced pressure to dryness. The residue was purified by column chromatography (SiO₂, Petroleum ether/Ethyl acetate=100/1 to 10/1) to give 103c (260 mg, 807 umol, 75.7% yield) as a yellow solid.

### Step 2:

To a solution of 103c (250 mg, 775 umol, 1.00 eq) in MeOH (5 mL) Rh(PPh₃)₃C1 (250 mg, 270 umol, 0.3 eq) was added in one portion at 20 °C under N₂ atmosphere. The system was degassed and then charged with H₂ three times. The mixture was stirred under H₂ (15 psi) at 30 °C for 1 hr. TLC (Petroleum ether/Ethyl acetate=5/1) indicated 103c was consumed completely and one new spot formed. After cooling, the mixture was filtered through a pad of Celite and the filter cake was washed with MeOH (5 mL×2). The filtrate was concentrated under reduced pressure to dryness. The residue was purified by column chromatography (SiO₂, Petroleum ether/Ethyl acetate=100/1 to 10/1) to give 103d (160 mg, 493 umol, 63.6% yield) as a yellow solid.

### Step 3:

To a solution of 103d (160 mg, 493 umol, 1.00 eq) in DCM (5 mL) TFA (1.5 mL) was added in one portion at 15 °C under N₂. The mixture was stirred at 15 °C for 1 hr. TLC (Petroleum ether/Ethyl acetate=3/1) indicated 103d was consumed completely and one new spot formed. The reaction mixture was diluted with CH₂Cl₂ (10 mL) and concentrated under reduced pressure to dryness below 10 °C. Then the mixture was re-dissolved in CH₂Cl₂ (5 mL) and Amberlyst A21 (0.1 g) was added and stirred at 25 °C for another 0.5 hr. After filtering, the cake was washed with CH₂Cl₂ (5 mL×2) and the filtrate was concentrated under reduced pressure to afford the titled product Ex.103 (31 mg, 28.0% yield) as a yellow solid. ¹H NMR: 400 MHz; δ: 1.19 (dd, J=12 Hz, 1H), 1.58-1.83 (m, 3H), 1.88-1.97 (m, 1H), 2.06-2.18 (m, 1H), 3.68 (td, J=12 Hz, 1H), 4.18 (dt, J=12 Hz, 1H) 4.81 (dd, J=12, 1.76 Hz, 1H) 7.19-7.34 (m, 2H) 7.77 (dd, J=16 Hz, 1H); HPLC: MS (M+H): 225.1

### Example 104: Preparation of 2-hydroxy-5-(spiro[2.3]hexan-5-yloxy)cyclohepta-2,4,6-trien-1-one (Ex.104)

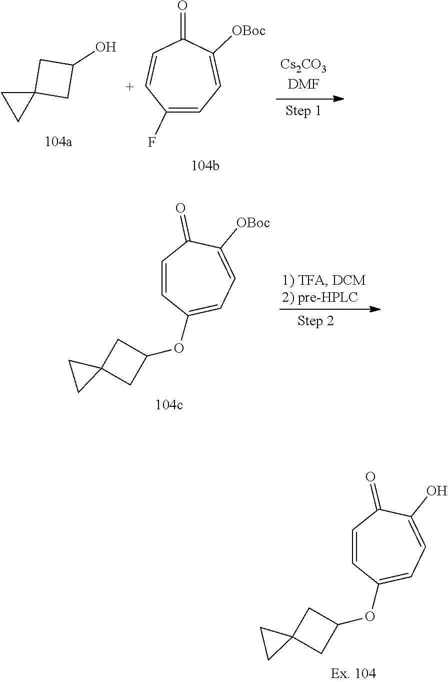

### Step 1:

To a solution of 104a in DMF (3 mL) Cs₂CO₃ (732 mg, 2.25 mmol, 3.00 eq) and tert-butyl 104b (180 mg, 749 umol, 1.00 eq) were added in one portion at 15 °C under N₂. The mixture was heated and stirred at 60 °C for 30 min. LC-MS showed 104b was consumed completely and one main peak with desired mass was detected. After cooling to room temperature, the reaction mixture was quenched by addition of H₂O (10 mL), and then extracted with ethyl acetate (10 mL×3). The combined organic layers were washed with H₂O (10 mL), brine (10 mL), dried over Na₂SO₄, filtered and concentrated under reduced pressure to give a residue. The residue was purified by prep-TLC (SiO₂, Petroleum ether:Ethyl acetate=3:1) to give compound 104c (120 mg, 50.3% yield) as a yellow solid.

### Step 2:

To a solution of 104c (0.1 g, 314 umol, 1 eq) in DCM (5 mL) TFA (1 mL) was added in one portion at 20 °C under N₂. The mixture was stirred at 20 °C for 1 hr. TLC (Petroleum ether:Ethyl acetate=3:1) indicated 104c was consumed completely and one new spot formed. The reaction mixture was concentrated under reduced pressure and purified by prep-HPLC (FA condition column: 3_Phenomenex Luna C18 75*30 mm*3 um; mobile phase: [water (0.2% FA)-ACN]; B %: 20%-70%, 9 min) to afford the titled product Ex.104 (28 mg, 40.8% yield) as a yellow solid. ¹H NMR: 400 MHz; δ: 0.43-0.51 (m, 2H) 0.53-0.60 (m, 2H) 2.37-2.44 (m, 2H) 2.46-2.54 (m, 2H) 4.91-4.98 (m, 1H) 7.01-7.11 (m, 2H) 7.29-7.35 (m, 2H); HPLC: MS (M+H): 219.2

### Example 105: Preparation of 3-(cyclobutylmethoxy)-2-hydroxycyclohepta-2,4,6-trien-1-one (Ex.105)

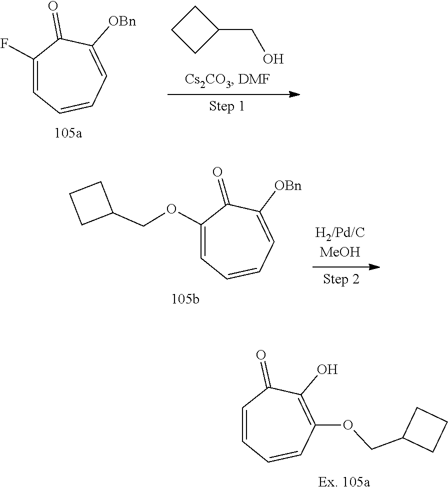 [SMILES: O=c1c(F)ccccc1OCc1ccccc1 ; OCC1CCC1 ; O=c1c(OCc2ccccc2)ccccc1OCC1CCC1 ; O=c1ccccc(OCC2CCC2)c1O]

### Step 1:

A mixture of 105a (500 mg, 2.17 mmol, 1.00 eq), cyclobutylmethanol (224 mg, 2.61 mmol, 245 uL, 1.20 eq) and Cs₂CO₃ (1.42 g, 4.34 mmol, 2.00 eq) in DMF (5 mL) was degassed and purged with N₂ for 3 times, and then the mixture was heated and stirred at 60 °C for 12 hr under N₂ atmosphere. LCMS showed 105a was consumed completely and one main peak with desired mass was detected. After cooling, the reaction mixture was quenched by addition of H₂O (10 mL) at 25 °C, and then extracted with EtOAc (20 mL×5). The combined organic layers were washed with brine (20 mL), dried over Na₂SO₄, filtered and concentrated under reduced pressure to give a residue. The residue was purified by column chromatography (SiO₂, Petroleum ether/Ethyl acetate=83/1 to 17/1) to give compound 105b (525 mg, 81.5% yield) as a brown oil.

### Step 2:

To a solution of 105b (520 mg, 1.75 mmol, 1.00 eq) in MeOH (0.5 mL) Pd/C (10%, 2.00 g) was added under N₂. The suspension was degassed under vacuum and purged with H₂ several times. The mixture was stirred under H₂ (15 psi) at 50 °C for 0.5 hours. LCMS showed 105b was consumed completely and one main peak with desired mass was detected. The mixture was filtered and concentrated under reduced pressure to give a residue. The residue was purified by prep-HPLC (FA condition) to afford the titled product Ex.105 (120 mg, 33.1% yield) as a yellow solid. ¹H NMR: 400 MHz; δ: 1.86-2.05 (m, 4H) 2.13-2.26 (m, 2H) 2.84-2.98 (m, 1H) 4.13 (d, J=6.8 Hz, 2H) 7.11-7.27 (m, 2H) 7.39-7.48 (m, 2H)

### Example 106: Preparation of 3-cyclopropoxy-7-fluoro-2-hydroxycyclohepta-2,4,6-trien-1-one (Ex.106)

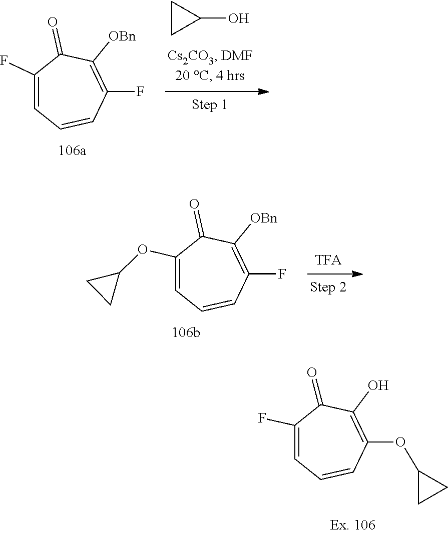

### Step 1:

To a mixture of 106a (320 mg, 1.29 mmol, 1.00 eq) and cyclopropanol (75.0 mg, 1.29 mmol, 1.00 eq) in DMF (4 mL) Cs₂CO₃ (840 mg, 2.58 mmol, 2.00 eq) was added in one portion at 20 °C under N₂. The mixture was stirred at 20 °C for 4 hrs. LCMS showed the starting materials was consumed and the desired MS was detected. Water (10 mL) was added to the reaction mixture and extracted with ethyl acetate (15 mL×2). The combined organic phase was washed with brine (10 mL×2), dried over anhydrous Na₂SO₄, filtered and concentrated in vacuum to dryness. The residue waspurified by silica gel chromatography (Petroleum ether/Ethyl acetate=100/1 to 100/28) to give 106b (0.16 g, 40.3% yield) as a yellow oil.

### Step 2:

106b (0.12 g, 419 umol, 1.00 eq) was added into TFA (2 mL) at 25 °C. The mixture was heated and stirred at 50 °C for 5 hrs. LCMS showed the starting materials was consumed and the desired MS was detected. The mixture was diluted with DCM (20 mL) and concentrated in vacuum to dryness. The residue was purified by prep-HPLC (column: 3_Phenomenex Luna C18 75*30 mm*3 um; mobile phase: [water (0.2% FA)-ACN]; B %: 5%-35%, 10 min) to afford the titled product Ex.106 (50.0 mg, 60.2% yield) as a yellow solid. ¹HNMR: 400 MHz; δ 7.79 (d, J=10.8 Hz, 1H), 7.35 (dd, J=11.6, 18.4 Hz, 1H), 7.16 (dt, J=4.0, 11.2 Hz, 1H), 4.03-3.89 (m, 1H), 0.98-0.90 (m, 2H), 0.89-0.83 (m, 2H); HPLC: MS: 197.1

### Examples 107 and 108: Preparation of (R)-3-(1-cyclopropylethoxy)-2-hydroxycyclohepta-2,4,6-trien-1-one (Ex.107) and (S)-3-(1-cyclopropylethoxy)-2-hydroxycyclohepta-2,4,6-trien-1-one (Ex.108)

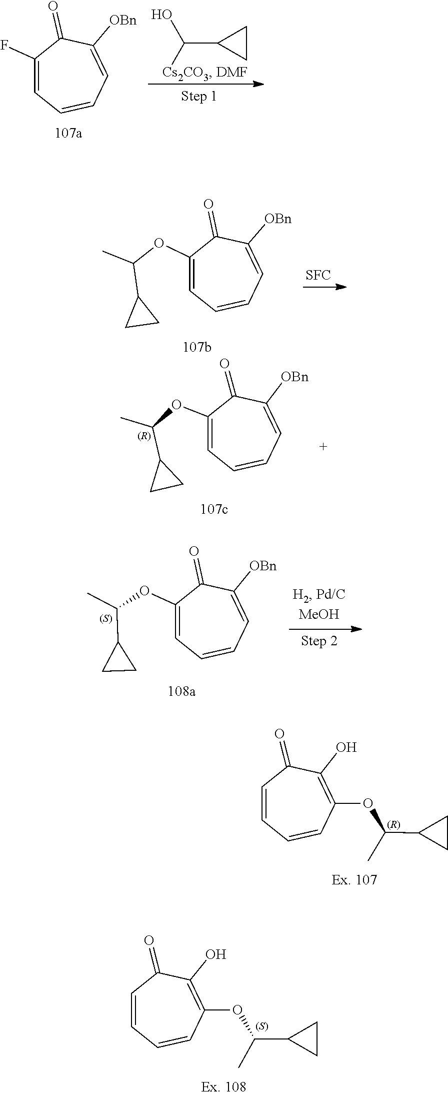

### Step 1:

To a solution of 107a (1.00 g, 4.34 mmol, 1.00 eq) in DMSO (20 mL) Cs₂CO₃ (5.66 g, 17.4 mmol, 4.00 eq) and 1-cyclopropylethanol (1.12 g, 13.0 mmol, 1.27 mL, 3.00 eq) were added in one portion at 25 °C under N₂. The mixture was heated and stirred at 120 °C for 1 hour. LCMS showed the reaction was completed. The reaction mixture was quenched by addition of H₂O (30 mL) at 15 °C, and then extracted with ethyl acetate (50 mL×3). The combined organic phases were washed with brine (30 mL), dried over anhydrous Na₂SO₄, filtered and concentrated under reduced pressure to give a residue. The residue was purified by column chromatography (SiO₂, Petroleum ether/Ethyl acetate=100/0 to 90/10) to give compound 107b (600 mg, 46.6% yield) as a yellow oil. From 107b, SFC was employed to isolate 107c and 108a.

### Step 2:

To a solution of 107c (246 mg, 830 umol, 1.00 eq) in MeOH (2.50 mL) Pd/C (10%, 140 mg) was added under N₂. The suspension was degassed under vacuum and purged with H₂ several times. The reaction mixture was stirred under H₂ (15 psi) at 50 °C for 0.5 hours. LCMS showed the starting material was consumed completely. After cooling, the mixture was filtered through a pad of Celite and the filter cake was washed with MeOH (10 mL×3). The filtrate was concentrated under reduced pressure to dryness. The residue was purified by prep-HPLC (FA condition) to afford the titled product Ex.107 (100 mg, 485 umol, 58.4% yield) as a yellow oil. ¹H NMR: 400 MHz; δ 0.18-0.41 (m, 2H) 0.47-0.63 (m, 2H) 1.11-1.28 (m, 1H) 1.46 (d, J==6.0 Hz, 3H) 4.03-4.17 (m, 1H) 6.91-7.04 (m, 1H) 7.16 (t, J=10.0 Hz, 1H) 7.32 (d, J=10.4 Hz, 1H) 7.39 (d, J=10.0 Hz, 1H); HPLC: MS: (M+1): 207.0

### Step 3:

To a solution of 108a (243 mg, 820 umol, 1.00 eq) in MeOH (2.50 mL) Pd/C (10%, 140 mg) was added under N₂. The suspension was degassed under vacuum and purged with H₂ several times. The reaction mixture was stirred under H₂ (15 psi) at 50 °C for 0.5 hours. LCMS showed the starting material was consumed completely. After cooling, the mixture was filtered through a pad of Celite and the filter cake was washed with MeOH (10 mL×3). The filtrate was concentrated under reduced pressure to dryness. The residue was purified by prep-HPLC (FA condition) to afford the titled product Ex.108 (100 mg, 59.1% yield) as a yellow oil. ¹H NMR: 400 MHz; 8:0.20-0.43 (m, 2H) 0.50-0.64 (m, 2H) 1.12-1.27 (m, 1H) 1.46 (d, J=6.0 Hz, 3H) 4.03-4.16 (m, 1H) 6.94-7.03 (m, 1H) 7.16 (t, J=10.0 Hz, 1H) 7.33 (d, J=10.4 Hz, 1H) 7.39 (d, J=10.0 Hz, 1H); HPLC: MS: (M+1): 207.0

### Example 109: Preparation of 7-fluoro-2-hydroxy-3-(spiro[2.3]hexan-5-yloxy)cyclohepta-2,4,6-trien-1-one (Ex.109)

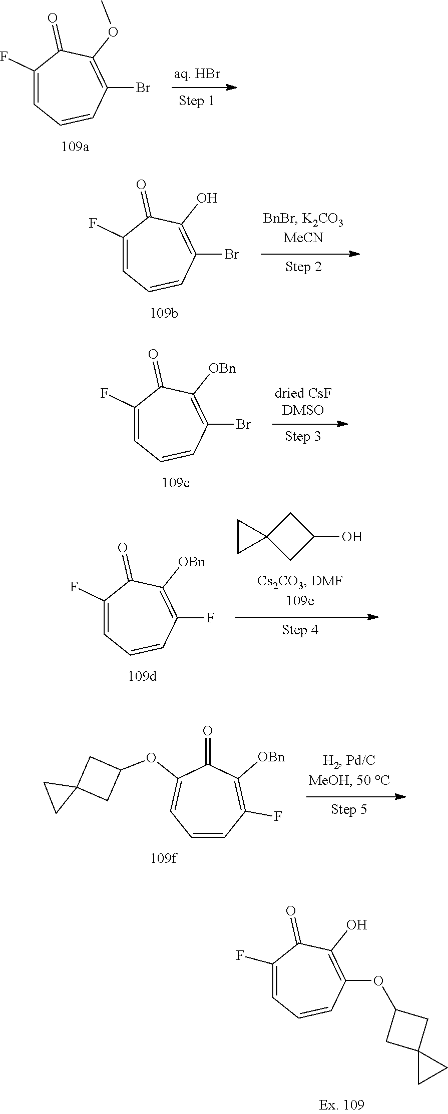

### Step 1:

109a (5.00 g, 21.4 mmol, 1.00 eq) was added into aq. HBr (30.0 mL, 50% purity) at 25 °C. The mixture was heated and stirred at 80 °C for 0.5 hour. LCMS showed the starting material was consumed completely and the desired MS was detected. After cooling to room temperature, water (50 mL) was added to the reaction mixture and extracted with dichloromethane (50 mL×3). The combined organic phases were washed with brine (40 mL×2), dried over anhydrous Na₂SO₄, filtered and concentrated in vacuum to get 109b (4.68 g, 92.6% yield) as a yellow solid.

### Step 2:

To a mixture of 109b (4.68 g, 21.3 mmol, 1.00 eq) and K₂CO₃ (5.91 g, 42.7 mmol, 2.00 eq) in MeCN (50 mL) bromomethylbenzene (25.6 mmol, 3.05 mL, 1.20 eq) was added in one portion at 25 °C. The mixture was heated and stirred at 90 °C for 5 hrs. TLC (petroleum ether/ethyl acetate=3/1) showed the starting material was consumed and one new spot observed. After cooling to room temperature, water (30 mL) was added to the reaction mixture and then extracted with ethyl acetate (50 mL×2). The combined organic phases were washed with brine (30 mL), dried over anhydrous Na₂SO₄, filtered and concentrated in vacuum to dryness. The residue was purified by silica gel chromatography (Petroleum ether/Ethyl acetate=100/1 to 100/20) to give 109c (1.50 g, 20.4% yield) as a yellow solid.

### Step 3:

To a solution of 109c (1.20 g, 3.88 mmol, 1.00 eq) in DMSO (12 mL) dried CsF (1.18 g, 7.76 mmol, 2.00 eq) was added under N₂. The reaction mixture was heated and stirred at 60 °C for 1.5 hrs. LCMS showed the starting materials was consumed and the desired MS was detected. After cooling to room temperature, water (10 mL) was added to the reaction mixture and extracted with ethyl acetate (20 mL×3). The combined organic phases were washed with brine (10 mL×2), dried over anhydrous Na₂SO₄, filtered and concentrated in vacuum to dryness. The residue was purified by silica gel chromatography (Petroleum ether/Ethyl acetate=100/1 to 100/28) to give 109d (320 mg, 31.5% yield) as a yellow solid.

### Step 4:

To a solution of 109e (48.0 mg, 483 umol, 1.00 eq) in THF (2.00 mL) NaH (29.0 mg, 725 umol, 60% purity, 1.50 eq) was added at 0 °C, and the mixture was stirred at 0 °C for 30 min. 109d (120 mg, 483 umol, 1.00 eq) in THF (2 mL) was added to the above mixture at 0 °C. The reaction mixture was stirred at 20 °C for 2 hrs. LCMS showed the starting materials was consumed and the desired MS was detected. The residue was poured into ice water (5 mL) and stirred for 5 min. The aqueous phase was extracted with ethyl acetate (10 mL×3). The combined organic phases were washed with brine (10 mL×2), dried over anhydrous Na₂SO₄, filtered and concentrated in vacuum to dryness to give 109f (170 mg, 89.4% yield) as a yellow oil.

### Step 5:

To a solution of 109f (170 mg, 521 umol, 1.00 eq) in MeOH (2.00 mL) Pd/C (276 mg, 260 umol, 10% purity, 0.5 eq) was added under N₂. The suspension was degassed under vacuum and purged with H₂ several times. The mixture was stirred under H₂ (15 psi) at 50 °C for 0.5 hr. LCMS showed the starting material was consumed completely and the desired MS was observed. The reaction mixture was filtered and concentrated in vacuum. The residue was purified by prep-HPLC (column: 3_Phenomenex Luna C18 75*30 mm*3 um; mobile phase: [water (0.2% FA)-ACN]; B %: 20%-50%, 9 min) to afford the titled product Ex.109 (40.0 mg, 32.5% yield) as a yellow solid. ¹HNMR: 400 MHz; δ: 7.31 (dd, J=11.2, 18.4 Hz, 1H), 7.23-7.15 (m, 1H), 7.14-7.01 (m, 1H), 5.09 (quin, J=6.8 Hz, 1H), 2.61-2.46 (m, 4H), 0.61-0.54 (m, 2H), 0.53-0.45 (m, 2H); HPLC: MS: 237.1

### Example 110: Preparation of 2-hydroxy-5-(1-hydroxy-3-phenylpropan-2-yl)cyclohepta-2,4,6-trien-1-one (Ex.110)

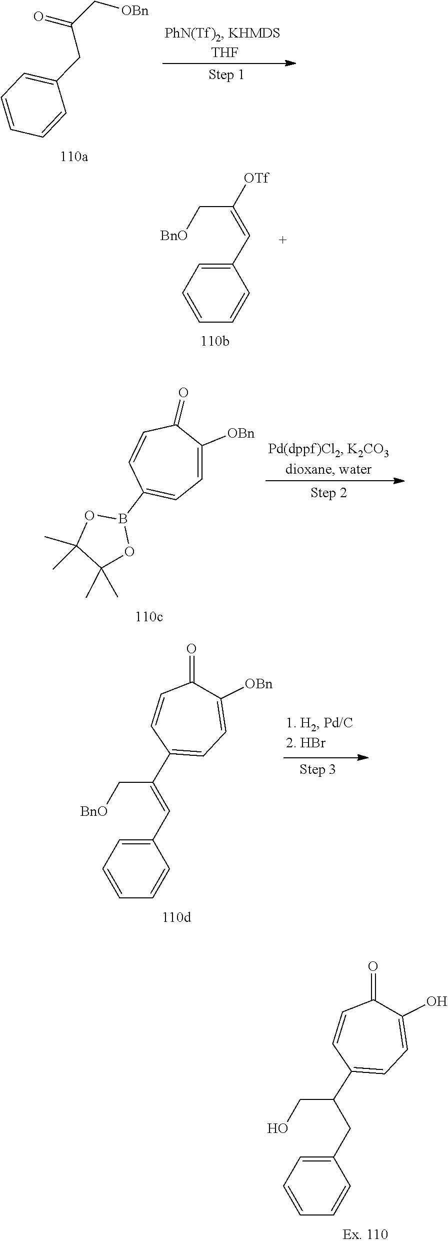 [SMILES: O=C(COCc1ccccc1)Cc1ccccc1 ; O=S(=O)(O/C(=C/c1ccccc1)COCc1ccccc1)C(F)(F)F ; CC1(C)OB(c2ccc(OCc3ccccc3)c(=O)cc2)OC1(C)C ; O=c1ccc(/C(=C/c2ccccc2)COCc2ccccc2)ccc1OCc1ccccc1 ; "THF" ; O=c1ccc(C(CO)Cc2ccccc2)ccc1O]

### Step 1:

To a solution of 110a (3.50 g, 14.5 mmol, 1.00 eq) in THF (40 mL) KHMDS (1.00 M in THF, 16.0 mL, 1.10 eq) was added drop-wise at −75 °C under N₂. After stirring for 30 min, a solution of PhN(Tf)₂ (6.24 g, 17.4 mmol, 1.20 eq) in THF (30 mL) was added to the above mixture. Then the reaction mixture was stirred at 20 °C for 1 hr under N₂. LCMS showed the reaction was completed. The reaction mixture was quenched with saturated aqueous NH₄Cl solution (100 mL) and then extracted with EtOAc (100 mL×3). The combined organic layers were washed with brine (200 mL), dried over Na₂SO₄, filtered and concentrated under reduced pressure to dryness to give compound 110b (7.00 g, crude) as a brown solid.

### Step 2:

To a mixture of 110c (1.00 g, 2.96 mmol, 1.00 eq) and 110b (1.65 g, 4.44 mmol, 1.50 eq) in dioxane (10 mL) and H₂O (2 mL) Pd(dppf)Cl₂ (241 mg, 295 umol, 0.100 eq) and K₂CO₃ (1.02 g, 7.39 mmol, 2.50 eq) were added at 20 °C under N₂ atmosphere. The system was degassed and then charged with nitrogen three times. The mixture was heated and stirred at 120 °C for 0.5 hr. TLC showed the starting material was consumed and a new spot was observed. After cooling, the mixture was filtered through a pad of Celite and the filter cake was washed with CH₂Cl₂ (30 mL×3). The filtrate was concentrated under reduced pressure to dryness. The residue was purified by silica gel chromatography (Petroleum ether/Ethyl acetate=90/10 to 75/25) to give compound 110d (1.10 g, 1.27 mmol, 21.4% yield, 50% purity) as a yellow oil.

### Step 3:

To a solution of 110d (800 mg, 1.84 mmol, 1.00 eq) in MeOH (20.0 mL) Pd/C (500 mg, 352 umol, 10% purity, 0.2 eq) was added under N₂. The suspension was degassed under vacuum and purged with H₂ several times. The reaction mixture was stirred under H₂ (15 psi) at 20 °C for 6 hr. LCMS showed the starting material was consumed and the desired MS was observed. The reaction mixture was filtered and the filter was concentrated to give a residue. HBr (10 mL, 50% purity) was added into the residue and the mixture was heated and stirred at 60 °C for 0.5 hr. The mixture was poured into water (10 mL) and extracted with ethyl acetate (10 mL×3). The combined organic phases were washed with brine (10 mL), dried over anhydrous Na₂SO₄, filtered and concentrated in vacuum. The residue was purified by prep-HPLC (column: 3_Phenomenex Luna C18 75*30 mm*3 um; mobile phase: [water (0.2% FA)-ACN]; B %: 20%-40%, 9 min) to afford the titled product Ex.110 (12.0 mg, 46.6 umol, 3.23% yield, 99.5% purity) as a yellow solid. ¹H NMR: 400 MHz; δ: 7.40-7.32 (m, 2H), 7.29-7.22 (m, 2H), 7.22-7.15 (m, 2H), 7.14-7.03 (m, 3H), 3.82-3.72 (m, 2H), 3.15-3.03 (m, 2H), 2.87-2.76 (m, 1H)

### Example 111: Preparation of 5-fluoro-2-hydroxy-3-(spiro[2.3]hexan-5-yloxy)cyclohepta-2,4,6-trien-1-one (Ex.111)

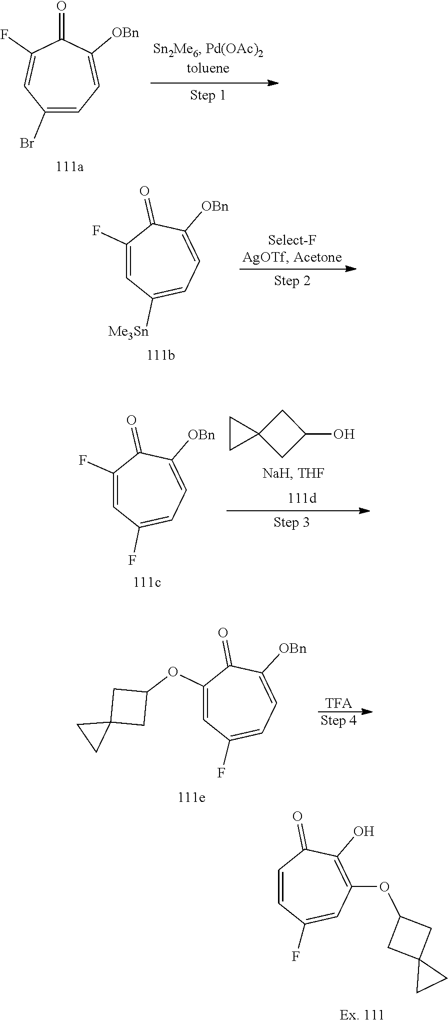

### Step 1:

To a mixture of tributyl(tributylstannyl)stannane (4.50 g, 7.76 mmol, 3.88 mL, 2.00 eq) and 111a (1.20 g, 3.88 mmol, 1.00 eq) in toluene (20 mL) Pd(OAc)₂ (87.2 mg, 388 umol, 0.1 eq) and tricyclohexylphosphane (217 mg, 776 umol, 0.2 eq) were added under N₂ at 25 °C. The system was degassed and then charged with nitrogen three times. Then the reaction mixture was heated and stirred at 120 °C for 1.5 hr. TLC (Petroleum ether:Ethyl acetate=3:1) indicated 111a was consumed completely and one new spot formed. MeOH (100 mL) was added and the mixture was concentrated under reduced pressure to give a residue. The residue was purified by column chromatography (SiO₂, Petroleum ether/Ethyl acetate=100/1 to 10/1) to give 111b (1.1 g, 2.12 mmol, 54.6% yield) as a yellow oil.

### Step 2:

To a mixture of 111b (850 mg, 1.64 mmol, 1.00 eq) and 1-(chloromethyl)-4-fluoro-1,4-diazoniabicyclo[2.2.2]octane dihexafluorophosphate (Select-F, 2.31 g, 4.91 mmol, 3.00 eq) in acetone (5 mL) a solution of silver trifluoromethanesulfonate (AgOTf, 1.26 g, 4.91 mmol, 3.00 eq) in acetone (1 mL) was added drop-wise at −30 °C, then the reaction mixture was stirred at −30 °C for another 30 min. LCMS showed 111b was consumed completely and one main peak with desired mass was detected. The reaction mixture was quenched by addition H₂O (10 mL) at 0 °C, and then extracted with ethyl acetate (10 mL×3). The combined organic layers were washed with brine (10 mL), dried over Na₂SO₄, filtered and concentrated under reduced pressure to give a residue. The residue was purified by prep-TLC (SiO₂, Petroleum ether/Ethyl acetate=3/1) to give 111c (159 mg, 640 umol, 39.1% yield) as a yellow solid.

### Step 3:

To a solution of 111d (37.0 mg, 377 umol, 1.10 eq) in THF (4 mL) NaH (17.8 mg, 445 umol, 60% purity, 1.30 eq) was added at 0 °C under N₂. After stirring for 10 min at 0 °C, 111c (85.0 mg, 342 umol, 1.00 eq) was added to the reaction mixture in one portion at 0 °C under N₂. The reaction mixture was stirred at 0 °C for 1 hr. LCMS showed 111c was consumed completely and one main peak with desired mass was detected. The reaction mixture was quenched by addition H₂O (10 mL) at 0 °C, and then extracted with ethyl acetate (10 mL×3). The combined organic layers were washed with brine (10 mL), dried over Na₂SO₄, filtered and concentrated under reduced pressure to give a residue. The residue was purified by column chromatography (SiO₂, Petroleum ether/Ethyl acetate=100/1 to 3/1) to give 111e (80 mg, 245 umol, 71.6% yield) as a yellow solid.

### Step 4:

A solution of 111e (80 mg, 245 umol, 1.00 eq) in TFA (2 mL) was stirred at 50 °C for 1 hr. LCMS showed the starting material was consumed completely and major desired MS observed. The reaction mixture was diluted with DCM (10 mL) and concentrated under reduced pressure to dryness. The residue was purified by prep-HPLC (FA condition column: 3_Phenomenex Luna C18 75*30 mm*3 um; mobile phase: [water(0.2% FA)-ACN]; B %: 28%-58%, 8 min) to afford the titled product Ex.111 (26 mg, 110 umol, 44.9% yield) as a yellow solid. ¹H NMR: 400 MHz; δ: 0.46-0.55 (m, 2H) 0.55-0.63 (m, 2H) 2.57 (d, J=6.4 Hz, 4H) 5.12 (quin, J=6.4 Hz, 1H) 7.02 (ddd, J=16.0, 10.8, 2.8 Hz, 1H) 7.10 (dd, J=18.4, 2.8 Hz, 1H) 7.37 (dd, J=10.8, 3.6 Hz, 1H); HPLC: MS: 237.1

### Example 112: Preparation of 2-hydroxy-5-((1-hydroxy-3-phenylpropan-2-yl)oxy)cyclohepta-2,4,6-trien-1-one (Ex.112)

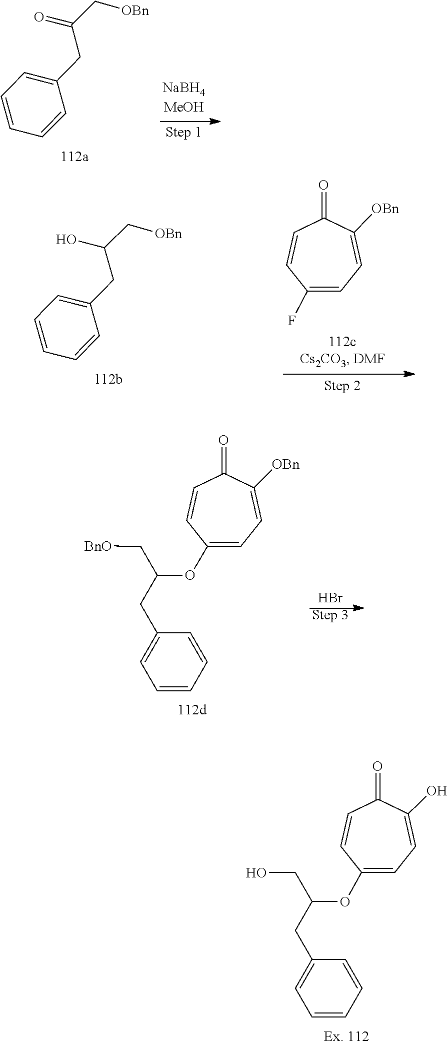 [SMILES: O=C(COCc1ccccc1)Cc1ccccc1 ; OC(COCc1ccccc1)Cc1ccccc1 ; O=c1ccc(F)ccc1OCc1ccccc1 ; O=c1ccc(OC(COCc2ccccc2)Cc2ccccc2)ccc1OCc1ccccc1 ; O=c1ccc(OC(CO)Cc2ccccc2)ccc1O]

### Step 1:

To a solution of 112a (5.00 g, 20.8 mmol, 1.00 eq) in MeOH (50.0 mL) NaBH₄ (865 mg, 22.8 mmol, 1.10 eq) was added in four portions at 0 °C under N₂. The reaction mixture was stirred at 20 °C for 0.5 hr. LCMS showed the starting material was consumed and desired mass was observed. The reaction mixture was quenched with aqueous HCl solution to pH=7, added water (20 mL) and extracted with ethyl acetate (20 mL×3). The combined organic phase was washed with brine (50 mL), dried over anhydrous Na₂SO₄, filtered and concentrated in vacuum to give a residue. The residue was purified by silica gel chromatography (Petroleum ether/Ethyl acetate=90/10 to 80/20) to give 112b (2.53 g, 10.4 mmol, 50.0% yield) as a brown oil.

### Step 2:

To a mixture of 112b (2.53 g, 10.4 mmol, 1.20 eq) and 112c (2.00 g, 8.69 mmol, 1.00 eq) in DMF (10.0 mL) Cs₂CO₃ (5.66 g, 17.3 mmol, 2.00 eq) was added at 20 °C. The reaction mixture was heated and stirred at 100 °C for 12 hr. LCMS showed the starting material was consumed and the desired MS was observed. After cooling, the reaction mixture was quenched water (20 mL) and then extracted with ethyl acetate (10 mL×3). The combined organic phases were washed with brine (30 mL), dried over anhydrous Na₂SO₄, filtered and concentrated in vacuum to give a residue. The residue was purified by silica gel chromatography (Petroleum ether/Ethyl acetate=80/20 to 70/30) to give 112d (0.3 g, 331 umol, 3.82% yield, 50% purity) as a yellow oil.

### Step 3:

112d (0.10 g, 220 umol, 1.00 eq) was added to aq. HBr (2.00 mL) at 20 °C and the reaction mixture was heated and stirred at 50 °C for 0.5 hr. LCMS showed the starting material was consumed and the desired MS was observed. The mixture was poured into water (5 mL) and extracted with ethyl acetate (5 mL×3). The combined organic phases were washed with brine (10 mL), dried over anhydrous Na₂SO₄, filtered and concentrated under reduced pressure to dryness. The residue was purified by prep-HPLC (column: Phenomenex Gemini-NX 150*30 mm*5 um; mobile phase: [water (0.1% TFA)-ACN]; B %: 25%-55%, 9 min) to afford the titled product Ex.112 (16.0 mg, 58.1 umol, 13.1% yield, 99.0% purity) as a yellow solid. ¹H NMR: 400 MHz; δ ppm 7.28-7.22 (m, 6H), 7.22-7.11 (m, 3H), 4.62-4.54 (m, 1H), 3.79-3.63 (m, 2H), 2.99 (dd, J=6.4, 10.8 Hz, 2H)

### Example 113: Preparation of 5-fluoro-2-hydroxy-3-(tetrahydro-2H-pyran-4-yl)cyclohepta-2,4,6-trien-1-one (Ex.113)

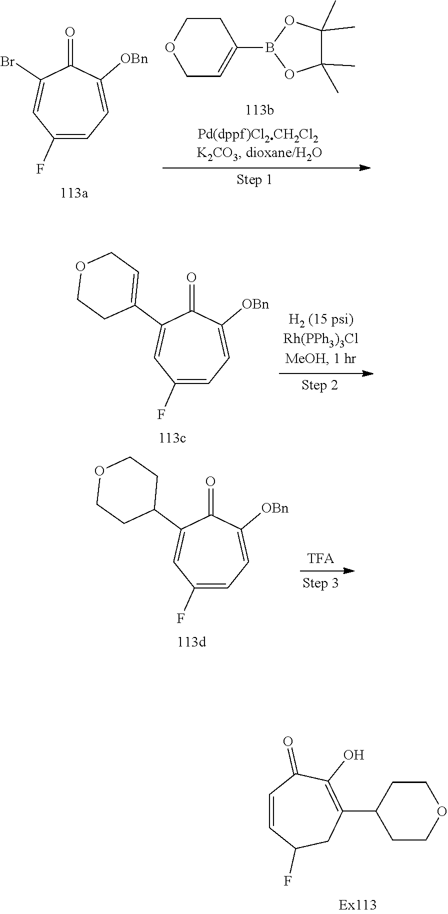

### Step 1:

To a mixture of 113a (110 mg, 356 umol, 1.00 eq) and 113b (112 mg, 534 umol, 1.50 eq) in dioxane (5 mL) and H₂O (0.5 mL) K₂CO₃ (98 mg, 712 umol, 2.00 eq) and Pd(dppf)Cl₂·CH₂Cl₂ (29 mg, 35.6 umol, 0.100 eq) were added in one portion at 20 °C under N₂ atmosphere, then the system was degassed and charged with nitrogen three times. The reaction mixture was heated and stirred at 118 °C for 30 min. TLC (Petroleum ether:Ethyl acetate=3:1) indicated 113a was consumed completely and one new spot formed. After cooling to room temperature, the reaction mixture was quenched by addition of H₂O (5 mL), and then extracted with ethyl acetate (10 mL×3). The combined organic layers were washed with brine (5 mL), dried over anhydrous Na₂SO₄, filtered and concentrated under reduced pressure to dryness. The residue was purified by column chromatography (SiO₂, Petroleum ether/Ethyl acetate=100/1 to 3/1) to give 113c (108 mg, 346 umol, 97.2% yield) as a yellow solid.

### Step 2:

To a solution of 113c (108 mg, 346 umol, 1.00 eq) in MeOH (5 mL) Rh(PPh₃)₃Cl (108 mg, 117 umol, 3.38e-1 eq) was added at 20 °C. The suspension was degassed under vacuum and purged with H₂ several times. The mixture was stirred under H₂ (15 psi) at 30 °C for 1 hour. LCMS showed the reaction was completed. After cooling, the mixture was filtered through a pad of Celite and the filter cake was washed with MeOH (10 mL×2). The filtrate was concentrated under reduced pressure to dryness. The residue was purified by column chromatography (SiO₂, Petroleum ether/Ethyl acetate=100/1 to 75/25) to give 113d (108 mg, 344 umol, 99.4% yield) as a yellow solid.

### Step 3:

A solution of 113d (105 mg, 334 umol, 1.00 eq) in TFA (4.20 g, 36.81 mmol, 2.73 mL, 110 eq) was heated and stirred at 50 °C for 1 hr under N₂ atmosphere. LCMS showed the reaction was completed. After cooling, the reaction mixture was diluted with DCM (10 mL) and concentrated under reduced pressure dryness. The residue was purified by prep-HPLC (FA condition column: 3_Phenomenex Luna C18 75*30 mm*3 um; mobile phase: [water (0.2% FA)-ACN]; B %: 25%-60%, 8 min) to afford the titled product Ex.113 (34 mg, 152 umol, 45.4% yield) as a yellow solid. ¹HNMR: 400 MHz; δ: 1.69-1.81 (m, 4H) 3.53-3.72 (m, 3H) 4.07 (dt, J=11.2, 2.8 Hz, 2H) 7.16-7.34 (m, 2H) 7.50 (dd, J=18.0, 2.8 Hz, 1H); HPLC: MS: 225.1

### Example 114: Preparation of 3-cyclobutoxy-5-fluoro-2-hydroxycyclohepta-2,4,6-trien-1-one (Ex.114)

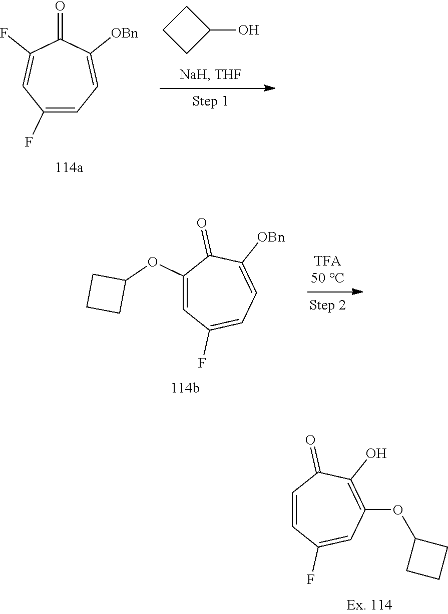

### Step 1:

To a solution of cyclobutanol (23 mg, 322 umol, 1.00 eq) in THF (3 mL) NaH (17 mg, 419 umol, 60% purity, 1.30 eq) was added at 0 °C under N₂ atmosphere. After stirring 10 min at 0 °C, 114a (80 mg, 322 umol, 1.00 eq) was added to the mixture in one portion at 0 °C under N₂ atmosphere. The reaction mixture was stirred at 0 °C for another 60 min. TLC (Petroleum ether:Ethyl acetate=3:1) indicated 114a was consumed completely and one new spot formed. The reaction mixture was quenched by addition of H₂O (10 mL) at 0 °C, and then extracted with ethyl acetate (10 mL×3). The combined organic layers were washed with brine (10 mL), dried over Na₂SO₄, filtered and concentrated under reduced pressure to dryness. The residue was purified by column chromatography (SiO₂, Petroleum ether/Ethyl acetate=100/1 to 3/1) to give 114b (94 mg, 313 umol, 97.1% yield) as a yellow solid.

### Step 2:

A solution of 114b (94 mg, 313 umol, 1.00 eq) in TFA (2.55 mL) was heated and stirred at 50 °C under N₂ atmosphere for 1 hr. LCMS showed the reaction was completed. The reaction mixture was concentrated under reduced pressure to give a residue, which was purified by prep-HPLC (FA condition column: 3_Phenomenex Luna C18 75*30 mm*3 um; mobile phase: [water (0.2% FA)-ACN]; B %: 30%-60%, 10 min) to afford the titled product Ex.114 (34.0 mg, 162 umol, 51.7% yield) as a yellow solid. ¹HNMR: 400 MHz; δ: 1.72-2.00 (m, 2H) 2.24-2.39 (m, 2H) 2.50-2.63 (m, 2H) 4.90-4.96 (m, 1H) 7.02 (ddd, J=15.6, 11.2, 2.8 Hz, 1H) 7.10 (dd, J=18.4, 2.8 Hz, 1H) 7.37 (dd, J=10.8, 3.6 Hz, 1H); HPLC: MS: 211.1

### Example 115: Preparation of 5-chloro-2-hydroxy-3-methylcyclohepta-2,4,6-trien-1-one (Ex.115)

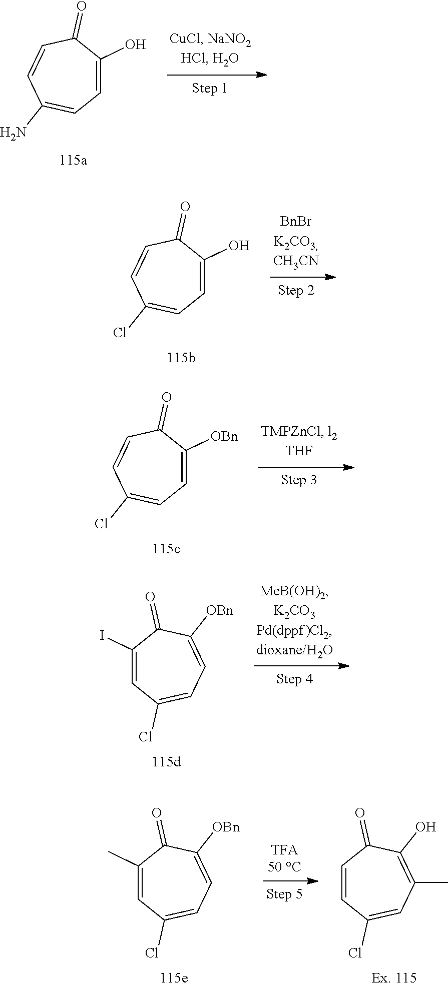 [SMILES: Nc1ccc(O)c(=O)cc1 ; O=c1ccc(Cl)ccc1O ; O=c1ccc(Cl)ccc1OCc1ccccc1 ; O=c1c(I)cc(Cl)ccc1OCc1ccccc1 ; Cc1cc(Cl)ccc(OCc2ccccc2)c1=O ; Cc1cc(Cl)ccc(=O)c1O]

### Step 1:

To a mixture of 115a (10.0 g, 72.9 mmol, 1.00 eq) and CuCl (14.4 g, 146 mmol, 2.00 eq) in aq. HCl (12 M, 50 mL) an aqueous solution of NaNO₂ (15.1 g, 219 mmol, 3.00 eq) in water (20 mL) was added drop-wise at 50 °C. After addition, the reaction was stirred at 50 °C for 12 hours. LC-MS showed 115a was consumed completely and one main peak with desired mass was detected. After cooling to room temperature, the reaction mixture was concentrated directly under reduced pressure to give crude 115b (11 g, crude) as a brown solid, which was used to next step.

### Step 2:

To a mixture of 115b (3.70 g, 23.6 mmol, 1.00 eq) and K₂CO₃ (6.53 g, 47.2 mmol, 2.00 eq) in acetonitrile (CH₃CN, 37 mL) bromomethylbenzene (BnBr, 6.06 g, 35.4 mmol, 1.50 eq) was added at 25 °C. Then the reaction mixture was heated and stirred at 90 °C for 4 hrs. TLC (Petroleum ether:Ethyl acetate=3:1, Rf=0.2) showed the starting material was consumed completely and one new spot observed. After cooling, water (50 mL) was added to the reaction mixture and then the mixture was extracted with ethyl acetate (100 mL×3). The combined organic phases were washed with brine (100 mL), dried over anhydrous Na₂SO₄, filtered and concentrated under reduced pressure. The residue was purified by column chromatography (SiO₂, Petroleum ether/Ethyl acetate=85/1 to 15/1) to give 115c (100 mg, 405 umol, 1.72% yield) as a yellow solid.

### Step 3:

To a solution of 115c (260 mg, 1.05 mmol, 1.00 eq) in THF (3 mL) chloro-(2,2,6,6-tetramethyl-1-piperidyl)zinc (TMPZnCl, 0.4 M in THF, 7.90 mL, 3.00 eq) was added at 0 °C under N₂. After stirring for 10 min, 12 (535 mg, 2.11 mmol, 2.00 eq) was added to above mixture in one portion at 0 °C. The reaction mixture was warmed and stirred at 25 °C for 30 min under N₂ atmosphere. TLC (Petroleum ether:Ethyl acetate=3:1, Rf=0.6) showed the starting material was consumed completely and one new spot observed. The reaction mixture was quenched by addition of H₂O (10 mL) at 25 °C, and then extracted with ethyl acetate (20 mL×4). The combined organic layers were washed with brine (10 mL), dried over anhydrous Na₂SO₄. filtered and concentrated under reduced pressure to give a residue. The residue was purified by column chromatography (SiO₂, Petroleum ether/Ethyl acetate=85/1 to 15/1) to give 115d (0.11 g, 295 umol, 28.0% yield) as a yellow solid.

### Step 4:

To a mixture of 115d (200 mg, 537 umol, 1.00 eq), K₂CO₃ (148 mg, 1.07 mmol, 2.00 eq) and methylboronic acid (161 mg, 2.68 mmol, 5.00 eq) in dioxane (2 mL) and H₂O (0.4 mL) Pd(dppf)Cl₂·CH₂Cl₂ (88 mg, 107 umol, 0.20 eq) was added under N₂ atmosphere. The system was degassed and then charged with nitrogen three times. The reaction mixture was heated and stirred at 100 °C for 0.5 hr. TLC (Petroleum ether:Ethyl acetate=1:1, Rf=0.3) showed the starting material was consumed completely and two new spots observed. After cooling, water (30 mL) was added to the reaction mixture and then the mixture was extracted with ethyl acetate (40 mL×3). The combined organic layers were washed with brine (30 mL), dried over anhydrous Na₂SO₄, filtered and concentrated under reduced pressure to give a residue. The residue was purified by column chromatography (SiO₂, Petroleum ether/Ethyl acetate=I/O to 3/1) to give 115e (108 mg, 414 umol, 77.2% yield) as a yellow oil.

### Step 5:

115e (100 mg, 383 umol, 1.00 eq) was added into TFA (2 mL) in one portion at 25 °C. The mixture was heated and stirred at 50 °C for 1 hr. LC-MS showed 115e was consumed completely and one main peak with desired mass was detected. The reaction mixture was concentrated directly under reduced pressure to give a residue. The residue was purified by prep-HPLC (FA condition; column: 3_Phenomenex Luna C18 75*30 mm*3 um; mobile phase: [water (0.2% FA)-ACN]; B %: 23%-53%, 10 min) to afford the titled product Ex.115 (10 mg, 58.3 umol, 15.2% yield, 99.4% purity) as a yellow solid. ¹H NMR: 400 MHz; 8 ppm 2.46 (s, 3H) 7.19 (d, J=11.2 Hz, 1H) 7.40 (dd, J=11.2, 2.0 Hz, 1H) 7.68 (s, 1H) 9.14-9.50 (m, 1H); HPLC: MS (M+H): 171.1

### Example 116: Preparation of 2-hydroxy-4-(spiro[2.3]hexan-5-yloxy)cyclohepta-2,4,6-trien-1-one (Ex.116)

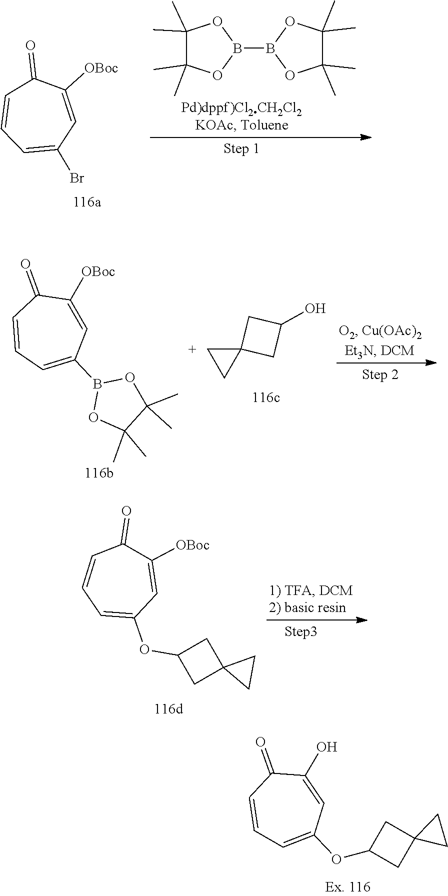

### Step 1:

To a mixture of 116a (1.00 g, 3.32 mmol, 1.00 eq) and 4,4,5,5-tetramethyl-2-(4,4,5,5-tetramethyl-1,3,2-dioxaborolan-2-yl)-1,3,2-dioxaborolane (928 mg, 3.65 mmol, 1.10 eq) in toluene (10 mL) KOAc (652 mg, 6.64 mmol, 2.00 eq) and Pd(dppf)Cl₂·CH₂Cl₂ (271 mg, 332 umol, 0.10 eq) was added at 25 °C under N₂ atmosphere. The system was degassed and then charged with nitrogen three times. The mixture was heated and stirred at 110 °C for 1 hr. TLC (petroleum ether:ethyl acetate=3:1, UV 254 nm as developer) showed the starting material was consumed and new spot observed. After cooling, the mixture was filtered and concentrated in vacuum to dryness. The residue was purified by silica gel chromatography (Petroleum ether/Ethyl acetate=100/1, 100/30) to give 116b (1.1 g, 2.94 mmol, 88.5% yield, 93% purity) as a yellow solid.

### Step 2:

To a mixture of 116b (231 mg, 662 umol, 1.30 eq) and 116c (50 mg, 509 umol, 1.00 eq) in dichloromethane (DCM) (4 mL) Cu(OAc)₂·H₂O (108 mg, 509 umol, 1.00 eq), triethylamine (TEA) (2.55 mmol, 355 μL, 5.00 eq) and 4 A molecular sieves were added at 25 °C. The suspension was degassed under vacuum and purged with O₂ three times. The mixture was stirred under O₂ (15 psi) at 25 °C for 72 hrs. LCMS showed the starting materials was consumed and the desired MS detected. The residue was poured into water (10 mL) and stirred for 5 min. The aqueous phase was extracted with ethyl acetate (10 mL×2). The combined organic phases were washed with brine (10 mL×2), dried over anhydrous Na₂SO₄, filtered and concentrated in vacuum. The residue was purified by silica gel chromatography (Petroleum ether/Ethyl acetate=100/1, 100/18) then purified by pre-HPLC (column: Phenomenex Luna C18 200*40 mm*Oum; mobile phase: [water (0.2% FA)-ACN]; B %: 40%-80%, 8 min) to give 116d (19 mg, 103 umol, 20.2% yield, 91% purity) as a yellow oil.

### Step 3:

To a solution of 116d (15 mg, 47.1 umol, 1.00 eq) in DCM (1 mL) TFA (0.2 mL) was added at 25 °C. The mixture was stirred at 25 °C for 0.5 hr. LCMS showed the starting materials was consumed completely and the desired MS was detected. The reaction mixture was diluted with CH₂Cl₂ (10 mL) and concentrated under reduced pressure to dryness below 10 °C. Then the mixture was re-dissolved in CH₂Cl₂ (5 mL) and added Amberlyst A21 (0.1 g) and stirred at 25 °C for another 0.5 hr. After filtering, the cake was washed with CH₂Cl₂ (5 mL×2) and the filtrate was concentrated under reduced pressure to afford the titled product Ex.116 (9 mg, 40.8 umol, 86.7% yield, 99% purity) as a yellow solid. ¹HNMR: 400 MHz; δ 7.35-7.25 (m, 1H), 7.00 (d, J=10.4 Hz, 1H), 6.86-6.69 (m, 2H), 4.98 (quin, J=6.4 Hz, 1H), 2.59-2.48 (m, 2H), 2.47-2.39 (m, 2H), 0.62-0.54 (m, 2H), 0.52-0.44 (m, 2H); HPLC: MS: 219.1

### C. Effects of Tropolone Derivatives on the Regulation of Fe (Iron) Transport

In certain embodiments, the effects of tropolone derivatives on the regulation of Fe transport were determined by separate assays including but not necessarily limited to: (1) ligand facilitated Fe(III) efflux from liposomes; and (2) shDMT1-Caco2 55Fe transport assay to assess ligand ability in transporting Fe (III).

### Assay 1: Ligand Facilitated Fe(III) Efflux from Liposomes

### Preparation of POPC (1-palmitoyl-2-oleoyl-sn-glycero-3-phosphocholine): Cholesterol Liposomes

A 1M buffer solution of MES and Tris was prepared by dissolving 121.14 grams of Tris base and 213.25 grams of MES hydrate in 500 mL of MilliQ water and adjusted to pH7.0 using an 18M HCl solution before bringing the total volume of the solution to 1 L. A 500 mM solution of FeCl₃ was prepared by dissolving 0.811 grams of anhydrous FeCl₃ in 10 mL of a 0.1M H₂SO₄ aqueous solution. Inside buffer is prepared in a 50 mL falcon tube, which was added with 25 mL of MilliQ water, 1.61 g of sodium citrate, 1.5 mL of the above FeCl₃ solution, and 2.5 mL of the 1M MES/Tris HCl buffer (pH=7.0), and finally additional MilliQ H₂O to bring to a 50 mL final volume. The inside buffer prepared will be a solution with final concentrations of 15 mM of FeCl₃, 125 mM of sodium citrate, and 50 mM of MES/Tris HCl at pH7.0.

Lipid solution is prepared by dissolving 206.9 mg of POPC (1-palmitoyl-2-oleoyl-sn-glycero-3-phosphocholine) and 8.6 mg of cholesterol in 10 mL of ethanol.

The lipids solution and the inside buffer solution are independently loaded into 10 mL luer lock syringes for loading into a virgin cartridge on a Precision Nanosystems NanoAssembler to prepare unilamaller liposomes with the following parameters: 7.5 mL total volume, 1.5:1 mixing ratio of inside buffer:lipids solution, 8 mL/min flow rate, ambient temperature, 0.35 mL start waste, and 0.05 mL of end waste. 7.5 mL of liposomes is harvested for purification on a 6-inch long, 1-inch diameter Sephadex G-50 column wetted in 600 mM sodium ascorbate and 50 mM MES/Tris HCl pH 7.0 buffer. This buffer also serves as the column running buffer. The crude liposome solution is carefully loaded with minimal volume of running buffer above the top of the Sphadex, allowed to enter the matrix, and the column is run with the continued addition of running buffer. The eluting liposomes, observed as a milky and turbid solution, are collected until free iron begins to elute (observed as a deep purple color) and the fractions are pooled for phosphorus quantitation.

### Determination of Phosphorus Content:

Phosphorus content of eluted liposomes was determined by the process outlined below. 10 μL of liposome elution and a running buffer blank are added to a 5 mL glass vial containing 450 μL of a 8.9 M aqueous H₂SO₄ solution, the mixture is heated to 225 °C for 25 minutes in an aluminum heat block to hydrolyze POPC and cooled for 5 minutes. 200 μL of a 30% hydrogen peroxide aqueous solution is added to each vial and heated to 225 °C for 25 minutes. After cooling, the phosphorus content was determined using an Abcam Phosphate Assay kit, with buffer subtracted phosphorus levels determined against a standard curve of phosphate included in the kit. Liposomes were diluted to 1 mM phosphate in Running Buffer for use in assays after accounting for dilutions made during phospholipid digestion.

### Determination of the Iron Transporting Rate Constant of the Ligands:

Determination of the rate at which a small molecule ligand liberates (transport) Ferric iron from liposomes is performed in clear bottom black 384-well plates on a Spectramax i3×set to read the absorbance at 562 nm in the kinetic mode every 60 seconds for 120 minutes. 1 μL of serially diluted DMSO stock solution of a small molecule ligand is added to wells in triplicate to give the final concentrations of 40, 20, 10, 5, 2.5, and 1.25 uM of the ligand at 80 μL final volume, followed by addition of 1 μL of 100 mM Ferrozine in water to give a final concentration of 1 mM ferrozine. 78 μL of liposomes diluted to 1 mM phosphorus in running buffer is added to wells as quickly as possible (using a digital repeater multichannel pipette) with the kinetic read initiated as rapidly as possible after addition of liposomes to all wells. Typically, eight compounds at six concentrations are tested in triplicate simultaneously.

Upon completion of kinetic read, the data for individual reads is fitted to a single phase association regression with the equation Y=(Y₀−Yₘₐₓ)^{(-kx)}+Y_{Max}, with Y being the 562 absorbance value and X being time in minutes. The k value from the individual replicate values is averaged from the triplicates runs for each compound concentration. The ligand's ability in liberating iron from within liposome is represented by the rate, k, at a given concentration. Rate k is considered as the efflux rate and ligands are ranked by the efflux rate at 10 μM ligand concentration at which they effect Ferric iron efflux. K_{efflux} at 10 μM is reported in Tables 1 and 2.

### Assay 2: ShDMT1-Caco2 ⁵⁵Fe Transport Assay to Assess Ligand Ability in Transporting Fe (III)

### Materials and Methods

Cells: DMT1-deficient Caco-2 cells (alias: “shDMT1,” or “4A” cells) were from Grillo et al. Science, 2017, and were cryopreserved in liquid nitrogen prior to use. Reagents and supplies: ⁵⁵FeCl₃ was obtained from PerkinElmer (Boston, MA). Iron(III) chloride (FeCl₃) hexahydrate was obtained from Sigma. Dulbecco's Modified Eagle Medium (DMEM), fetal bovine serum (FBS), L-glutamine, MEM nonessential amino acids, penicillin-streptomycin, G418, formic acid, methanol, high-purity water, ammonium formate, dimethyl sulfoxide (DMSO) were purchased from Fisher Scientific. Propranolol, atenolol and carbutamide were obtained from Sigma-Aldrich Chemical Company (St Louis, MO). Scintillation cocktail was obtained from Research Product International Co. (Mount Prospect, IL). Stericup filter system (PES membrane, 0.22 um pore size) was purchased from Fisher Scientific. Coming item #3378 24-well transwell insert plates.

Test articles: known compounds hinokitiol and deferiprone were purchased from Sigma. They are tested side by side with the small molecule ligands disclosed in this application. DMSO stocks (10 mM, which is 1,000× of the 10 μM dose level) of hinokitiol or test articles were prepared. A stock solution of 25 mM deferiprone in DMSO was prepared. The DMSO stock solutions were stored at −20 °C or below when not in use.

The growth medium was prepared according to the following table

| | | | | For 1 L |
|---|---|---|---|---|
| Component | Stock | | Final | (mL) |
| DMEM | — | | — | 1,000 |
| FBS | — | | 10% total | 116.2 |
| Glutamine | 200 | mM | 4 mM (2%) | 23.2 |
| PEN-STREP | 1000 | μg/mL | 100 μg/mL (1%) | 11.6 |
| N.E. Amino Acids | — | | 1% total | 11.6 |
| Geneticin G418 disulfate | | | 800 mg/L | |

Apical media (serum free DMEM, 10 mM MES, pH 6.5) was prepared. Apical master mix media was prepared fresh with the addition of 200 nM ⁵⁵Fe before each experiment. For the negative control propranolol and atenolol wells, 200 nM of non-radioactive iron was used.

The basolateral media was serum-free DMEM, 10 mM HEPES, 2% bovine serum albumin (BSA), pH 7.4.

For each experiment, cells were seeded into the 24-well transwell plates (0.5 mL of 50,000 cells/mL) with growth media. The basolateral companion plate was loaded with 1 mL of growth media. After 12-24 hr, both the apical and basolateral chamber were replaced with growth media. The apical media was changed 3× per week for 21-28 days, including a media change exactly 48 hr before the assay date.

On the assay day, the TEER values were measured and the average TEER value was obtained. Individual wells with TEER value>35% lower than the average of all wells were excluded. For the qualified wells, the apical layer (twice) and basolaterial (once) chambers were washed with PBS. The basolateral companion plate was then filled with 1 mL of basolateral media. Via addition down the side-wall of the apical well, 300 μL per well of the apical assay master mix, with the indicated dose level of test article, was added. Each dose was tested in triplicate. The plates were incubated (5% CO₂ and 90% humidity at 37 °C) for the indicated timepoints. At each indicated timepoint, the basolateral supernatant was gently mixed via pipetting, and 200 μL of the basolateral supernatant was transferred to scintillation vials. To each scintillation counting vial, 5 mL of scintillation cocktail fluid was added. For each scintillation vial, the radioactivity (CPM) was determined with liquid scintillation counter LS6500. The counting time per vial was 5 min.

### Data Processing:

All raw CPM values are divided by the average value of blank DMSO solution to give a fold of change above the DMSO value. The mean and standard deviation of each compound at each concentration level and at each time point measured was calculated to give the fold change (fc) value. For rank order compounds, the fc value of a ligand at 4 h time point is further divided by the fold change (fc) value of hinokitiol (a positive control compound) at an equimolar concentration to arrive at the FC (normalized fold of change) value. Data reported in Tables 1 and 2 is 4 hr time point FC=fc-ligand @3 uM/fc-hino @3 μM and FC hino=1.

### Example 117. Experimental Results of (1) Ligand Facilitated Fe(III) Efflux from Liposomes; and (2) shDMT1-Caco2 55Fe Transport Assay to Assess Ligand Ability in Transporting Fe (III)

TABLE 1
Results of Assays (1)-(2) for Selected Compounds

| | | Caco2 |
|---|---|---|
| | | shDMT1 |
| | Liposome Fe | transport |
| | transport | FC @ 3 μM, |
| Structure | k_{efflux} at 10 μM | 4 h |
| 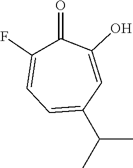 | 10-25 | 0.01-0.5 |
| 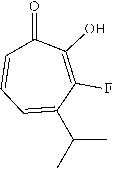 | 10-25 | 0.01-0.5 |
| 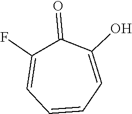 | 0.1-5 | 0.01-0.5 |
| 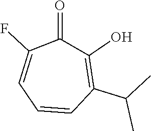 | 10-25 | 0.01-0.5 |
| 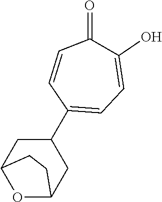 [SMILES: O=c1ccc(C2CC3CCC(C2)O3)ccc1O] | 0.1-5 | 0.01-0.5 |
| 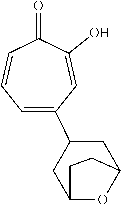 [SMILES: O=c1cccc(C2CC3CCC(C2)O3)cc1O] | 7.5-10 | 0.01-0.5 |
| 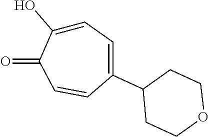 [SMILES: O=c1ccc(C2CCOCC2)ccc1O] | 0.1-5 | 0.01-0.5 |
| 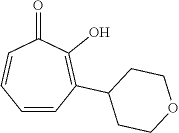 [SMILES: O=c1ccccc(C2CCOCC2)c1O] | 0.1-5 | 1-5 |
| 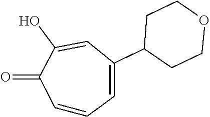 [SMILES: O=c1cccc(C2CCOCC2)cc1O] | 10-25 | 0.01-0.5 |
| 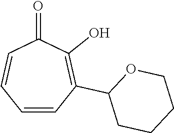 [SMILES: O=c1ccccc(C2CCCCO2)c1O] | 0.1-5 | 1-5 |
| 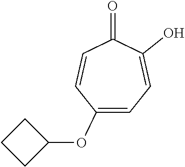 [SMILES: O=c1ccc(OC2CCC2)ccc1O] | 10-25 | 1-5 |
| 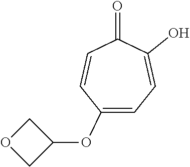 [SMILES: O=c1ccc(OC2COC2)ccc1O] | 5-7.5 | 0.01-0.5 |
| 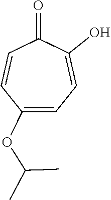 | 7.5-10 | 0.01-0.5 |
| 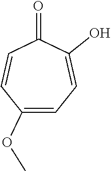 | 5-7.5 | N/A |
| 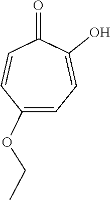 | 7.5-10 | 0.01-0.5 |
| 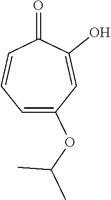 | 5-7.5 | 0.5-1 |
| 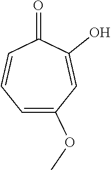 | 5-7.5 | 0.01-0.5 |
| 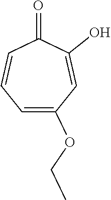 | 5-7.5 | 0.5-1 |
| 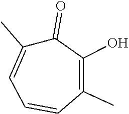 | 10-25 | 1-5 |
| 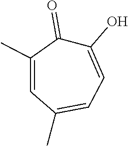 | 5-7.5 | 1-5 |
| 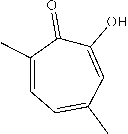 | 0.1-5 | 1-5 |
| 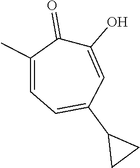 | 7.5-10 | 0.5-1 |
| 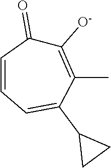 | 0.1-5 | 0.5-1 |
| 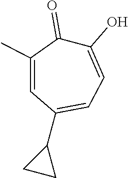 | 0.1-5 | 1-5 |
| 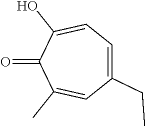 | 5-7.5 | 1-5 |
| 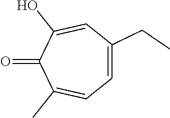 | 5-7.5 | 0.5-1 |
| 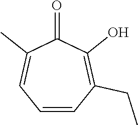 | 5-7.5 | 0.01-0.5 |
| 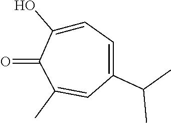 | 10-25 | 1-5 |
| 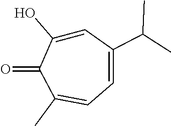 | 0.1-5 | 0.5-1 |
| 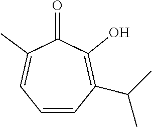 | 5-7.5 | 0.5-1 |
| 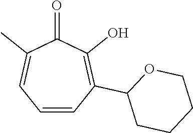 [SMILES: Cc1cccc(C2CCCCO2)c(O)c1=O] | <.1 | N/A |
| 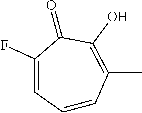 | 10-25 | 0.01-0.5 |
| 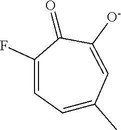 | 0.1-5 | 0.01-0.5 |
| 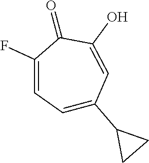 [SMILES: O=c1c(O)cc(C2CC2)ccc1F] | 7.5-10 | 0.01-0.5 |
| 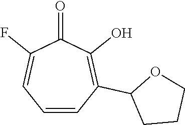 [SMILES: O=c1c(F)cccc(C2CCCO2)c1O] | 5-7.5 | 0.01-0.5 |
| 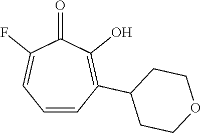 [SMILES: O=c1c(F)cccc(C2CCOCC2)c1O] | 5-7.5 | 0.01-0.5 |
| 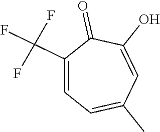 | 7.5-10 | 0.01-0.5 |
| 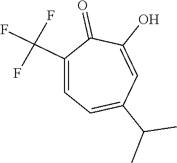 [SMILES: CC(C)c1ccc(C(F)(F)F)c(=O)c(O)c1] | 5-7.5 | 0.01-0.5 |
| 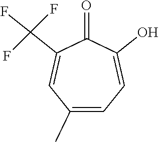 | 10-25 | 0.01-0.5 |
| 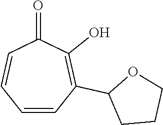 [SMILES: O=c1ccccc(C2CCCO2)c1O] | 10-25 | 1-5 |
| 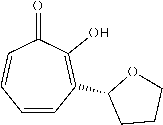 [SMILES: O=c1ccccc([C@H]2CCCO2)c1O] | 7.5-10 | 0.1-5 |
| 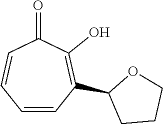 [SMILES: O=c1ccccc([C@@H]2CCCO2)c1O] | 7.5-10 | 0.1-5 |
| 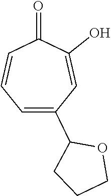 [SMILES: O=c1cccc(C2CCCO2)cc1O] | 7.5-10 | 0.01-0.5 |
| 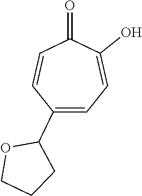 [SMILES: O=c1ccc(C2CCCO2)ccc1O] | 7.5-10 | 0.01-0.5 |
| 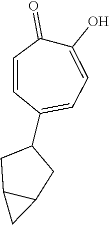 [SMILES: O=c1ccc(C2CC3CC3C2)ccc1O] | 5-7.5 | 0.01-0.5 |
| 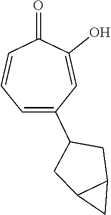 [SMILES: O=c1cccc(C2CC3CC3C2)cc1O] | 0.1-5 | N/A |
| 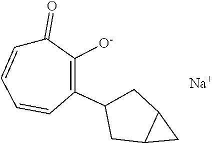 [SMILES: O=c1ccccc(C2CC3CC3C2)c1[O-] ; [Na+]] | 0.1-5 | N/A |
| 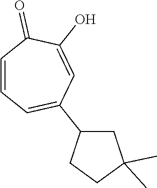 [SMILES: CC1(C)CCC(c2cccc(=O)c(O)c2)C1] | 5-7.5 | 0.01-0.5 |
| 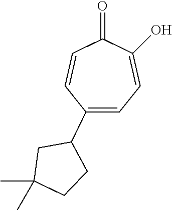 [SMILES: CC1(C)CCC(c2ccc(O)c(=O)cc2)C1] | 0.1-5 | N/A |
| 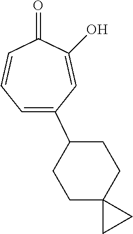 [SMILES: O=c1cccc(C2CCC3(CC2)CC3)cc1O] | 0.1-5 | N/A |
| 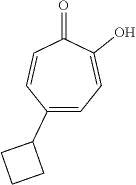 [SMILES: O=c1ccc(C2CCC2)ccc1O] | 10-25 | 0.01-0.5 |
| 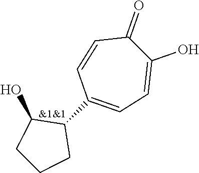 [SMILES: O=c1ccc([C@@H]2CCC[C@H]2O)ccc1O] | 7.5-10 | 0.01-0.5 |
| 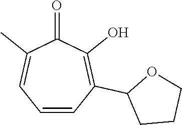 | 10-25 | N/A |
| 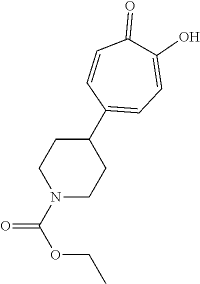 [SMILES: CCOC(=O)N1CCC(c2ccc(O)c(=O)cc2)CC1] | 5-7.5 | 0.01-0.5 |
| 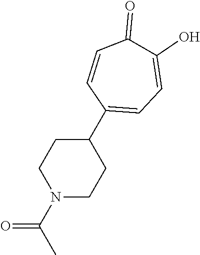 [SMILES: CC(=O)N1CCC(c2ccc(O)c(=O)cc2)CC1] | 5-7.5 | 0.01-0.5 |
| 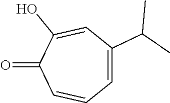 | 7.77 | 1 |
| hinokitiol | | |
| 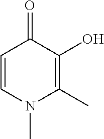 | 0 | 0.02-0.03 |
| deferiprone. | | |

Reference compounds: hinokitiol; and deferiprone.

Reference compounds: hinokitiol; and deferiprone.

TABLE 2
Results of Assays (1)-(2) for Additional Selected Compounds

| | Liposome Fe | Caco2 shDMT1 |
|---|---|---|
| | transport | transport |
| Structure | k_{efflux} at 10 μM | FC @ 3 μM, 4 h |
| 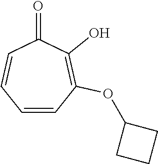 [SMILES: O=c1ccccc(OC2CCC2)c1O] | 7.5-10 | 0.01-0.5 |
| 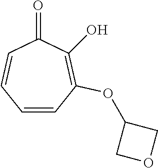 [SMILES: O=c1ccccc(OC2COC2)c1O] | 0.1-5 | 0.01-0.5 |
| 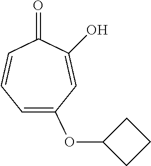 [SMILES: O=c1cccc(OC2CCC2)cc1O] | 0.1-5 | N/A |
| 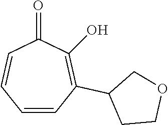 [SMILES: O=c1ccccc(C2CCOC2)c1O] | <0.1 | 0.01-0.5 |
| 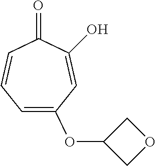 [SMILES: O=c1cccc(OC2COC2)cc1O] | 0.1-5 | 0.01-0.5 |
| 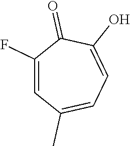 | 0.1-5 | 0.01-0.5 |
| 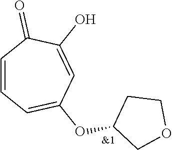 [SMILES: O=c1cccc(O[C@@H]2CCOC2)cc1O] | 0.1-5 | 0.01-0.5 |
| 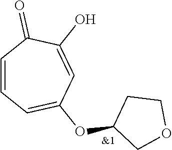 [SMILES: O=c1cccc(O[C@H]2CCOC2)cc1O] | 5-7.5 | N/A |
| 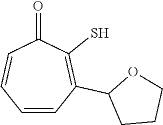 [SMILES: O=c1ccccc(C2CCCO2)c1S] | 7.5-10 | 0.01-0.5 |
| 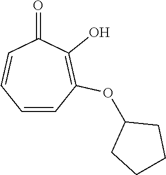 [SMILES: O=c1ccccc(OC2CCCC2)c1O] | 7.5-10 | N/A |
| 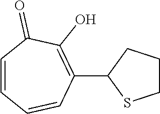 [SMILES: O=c1ccccc(C2CCCS2)c1O] | 0.1-05 | N/A |
| 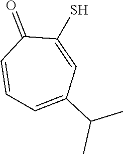 | 0.1-5 | N/A |
| 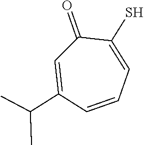 | 0.1-5 | N/A |
| 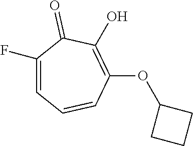 [SMILES: O=c1c(F)cccc(OC2CCC2)c1O] | 10-25 | N/A |
| 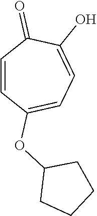 [SMILES: O=c1ccc(OC2CCCC2)ccc1O] | 5-7.5 | N/A |
| 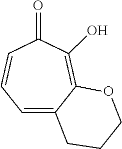 [SMILES: O=c1cccc2c(c1O)OCCC2] | 5-7.5 | N/A |
| 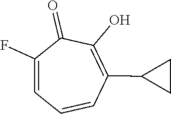 [SMILES: O=c1c(F)cccc(C2CC2)c1O] | 7.5-10 | N/A |
| 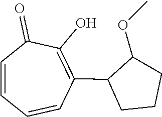 [SMILES: COC1CCCC1c1ccccc(=O)c1O] | 5-7.5 | N/A |
| 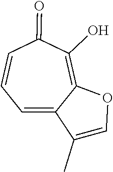 | 7.5-10 | N/A |
| 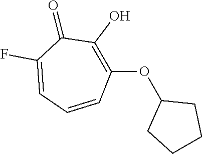 [SMILES: O=c1c(F)cccc(OC2CCCC2)c1O] | 7.5-10 | N/A |
| 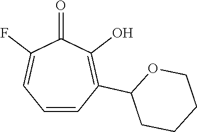 [SMILES: O=c1c(F)cccc(C2CCCCO2)c1O] | 5-7.5 | N/A |
| 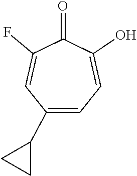 [SMILES: O=c1c(O)ccc(C2CC2)cc1F] | 10-25 | N/A |
| 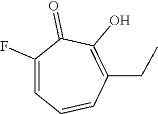 | 10-25 | 0.01-0.5 |
| 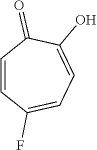 | 7.5-10 | N/A |
| 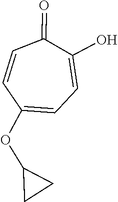 | 10-25 | 0.5-1 |
| 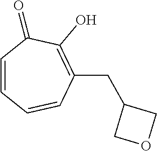 [SMILES: O=c1ccccc(CC2COC2)c1O] | <0.1 | N/A |
| 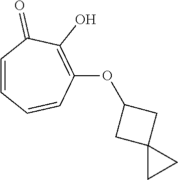 [SMILES: O=c1ccccc(OC2CC3(CC3)C2)c1O] | 10-25 | 1-5 |
| 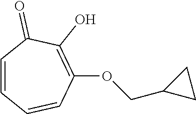 [SMILES: O=c1ccccc(OCC2CC2)c1O] | 10-25 | 1-5 |
| 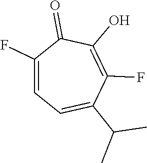 | 7.5-10 | 0.01-0.5 |
| 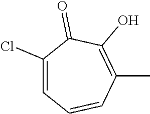 | 5-7.5 | 0.01-0.5 |
| 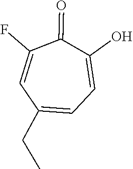 | 0.1-5 | 0.01-0.5 |
| 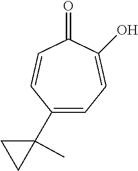 | 0.1-5 | N/A |
| 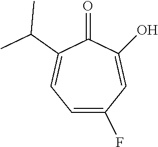 | 0.1-5 | N/A |
| 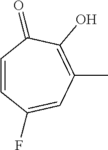 | 5-7.5 | N/A |
| 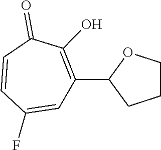 [SMILES: O=c1ccc(F)cc(C2CCCO2)c1O] | 5-7.5 | N/A |
| 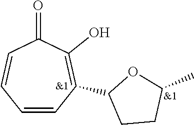 | 5-7.5 | N/A |
| 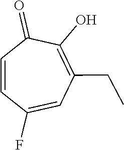 | 7.5-10 | 1-5 |
| 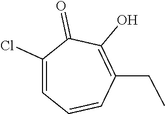 | 7.5-10 | 0.01-0.5 |
| 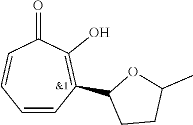 | 5-7.5 | N/A |
| 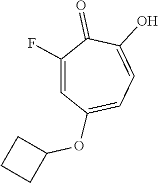 [SMILES: O=c1c(O)ccc(OC2CCC2)cc1F] | 10-25 | 0.01-0.5 |
| 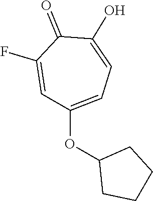 [SMILES: O=c1c(O)ccc(OC2CCCC2)cc1F] | 5-7.5 | N/A |
| 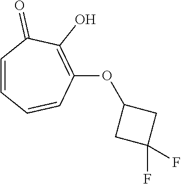 [SMILES: O=c1ccccc(OC2CC(F)(F)C2)c1O] | 7.5-10 | 1-5 |
| 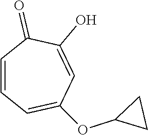 [SMILES: O=c1cccc(OC2CC2)cc1O] | 5-7.5 | 1-5 |
| 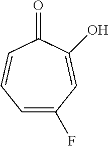 | 0.1-5 | N/A |
| 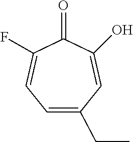 | 0.1-5 | N/A |
| 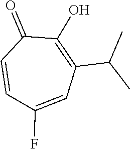 | 0.1-5 | N/A |
| 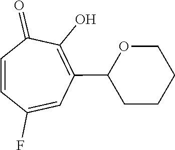 [SMILES: O=c1ccc(F)cc(C2CCCCO2)c1O] | 0.1-5 | N/A |
| 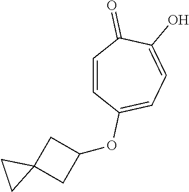 [SMILES: O=c1ccc(OC2CC3(CC3)C2)ccc1O] | 0.1-5 | N/A |
| 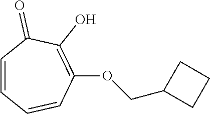 [SMILES: O=c1ccccc(OCC2CCC2)c1O] | 5-7.5 | N/A |
| 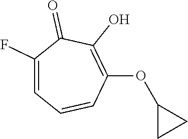 [SMILES: O=c1c(F)cccc(OC2CC2)c1O] | 0.1-5 | N/A |
| 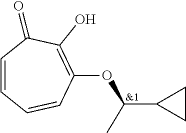 | 5-7.5 | N/A |
| 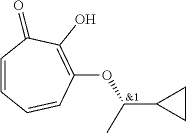 | 5-7.5 | N/A |
| 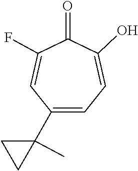 | 5-7.5 | N/A |
| 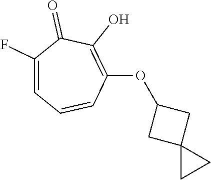 [SMILES: O=c1c(F)cccc(OC2CC3(CC3)C2)c1O] | 0.1-5 | N/A |
| 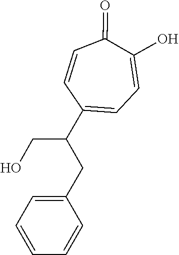 [SMILES: O=c1ccc(C(CO)Cc2ccccc2)ccc1O] | 0.1-5 | N/A |
| 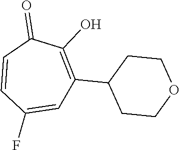 [SMILES: O=c1ccc(F)cc(C2CCOCC2)c1O] | 7.5-10 | N/A |
| 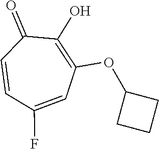 [SMILES: O=c1ccc(F)cc(OC2CCC2)c1O] | 0.1-5 | N/A |
| 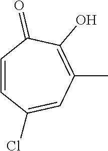 | 5-7.5 | N/A |
| 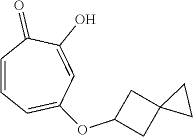 [SMILES: O=c1cccc(OC2CC3(CC3)C2)cc1O] | 5-7.5 | N/A |
| 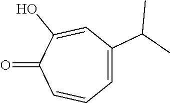 | 7.77 | 1 |
| hinokitiol | | |
| 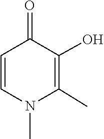 | 0 | 0.02-0.03 |
| deferiprone | | |

Reference compounds: hinokitiol; and deferiprone

Reference compounds: hinokitiol; and deferiprone

### D. Effects of Tropolone Derivatives on Anemia of Inflammation

In certain embodiments, the effects of the compounds of the present disclosure are evaluated according to assay 3.

### Assay 3: Turpentine Oil Induced Anemia of Inflammation

This is a model of anemia of inflammation by turpentine oil injection in combination with phlebotomy.

Conditioning: Mice receive 3 doses of turpentine oil subcutaneously within 2 weeks. Mice are anesthetized and then administered 0.1 mL/20 g body weight of turpentine oil (TO) via subcutaneous injection in the scapular fat area using syringe and 27 G needle on Day 1, 7, and 14. On day 14, after the final injection of turpentine oil, a controlled 10% hemorrhage (approximately 200 uL) is accomplished via anesthetized orbital sinus bleed to increase the magnitude of anemia. The bleeding uses an orbital blood sampling technique, 200 uL capillary bleeding device (essentially a vacutainer tube with a glass capillary tube attached). Clean gauze lightly pre-soaked with sterile saline is held over the eye immediately following hemorrhage to promote hemostasis.

Treatment: On day 15, 16 hours post last dose of turpentione oil, sham group and TO plus bleeding group are terminated. These two groups are used as part of control.

On day 15, vehicle group and compound treatment groups receive proper vehicle or compounds at a specified dose accordingly, treatments can be oral once a day or oral twice a day. The treatment continues for 7 days, and then is terminated on day 21.

On day 21, 3 hours post last treatment of test articles, the vehicle and treatment group are terminated. At the time of blood collection and tissue harvest, mice are weighed then anesthetized with isoflurane anesthesia (3-4% with oxygen to a surgical plane of anesthesia). Depth of anesthesia is monitored by toe pinch method. Blood is collected through cardiac puncture which is a terminal procedure. Blood samples are placed into tubes with EDTA anticoagulated to undergo CBC analysis. The remaining blood is placed into serum separator to collect serum for Total Iron, Ferritin and TIBC testing by clinical chemical analyzer and, in some instances, a proinflammatory cytokine panel, including e.g., cytokines selected from IL-6, TNF-α, IL-10, IL-1α, IL-4, IL-6, IL-13, IFNα and IL-1β or a 9-cytokine panel such as those commercially available from Luminex. Ferritin measurement allows for assessment of transferrin saturation, a significant and clinically relevant measure of correction of anemia due to anemia of inflammation. Leftover serum is used for assessment of drug concentration at termination. After blood sampling the tissues of duodenum, spleen and liver are snap frozen (in multiple aliquots per organ) in liquid nitrogen and stored at −80 °C for determination of iron content and gene expression assessments, e.g., hepcidin gene expression and Ferroportin gene and protein expression assessments.

Data analysis: The iron mobilization impact of compounds is assessed based on the serum iron level, transferrin saturation and TIBC; the ability of test article to restore normal iron homeostasis and metabolism, and subsequent rescue of anemia, is assessed by comparing the hemoglobin and hematocrit levels of test article treated groups with vehicle treated group.

### Example 118. Experimental Results of Assay 3, Turpentine Oil Induced Anemia of Inflammation

Test articles were evaluated in the described mouse model of turpentine oil (TO)-induced anemia of inflammation (A of 1). Sham animals (i.e., mice that did not receive TO or controlled phlebotomy) served as a negative anemia control and were assayed 16 hours after the time point that the test article-treated groups received final TO administration (i.e., 6 days before the end of the study). Animals that received TO, controlled phlebotomy, and vehicle and were assayed at the end of the study served as the positively-induced anemia group.

The anemia group (TO+bleed) displayed an average reduction in hemoglobin (Hgb) of 2.28 g/dL, roughly equivalent (as a percent of sham/normal hemoglobin concentration) to about a 20% reduction. Test compounds were administered to animals that received TO and controlled phlebotomy. Animals were dosed via oral gavage (PO) at 100 mg/kg QD or 30 mg/kg BID as indicated below. When included as a comparator, hinokitiol, dosed at 100 mg/kg QD PO, yielded increases in Hgb ranging from +0.4 to +1.0 g/dL over multiple separate experiments.

With hinokitiol being included as a comparator, various test articles were evaluated in the TO model. Whereas multiple test articles, when dosed at 100 mg/kg or higher, did not yield comparable or higher levels of Hgb than hinokitiol in their respective studies, multiple molecules, including e.g., Ex.1, Ex.32, and Ex.42 (dosed at 100 mg/kg or less), unexpectedly yielded comparable or higher levels of Hgb than hinokitiol in the respective studies. In separate studies, multiple compounds were evaluated in the TO model without hinokitiol as a comparator. Compounds were dosed at either 100 mg/kg QD or 30 mg/kg BID. In these tests, while some compounds did not increase Hgb levels by at least 0.4 g/dL higher than vehicle, multiple compounds, such as Ex.11 and Ex.16, increased mean Hgb levels at least +0.4 g/dL higher than vehicle in the respective studies.

Collectively, the results of this example, together with results of other assays described herein, demonstrate that compounds of the present disclosure display iron transporting activity, in both in vitro and in vivo assays, that is comparable to or better than hinokitiol.

### Example 119. Compounds of the Present Disclosure have Advantageous Absorption, Distribution, Metabolism, Excretion (ADME) and Drug Metabolism and Pharmacokinetics (DMPK) Properties

Compounds of the present disclosure were further evaluated in standard in vitro and in vivo Absorption, Distribution, Metabolism, Excretion (ADME) and Drug Metabolism and Pharmacokinetics (DMPK) assays to assess drug profiles important for further drug development.

Such assessments included human and mouse liver microsomal stability assays as well as 6-hour mouse PK studies. Compounds of the present disclosure demonstrated desirable ADME and DMPK characteristics, including e.g., improved in vitro liver microsomal stability and/or in vivo PK properties as compared to hinokitiol. For example, as shown in Table 3, numerous compounds, including Ex. 1, Ex. 2, Ex. 4, Ex. 12, Ex. 32, Ex. 36, Ex. 40, Ex. 41, Ex. 42, and Ex. 56 displayed a longer clearance half-life (t_{1/2}) and lower intrinsic clearance (CLᵢₙₜ), or both as compared to hinokitiol in both mouse and human liver microsomal stability assays.

TABLE 3
Liver Microsomal Stability Results for Selected Compounds

| | Human | | Mouse | |
|---|---|---|---|---|
| | | CLᵢₙₜ | | CLᵢₙₜ |
| | t_{1/2} | (μL/min/mg | t_{1/2} | (μL/min/mg |
| Compound | (min) | protein) | (min) | protein) |
| Hinokitiol | 11.59 | 119.59 | 29.68 | 46.70 |
| 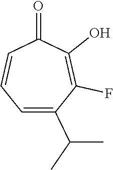 | >150 | <24.99 | >150 | <24.99 |
| Ex. 2 | | | | |
| 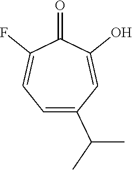 | 25-49.99 | 25-49.99 | >150 | <24.99 |
| Ex. 1 | | | | |
| 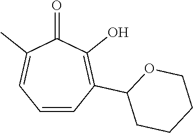 [SMILES: Cc1cccc(C2CCCCO2)c(O)c1=O] | 50-99.99 | 25-49.99 | 25-49.99 | 25-49.99 |
| Ex. 40 | | | | |
| 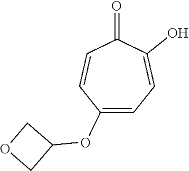 [SMILES: O=c1ccc(OC2COC2)ccc1O] | ∞* | 0.00* | ∞* | 0.00* |
| Ex. 12 | | | | |
| 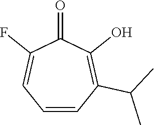 | 25-49.99 | >50 | 25-49.99 | 25-49.99 |
| Ex. 4 | | | | |
| 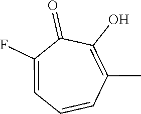 | >150 | <24.99 | >150 | <24.99 |
| Ex. 32 | | | | |
| 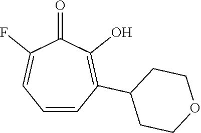 [SMILES: O=c1c(F)cccc(C2CCOCC2)c1O] | ∞* | 0.00* | >150 | <24.99 |
| Ex. 36 | | | | |
| 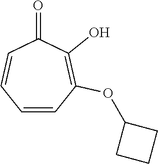 [SMILES: O=c1ccccc(OC2CCC2)c1O] | 100-149.99 | <24.99 | 25-49.99 | 25-49.99 |
| Ex. 56 | | | | |
| 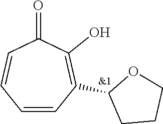 [SMILES: O=c1ccccc([C@H]2CCCO2)c1O] | 25-49.99 | 25-49.99 | 25-49.99 | 25-49.99 |
| Ex. 41 | | | | |
| 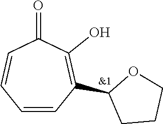 [SMILES: O=c1ccccc([C@@H]2CCCO2)c1O] | 50-99.99 | <24.99 | 25-49.99 | 25-49.99 |
| Ex. 42 | | | | |

*∞ t_{1/2} and 0.00 CLᵢₙₜ indicate stability beyond the length of the assay

In addition, as shown in Table 4, numerous compounds, including Ex. 1, Ex. 2, Ex. 4, Ex. 6, Ex. 8, Ex. 12, Ex. 27, Ex. 28, Ex. 30, Ex. 32, Ex. 34, Ex. 36, Ex. 40, Ex. 41, Ex. 42, Ex. 56, Ex. 82, and Ex.83, displayed one or more improved mouse 6-hour PK properties as compared to hinokitiol.

TABLE 4
Mouse 6-hour PK Results

| | Cₘₐₓ | AUCₗₐₛₜ 0-6 hr | t_{1/2} |
|---|---|---|---|
| Compound | (ng/mL) | (hr*ng/mL) | (hr) |
| Hinokitiol | 956 | 584 | 1.11 |
| 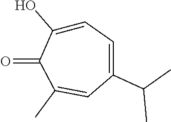 | <499 | <499 | 1.25-4.99 |
| Ex. 28 | | | |
| 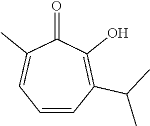 | <499 | <499 | 5-9.99 |
| Ex. 30 | | | |
| 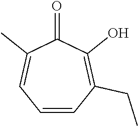 | 500-1499 | 500-1499 | 1.25-4.99 |
| Ex. 27 | | | |
| 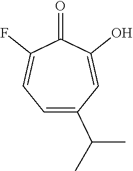 | 5000-19999 | 5000-19999 | 1.25-4.99 |
| Ex. 1 | | | |
| 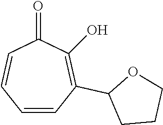 [SMILES: O=c1ccccc(C2CCCO2)c1O] | 1500-4999 | 500-1499 | <1.25 |
| Ex. 40 | | | |
| 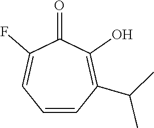 | 5000-19999 | >20000 | 1.25-4.99 |
| Ex. 4 | | | |
| 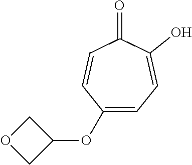 [SMILES: O=c1ccc(OC2COC2)ccc1O] | 500-1499 | <499 | 1.25-4.99 |
| Ex. 12 | | | |
| 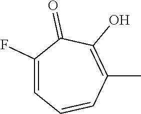 | 5000-19999 | >20000 | 1.25-4.99 |
| Ex. 32 | | | |
| 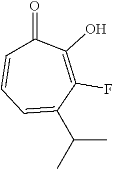 | 5000-19999 | 5000-19999 | 1.25-4.99 |
| Ex. 2 | | | |
| 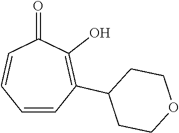 [SMILES: O=c1ccccc(C2CCOCC2)c1O] | 500-1499 | 500-1499 | 1.25-4.99 |
| Ex. 8 | | | |
| 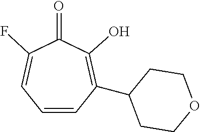 [SMILES: O=c1c(F)cccc(C2CCOCC2)c1O] | >20000 | >20000 | 1.25-4.99 |
| Ex. 36 | | | |
| 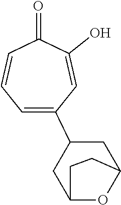 [SMILES: O=c1cccc(C2CC3CCC(C2)O3)cc1O] | 500-1499 | 500-1499 | 1.25-4.99 |
| Ex. 6 | | | |
| 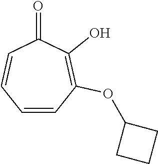 [SMILES: O=c1ccccc(OC2CCC2)c1O] | 1500-4999 | 1500-4999 | <1.25 |
| Ex. 56 | | | |
| 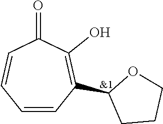 [SMILES: O=c1ccccc([C@@H]2CCCO2)c1O] | 1500-4999 | 500-1499 | <1.25 |
| Ex. 41 | | | |
| 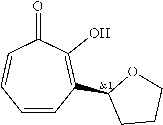 [SMILES: O=c1ccccc([C@@H]2CCCO2)c1O] | 1500-4999 | 500-1499 | <1.25 |
| Ex. 42 | | | |
| 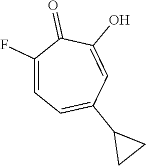 [SMILES: O=c1c(O)cc(C2CC2)ccc1F] | 5000-19999 | >20000 | 1.25-4.99 |
| Ex. 34 | | | |
| 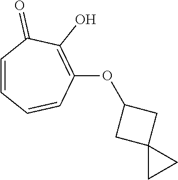 [SMILES: O=c1ccccc(OC2CC3(CC3)C2)c1O] | <499 | 500-1499 | <1.25 |
| Ex. 82 | | | |
| 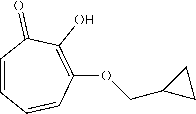 [SMILES: O=c1ccccc(OCC2CC2)c1O] | 1500-4999 | 500-1499 | <1.25 |
| Ex. 83 | | | |

Accordingly, the results presented above demonstrate that compounds of the present disclosure have desirable ADME and DMPK characteristics, including e.g., where such characteristics are improved as compared to hinokitiol.

### EQUIVALENTS

The foregoing written specification is considered to be sufficient to enable one skilled in the art to practice the present disclosure. The present disclosure is not to be limited in scope by examples provided, since the examples are intended as a single illustration of one aspect of the present disclosure and other functionally equivalent embodiments are within the scope of the present disclosure. Various modifications of the present disclosure in addition to those shown and described herein will become apparent to those skilled in the art from the foregoing description and fall within the scope of the appended claims. The advantages and objects of the present disclosure are not necessarily encompassed by each embodiment of the present disclosure.

What is claimed is:

1. A compound or a tautomer thereof, or a pharmaceutically acceptable salt of either, represented by Formula Ia: 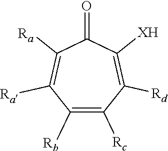
wherein:
X represents oxygen or sulfur;
Rₐ represents hydrogen, halo, alkyl, substituted alkyl, heteroalkyl, alkoxy, substituted alkoxy, alkoxyalkyl, substituted alkoxyalkyl, cycloalkyl, substituted cycloalkyl, heterocycloalkyl, substituted heterocycloalkyl, alkenyl, substituted alkenyl, heteroalkenyl, cycloalkenyl, substituted cycloalkenyl, heterocycloalkenyl, substituted heterocycloalkenyl, alkynyl, substituted alkynyl, or heteroalkynyl;
Rₐ′ represents hydrogen, halo, alkyl, or substituted alkyl; and R_{b}, R_{c}, and R_{d} are independently selected from the group consisting of hydrogen, halo, alkyl, substituted alkyl, heteroalkyl, alkylcycloalkyl, alkylheterocycloalkyl, alkoxy, substituted alkoxy, alkoxyalkyl, substituted alkoxyalkyl, cycloalkyloxy, substituted cycloalkyloxy, heterocycloalkyloxy, substituted heterocycloalkyloxy, cycloalkyl, substituted cycloalkyl, heterocycloalkyl, substituted heterocycloalkyl, alkenyl, substituted alkenyl, heteroalkenyl, cycloalkenyl, substituted cycloalkenyl, heterocycloalkenyl, substituted heterocycloalkenyl, alkynyl, substituted alkynyl, and heteroalkynyl; provided that Rₐ, R_{b}, R_{c}, and R_{d} are not all hydrogen.

2. The compound or a tautomer of claim 1, wherein Rₐ′ is hydrogen.

3. The compound or a tautomer of claim 1, wherein Rₐ′ is halo.

4. The compound or tautomer of claim 1, wherein Rₐ′ is alkyl, or substituted alkyl.

5. The compound or tautomer of any one of claims 1-4, wherein at least one of Rₐ, R_{b}, R_{c}, and R_{d} is selected from the group consisting of halo, alkyl, substituted alkyl, heteroalkyl, alkylcycloalkyl, alkylheterocycloalkyl, alkoxy, substituted alkoxy, alkoxyalkyl, substituted alkoxyalkyl, cycloalkyloxy, substituted cycloalkyloxy, heterocycloalkyloxy, substituted heterocycloalkyloxy, cycloalkyl, substituted cycloalkyl, heterocycloalkyl, and substituted heterocycloalkyl.

6. The compound or a tautomer of claim 1 or 2, wherein the compound or a tautomer thereof is represented by Formula Ib: 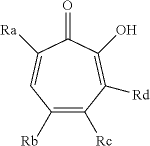
wherein:
Rₐ represents hydrogen, halo, alkyl, substituted alkyl, heteroalkyl, alkoxy, substituted alkoxy, alkoxyalkyl, substituted alkoxyalkyl, cycloalkyl, substituted cycloalkyl, heterocycloalkyl, substituted heterocycloalkyl, alkenyl, substituted alkenyl, heteroalkenyl, cycloalkenyl, substituted cycloalkenyl, heterocycloalkenyl, substituted heterocycloalkenyl, alkynyl, substituted alkynyl, or heteroalkynyl; and
R_{b}, R_{c}, and R_{d} are independently selected from the group consisting of hydrogen, halo, alkyl, substituted alkyl, heteroalkyl, alkoxy, substituted alkoxy, alkoxyalkyl, substituted alkoxyalkyl, cycloalkyl, substituted cycloalkyl, heterocycloalkyl, substituted heterocycloalkyl, alkenyl, substituted alkenyl, heteroalkenyl, cycloalkenyl, substituted cycloalkenyl, heterocycloalkenyl, substituted heterocycloalkenyl, alkynyl, substituted alkynyl, and heteroalkynyl;
provided that Rₐ, R_{b}, R_{c}, and R_{d} are not all hydrogen.

7. The compound or tautomer of any one of claims 1-6, wherein:
each occurrence of heterocycloalkyl is independently selected from the group consisting of: 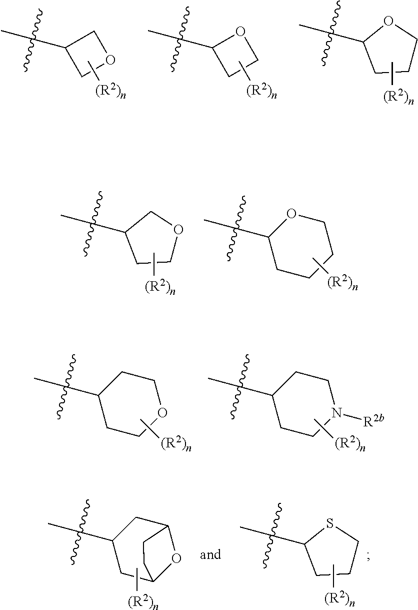
n is independently for each occurrence an integer selected from 0-5 inclusive;
R^{2b} is independently for each occurrence hydrogen, alkyl, substituted alkyl, heteroalkyl, hydroxy, alkoxy, substituted alkoxy, alkoxyalkyl, substituted alkoxyalkyl, cycloalkyl, substituted cycloalkyl, alkenyl, substituted alkenyl, cycloalkenyl, substituted cycloalkenyl, alkynyl, and substituted alkynyl; and
each instance of R² independently represents —F, alkyl, haloalkyl, or alkoxy; or two geminal instances of R² represent carbonyl.

8. The compound or tautomer of any one of claims 1-7, wherein Rₐ represents —F, —CF₃, (C2-C15)alkyl, or substituted (C1-C15)alkyl.

9. The compound or tautomer of claim 8, wherein:
R_{b}, R_{c}, and R_{d} independently represent hydrogen, (C1-C15)alkyl, 3-12 membered cycloalkyl, 3-12 membered heterocycloalkyl, (C1-C15)alkylene-R₁, 3-12 membered cycloalkyl-R₁, or 3-12 membered heterocycloalkyl-R₁; and
R₁ represents independently for each occurrence halo, alkyl, alkoxy, or hydroxyl.

10. The compound or tautomer of claim 9, wherein said compound is selected from the group consisting of:

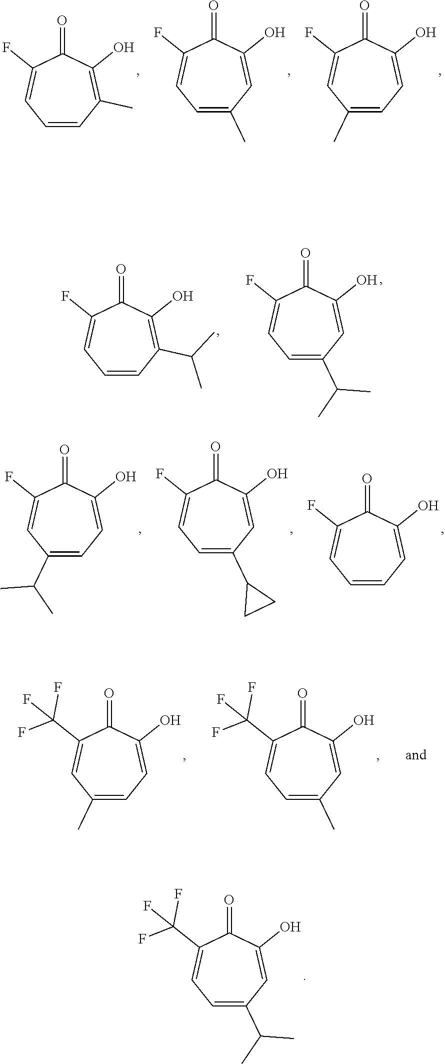

11. The compound or tautomer of claim 9, wherein said compound is selected from the group consisting of:

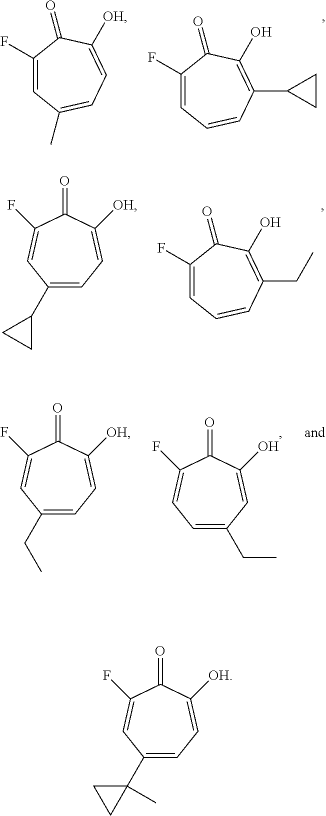

12. The compound or tautomer of any one of claims claim 1-7, wherein Rₐ represents —Cl, —F, —CF₃, (C2-C15)alkyl, or substituted (C1-C15)alkyl.

13. The compound or tautomer of claim 12, wherein:
R_{b}, R_{c}, and R_{d} independently represent hydrogen, halo (C1-C15)alkyl, 3-12 membered cycloalkyl, 3-12 membered heterocycloalkyl, (C1-C15)alkylene-R₁, 3-12 membered cycloalkyl-R₁, or 3-12 membered heterocycloalkyl-R₁; and
R₁ represents independently for each occurrence halo, alkyl, alkoxy, or hydroxyl.

14. The compound or tautomer of claim 13, wherein said compound is selected from the group consisting of:

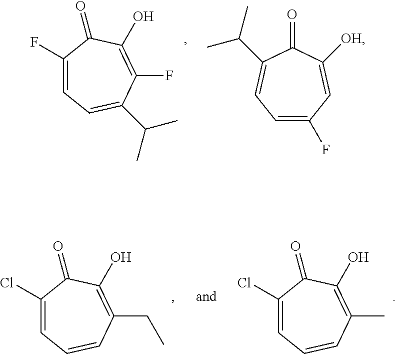

15. The compound or tautomer of any one of claims 1-7, wherein:
R_{b}, R_{c}, and R_{d} independently represent hydrogen or 3-12 membered heterocycloalkyl optionally substituted with one or two instances of R₂;
wherein said heterocycloalkyl comprises one or two oxygen atoms, one or two nitrogen atoms, one or two sulfur atoms, or any combination of two atoms selected from the group consisting of oxygen, nitrogen, and sulfur atoms; and
each instance of R₂ independently represents —F, alkyl, haloalkyl, carbonyl, or alkoxy.

16. The compound or tautomer of claim 15, wherein said compound is selected from the group consisting of:

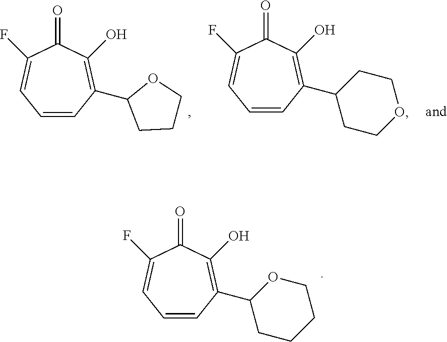 [SMILES: O=c1c(F)cccc(C2CCCO2)c1O ; O=c1c(F)cccc(C2CCOCC2)c1O ; O=c1c(F)cccc(C2CCCCO2)c1O]

17. The compound or tautomer of claim 15, wherein:
said heterocycloalkyl comprises one or two nitrogen atoms; and at least one of said nitrogen atoms is represented by N(R_{2b});
R_{2b} independently represents hydrogen, —C(O)R₅, or —C(O)OR₅; and
R₅ independently represents hydrogen, alkyl, or substituted alkyl.

18. The compound or tautomer of any one of claims 1-7, wherein:
R_{b}, R_{c}, and R_{d} independently represent hydrogen or —OR₃; and
R₃ independently represents (C1-C15)alkyl, 3-12 membered cycloalkyl or bicycloalkyl, 3-12 membered heterocycloalkyl or heterobicylcloalkyl comprises one or two oxygen atoms, one or two nitrogen atoms, one or two sulfur atoms, or any combination of two atoms selected from the group consisting of oxygen, nitrogen, and sulfur atoms.

19. The compound or tautomer of claim 18, wherein said compound is selected from the group consisting of:

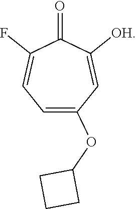 [SMILES: O=c1c(O)cc(OC2CCC2)ccc1F]

20. The compound or tautomer of claim 18, wherein said compound is selected from the group consisting of:

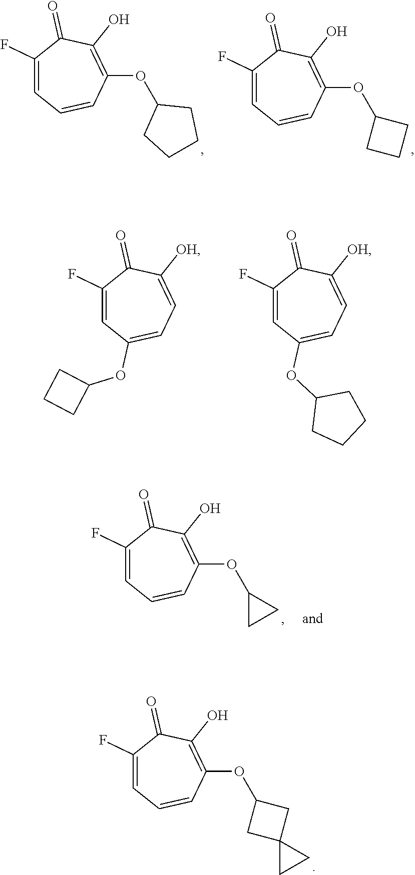 [SMILES: O=c1c(F)cccc(OC2CCCC2)c1O ; O=c1c(F)cccc(OC2CCC2)c1O ; O=c1c(O)ccc(OC2CCC2)cc1F ; O=c1c(O)ccc(OC2CCCC2)cc1F ; O=c1c(F)cccc(OC2CC2)c1O ; O=c1c(F)cccc(OC2CC3(CC3)C2)c1O]

21. The compound or tautomer of claim 18, wherein each instance of R₃ is optionally substituted with R₄; and R₄ independently represents alkyl, halogen-substituted alkyl, alkoxy, or hydroxy.

22. The compound or tautomer of any one of claims 1-7, wherein Rₐ represents —CH₃.

23. The compound or tautomer of claim 22, wherein:
R_{b}, R_{c}, and R_{d} independently represent hydrogen, (C1-C15)alkyl, 3-12 membered cycloalkyl, 3-12 membered heterocycloalkyl, (C1-C15)alkylene-R₁, 3-12 membered cycloalkyl-R₁, or 3-12 membered heterocycloalkyl-R₁; and
R₁ represents independently for each occurrence halo, alkyl, alkoxy, or hydroxy.

24. The compound or tautomer of claim 23, wherein said compound is selected from the group consisting of:

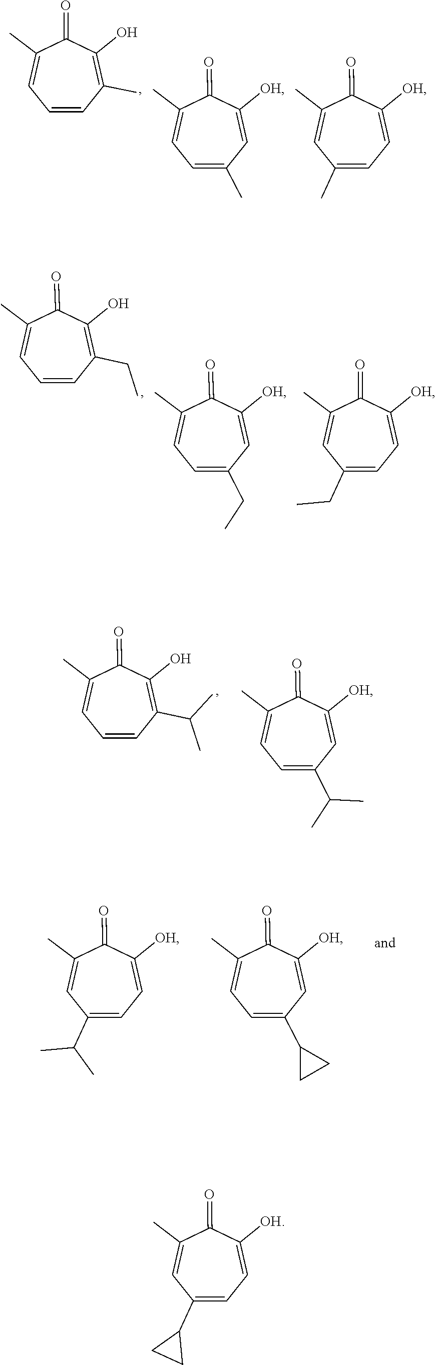

25. The compound or tautomer of claim 22, wherein:
R_{b}, R_{c}, and R_{d} independently represent hydrogen or 3-12 membered heterocycloalkyl optionally substituted with one or two instances of R₂;
wherein said heterocycloalkyl comprises one or two oxygen atoms, one or two nitrogen atoms, one or two sulfur atoms, or any combination of two atoms selected from the group consisting of oxygen, nitrogen, and sulfur atoms; and
each instance of R₂ independently represents —F, alkyl, haloalkyl, carbonyl, or alkoxy.

26. The compound or tautomer of claim 25, wherein said compound is selected from the group consisting of:

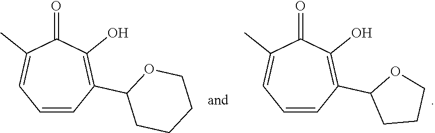 [SMILES: Cc1cccc(C2CCCCO2)c(O)c1=O ; Cc1cccc(C2CCCO2)c(O)c1=O]

27. The compound or tautomer of claim 25, wherein:
said heterocycloalkyl comprises one or two nitrogen atoms; and at least one of said nitrogen atoms is represented by N(R_{2b});
R_{2b} independently represents hydrogen, —C(O)R₅, or —C(O)OR₅; and
R₅ independently represents hydrogen, alkyl, or substituted alkyl.

28. The compound or tautomer of claim 22, wherein:
R_{b}, R_{c}, and R_{d} independently represent hydrogen or —OR₃; and
R₃ independently represents (C1-C15)alkyl, 3-12 membered cycloalkyl or bicycloalkyl, 3-12 membered heterocycloalkyl or heterobicylcloalkyl comprising one or two oxygen atoms, one or two nitrogen atoms, one or two sulfur atoms, or any combination of two atoms selected from the group consisting of oxygen, nitrogen, and sulfur atoms.

29. The compound or tautomer of claim 28, wherein each instance of R₃ is optionally substituted with R₄; and R₄ independently represents alkyl, halogen-substituted alkyl, alkoxy, or hydroxy.

30. The compound or tautomer of any one of claims 1-7, wherein Rₐ represents hydrogen.

31. The compound or tautomer of claim 30, wherein R_{b}, R_{c}, and Re independently represent hydrogen, (C1-C15)alkyl, —F or —CF₃.

32. The compound or tautomer of claim 30 or 31, wherein said compound is selected from the group consisting of:

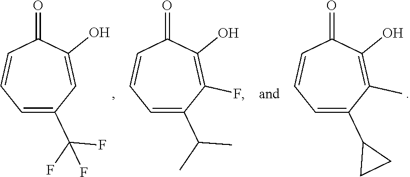

33. The compound or tautomer of claim 31, wherein said compound is selected from the group consisting of:

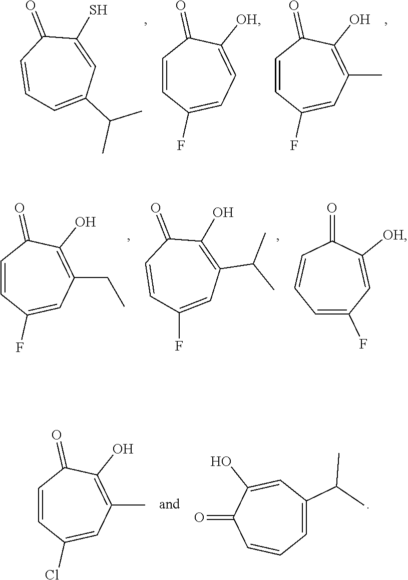

34. The compound or tautomer of claim 30, wherein R_{b}, R_{c}, and R_{d} independently represent hydrogen, (C1-C15)alkyl, —Cl, —F or —CF₃.

35. The compound or tautomer of claim 34, wherein said compound is

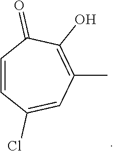

36. The compound or tautomer of claim 30, wherein:
R_{b}, R_{c}, and R_{d} independently represent hydrogen, 3-12 membered cycloalkyl or or 3-12 membered heterocycloalkyl each optionally substituted with one or two instances of R₂;
wherein said heterocycloalkyl comprises one or two oxygen atoms, one or two nitrogen atoms, one or two sulfur atoms, or any combination of two atoms selected from the group consisting of oxygen, nitrogen, and sulfur atoms; and
each instance of R₂ independently represents —F, alkyl, haloalkyl, carbonyl, or alkoxy.

37. The compound or tautomer of claim 36, wherein said compound is selected from the group consisting of:

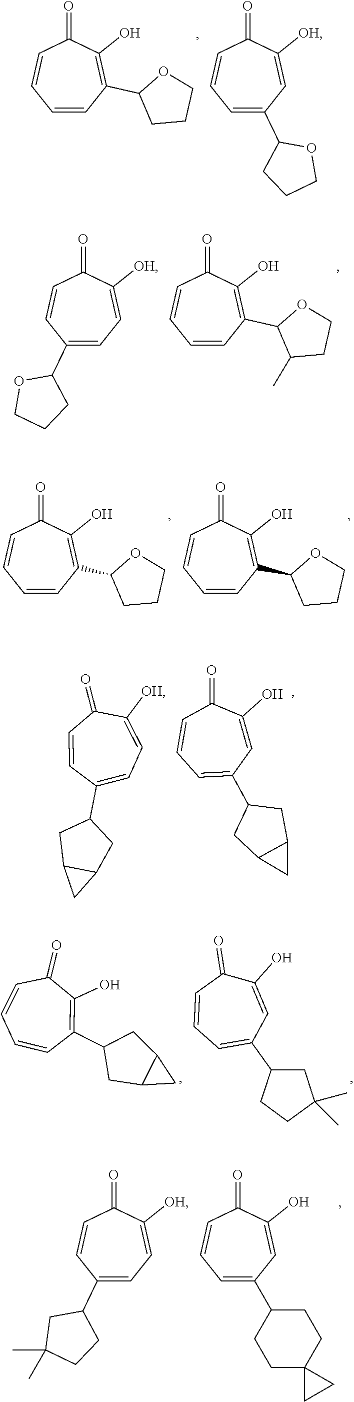

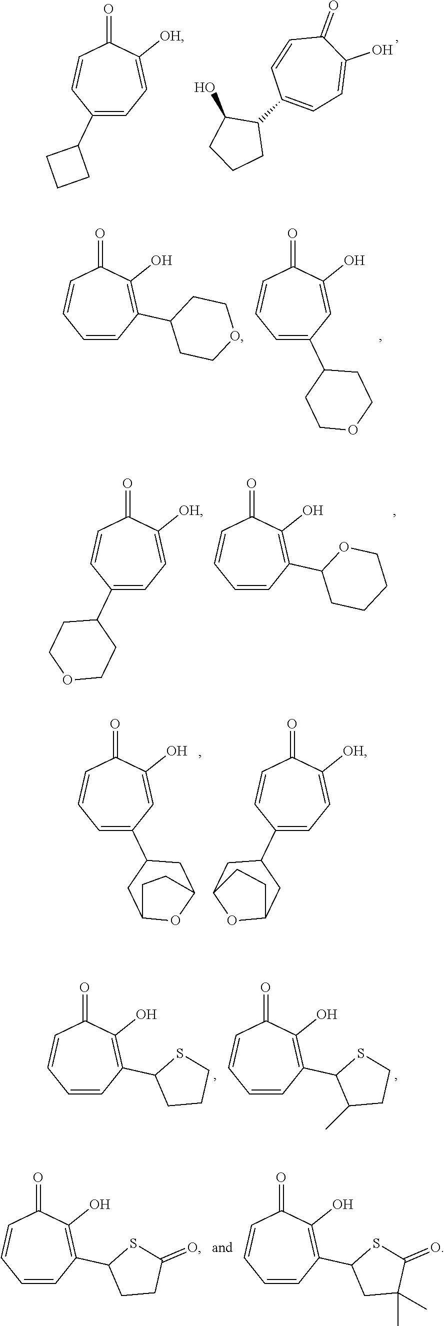

38. The compound or tautomer of claim 36, wherein said compound is selected from the group consisting of:

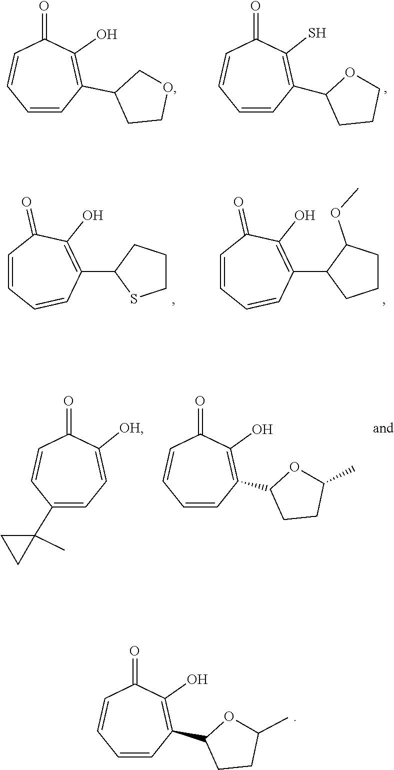

39. The compound or tautomer of claim 30, wherein:
R_{b} is halo;
R_{c} and R_{d} independently represent hydrogen, 3-12 membered cycloalkyl, 3-12 membered heterocycloalkyl, cycloalkyloxy, or heterocycloalkyloxy,
each cycloalkyl heterocycloalkyl, cycloalkyloxy, or heterocycloalkyloxy is optionally substituted with one or two instances of R₂;
wherein said heterocycloalkyl comprises one or two oxygen atoms, one or two nitrogen atoms, one or two sulfur atoms, or any combination of two atoms selected from the group consisting of oxygen, nitrogen, and sulfur atoms; and
each instance of R₂ independently represents —F, alkyl, haloalkyl, carbonyl, or alkoxy.

40. The compound or tautomer of claim 39, wherein said compound is selected from the group consisting of:

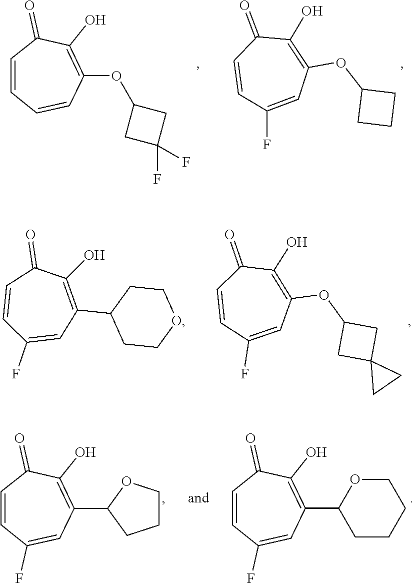

41. The compound or tautomer of claim 36, wherein:
said heterocycloalkyl comprises one or two nitrogen atoms; and at least one of said nitrogen atoms is represented by N(R_{2b});
R_{2b} independently represents hydrogen, —C(O)R₅, or —C(O)OR₅; and
R₅ independently represents hydrogen, alkyl, or substituted alkyl.

42. The compound or tautomer of claim 41, wherein said compound is selected from the group consisting of:

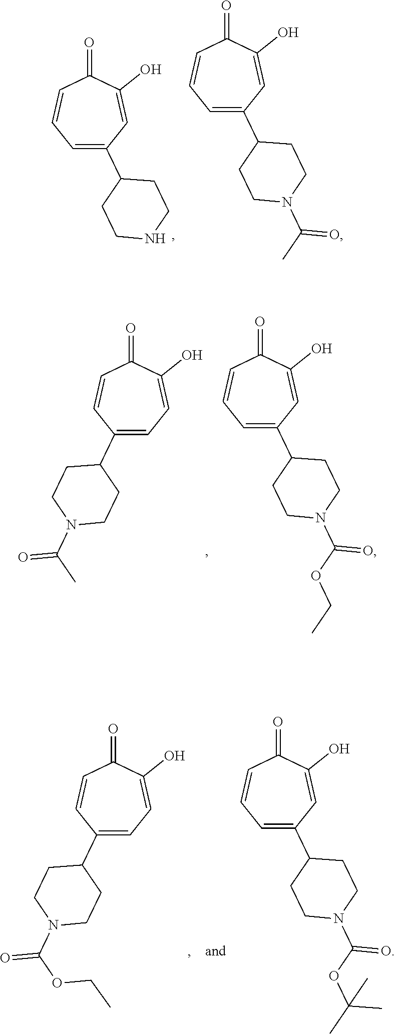

43. The compound or tautomer of claim 30, wherein:
R_{b}, R_{c}, and R_{d} independently represent hydrogen or —OR₃; and
R₃ independently represents (C1-C15)alkyl, 3-12 membered cycloalkyl or bicycloalkyl, or 3-12 membered heterocycloalkyl or heterobicylcloalkyl comprising one or two oxygen atoms, one or two nitrogen atoms, one or two sulfur atoms, or any combination of two atoms selected from the group consisting of oxygen, nitrogen, and sulfur atoms.

44. The compound or tautomer of claim 43, wherein said compound is selected from the group consisting of:

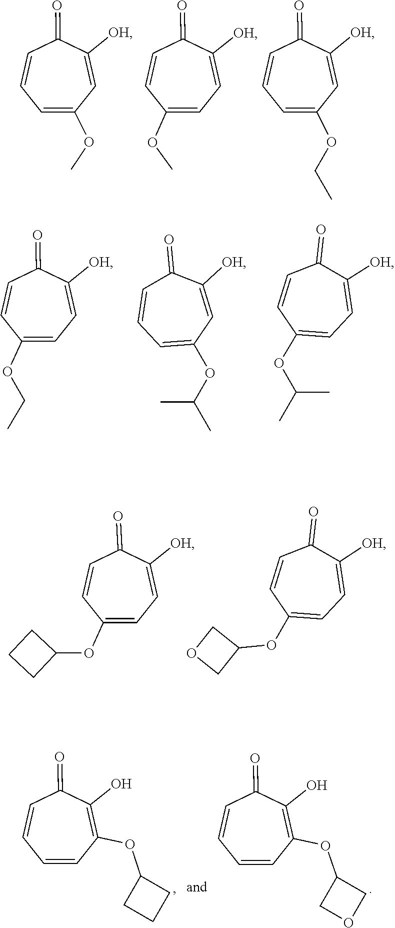

45. The compound or tautomer of claim 43, wherein each instance of R₃ is optionally substituted with R₄; and R₄ independently represents alkyl, halogen-substituted alkyl, alkoxy, or hydroxy.

46. The compound or tautomer of claim 43, wherein said compound is selected from the group consisting of:

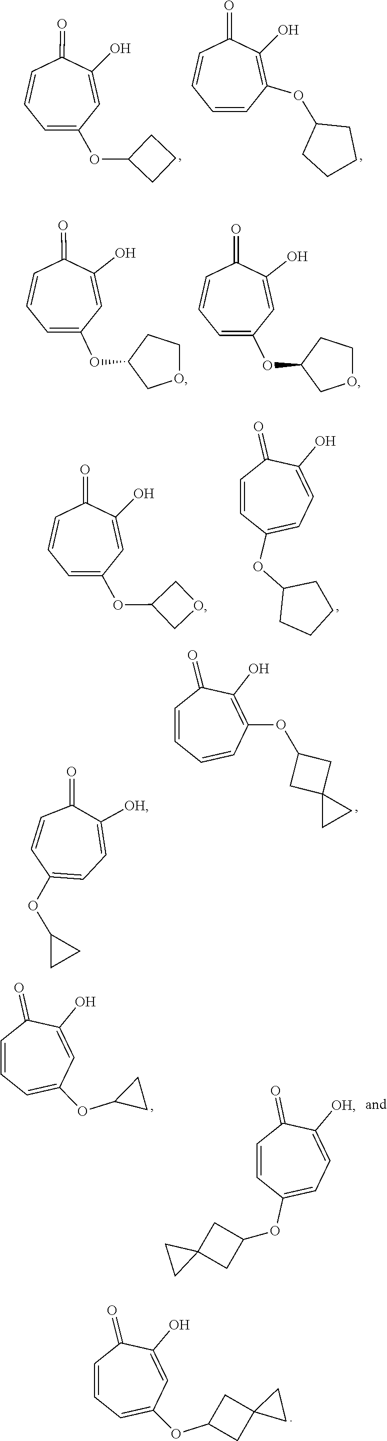

47. The compound or tautomer of claim 30, wherein:
R_{b}, R_{c}, and R_{d} independently represent hydrogen or -alkyl-R₃ or —O-alkyl-R₃; and
R₃ independently represents aryl, substituted aryl, 3-12 membered cycloalkyl or bicycloalkyl, or a 3-12 membered heterocycloalkyl or heterobicylcloalkyl comprising one or two oxygen atoms, one or two nitrogen atoms, one or two sulfur atoms, or any combination of two atoms selected from the group consisting of oxygen, nitrogen, and sulfur atoms.

48. The compound or tautomer of claim 47, wherein said compound is selected from the group consisting of:

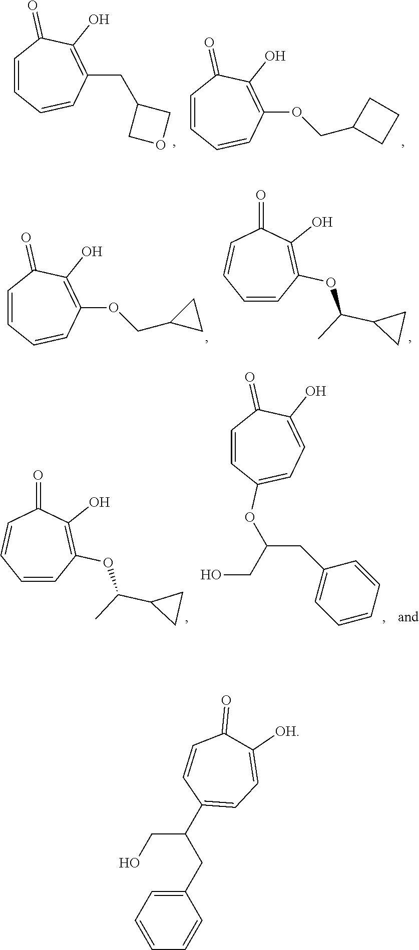

49. The compound or tautomer of claim 30 or 31, wherein Rₐ′ is halo or alkyl.

50. The compound or tautomer of claim 47, wherein said compound is

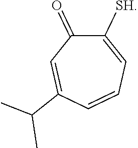

51. A compound or a tautomer thereof, or a pharmaceutically acceptable salt of either, represented by Formula IIa, Formula IIb, or Formula IIc: 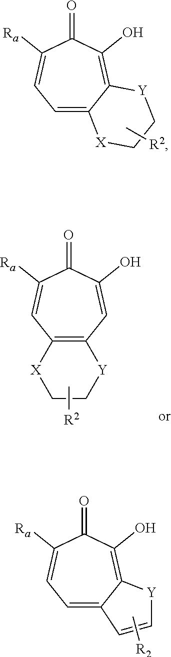
wherein:
Rₐ represents hydrogen, halo, alkyl, substituted alkyl, heteroalkyl, alkoxy, substituted alkoxy, alkoxyalkyl, substituted alkoxyalkyl, cycloalkyl, substituted cycloalkyl, heterocycloalkyl, substituted heterocycloalkyl, alkenyl, substituted alkenyl, heteroalkenyl, cycloalkenyl, substituted cycloalkenyl, heterocycloalkenyl, substituted heterocycloalkenyl, alkynyl, substituted alkynyl, or heteroalkynyl;
X and Y independently represent O, S, NH, or CR₅R₆;
R₂ represents —F, alkyl, haloalkyl, or alkoxy; and
R₅ and R₆ represent independently for each occurrence H, (C1-C15) alkyl, or substituted (C1-C15)alkyl;
provided the compound is not

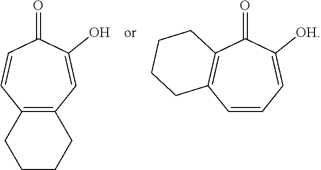 [SMILES: O=c1ccc2c(cc1O)CCCC2 ; O=c1c(O)cccc2c1CCCC2]

52. The compound or a tautomer of claim 50, wherein the compound or a tautomer thereof is represented by Formula IIa or Formula IIb.

53. The compound or tautomer of claim 52, wherein Rₐ represents hydrogen.

54. The compound or tautomer of claim 53, wherein said compound is selected from the group consisting of:

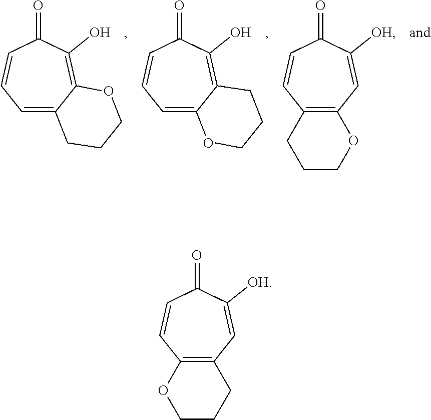

55. The compound or tautomer of claim 53, wherein said compound is

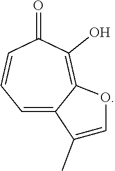

56. The compound or tautomer of claim any one of claims 1-55, wherein the compound has a human liver microsomal clearance half-life (t_{1/2}) of greater than 9 minutes, greater than 12 minutes, greater than 25 minutes, greater than 50 minutes, greater than 100 minutes, or greater than 150 minutes.

57. The compound or tautomer of claim any one of claims 1-55, wherein the compound has a human liver microsomal intrinsic clearance (CLᵢₙₜ) of less than 120 μL/min/mg protein, less than 50 μL/min/mg protein, less than 46 μL/min/mg protein, less than 43 μL/min/mg protein, or less than 25 μL/min/mg protein.

58. A pharmaceutical composition, comprising a compound or tautomer of any one of claims 1-57; and a pharmaceutically acceptable carrier.

59. A method of treating a disease or condition associated with iron dysregulation or dysfunctional iron homeostasis, comprising administering to a subject in need thereof a therapeutically effective amount of a compound or tautomer of any one of claims 1-57 or the pharmaceutical composition of claim 58.

60. The method of claim 59, wherein the disease or condition associated with iron dysregulation or dysfunctional iron homeostasis is selected from the group consisting of anemia, iron deficiency anemia, anemia of inflammation, anemia of chronic inflammatory disorders, anemia of chronic kidney disease, anemia in inflammatory bowel disease, chemotherapy-induced anemia, cancer associated anemia, primary hemochromatosis, secondary hemochromatosis, liver failure, a CNS disease, Parkinson's disease, and Alzheimer's disease.

61. The method of claim 60, wherein the disease or condition associated with iron dysregulation or dysfunctional iron homeostasis is liver failure; and the liver failure is acute.

62. The method of claim 60, wherein the disease or condition associated with iron dysregulation or dysfunctional iron homeostasis is liver failure; and the liver failure is chronic.

63. The method of claim 60, wherein the disease or condition associated with iron dysregulation or dysfunctional iron homeostasis is Parkinson's disease.

64. The method of claim 60, wherein the disease or condition associated with iron dysregulation or dysfunctional iron homeostasis is Alzheimer's disease.

66. The method of claim 60, wherein the disease or condition associated with iron dysregulation or dysfunctional iron homeostasis is anemia.

67. The method of claim 60, wherein the disease or condition associated with iron dysregulation or dysfunctional iron homeostasis is selected from the group consisting of anemia of chronic inflammation, inflammatory bowel disease, chronic heart failure, chronic obstructive pulmonary disease, rheumatoid arthritis, and lupus.

68. The method of claim 67, wherein the anemia of chronic inflammation is anemia of chronic kidney disease.

69. The method of claim 60, wherein the disease or condition associated with iron dysregulation or dysfunctional iron homeostasis is primary hemochromatosis or secondary hemochromatosis.

70. The method of claim 60, wherein the disease or condition associated with iron dysregulation or dysfunctional iron homeostasis is iron deficiency anemia.

71. The method of claim 60, wherein the disease or condition associated with iron dysregulation or dysfunctional iron homeostasis is a CNS disease; and the CNS disease is Friedreich's Ataxia.
